SEQUENCE LISTING (113 sequences)

SEQ ID NO: 1 - protein, 465 aa, Oryza sativa
MDPSPAVDPSPAVDPSPAAETRRRATGKGGKQRGGKQLGLKRPPPISVPATPPPAATSSSPAAPTAIPPRPPQSSPIFVPDSPNPSPAAPTSSLASGTSTARPPQPQGGGWGPTSTISPNFASFFGNQQDPNSCLVRGYPPGGFVNFIQQNCPPQPQQQGENFHFVGHNMGFNPISPQPPSAYGTPTPQATNQGTSTNIMIDEEDNNDDSRAAKKRWTHEEEERLASAWLNASKDSIHGNDKKGDTFWKEVTDEFNKKGNGKRRREINQLKVHWSRLKSAISEFNDYWSTVTQMHTSGYSDDMLEKEAQRLYANRFGKPFALVHWWKILKREPKWCAQFEKRKRKSEMDAVPEQQKRPIGREAAKSERKRKRKKENVMEGIVLLGDNVQKIIKVTQDRKLEREKVTEAQIHISNVNLKAAEQQKEAKMFEVYNSLLTQDTSNMSEEQKARRDKALQKLEEKLFAD

SEQ ID NO: 2 - DNA, 1398 bp, Oryza sativa
atggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactag

SEQ ID NO: 3 - protein, 482 aa, Oryza sativa
MQSLAISLLLSETHSLFSHTKTSSLLSLLFLSSSKMSEQNTDGSQVPVNLLDEFLAEDEIIDDLLTEATVVVQSTIEGLQNEASDHRHHPRKHIKRPREEAHQQLVNDYFSENPLYPSKIFRRRFRMSRPLFLRIVEALGQWSVYFTQRVDAVNRKGLSPLQKCTAAIRQLATGSGADELDEYLKIGETTAMEAMKNFVKGLQDVFGERYLRRPTMEDTERLLQLGEKRGFPGMFGSIDCMHWHWERCPVAWKGQFTRGDQKVPTLILEAVASHDLWIWHAFFGAAGSNNDINVLNQSTVFIKELKGQAPRVQYMVNGNQYNTGYFLADGIYPEWAVFVKSIRLPNTEKEKLYADMQEGARKDIERAFGVLQRRFCILKRPARLYDRGVLRDVVLACIILHNMIVEDEKETRIIEEDADANVPPSSSTVQEPEFSPEQNTPFDRVLEKDISIRDRAAHNRLKKDLVEHIWNKFGGAAHRTGN

SEQ ID NO: 4 - DNA, 1446 bp, Oryza sativa
atgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaat

SEQ ID NO: 5 - protein, 1399 aa, Streptococcus pyogenes
APKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 6 - DNA, 4200 bp, Streptococcus pyogenes
gctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtga

SEQ ID NO: 7 - DNA, 12 bp, Oryza sativa
ggccagtcacaa

SEQ ID NO: 8 - DNA, 12 bp, Oryza sativa
ttgtgactggcc

SEQ ID NO: 9 - DNA, 15 bp, Artificial Sequence
  Synthetic
ttaggccagtcacaa

SEQ ID NO: 10 - DNA, 15 bp, Artificial Sequence
  Synthetic
ttgtgactggcctta

SEQ ID NO: 11 - DNA, 68 bp, Arabidopsis benthamiana
ccatcttgggcctcaacataagcctgaccgccgaccatggctggcaaaagtccaatagcaaactttat

SEQ ID NO: 12 - DNA, 23 bp, Artificial Sequence
  Synthetic
cataagcctgaggccagtcacaa

SEQ ID NO: 13 - DNA, 38 bp, Artificial Sequence
  Synthetic
ttgtgactggccttagcgccgaccatggctggcaaaag

SEQ ID NO: 14 - DNA, 23 bp, Artificial Sequence
  Synthetic
cataagcctgaggccagtcacaa

SEQ ID NO: 15 - DNA, 38 bp, Artificial Sequence; identical to SEQ ID NO: 13
  Synthetic

SEQ ID NO: 16 - DNA, 23 bp, Artificial Sequence
  Synthetic
cataagcctgaggccagtcacaa

SEQ ID NO: 17 - DNA, 34 bp, Artificial Sequence
  Synthetic
ttgtgactggcctgccgaccatggctggcaaaag

SEQ ID NO: 18 - DNA, 27 bp, Artificial Sequence
  Synthetic
cataagcctgacttaggccagtcacaa

SEQ ID NO: 19 - DNA, 34 bp, Artificial Sequence; identical to SEQ ID NO: 17
  Synthetic

SEQ ID NO: 20 - DNA, 27 bp, Artificial Sequence
  Synthetic
cataagcctgacttaggccagtcacaa

SEQ ID NO: 21 - DNA, 33 bp, Artificial Sequence
  Synthetic
ttgtgactggccgccgaccatggctggcaaaag

SEQ ID NO: 22 - DNA, 25 bp, Artificial Sequence
  Synthetic
cataagcctgacaggccagtcacaa

SEQ ID NO: 23 - DNA, 33 bp, Artificial Sequence; identical to SEQ ID NO: 21
  Synthetic

SEQ ID NO: 24 - DNA, 25 bp, Artificial Sequence
  Synthetic
cataagcctgacaggccagtcacaa

SEQ ID NO: 25 - DNA, 36 bp, Artificial Sequence
  Synthetic
ttgtgactggccttaaccgaccatggctggcaaaag

SEQ ID NO: 26 - DNA, 28 bp, Artificial Sequence
  Synthetic
cataagcctgacgttaggccagtcacaa

SEQ ID NO: 27 - DNA, 37 bp, Artificial Sequence
  Synthetic
ttgtgactggccttacgccgaccatggctggcaaaag

SEQ ID NO: 28 - DNA, 17 bp, Artificial Sequence
  Synthetic
cataagcctgactgtgt

SEQ ID NO: 29 - DNA, 33 bp, Artificial Sequence; identical to SEQ ID NO: 21
  Synthetic

SEQ ID NO: 30 - DNA, 25 bp, Artificial Sequence
  Synthetic
cataagcctgataggccagtcacaa

SEQ ID NO: 31 - DNA, 27 bp, Artificial Sequence
  Synthetic
ttgtgactggcctatggctggcaaaag

SEQ ID NO: 32 - DNA, 25 bp, Artificial Sequence
  Synthetic
cataagcctgataggccagtcacaa

SEQ ID NO: 33 - DNA, 27 bp, Artificial Sequence
  Synthetic
ttgtgactggcctatggctggcaaaag

SEQ ID NO: 34 - DNA, 25 bp, Artificial Sequence
  Synthetic
cataagcctgataggccagtcacaa

SEQ ID NO: 35 - DNA, 27 bp, Artificial Sequence
  Synthetic
ttgtgactggcctatggctggcaaaag

SEQ ID NO: 36 - DNA, 25 bp, Artificial Sequence
  Synthetic
cataagcctgataggccagtcacaa

SEQ ID NO: 37 - DNA, 27 bp, Artificial Sequence
  Synthetic
ttgtgactggcctatggctggcaaaag

SEQ ID NO: 38 - DNA, 27 bp, Artificial Sequence
  Synthetic
cataagcctgactaaggccagtcacaa

SEQ ID NO: 39 - DNA, 27 bp, Artificial Sequence
  Synthetic
ttgtgactggcctatggctggcaaaag

SEQ ID NO: 40 - DNA, 27 bp, Artificial Sequence
  Synthetic
cataagcctgactaaggccagtcacaa

SEQ ID NO: 41 - DNA, 36 bp, Artificial Sequence
  Synthetic
ttgtgactggccttcgccgaccatggctggcaaaag

SEQ ID NO: 42 - DNA, 24 bp, Artificial Sequence
  Synthetic
cataagcctgaaggccagtcacaa

SEQ ID NO: 43 - DNA, 36 bp, Artificial Sequence; identical to SEQ ID NO: 41
  Synthetic

SEQ ID NO: 44 - DNA, 24 bp, Artificial Sequence
  Synthetic
cataagcctgaaggccagtcacaa

SEQ ID NO: 45 - DNA, 36 bp, Artificial Sequence; identical to SEQ ID NO: 41
  Synthetic

SEQ ID NO: 46 - DNA, 28 bp, Artificial Sequence
  Synthetic
cataagcctgacttaaggccagtcacaa

SEQ ID NO: 47 - DNA, 36 bp, Artificial Sequence; identical to SEQ ID NO: 41
  Synthetic

SEQ ID NO: 48 - DNA, 31 bp, Artificial Sequence
  Synthetic
caacataagcctgacaggccagtcacaatgg

SEQ ID NO: 49 - DNA, 15 bp, Artificial Sequence
  Synthetic
ccattgtgactggcc

SEQ ID NO: 50 - DNA, 14 bp, Artificial Sequence
  Synthetic
gccgaccatggctg

SEQ ID NO: 51 - DNA, 15 bp, Artificial Sequence
  Synthetic
caacataagcctgac

SEQ ID NO: 52 - DNA, 15 bp, Artificial Sequence
  Synthetic
ggccagtcacaatgg

SEQ ID NO: 53 - DNA, 30 bp, Artificial Sequence
  Synthetic
ccattgtgactggcccgccgaccatggctg

SEQ ID NO: 54 - DNA, 32 bp, Artificial Sequence
  Synthetic
ccgttgtttccacgtaaggccagtcacaatgg

SEQ ID NO: 55 - DNA, 30 bp, Artificial Sequence
  Synthetic
ccattgtgactggccatcttcggccatgaa

SEQ ID NO: 56 - DNA, 15 bp, Artificial Sequence
  Synthetic
ccgttgtttccacgt

SEQ ID NO: 57 - DNA, 15 bp, Artificial Sequence
  Synthetic
ggccagtcacaatgg

SEQ ID NO: 58 - DNA, 29 bp, Artificial Sequence
  Synthetic
ccatgtgactggccatcttcggccatgaa

SEQ ID NO: 59 - DNA, 14 bp, Artificial Sequence
  Synthetic
tacaggagtagttc

SEQ ID NO: 60 - DNA, 14 bp, Artificial Sequence
  Synthetic
gccagtcacaatgg

SEQ ID NO: 61 - DNA, 30 bp, Artificial Sequence
  Synthetic
ccattgtgactggcctcgtggccttagtaa

SEQ ID NO: 62 - DNA, 30 bp, Artificial Sequence
  Synthetic
tacaggagtagttcaggccagtcacaatgg

SEQ ID NO: 63 - DNA, 30 bp, Artificial Sequence; identical to SEQ ID NO: 61
  Synthetic

SEQ ID NO: 64 - protein, 5 aa, Artificial Sequence
  Synthetic
GSSSS

SEQ ID NO: 65 - DNA, 20 bp, Artificial Sequence
  Synthetic
gccagccatggtcggcggtc

SEQ ID NO: 66 - DNA, 20 bp, Artificial Sequence
  Synthetic
gcttcatggccgaagatacg

SEQ ID NO: 67 - DNA, 20 bp, Artificial Sequence
  Synthetic
gttacaggagtagttcatcg

SEQ ID NO: 68 - protein, 8 aa, Artificial Sequence
  Synthetic
GGSGGGSG

SEQ ID NO: 69 - protein, 5 aa, Artificial Sequence
  Synthetic
GGGGS

SEQ ID NO: 70 - protein, 12 aa, Artificial Sequence
  Synthetic
AEAAAKEAAAKA

SEQ ID NO: 71 - protein, 17 aa, Artificial Sequence
  Synthetic
AEAAAKEAAAKEAAAKA

SEQ ID NO: 72 - protein, 7 aa, Artificial Sequence
  Synthetic
PAPAPAP

SEQ ID NO: 73 - protein, 8 aa, Artificial Sequence
  Synthetic
GIHGVPAA

SEQ ID NO: 74 - DNA, 23463 bp, Artificial Sequence
  Synthetic
  Agro tDNA cut site (1)..(25): /label=“RB”
  regulatory (42)..(297): /label=“NOS Terminator”
  misc_feature (317)..(1105): /label=“eGFP5-ere”
  misc_feature (1132)..(1134): /label=“TSD”
  Transposon (1135)..(1564): /label=“mPing”
  misc_feature (1565)..(1567): /label=“TSD”
  promoter (1581)..(2414): /label=“CaMV Promoter”
  misc_feature (2632)..(3055): /label=“U6-26promoter”
  misc_feature (3056)..(3075): /label=“gRNA to PDS3 exon”
  misc_feature (3076)..(3151): /label=“gRNA scaffold”
  misc_feature (3076)..(3151): /label=“gRNA scaffold”
  misc_feature (3152)..(3343): /label=“U6-26 terminator”
  promoter (3359)..(5045): /label=“Rps5a”
  misc_feature (5082)..(6479): /label=“ORF1”
  terminator (6543)..(7268): /label=“OCS terminator”
  promoter (7451)..(8370): /label=“GmUbi3 Promoter”
  misc_feature (8392)..(9837): /label=“Pong TPase LA”
  misc_feature (9841)..(9855): /label=“G4S linker”
  misc_feature (9859)..(9879): /label=“SV40 NLS”
  misc_feature (9883)..(14052): /label=“Cas9”
  misc_feature (14005)..(14052): /label=“NLS”
  terminator (14080)..(14807): /label=“OCS Terminator”
  promoter (15058)..(15799): /label=“CaMVd35S_promoter”
  gene (15890)..(16885): /label=“hygroB (variant)”
  misc_feature (17503)..(17525): /label=“LB”
  gene (17641)..(18435): /label=“KanR1”
  origin (18506)..(19118): /label=“pBR322_origin”
gtttacccgccaatatatcctgtcaaacactgatagtttttcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcttaaagctcatcatgtttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatgatcagcgagttgcacgccgccgtcttcgatgttgtggcgggtcttgaagttggctttgatgccgttcttttgcttgtcggccatgatgtatacgttgtgggagttgtagttgtattccaacttgtggccgaggatgtttccgtcctccttgaaatcgattcccttaagctcgatcctgttgacgagggtgtctccctcaaacttgacttcagcacgtgtcttgtagttcccgtcgtccttgaagaagatggtcctctcctgcacgtatccctcaggcatggcgctcttgaagaagtcgtgccgcttcatatgatctgggtatcttgaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactgaattcggccgaggataatgataggagaagtgaaaagatgagaaagagaaaaagattagtcttcattgttatatctccttggatcctctagattaggccagtcacaatggctagtgtcattgcacggctacccaaaatattataccatcttctctcaaatgaaatcttttatgaaacaatccccacagtggaggggtttcactttgacgtttccaagactaagcaaagcatttaattgatacaagttgctgggatcatttgtacccaaaatccggcgcggcgcgggagaatgcggaggtcgcacggcggaggcggacgcaagagatccggtgaatgaaacgaatcggcctcaacgggggtttcactctgttaccgaggacttggaaacgacgctgacgagtttcaccaggatgaaactctttccttctctctcatccccatttcatgcaaataatcattttttattcagtcttacccctattaaatgtgcatgacacaccagtgaaacccccattgtgactggccttatctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaattgaaaaggctaatctgggaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgccagccatggtcggcggtcgttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcggtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttag

SEQ ID NO: 75 - DNA, 21092 bp, Artificial Sequence
  Synthetic
  Agro tDNA cut site (1)..(25): /label=“RB ”
  misc_feature (254)..(677): /label=“U6-26 promoter”
  misc_feature (678)..(697): /label=“gRNA”
  misc_feature (698)..(773): /label=“gRNA scaffold”
  misc_feature (774)..(965): /label=“U6-26 terminator”
  promoter (981)..(2667): /label=“Rps5a promoter”
  misc_feature (2704)..(4101): /label=“Pong ORF1”
  CDS (2704)..(4101): /label=“Translation 2704-4101”
  terminator (4165)..(4890): /label=“OCS terminator”
  promoter (5073)..(5992): /label=“GmUbi3 promoter”
  misc_feature (7463)..(7477): /label=“G4S linker”
  misc_feature (7481)..(7501): /label=“NLS”
  misc_feature (7505)..(11626): /label=“Cas9”
  misc_feature (11627)..(11674): /label=“NLS”
  terminator (11702)..(12429): /label=“OCS terminator”
  promoter (12680)..(13420): /label=“CaMV 35S promoter”
  gene (13510)..(14505): /label=“HygR”
  CDS (13510)..(14505): /label=“Translation 13510-14505”
  misc_feature (15124)..(15146): /label=“LB ”
  gene (15262)..(16056): /label=“KanR”
  origin (16127)..(16746): /label=“pBR322_origin”
gtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgccagccatggtcggcggtcgttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccgggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttag

SEQ ID NO: 76 - protein, 465 aa, Artificial Sequence; identical to SEQ ID NO: 1
  Synthetic Construct

SEQ ID NO: 77 - protein, 332 aa, Artificial Sequence
  Synthetic Construct
LTATSVEKFLIEKFDSVSDLMQLSEGEESRAFSFDVGGRGYVLRVNSCADGFYKDRYVYRHFASAALPIPEVLDIGEFSESLTYCISRRSQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRP

SEQ ID NO: 78 - protein, 5 aa, Artificial Sequence
  Synthetic
EAAAK

SEQ ID NO: 79 - protein, 10 aa, Artificial Sequence
  Synthetic
EAAAKEAAAK

SEQ ID NO: 80 - protein, 15 aa, Artificial Sequence
  Synthetic
EAAAKEAAAKEAAAK

SEQ ID NO: 81 - protein, 20 aa, Artificial Sequence
  Synthetic
EAAAKEAAAKEAAAKEAAAK

SEQ ID NO: 82 - DNA, 20 bp, Artificial Sequence
  Synthetic
ggaactgacacacgacatga

SEQ ID NO: 83 - DNA, 444 bp, Artificial Sequence
  Synthetic
ggccagtcacaatggctagtgtcattgcacggctacccaaaatattataccatcttctctcaaatgaaatcttttatgaaacaatccccacagtggaggggtttcttgaacgttccaagactaagcaaagcatttaattgatacaagttcgcgaagattcatttgtacccaaaatccggcgcggcgcgggagaatgttctggaaggtcgcacggcggaggcggacgcaagagatccggtgaatgttcaagaatcggcctcaacgggggtttcactctgttaccgaggaactttctggaaacgacgctgacgagtttcaccaggatgaaactctttccagaaagttctctctcatccccatttcatgcaaataatcattttttattcagtcttacccctattaaatgtgcatgacacaccagtgaaacccccattgtgactggcc

SEQ ID NO: 84 - DNA, 13 bp, Artificial Sequence
  Synthetic
ttcttgaacgttc

SEQ ID NO: 85 - DNA, 13 bp, Artificial Sequence
  Synthetic
ttcgcgaagattc

SEQ ID NO: 86 - DNA, 13 bp, Artificial Sequence
  Synthetic
ttccagaacattc

SEQ ID NO: 87 - DNA, 13 bp, Artificial Sequence
  Synthetic
ttcttgaacattc

SEQ ID NO: 88 - DNA, 13 bp, Artificial Sequence
  Synthetic
ttccagaaagttc

SEQ ID NO: 89 - DNA, 13 bp, Artificial Sequence
  Synthetic
ttccagaaagttc

SEQ ID NO: 90 - DNA, 9 bp, Artificial Sequence
  Synthetic
ggsggsggs

SEQ ID NO: 91 - DNA, 22510 bp, Artificial Sequence
  Synthetic
  Agro tDNA cut site (1)..(25): /label=“RB”
  misc_feature (254)..(677): /label=“U6-26promoter”
  misc_feature (678)..(697): /label=“gRNA to ADH1”
  misc_feature (698)..(773): /label=“gRNA scaffold”
  misc_feature (774)..(965): /label=“U6-26 terminator”
  promoter (981)..(2667): /label=“Rps5a”
  gene (2683)..(4121): /label=“ORF1SC1”
  terminator (4165)..(4890): /label=“OCS terminator”
  promoter (5073)..(5992): /label=“GmUbi3 Promoter”
  gene (6014)..(7462): /label=“Pong TPase LA”
  terminator (7488)..(8215): /label=“OCS Terminator”
  promoter (8218)..(8942): /label=“AtUBQ10 promoter”
  CDS (8955)..(13226): /label=“Translation 8955-13226”
  misc_feature (8958)..(8978): /label=“FLAG”
  misc_feature (8979)..(8999): /label=“FLAG”
  misc_feature (9000)..(9023): /label=“FLAG”
  misc_feature (9030)..(9050): /label=“SV40 NLS”
  misc_feature (9075)..(13226): /label=“Cas9 Nickase (D10A)”
  misc_feature (9099)..(9101): /label=“D10A”
  misc_feature (13176)..(13223): /label=“NLS”
  misc_feature (13232)..(13856): /label=“Rbs Term”
  promoter (14105)..(14846): /label=“CaMVd35S_promoter”
  gene (14937)..(15932): /label=“hygroB (variant)”
  misc_feature (16550)..(16572): /label=“LB R”
  gene (16688)..(17482): /label=“KanR1”
  origin (17553)..(18165): /label=“pBR322_origin”
gtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgcttcatggccgaagatacggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattaattaattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgaggcgcgcctctagatggattacaaggaccacgacggggattacaaggaccacgacattgattacaaggatgatgatgacaagatggctccgaagaagaagaggaaggttggcatccacggggtgccagctgctgacaagaagtactcgatcggcctcgctattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgagctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcggtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttag

SEQ ID NO: 92 - protein, 1423 aa, Artificial Sequence
  Synthetic Construct
MDYKDHDGDYKDHDIDYKDDDDKMAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 93 - DNA, 14122 bp, Artificial Sequence
  Synthetic
  misc_feature (1)..(26): /label=“LB”
  regulatory (665)..(920): /label=“NOS Terminator”
  misc_feature (940)..(1728): /label=“eGFP5-er”
  Transposon (1758)..(2187): /label=“mPing”
  promoter (2204)..(3037): /label=“CaMV Promoter”
  regulatory (3734)..(3989): /label=“NOS Terminator”
  misc_feature (4379)..(5176): /label=“Kan Resistance”
  regulatory (5186)..(5492): /label=“NOS Promoter”
  Agro tDNA cut site (5533)..(5557): /label=“RB”
tggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcttaaagctcatcatgtttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatgatcagcgagttgcacgccgccgtcttcgatgttgtggcgggtcttgaagttggctttgatgccgttcttttgcttgtcggccatgatgtatacgttgtgggagttgtagttgtattccaacttgtggccgaggatgtttccgtcctccttgaaatcgattcccttaagctcgatcctgttgacgagggtgtctccctcaaacttgacttcagcacgtgtcttgtagttcccgtcgtccttgaagaagatggtcctctcctgcacgtatccctcaggcatggcgctcttgaagaagtcgtgccgcttcatatgatctgggtatcttgaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactgaattcggccgaggataatgataggagaagtgaaaagatgagaaagagaaaaagattagtcttcattgttatatctccttggatcctctagattaggccagtcacaatggctagtgtcattgcacggctacccaaaatattataccatcttctctcaaatgaaatcttttatgaaacaatccccacagtggaggggtttcactttgacgtttccaagactaagcaaagcatttaattgatacaagttgctgggatcatttgtacccaaaatccggcgcggcgcgggagaatgcggaggtcgcacggcggaggcggacgcaagagatccggtgaatgaaacgaatcggcctcaacgggggtttcactctgttaccgaggacttggaaacgacgctgacgagtttcaccaggatgaaactctttccttctctctcatccccatttcatgcaaataatcattttttattcagtcttacccctattaaatgtgcatgacacaccagtgaaacccccattgtgactggccttatctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaattgaaaaggctaatctggggacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 94 - DNA, 21085 bp, Artificial Sequence
  Synthetic
  Agro tDNA cut site (1)..(25): /label=“RB”
  misc_feature (254)..(677)
  misc_feature (678)..(697): /label=“gRNA to ACT8 promoter”
  misc_feature (698)..(773): /label=“gRNA scaffold”
  misc_feature (774)..(965): /label=“U6-26 terminator”
  promoter (981)..(2667): /label=“Rps5a”
  misc_feature (2704)..(4101): /label=“ORF1”
  terminator (4165)..(4890): /label=“OCS terminator”
  promoter (5073)..(5992): /label=“GmUbi3 Promoter”
  misc_feature (6014)..(7459): /label=“Pong TPase LA”
  CDS (6014)..(11677): /label=“Translation 6014-11677”
  misc_feature (7463)..(7477): /label=“G4S linker”
  misc_feature (7481)..(7501): /label=“SV40 NLS”
  misc_feature (7505)..(11674): /label=“Cas9”
  misc_feature (11627)..(11674): /label=“NLS”
  terminator (11702)..(12429): /label=“OCS Terminator”
  promoter (12680)..(13421): /label=“CaMVd35S_promoter”
  gene (13512)..(14507): /label=“hygroB (variant)”
  misc_feature (15125)..(15147): /label=“LB”
  gene (15263)..(16057): /label=“KanR1”
  origin (16128)..(16740): /label=“pBR322_origin”
gtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttacaggagtagttcatcggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcggtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttag

SEQ ID NO: 95 - protein, 1887 aa, Artificial Sequence
  Synthetic Construct
MQSLAISLLLSETHSLFSHTKTSSLLSLLFLSSSKMSEQNTDGSQVPVNLLDEFLAEDEIIDDLLTEATVVVQSTIEGLQNEASDHRHHPRKHIKRPREEAHQQLVNDYFSENPLYPSKIFRRRFRMSRPLFLRIVEALGQWSVYFTQRVDAVNRKGLSPLQKCTAAIRQLATGSGADELDEYLKIGETTAMEAMKNFVKGLQDVFGERYLRRPTMEDTERLLQLGEKRGFPGMFGSIDCMHWHWERCPVAWKGQFTRGDQKVPTLILEAVASHDLWIWHAFFGAAGSNNDINVLNQSTVFIKELKGQAPRVQYMVNGNQYNTGYFLADGIYPEWAVFVKSIRLPNTEKEKLYADMQEGARKDIERAFGVLQRRFCILKRPARLYDRGVLRDVVLACIILHNMIVEDEKETRIIEEDADANVPPSSSTVQEPEFSPEQNTPFDRVLEKDISIRDRAAHNRLKKDLVEHIWNKFGGAAHRTGNYGGGGSAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 96 - DNA, 21560 bp, Artificial Sequence
  Synthetic
  Agro tDNA cut site (1)..(25): /label=“RB”
  misc_feature (69)..(83): /label=“TIR”
  Transposon (69)..(498): /label=“mPing”
  misc_feature (484)..(498): /label=“TIR”
  misc_feature (729)..(1152): /label=“U6-26promoter”
  misc_feature (1153)..(1172): /label=“gRNA to ACT8 promoter”
  misc_feature (1173)..(1248): /label=“gRNA scaffold”
  misc_feature (1249)..(1440): /label=“U6-26 terminator”
  promoter (1249)..(1440): /label=“Rps5a”
  misc_feature (3179)..(4576): /label=“ORF1”
  terminator (4640)..(5365): /label=“OCS terminator”
  promoter (5548)..(6467): /label=“GmUbi3 Promoter”
  misc_feature (6489)..(7934): /label=“Pong TPase LA”
  CDS (6489)..(12149): /label=“Translation 6489-12149”
  misc_feature (7938)..(7952): /label=“G4S linker”
  misc_feature (7956)..(7976): /label=“SV40 NLS”
  misc_feature (7980)..(12149): /label=“Cas9”
  misc_feature (12102)..(12149): /label=“NLS”
  terminator (12177)..(12904)
  promoter (13155)..(13896): /label=“CaMVd35S_promoter”
  gene (13987)..(14982): /label=“hygroB (variant)”
  misc_feature (13987)..(14982): /label=“LB”
  gene (15738)..(16532): /label=“KanR1”
  origin (16603)..(17215): /label=“pBR322_origin”
gtttacccgccaatatatcctgtcaaacactgatagttttgttatatctccttggatcctctagattaggccagtcacaatggctagtgtcattgcacggctacccaaaatattataccatcttctctcaaatgaaatcttttatgaaacaatccccacagtggaggggtttcactttgacgtttccaagactaagcaaagcatttaattgatacaagttgctgggatcatttgtacccaaaatccggcgcggcgcgggagaatgcggaggtcgcacggcggaggcggacgcaagagatccggtgaatgaaacgaatcggcctcaacgggggtttcactctgttaccgaggacttggaaacgacgctgacgagtttcaccaggatgaaactctttccttctctctcatccccatttcatgcaaataatcattttttattcagtcttacccctattaaatgtgcatgacacaccagtgaaacccccattgtgactggccttatctagagtcccccaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttacaggagtagttcatcggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcggtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttag

SEQ ID NO: 97 - protein, 1887 aa, Artificial Sequence; identical to SEQ ID NO: 95
  Synthetic Construct

SEQ ID NO: 98 - DNA, 21585 bp, Artificial Sequence
  Synthetic
  Agro tDNA cut site (1)..(25): /label=“RB”
  misc_feature (69)..(83): /label=“TIR”
  Transposon (69)..(512): /label=“mPing”
  misc_feature (171)..(183): /label=“HSE”
  misc_feature (216)..(228): /label=“HSE”
  misc_feature (260)..(272): /label=“HSE”
  misc_feature (308)..(320): /label=“HSE”
  misc_feature (355)..(367): /label=“HSE”
  misc_feature (498)..(512): /label=“TIR”
  misc_feature (754)..(1177): /label=“U6-26promoter”
  misc_feature (1178)..(1197): /label=“gRNA to ACT8 promoter”
  misc_feature (1198)..(1273): /label=“gRNA scaffold”
  misc_feature (1274)..(1465): /label=“U6-26 terminator”
  promoter (1481)..(3167): /label=“Rps5a”
  misc_feature (3204)..(4601): /label=“ORF1”
  terminator (4665)..(5390)
  promoter (5573)..(6492): /label=“GmUbi3 Promoter”
  misc_feature (6514)..(7959): /label=“Pong TPase LA”
  misc_feature (7963)..(7977): /label=“G4S linker”
  misc_feature (7981)..(8001): /label=“SV40 NLS”
  misc_feature (8005)..(12174): /label=“Cas9”
  misc_feature (12127)..(12174): /label=“NLS”
  terminator (12202)..(12929): /label=“OCS Terminator”
  promoter (13180)..(13921): /label=“CaMVd35S_promoter”
  gene (14012)..(15007): /label=“hygroB (variant)”
  misc_feature (15625)..(15647): /label=“LB”
  gene (15763)..(16557): /label=“KanR1”
  origin (16628)..(17240): /label=“pBR322_origin”
gtttacccgccaatatatcctgtcaaacactgatagtttcacgtgatctccttggatcctctagattaggccagtcacaatggctagtgtcattgcacggctacccaaaatattataccatcttctctcaaatgaaatcttttatgaaacaatccccacagtggaggggtttcttgaacgttccaagactaagcaaagcatttaattgatacaagttcgcgaagattcatttgtacccaaaatccggcgcggcgcgggagaatgttctggaaggtcgcacggcggaggcggacgcaagagatccggtgaatgttcaagaatcggcctcaacgggggtttcactctgttaccgaggaactttctggaaacgacgctgacgagtttcaccaggatgaaactctttccagaaagttctctctcatccccatttcatgcaaataatcattttttattcagtcttacccctattaaatgtgcatgacacaccagtgaaacccccattgtgactggccttatctagagtcccccatactaggcctaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttacaggagtagttcatcggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcggtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttag

SEQ ID NO: 99 - DNA, 23380 bp, Artificial Sequence
  Synthetic
  CDS (825)..(1373): /label=“BlpR”
  promoter (1565)..(1744): /label=“NOS promoter”
  misc_feature (2201)..(2215): /label=“TIR”
  Transposon (2201)..(2630): /label=“mPing”
  misc_feature (2616)..(2630): /label=“TIR”
  misc_feature (3305)..(3380): /label=“gRNA scaffold”
  misc_feature (3381)..(3572): /label=“U6-26 terminator”
  promoter (3593)..(5279): /label=“Rps5a”
  gene (5295)..(6733): /label=“ORF1SC1”
  terminator (6777)..(7502): /label=“OCS terminator”
  promoter (7685)..(8604): /label=“GmUbi3 Promoter”
  gene (8626)..(10074): /label=“Pong TPase LA”
  terminator (10100)..(10827): /label=“OCS Terminator”
  promoter (10857)..(11581): /label=“AtUBQ10 promoter”
  misc_feature (11597)..(11617): /label=“FLAG”
  misc_feature (11618)..(11638): /label=“FLAG”
  misc_feature (11639)..(11662): /label=“FLAG”
  misc_feature (11669)..(11689): /label=“SV40 NLS”
  misc_feature (11693)..(15865): /label=“Cas9”
  misc_feature (15815)..(15862): /label=“NLS”
  misc_feature (15871)..(16495): /label=“Rbs Term”
  misc_feature (16818)..(16842): /label=“RB T-DNA repeat”
  CDS (18173)..(18802): /label=“pVS1 StaA”
  CDS (19231)..(20304): /label=“pVS1 RepA”
  rep_origin (20370)..(20564): /label=“pVS1 oriV”
  misc_feature (20908)..(21048): /label=“bom”
  rep_origin (21234)..(21822): /label=“ori”
  CDS (22068)..(22859): /label=“SmR”
  misc_feature (23380)..(23380): /label=“LB T-DNA repeat”
  misc_feature (32856)..(3304): /label=“gRNA to DD20”
ggcaggatatattgtggtgtaaacaaattgacgcttagacaacttaataacacattgcggacgtttttaatgtactgaattaacgccgaattgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctaattcgcttcaagacgtgctcaaatcactatttccacacccctatatttctattgcactcccttttaactgttttttattacaaaaatgccctggaaaatgcactccctttttgtgtttgtttttttgtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccgcgtaagagacaacaagtaggtacgtggagtgtcttaggtgacttacccacataacataaagtgacattaacaaacatagctaatgctcctatttgaatagtgcatatcagcataccttattacatatagataggagcaaactctagctagattgttgagcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccccgttcgtaaatggtgaaaattttcagaaaattgcttttgctttaaaagaaatgatttaaattgctgcaatagaagtagaatgcttgattgcttgagattcgtttgttttgtatatgttgtgttgagaattaattctcgagcctagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcaggtcgactctagtgttatatctccttggatcctctagattaggccagtcacaatggctagtgtcattgcacggctacccaaaatattataccatcttctctcaaatgaaatcttttatgaaacaatccccacagtggaggggtttcactttgacgtttccaagactaagcaaagcatttaattgatacaagttgctgggatcatttgtacccaaaatccggcgcggcgcgggagaatgcggaggtcgcacggcggaggcggacgcaagagatccggtgaatgaaacgaatcggcctcaacgggggtttcactctgttaccgaggacttggaaacgacgctgacgagtttcaccaggatgaaactctttccttctctctcatccccatttcatgcaaataatcattttttattcagtcttacccctattaaatgtgcatgacacaccagtgaaacccccattgtgactggccttatctagagtcccccaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattggaactgacacacgacatgagttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatggttatactaggagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattaattaattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctagtgatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgaggcgcgcctctagatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgagctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacgctagaggatccccgggtaccgagctcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttaaagtactttaaagtactttaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggt

SEQ ID NO: 100 - protein, 183 aa, Artificial Sequence
  Synthetic Construct
DLGDGQDRTGRYRQAEVQLPETHVMPVPVLEAGRPQHAAGGISERLVHAHARVVGQPDDSDHALEALCLQGLQQVGVERGAQSRPLVAGGDVHGRLGRPVVGVACLPGARVGDAGDLAVHLGDEPGIALPQTDEVVRPLLRFLRLGTEVDRACLDVVVDDGADRRHVRLGGTADVGRASFWAH

SEQ ID NO: 101 - protein, 209 aa, Artificial Sequence
  Synthetic Construct
MKVIAVLNQKGGSGKTTIATHLARALQLAGADVLLVDSDPQGSARDWAAVREDQPLTVVGIDRPTIDRDVKAIGRRDFVVIDGAPQAADLAVSAIKAADFVLIPVQPSPYDIWATADLVELVKQRIEVTDGRLQAAFVVSRAIKGTRIGGEVAEALAGYELPILESRITQRVSYPGTAAAGTTVLESEPEGDAAREVQALAAEIKSKLI

SEQ ID NO: 102 - protein, 357 aa, Artificial Sequence
  Synthetic Construct
VSGRKPSGPVQIGAALGDDLVEKLKAAQAAQRQRIEAEARPGESWQAAADRIRKESRQPPAAGAPSIRKPPKGDEQPDFFVPMLYDVGTRDSRSIMDVAVFRLSKRDRRAGEVIRYELPDGHVEVSAGPAGMASVWDYDLVLMAVSHLTESMNRYREGKGDKPGRVFRPHVADVLKFCRRADGGKQKDDLVETCIRLNTTHVAMQRTKKAKNGRLVTVSEGEALISRYKIVKSETGRPEYIEIELADWMYREITEGKNPDVLTVHPDYFLIDPGIGRFLYRLARRAAGKAEARWLFKTIYERSGSAGEFKKFCFTVRKLIGSNDLPEYDLKEEAGQAGPILVMRYRNLIEGEASAGS

SEQ ID NO: 103 - protein, 92 aa, Artificial Sequence
  Synthetic Construct
LFADYLGDLAFHVVDKFFQLICARGQAIFFLSKISLSSFKYDGLILGRQALHCPVGSDILRRDFAGYCAVPNAGQRKHYISLIASPVGRRVP

SEQ ID NO: 104 - protein, 147 aa, Artificial Sequence
  Synthetic Construct
ILFRNRIKEFLRRWTYQGNAMFSCFCQQDSQINVDRGWLEDTCKNVIALPFSKLQFALSWITPRNDVVVHNNGDFYSAENLALSRGSRSFQKVVDQSSPRCFIKPYGHRNQQINITVWLQAAIHCGAVQMYGQQRRFEMALDDANYL

SEQ ID NO: 105 - protein, 12 aa, Artificial Sequence
  Synthetic Construct
LSRYFGDHRFPH

SEQ ID NO: 106 - DNA, 23836 bp, Artificial Sequence
  Synthetic
  misc_feature (1)..(25): /label=“LB T-DNA repeat”
  CDS (826)..(1374): /label=“BlpR”
  promoter (1566)..(1746): /label=“NOS promoter”
  regulatory (2173)..(2428): /label=“NOS Terminator”
  misc_feature (2448)..(3236): /label=“eGFP5-er”
  Transposon (3266)..(3695): /label=“mPing”
  promoter (3712)..(4545): /label=“CaMV Promoter”
  misc_feature (4763)..(5186): /label=“U6-26promoter”
  misc_feature (5187)..(5206): /label=“gRNA to DD20”
  misc_feature (5207)..(5282): /label=“gRNA scaffold”
  misc_feature (5283)..(5474): /label=“U6-26 terminator”
  promoter (5490)..(7176): /label=“Rps5a”
  misc_feature (7213)..(8610): /label=“ORF1”
  terminator (8674)..(9399): /label=“OCS terminator”
  promoter (9582)..(10501): /label=“GmUbi3 Promoter”
  misc_feature (10523)..(11968): /label=“Pong TPase LA”
  CDS (10523)..(16186): /label=“Translation 10523-16186”
  misc_feature (11972)..(11986): /label=“G4S linker”
  misc_feature (11990)..(12010): /label=“SV40 NLS”
  misc_feature (12014)..(16183): /label=“Cas9”
  misc_feature (16136)..(16183): /label=“NLS”
  terminator (16211)..(16938): /label=“OCS Terminator”
  misc_feature (17275)..(17299): /label=“RB T-DNA repeat”
  CDS (18630)..(19259): /label=“pVS1 StaA”
  CDS (19688)..(20761): /label=“pVS1 RepA”
  rep_origin (20827)..(21021): /label=“pVS1 oriV”
  misc_feature (21365)..(21505): /label=“bom”
  rep_origin (21691)..(22279): /label=“ori”
  CDS (22525)..(23316): /label=“SmR”
tggcaggatatattgtggtgtaaacaaattgacgcttagacaacttaataacacattgcggacgtttttaatgtactgaattaacgccgaattgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctaattcgcttcaagacgtgctcaaatcactatttccacacccctatatttctattgcactcccttttaactgttttttattacaaaaatgccctggaaaatgcactccctttttgtgtttgtttttttgtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccgcgtaagagacaacaagtaggtacgtggagtgtcttaggtgacttacccacataacataaagtgacattaacaaacatagctaatgctcctatttgaatagtgcatatcagcataccttattacatatagataggagcaaactctagctagattgttgagcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccccgttcgtaaatggtgaaaattttcagaaaattgcttttgctttaaaagaaatgatttaaattgctgcaatagaagtagaatgcttgattgcttgagattcgtttgttttgtatatgttgtgttgagaattaattctcgagcctagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcaggtcgactctagcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcttaaagctcatcatgtttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatgatcagcgagttgcacgccgccgtcttcgatgttgtggcgggtcttgaagttggctttgatgccgttcttttgcttgtcggccatgatgtatacgttgtgggagttgtagttgtattccaacttgtggccgaggatgtttccgtcctccttgaaatcgattcccttaagctcgatcctgttgacgagggtgtctccctcaaacttgacttcagcacgtgtcttgtagttcccgtcgtccttgaagaagatggtcctctcctgcacgtatccctcaggcatggcgctcttgaagaagtcgtgccgcttcatatgatctgggtatcttgaaaagcattgaacaccataagagaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttactgaattcggccgaggataatgataggagaagtgaaaagatgagaaagagaaaaagattagtcttcattgttatatctccttggatcctctagattaggccagtcacaatggctagtgtcattgcacggctacccaaaatattataccatcttctctcaaatgaaatcttttatgaaacaatccccacagtggaggggtttcactttgacgtttccaagactaagcaaagcatttaattgatacaagttgctgggatcatttgtacccaaaatccggcgcggcgcgggagaatgcggaggtcgcacggcggaggcggacgcaagagatccggtgaatgaaacgaatcggcctcaacgggggtttcactctgttaccgaggacttggaaacgacgctgacgagtttcaccaggatgaaactctttccttctctctcatccccatttcatgcaaataatcattttttattcagtcttacccctattaaatgtgcatgacacaccagtgaaacccccattgtgactggccttatctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgccttggtttgtgaagcaagccttgaatcgtccatactggaatagtacttctgatcttgagaaatatatctttctctgtgttcttgatgcagttagtcctgaatcttttgactgcatctttaaccttcttgggaaggtatttgatttcctggagattattgctcgggtagatcgtcttgatgagacctgctgcgtaagcctctctaaccatctgtgggttagcattctttctgaaattgaaaaggctaatctgggaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattggaactgacacacgacatgagttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttccatggatccgtcgccggccgtggatccgtcgccggccgtggatccgtcgccggctgctgaaacccggcggcgtgcaaccgggaaaggaggcaaacagcgcgggggcaagcaactaggattgaagaggccgccgccgatttctgtcccggccaccccgcctcctgctgcgacgtcttcatcccctgctgcgccgacggccatcccaccacgaccaccgcaatcttcgccgattttcgtccccgattcgccgaatccgtcaccggctgcgccgacctcctctcttgcttcggggacatcgacggcaaggccaccgcaaccacaaggaggaggatggggaccaacatcgaccatttccccaaactttgcatctttctttggaaaccaacaagacccaaattcatgtttggtcaggggttatcctccaggagggtttgtcaattttattcaacaaaattgtccgccgcagccacaacagcaaggtgaaaattttcatttcgttggtcacaatatggggttcaacccaatatctccacagccaccaagtgcctacggaacaccaacaccccaagctacgaaccaaggcacttcaacaaacattatgattgatgaagaggacaacaatgatgacagtagggcagcaaagaaaagatggactcatgaagaggaagagagactggccagtgcttggttgaatgcttctaaagactcaattcatgggaatgataagaaaggtgatacattttggaaggaagtcactgatgaatttaacaagaaagggaatggaaaacgtaggagggaaattaaccaactgaaggttcactggtcaaggttgaagtcagcgatctctgagttcaatgactattggagtacggttactcaaatgcatacaagcggatactcagacgacatgcttgagaaagaggcacagaggctgtatgcaaacaggtttggaaaaccttttgcgttggtccattggtggaagatactcaaaagagagcccaaatggtgtgctcagtttgaaaagaggaaaaggaagagcgaaatggatgctgttccagaacagcagaaacgtcctattggtagagaagcagcaaagtctgagcgcaaaagaaagcgcaagaaagaaaatgttatggaaggcattgtcctcctaggggacaatgtccagaaaattatcaaagtgacgcaagatcggaagctggagcgtgagaaggtcactgaagcacagattcacatttcaaacgtaaatttgaaggcagcagaacagcaaaaagaagcaaagatgtttgaggtatacaattccctgctcactcaagatacaagtaacatgtctgaagaacagaaggctcgccgagacaaggcattacaaaagctggaggaaaagttatttgctgactagtgacccagctttcttgtacaaagtggtgcctaggtgagtctagagagttgattaagacccgggactggtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacaggtaccttcaaacggcgcgccatgcagagtttagccatctctctactcctctcagaaactcattccctcttttctcatacgaagacctcctcccttttatctttactgtttctctcttcttcaaagatgtctgagcaaaatactgatggaagtcaagttccagtgaacttgttggatgagttcctggctgaggatgagatcatagatgatcttctcactgaagccacggtggtagtacagtccactatagaaggtcttcaaaacgaggcttctgaccatcgacatcatccgaggaagcacatcaagaggccacgagaggaagcacatcagcaactggtgaatgattacttttcagaaaatcctctttacccttccaaaatttttcgtcgaagatttcgtatgtctaggccactttttcttcgcatcgttgaggcattaggccagtggtcagtgtatttcacacaaagggtggatgctgttaatcggaaaggactcagtccactgcaaaagtgtactgcagctattcgccagttggctactggtagtggcgcagatgaactagatgaatatctgaagataggagagactacagcaatggaggcaatgaagaattttgtcaaaggtcttcaagatgtgtttggtgagaggtatcttaggcgccccactatggaagataccgaacggcttctccaacttggtgagaaacgtggttttcctggaatgttcggcagcattgactgcatgcactggcattgggaaagatgcccagtagcatggaagggtcagttcactcgtggagatcagaaagtgccaaccctgattcttgaggctgtggcatcgcatgatctttggatttggcatgcattttttggagcagcgggttccaacaatgatatcaatgtattgaaccaatctactgtatttatcaaggagctcaaaggacaagctcctagagtccagtacatggtaaatgggaatcaatacaatactgggtattttcttgctgatggaatctaccctgaatgggcagtgtttgttaagtcaatacgactcccaaacactgaaaaggagaaattgtatgcagatatgcaagaaggggcaagaaaagatatcgagagagcctttggtgtattgcagcgaagattttgcatcttaaaacgaccagctcgtctatatgatcgaggtgtactgcgagatgttgttctagcttgcatcatacttcacaatatgatagttgaagatgagaaggaaaccagaattattgaagaagatgcagatgcaaatgtgcctcctagttcatcaaccgttcaggaacctgagttctctcctgaacagaacacaccatttgatagagttttagaaaaagatatttctatccgagatcgagcggctcataaccgacttaagaaagatttggtggaacacatttggaataagtttggtggtgctgcacatagaactggaaattatggcgggggaggtagcgctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgataattgacattctaatctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagatcgcgaattcgtaatcatgtcatagctagaggatccccgggtaccgagctcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttaaagtactttaaagtactttaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 107 - protein, 183 aa, Artificial Sequence; identical to SEQ ID NO: 100
  Synthetic Construct

SEQ ID NO: 108 - protein, 1887 aa, Artificial Sequence; identical to SEQ ID NO: 95
  Synthetic Construct

SEQ ID NO: 109 - protein, 209 aa, Artificial Sequence; identical to SEQ ID NO: 101
  Synthetic Construct

SEQ ID NO: 110 - protein, 357 aa, Artificial Sequence; identical to SEQ ID NO: 102
  Synthetic Construct

SEQ ID NO: 111 - protein, 92 aa, Artificial Sequence; identical to SEQ ID NO: 103
  Synthetic Construct

SEQ ID NO: 112 - protein, 147 aa, Artificial Sequence; identical to SEQ ID NO: 104
  Synthetic Construct

SEQ ID NO: 113 - protein, 12 aa, Artificial Sequence
  Synthetic Construct
LSRYFGDHRFPH

(19) United States
(12) Patent Application Publication
(10) Pub. No.: US 2024/0150795 A1
(43) Pub. Date: May 9, 2024

(54) TARGETED INSERTION VIA TRANSPORTATION

(71) Applicant: Donald Danforth Plant Science Center, St. Louis, MO (US)

(72) Inventors: Keith R. Slotkin, St. Louis, MO (US); Peng Liu, St. Louis, MO (US)

(21) Appl. No.: 18/282,139

(22) PCT Filed: Mar. 15, 2022
(86) PCT No.: PCT/US22/20453

§ 371 Date: Sep. 14, 2023

Related U.S. Application Data
(60) Provisional application No. 63/220,148, filed on Jul. 9, 2021.
(60) Provisional application No. 63/161,155, filed on Mar. 15, 2021.

(51) Int. Cl. C12N 15/90; C12N 9/12; C12N 9/22; C12N 15/82

(52) U.S. Cl. CPC C12N 15/902; C12N 9/1241; C12N 9/22; C12N 15/8213; C12Y 207/07; C12Y 301/00; C12N 2310/20

(57) ABSTRACT

The present disclosure provides systems and methods for accurately inserting a donor polynucleotide into a target nucleic acid locus. A programmable targeting nuclease, a transposase, and a donor polynucleotide flanked by transposition sequences compatible with the transposase make up the system

### CROSS-REFERENCE TO RELATED APPLICATIONS

This application claims priority from Provisional Application No. 63/161,155, filed Mar. 15, 2021, and Provisional Application No. 63/220,148, filed Jul. 9, 2021, the contents of both of which are hereby incorporated by reference in their entirety.

### SEQUENCE LISTING

This application contains a Sequence Listing that has been submitted in ASCII format via EFS-Web and is hereby incorporated by reference in its entirety. The ASCII copy is named 077875-719495-US-Sequence-Listing.txt, and is 439 kilobytes in size.

### FIELD OF THE INVENTION

The present disclosure provides systems and methods of accurately inserting a donor polynucleotide into a target nucleic acid locus.

### BACKGROUND OF THE INVENTION

Genome editing is a revolutionary technology that promises the ability to improve or overcome current deficiencies in the genetic code as well as to introduce novel functionality. However, some applications of the technology do not always generate completely reliable results. For instance, transgene integration into or near genes can generate new mutations or alter the regulation of nearby genes, while insertions into heterochromatic regions are often not permissive to the desired high levels of transgene expression or do not provide stable expression over multiple generations. Further, in most instances, when performing transgenesis, the transgene frequently inserts into the nuclear genome in a random location. This can lead to new mutations at the insertion locus and at unintended insertion points, gene silencing, and general inconsistencies in experiments or products. For instance, in plants, where the frequency of homologous recombination is less than 1%, efficient and accurate insertion of transgenes is possible only in theory and is often associated with uncontrolled deletions of neighboring regions, as well as rearrangement of the transgene sequences. In fact, in a typical scenario, it simply is not possible to obtain the optimal, desired change. Additionally, although recently developed tools such as CRISPR systems have allowed biologists to target random genetic modifications to specific regions of genomes, accurate nucleic insertions in target loci is still a major challenge. In plants, this is because homologous recombination (HR) and Homology-Directed Repair (HDR) of donor sequences into the targeted locus occurs at a very low frequency.

Therefore, a long-felt need exists for improved and effective means of inserting polynucleotides into a user-defined location in the genome, especially in organisms where the frequency of homologous recombination (HR) is low, including plants.

### SUMMARY OF THE INVENTION

One aspect of the present disclosure encompasses an engineered system for generating a genetically modified cell. The engineered system comprises a nucleic acid expression construct for expressing a tranposase, wherein the expression construct comprises a promoter operably linked to a nucleic acid sequence encoding the transposase. The engineered system also comprises a nucleic acid construct comprising a donor polynucleotide comprising nucleic acid transposition sequences compatible with the transposase; and a nucleic acid expression construct for expressing a programmable targeting nuclease, wherein the expression construct comprises a promoter operably linked to a nucleic acid sequence encoding the programmable targeting nuclease. The targeting nuclease is engineered to introduce a cut in a target nucleic acid locus thereby guiding insertion of the donor polynucleotide at the target nucleic acid locus by the transposase to generate a genetically modified cell comprising the donor polynucleotide inserted at the target nucleic acid locus.

The transposase can be linked or not linked to the targeting nuclease. The system can further comprise a nucleic acid expression construct comprising a promoter operably linked to a polynucleotide sequence encoding a reporter, wherein the donor polynucleotide is inserted in the nucleic acid expression construct, wherein the reporter is inactivated by the inserted nucleic acid construct comprising the donor polynucleotide, and wherein the reporter is activated by excision of the inserted nucleic acid construct comprising the donor polynucleotide from the expression construct comprising a promoter operably linked to a polynucleotide sequence encoding a reporter by the transposase. In some aspects, the reporter is GFP, and wherein the nucleic acid expression construct comprises a nucleic acid sequence comprising at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74.

The transposase can be a split transposase. In some aspects, the transposase is a Pong or Pong-like transposase comprising a Pong ORF1 protein and a Pong ORF2 protein. In some aspects, the nucleic acid sequence encoding the Pong transposase comprises a Pong ORF1 protein, wherein the Pong ORF1 protein comprises an amino acid sequence comprising at least about 75% or more, at least about 85% or more, at least about 95% or more, or 100% sequence identity with the amino acid sequence of SEQ ID NO: 1, and wherein a nucleic acid sequence encoding the Pong ORF1 protein comprises at least about 75% or more, at least about 85% or more, at least about 95% or more, or 100% sequence identity with the nucleic acid sequence of SEQ ID NO: 2; and a Pong ORF2 protein, wherein the Pong ORF2 protein comprises an amino acid sequence comprising at least about 75% or more, at least about 85% or more, at least about 95% or more, or 100% sequence identity with the amino acid sequence of SEQ ID NO: 3, and wherein a nucleic acid sequence encoding the Pong ORF2 protein comprises at least about 75% or more, at least about 85% or more, at least about 95% or more, or 100% sequence identity with the nucleic acid sequence of SEQ ID NO: 4.

In some aspects, the transposition sequences are transposition sequences of a miniature inverted-repeat transposable element (MITE), and the MITE is an mPing MITE. In some aspects, transposition sequences of the mPing MITE comprise mPing inverted repeat 1 and inverted repeat 2, wherein mPing inverted repeat 1 comprises a nucleotide sequence comprising at least about 75% or more, at least about 85% or more, at least about 95% or more, or 100% sequence identity with the nucleic acid sequence of SEQ ID NO: 7, and mPing inverted repeat 2 comprises a nucleotide sequence comprising at least about 75% or more, at least about 85% or more, at least about 95% or more, or 100% sequence identity with the nucleic acid sequence of SEQ ID NO: 8.

The programmable targeting nuclease can comprise a programmable, sequence-specific nucleic acid-binding domain and a nuclease domain. The programmable targeting nuclease can be an RNA-guided clustered regularly interspersed short palindromic repeats (CRISPR)/CRISPR-associated (Cas) (CRISPR/Cas) nuclease system, a zinc finger nuclease (ZFN), a transcription activator-like effector nuclease (TALEN), a meganuclease, a ssDNA-guided Argonaute endonuclease, a meganuclease, a rare-cutting endonuclease, or any combination thereof. In some aspects, the programmable targeting nuclease is a CRISPR/Cas nuclease system comprising a nuclease and a guide RNA (gRNA). In some aspects, the programmable targeting nuclease comprises a Cas9 nuclease comprising an amino acid sequence comprising at least about 75% or more, at least about 85% or more, at least about 95% or more, or 100% sequence identity with the amino acid sequence of SEQ ID NO: 5, and wherein the Cas9 nuclease is encoded by a nucleic acid sequence comprising at least about 75% or more, at least about 85% or more, at least about 95% or more, or 100% sequence identity with the nucleic acid sequence of SEQ ID NO: 6. The gRNA can comprise a nucleic acid sequence of SEQ ID NO: 65, SEQ ID NO: 66, SEQ ID NO: 67, SEQ ID NO: 80, or any combination thereof.

In some aspects, the transposase is a Pong transposase, wherein the nucleic acid transposition sequences are mPing inverted repeat 1 and inverted repeat 2, and the programmable targeting nuclease comprises a Cas9 nuclease and a gRNA, wherein the gRNA comprises a nucleic acid sequence of SEQ ID NO: 65, SEQ ID NO: 66, SEQ ID NO: 67, SEQ ID NO: 80, or any combination thereof.

In some aspects, the nucleic acid construct comprising the donor polynucleotide comprises a nucleic acid sequence comprising at least about 75% or more, at least about 85% or more, at least about 95% or more, or 100% sequence identity with the nucleic acid sequence starting at nucleotide 69 to nucleotide 498 of SEQ ID NO: 92. The system can further comprises a nucleic acid expression construct comprising a promoter operably linked to a polynucleotide sequence encoding a GFP reporter, wherein the donor polynucleotide is inserted in the nucleic acid expression construct, and wherein the nucleic acid expression construct comprises at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74. In some aspects, the nucleic acid construct comprising the donor polynucleotide comprises a nucleotide sequence comprising heat shock element (HSE) sequences flanked by mPing inverted repeat 1 and inverted repeat 2, and wherein the nucleic acid construct comprising the donor polynucleotide comprises at least about 75% or more, at least about 85% or more, at least about 95% or more, or 100% sequence identity with the nucleic acid sequence of SEQ ID NO: 81. The Cas9 nuclease can be deCas9 nickase, wherein the engineered system can comprise a nucleic acid expression construct for expressing the deCas9 nickase comprises a nucleic acid sequence comprising at least about 75% or more, at least about 85% or more, at least about 95% or more, or 100% sequence identity with the nucleic acid sequence starting at nucleotide 8218 to 13856 of SEQ ID NO: 89. In some aspects, the engineered system comprises a nucleic acid expression construct for expressing a Cas9 nuclease comprises a nucleic acid sequence comprising at least about 75% or more, at least about 85% or more, at least about 95% or more, or 100% sequence identity with the nucleic acid sequence starting at base 10857 to base 16495 of SEQ ID NO: 94.

In some aspects, the Cas9 nuclease is not fused to the Pong ORF2 protein, wherein the engineered system comprises a nucleic acid expression construct for expressing a Pong ORF2 protein comprises a nucleic acid sequence comprising at least about 75% or more, at least about 85% or more, at least about 95% or more, or 100% sequence identity with the nucleic acid sequence starting at base 5073 to base 8215 of SEQ ID NO: 89. In other aspects, the Cas9 nuclease is fused to the Pong ORF2 protein, wherein the system comprises a nucleic acid nucleic acid expression construct for expressing a Pong ORF1 protein and an expression construct for expressing a Pong ORF2 protein fused to the Cas9 nuclease, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 3359 to base 7268 of SEQ ID NO: 74, and wherein an expression construct for expressing a Pong ORF2 protein fused to the Cas9 nuclease comprises a nucleic acid sequence comprising at least about 75% or more, at least about 85% or more, at least about 95% or more, or 100% sequence identity with the nucleic acid sequence starting at base 7451 to base 14807 of SEQ ID NO: 74.

In some aspects, the expression construct for expressing a gRNA comprises a nucleic acid sequence comprising at least about 75% or more, at least about 85% or more, at least about 95% or more, or 100% sequence identity with the nucleic acid sequence starting at base 2632 to base 3343 of SEQ ID NO: 74. In other aspects, the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 89. In yet other aspects, the expression construct for expressing the gRNA comprises a nucleic acid sequence comprising at least about 75% or more, at least about 85% or more, at least about 95% or more, or 100% sequence identity with the nucleic acid sequence starting at base 729 to base 1440 of SEQ ID NO: 92.

In some aspects, the system comprises a nucleic acid construct comprising: a nucleic acid nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 5073 to base 8215 of SEQ ID NO: 89, a nucleic acid nucleic acid expression construct for expressing a Pong ORF2 protein fused to Cas9 nuclease, wherein the expression construct for expressing the Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 7451 to base 14807 of SEQ ID NO: 74; a nucleic acid expression construct comprising a promoter operably linked to a polynucleotide sequence encoding GFP, further comprising the donor polynucleotide inserted in the nucleic acid expression construct, wherein the nucleic acid expression construct comprises a nucleic acid sequence comprising at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74; and an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 2632 to base 3343 of SEQ ID NO: 74.

In other aspects, the system comprises a nucleic acid construct comprising: a nucleic acid nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 1456 to base 5362 of SEQ ID NO: 92; a nucleic acid nucleic acid expression construct for expressing a Pong ORF2 protein fused to Cas9 nuclease, wherein the expression construct for expressing the Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 5548 to base 12904 of SEQ ID NO: 92; a nucleic acid construct comprising the donor polynucleotide, wherein the nucleic acid construct comprises a nucleic acid sequence comprising at least about 75% or more, at least about 85% or more, at least about 95% or more, or 100% sequence identity with the nucleic acid sequence starting at nucleotide 69 to nucleotide 498 of SEQ ID NO: 92; and an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 729 to base 1440 of SEQ ID NO: 92.

In yet other aspects, the system comprises a nucleic acid construct comprising: a nucleic acid nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 1481 to base 5390 of SEQ ID NO: 93; a nucleic acid nucleic acid expression construct for expressing a Pong ORF2 protein fused to Cas9 nuclease, wherein the expression construct for expressing the Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 1481 to base 5390 of SEQ ID NO: 93; a nucleic acid construct comprising the donor polynucleotide, wherein the donor polynucleotide comprises a nucleotide sequence comprising HSE sequences flanked by mPing inverted repeat 1 and inverted repeat 2, and wherein the nucleic acid construct comprises at least about 75% or more, at least about 85% or more, at least about 95% or more, or 100% sequence identity with the nucleic acid sequence starting at base 69 to base 512 of SEQ ID NO: 93; and an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 754 to base 1465 of SEQ ID NO: 93.

In additional aspects, the system comprises a nucleic acid construct comprising: a nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 981 to base 4890 of SEQ ID NO: 75; a nucleic acid expression construct for expressing a Pong ORF2 protein fused to Cas9 nuclease, wherein the expression construct for expressing the Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 5073 to base 12429 of SEQ ID NO: 75; and an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 75. In some aspects, the system comprises a nucleic acid construct comprising: a nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 981 to base 4890 of SEQ ID NO: 89; a nucleic acid expression construct for expressing a Pong ORF2 protein, wherein the expression construct for expressing the Pong ORF2 protein comprises a nucleic acid sequence comprising at least about 75% or more, at least about 85% or more, at least about 95% or more, or 100% sequence identity with the nucleic acid sequence starting at base 5073 to base 8215 of SEQ ID NO: 89; a nucleic acid expression construct for expressing the deCas9 nickase comprises a nucleic acid sequence comprising at least about 75% or more, at least about 85% or more, at least about 95% or more, or 100% sequence identity with the nucleic acid sequence starting at nucleotide 8218 to nucleotide 13856 of SEQ ID NO: 89; and an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 89. In some aspects, the system further comprises a donor nucleic acid construct comprising a promoter operably linked to a polynucleotide sequence encoding a GFP reporter, wherein the donor polynucleotide is inserted in the nucleic acid expression construct, wherein the nucleic acid expression construct comprises at least about 75% or more, at least about 85% or more, at least about 95% or more, or 100% sequence identity with the nucleic acid sequence starting at base 3037 clockwise to base 665 of SEQ ID NO: 90.

In some aspects, the system comprises a helper nucleic acid construct and a donor nucleic acid construct. The helper nucleic acid construct can comprise a nucleic acid nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 981 to base 4890 of SEQ ID NO: 91; a nucleic acid nucleic acid expression construct for expressing a Pong ORF2 protein fused to Cas9 nuclease, wherein the expression construct for expressing the Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 5073 to base 12429 of SEQ ID NO: 91; and an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 91. The donor nucleic acid construct can comprise a promoter operably linked to a polynucleotide sequence encoding a GFP reporter, wherein the donor polynucleotide is inserted in the nucleic acid expression construct, and wherein the nucleic acid expression construct comprises at least about 75% or more, at least about 85% or more, at least about 95% or more, or 100% sequence identity with the nucleic acid sequence starting at base 3037 clockwise to base 665 of SEQ ID NO: 90.

In some aspects, the system comprises a nucleic acid construct comprising: a nucleic acid nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 3593 to base 7502 of SEQ ID NO: 94; a nucleic acid nucleic acid expression construct for expressing a Pong ORF2 protein, wherein the expression construct for expressing the Pong ORF2 protein comprises a nucleic acid sequence comprising at least about 75% or more, at least about 85% or more, at least about 95% or more, or 100% sequence identity with the nucleic acid sequence starting at base 7685 to base 10827 of SEQ ID NO: 94; a nucleic acid expression construct for expressing a Cas9 nuclease comprises a nucleic acid sequence comprising at least about 75% or more, at least about 85% or more, at least about 95% or more, or 100% sequence identity with the nucleic acid sequence starting at base 10857 to base 16495 of SEQ ID NO: 94; a nucleic acid construct comprising the donor polynucleotide, wherein the nucleic acid construct comprising the donor polynucleotide comprises a nucleic acid sequence comprising at least about 75% or more, at least about 85% or more, at least about 95% or more, or 100% sequence identity with the nucleic acid sequence starting at base 2201 to base 2630 of SEQ ID NO: 94; and an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 2861 to base 3572 of SEQ ID NO: 94.

In other aspects, the system comprises a nucleic acid construct comprising: a nucleic acid nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 5490 to base 9399 of SEQ ID NO: 95; a nucleic acid nucleic acid expression construct for expressing a Pong ORF2 protein fused to Cas9 nuclease, wherein the expression construct for expressing the Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 9582 to base 16938 of SEQ ID NO: 95; a nucleic acid expression construct comprising a promoter operably linked to a polynucleotide sequence encoding GFP, further comprising the donor polynucleotide inserted in the nucleic acid expression construct, wherein the nucleic acid expression construct comprises a nucleic acid sequence comprising at least about 75% or more, at least about 85% or more, at least about 95% or more, or 100% sequence identity with the nucleic acid sequence starting at base 4545 to base 2173 of SEQ ID NO: 95; and an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 4763 to base 5474 of SEQ ID NO: 95.

In some aspects, the target nucleic acid locus is in a nuclear, organellar, or extrachromosomal nucleic acid sequence and can be in a protein-coding gene, an RNA coding gene, or an intergenic region.

The cell can be a eukaryotic cell. In some aspects, the cell is a plant cell, and can be an Arabidopsis sp. or a soybean plant.

Another aspect of the present disclosure encompasses one or more nucleic acid constructs encoding an engineered nucleic acid modification system as described above.

Yet another aspect of the present disclosure encompasses a cell comprising an engineered system or one or more nucleic acid constructs described above. The cell can be a eukaryotic cell. In some aspects, the cell is a plant cell, and can be an Arabidopsis sp. or a soybean plant.

An additional aspect of the instant disclosure encompasses a method of inserting a donor polynucleotide into a target nucleic acid locus in a cell. The method comprises introducing one or more nucleic acid constructs described above into the cell; maintaining the cell under conditions and for a time sufficient for the donor polynucleotide to be inserted in the target locus; and optionally identifying an insertion of the donor polynucleotide in the nucleic acid locus in the cell. The cell can be a eukaryotic cell. In some aspects, the cell is a plant cell, and can be an Arabidopsis sp. or a soybean plant. In some aspects, the cell is ex vivo.

One aspect of the present disclosure encompasses a method of altering the expression of a gene of interest. The method comprises using a method described above to insert an array of six heat-shock enhancer elements flanked by mPing transposition sequences into a promoter of the gene of interest. The gene of interest can be an Arabidopsis ACT8 gene.

Another aspect of the instant disclosure encompasses a kit for generating a genetically modified cell. The kit comprises one or more engineered systems described above or one or more nucleic acid constructs described above, wherein each of the engineered systems generates an engineered cell comprising an accurate insertion of the donor polynucleotide into the target nucleic acid locus. In some aspects, the kit comprises one or more cells comprising one or more engineered systems, one or more nucleic acid constructs, or combinations thereof. The method comprises using a method described above to insert an array of six heat-shock enhancer elements flanked by mPing transposition sequences into a promoter of the gene of interest.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a diagram depicting an engineered system excising a donor polynucleotide from a donor site in a plant, and inserting the excised donor polynucleotide into a locus in the Arabidopsis PDS3 gene.

FIG. 2 depicts a schematic overview of twelve different transgenes comprising Cas9 and derivative proteins fused either to the N- or C-terminus of Pong transposase ORF1 (blue) or to the N- or C-terminus of Pong ORF2 (orange) protein coding regions. Three different versions of Cas9 were used: double-strand cleavage Cas9, the single stranded nickase deCas9, and the catalytically dead dCas9.

FIG. 3A. The functional verification of ORF1/2 and Cas9 fusion proteins. GFP fluorescence was detected for all 12 fusion proteins as well as the ORF1/ORF2 positive control, since mPing excision from the GFP donor site restores the GFP expression. The negative control without ORF1/ORF2 (−ORF1 −ORF2) was not able to excise mPing.

FIG. 3B. The functional verification of ORF1/2 and Cas9 fusion proteins. A functional CRISPR/Cas9 system when fused to ORF1/2 was verified through the observation of white seedlings and sectors in plants generated from the Cas9 targeting of the Arabidopsis PDS3 gene with all four Cas9 fusion proteins. Three examples of individual plants are shown.

FIG. 4A. Screening insertions. PCR strategy to detect targeted insertions into the PDS3 gene. mPing can insert in the forward or reverse orientation relative to PDS3.

FIG. 4B. Screening insertions. PCR with negative controls: a line lacking the ORF1/ORF2 proteins (mPing only), lacking Cas9 (mPing+ ORF1/ORF2) and a no template PCR (−). The expected amplification sizes are indicated by black arrowheads. The correct PCR products validated by Sanger sequencing are marked with red arrows.

FIG. 4C. Screening insertions. Replicate of the PCR from clone #2 in FIG. 4B. This PCR displays the correct sized and sequenced bands (red arrows) in each reaction.

FIG. 5 depicts nucleic acid sequences at insertion sites of 9 unique transposition events. The sequence of the mPing transposable element is green. The target site duplication sequence is red. The guide RNA target site is grey highlighted. The PDS gene is unhighlighted black. For simplicity, only the mPing/PDS3 junction of these sequences are shown.

FIG. 6A. PCR strategy to determine if any transgenic DNA would insert at a Cas9 cleavage site. The PCR shows no bands of expected size (black arrowheads), which demonstrates that mPing insertion from FIG. 4 is a product of transposition, and not random.

FIG. 6B. Testing if the single components of the system could recapitulate the results. No Cas9 and ORF1/2 (mPing only), no Cas9 (+ORF1/2), and no ORF1/2 (+Cas9) controls each failed to produce the expected band and therefore cannot generate targeted insertions. Having Cas9 and ORF1/2, but in an un-fused configuration, produced targeted insertion. The lane to the far right is clone #2 from FIG. 4, which is used as a positive control in this experiment. The four gels represent the same four PCR assays from FIG. 4A. Black arrowheads denote the expected size of the targeted insertion in each PCR.

FIG. 7A is a diagram showing the three systems designed with gRNAs targeted to three different target loci: the PDS3 gene, the ADH1 gene, and the promoter of ACT8 gene.

FIG. 7B are the Sanger sequencing results of junctions of target insertions into the PDS3 gene, the ADH1 gene, and the promoter of ACT8 gene. The sequence below mPing is the expected sequence of a perfect “seamless” insertion. The chromatograms above the sequence show the sequences at the insertion sites. The highlighted bases are 1-2 nucleotide insertions or deletions.

FIG. 8A depicts a PCR strategy to detect targeted insertions into the PDS3 gene. mPing can insert in either the forward direction (above the PDS3 region) or reverse direction (below the PSD3 region). The location of 4 PCR primers (R,L,U,D) are shown for orientation.

FIG. 8B depicts an agarose gel run of PCR products using primers from FIG. 8A from systems comprising ORF1 and 2 fused or unfused to Cas9 nuclease. Arrowheads denote the correct size of the PCR products for each set of primers. No Cas9 and ORF1/2 (“mPing only”), no Cas9 (“+ORF1/2”), and no ORF1/2 (“+Cas9”) are negative controls and showed no bands.

FIG. 9A is a diagram of a vector that contains the CRISPR/Cas9 system (including gRNA), the mPing donor element, and ORF1 and ORF2 transposase proteins.

FIG. 9B depicts a PCR strategy to detect targeted insertions into the PDS3 gene using the vector of FIG. 9A. mPing can insert in either the forward direction (above the PDS3 region) or reverse direction (below the PSD3 region). The location of 4 PCR primers (R,L,U,D) are shown for orientation.

FIG. 9C depicts PCR detection of mPing targeted insertion in the Arabidopsis genome using the vector in FIG. 9A. PCR detection used primer sets from FIG. 9B.

FIG. 10 depicts targeted insertion based on the Pong/mPing transposon system. Fusion of the Pong transposase ORFs with Cas9 provides the transposase sequence specificity for the insertion of the non-autonomous mPing element. The mPing element is excised out of a donor site provided on the transgene, generating fluorescence. mPing insertion at the target site is screened for by PCR.

FIG. 11 depicts the Experimental Design of Protein Fusions and Testing. Twelve different transgenes where created and transformed into Arabidopsis. Cas9 and derivative proteins where fused either to the Pong transposase ORF1 (blue) or ORF2 (orange) protein coding regions. Both N- and C-terminal fusions were created. Three different versions of Cas9 were used: double-strand cleavage Cas9, the single stranded nickase deCas9, and the catalytically dead dCas9. When a functional transposase protein is generated by expression of ORF1 and ORF2, it excises the mPing transposable element out of the 35S-GFP donor location, producing fluorescence. The goal of this project was to demonstrate user-defined targeted insertion of the mPing transposable element by programming the CRISPR-Cas9 system with a custom guide RNA.

FIG. 12A depicts photographs showing fluorescence generated upon excision of mPing from the 35S:GFP donor site. mPing only transposes in the presence of both ORF1 and ORF2 transposase proteins, and fusing ORF2 to Cas9 still results in mPing excision.

FIG. 12B depicts a northern blot showing excision as in FIG. 12A assayed by PCR using primers at the 35S:GFP donor site. A smaller sized band is generated upon mPing excision. insertion site identified by Sanger sequencing targeted insertion events.

FIG. 12C depicts a PCR assay to detect targeted insertion of mPing at PDS3 gene. Primer names (U,L,R,D) and locations are listed above. Targeted insertion is detected via PCR in plants that have all three proteins: ORF1, ORF2 and Cas9. Targeted insertions are detected when ORF2 and Cas9 are physically fused, or when unfused but present in the same cells.

FIG. 12D depicts a cartoon of mPing excision and targeted insertion when ORF2 is fused to Cas9.

FIG. 12E depicts an example of a Sanger sequence read of the junction between the PDS3 gene and the targeted insertion of mPing.

FIG. 12F depict sequence analysis of 17 distinct insertion events of mPing at PDS3. mPing sequences are shown in yellow, and the target site duplication of TTA/TAA from the donor site is shown in red. Within the PDS3 target site, the gRNA targeted sequence is shown in grey. The mPing is inserted between the third and fourth base of the gRNA target sequence (black arrowhead). The variation of the sequence found on either end of the insertion site is shown.

FIG. 12G depicts a plot showing the number of SNPs at the insertion site identified by Sanger sequencing targeted insertion events.

FIG. 13A depicts photographs showing the functional verification of ORF1/2 and Cas9 fusion proteins. GFP fluorescence was detected for all 12 fusion proteins as well as the ORF1/ORF2 positive control, since mPing excision from the GFP donor site restores the GFP expression. The negative control without ORF1/ORF2 (−ORF1 −ORF2) was not able to excise mPing.

FIG. 13B depict the functional verification of ORF1/2 and Cas9 fusion proteins. A functional CRISPR/Cas9 system when fused to ORF1/2 was verified through the observation of white seedlings and sectors in plants with all four Cas9 fusion proteins. Three examples of individual plants are shown.

FIG. 14A depicts a PCR strategy to detect targeted insertions into the PDS3 gene. mPing can insert in the forward or reverse orientation relative to PDS3.

FIG. 14B depicts an electrophoresis gel of PCR products with negative controls: a line lacking the ORF1/ORF2 proteins (mPing only), lacking Cas9 (mPing+ORF1/ORF2) and a no template PCR (−). The expected amplification sizes are indicated by black arrowheads. The correct PCR products are marked with red arrows.

FIG. 14C depicts screening insertions. Replicate of the PCR from clone #2. This PCR displays the correct sized bands (red arrows) in each reaction.

FIG. 15 depicts the comparison of the number of base deletions (left of zero on the X-axis) and insertions (right of zero on the X-axis) for two configurations of Cas9 and ORF2: fused and unfused. Insertions of mPing (red) into PDS3 (blue) were subject to amplicon deep sequencing and each junction analyzed separately. Since mPing can insert in either orientation (black arrows within red mPing elements), four distinct junction points are analyzed. The size of the black filled circle represents the percentage of deep sequenced reads.

FIG. 16A depict additional controls. PCR strategy to determine if any transgenic DNA would insert at a Cas9 cleavage site. The PCR shows no bands, which demonstrates that mPing insertion from FIGS. 12A-13B is a product of transposition, and not random.

FIG. 16B depict additional controls. Testing if the single components of our system could recapitulate our results. No Cas9 and ORF1/2 (mPing only), no Cas9 (+ORF1/2), and no ORF1/2 (+Cas9) controls each failed to produce the expected band and therefore cannot generate targeted insertions. Having Cas9 and ORF1/2, but in an un-fused configuration, produced targeted insertion. The lane to the far right is clone #2 from FIGS. 12-12G, which is used as a positive control in this experiment. The four gels represent the same four PCR assays from FIG. 12A. Black arrowheads denote the expected size of the targeted insertion in each PCR.

FIG. 17A depicts an overview of targeted insertion at 3 distinct loci. By switching the CRISPR gRNA, distinct regions of the genome are targeted for mPing insertion.

FIG. 17B depicts how mPing can insert into DNA for both directions. Arrows indicate primers used to detect target insertions: U, upstream of target gene; D, downstream of target gene; R, right end of mPing; L, left end of mPing. PCR products were then purified and sequenced.

FIG. 17C depicts sanger sequencing chromatograms for junctions of target insertions into an additional target besides PDS3: ADH1.

FIG. 17D depicts sanger sequencing chromatograms for junctions of target insertions into an additional target besides PDS3: ACT8 promoter.

FIG. 18 depicts analysis of the left and right junctions of mPing targeted insertions upstream of the ACT8 gene in T2 plants with Cas9 fused to ORF2. Single individual T2 plants were assayed one-by-one, and 8 plants were confirmed by Sanger sequencing to have targeted insertions of mPing.

FIG. 19A. Addition of 6 heat shock element (HSE) sequences into mPing and targeted insertion upstream of the ACT8 gene.

FIG. 19B. mPing element excision from the donor location demonstrating that the modified mPing-HSE element could excise properly. The SspI digest is performed to improve the assay's sensitivity.

FIG. 19C PCR strategy to detect targeted insertions (top) and PCR assay for targeted insertions (bottom). Both a pool of T2 plants was assayed, as well as four individual T2 generation plants. Bands with arrow heads are the correct size and were Sanger sequenced to demonstrate the correct targeted insertion into the promoter region of the ACT8 gene.

FIG. 20 depicts a map of the vector testing the ability of unfused Cas9 Nickase to direct targeted insertions of mPing. Targeted insertion into ADH1 has been detected at a low frequency and sequenced. This insertion shows the left junction of mPing at ADH1 with a 14 bp deletion.

FIG. 21A Vector maps of TDNAs used for a two-step (two-component) transformation. The donor vector was transformed into Arabidopsis first, and a stable transgenic line was used for a second transformation using the helper vector.

FIG. 21B The one-component vector containing both donor TE (mPing) and helpers (ORF1, ORF2-Cas9) was also tested to be able to direct targeted insertion. Blue triangles are LB and RB ends of the T-DNA. Arrows denote promoters, and black boxes are terminators. The mPing donor TE is shown in red.

FIG. 22 depicts experimental design to use targeted transposition of a modified mPing element in order to transcriptionally rewire the ACT8 gene. The goal is to engineer the ACT8 gene have transcriptional activation during heat stress.

FIG. 23A depicts the transposase-mediated targeted insertion of mPing into the soybean (Glycine max) crop genome. Soybean transformation vector with a gRNA that targets the “DD20” region of the soybean genome, and unfused ORF2 and Cas9.

FIG. 23B depicts the transposase-mediated targeted insertion of mPing into the soybean (Glycine max) crop genome. Similar vector as in FIG. 23A, but with a fused ORF2 and Cas9.

FIG. 23C depicts the transposase-mediated targeted insertion of mPing into the soybean (Glycine max) crop genome. The overall goal of targeted insertion of mPing into the DD20 region of the soybean genome.

FIG. 23D depicts the transposase-mediated targeted insertion of mPing into the soybean (Glycine max) crop genome. PCR primer strategy to detect targeted insertion (top) and PCR gel (bottom). Bands with red arrowheads are the correct size and were validated by Sanger sequencing. Two out of nine transgenic soybean plants showed targeted insertion of mPing.

FIG. 23E depicts the transposase-mediated targeted insertion of mPing into the soybean (Glycine max) crop genome. Sanger sequence example of a targeted insertion into the soybean genome (plant RO #8 from FIG. 23D).

### DETAILED DESCRIPTION

The present disclosure encompasses engineered systems and methods of using the engineered systems for generating genetically modified cells and organisms. Unlike currently available insertion systems that rely on homologous recombination or homology-directed repair for inserting a nucleic acid sequence, the systems and methods of the disclosure can efficiently mediate controlled and targeted insertion of a polynucleotide of choice to generate a genetically modified cell having an insertion of the polynucleotide at a target nucleic acid locus in a gene of interest. Importantly, the disclosed systems and methods can efficiently mediate targeted insertion of polynucleotides even in organisms where such genetic manipulation is known to be problematic, including plants. Further, the compositions and methods can insert polynucleotides without introducing unwanted mutations in the transferred polynucleotide or in the nucleic acid sequences at the target nucleic acid locus. The system can accomplish that by combining the targeting capabilities of a targeting nuclease, with the insertion capability and ability to seamlessly resolve the junction without mutation of a transposase. This bypasses the host-encoded homologous recombination step or damage repair pathways normally used when a polynucleotide is introduced. Surprisingly and unexpectedly, the systems can simultaneously target more than one locus.

I. Composition

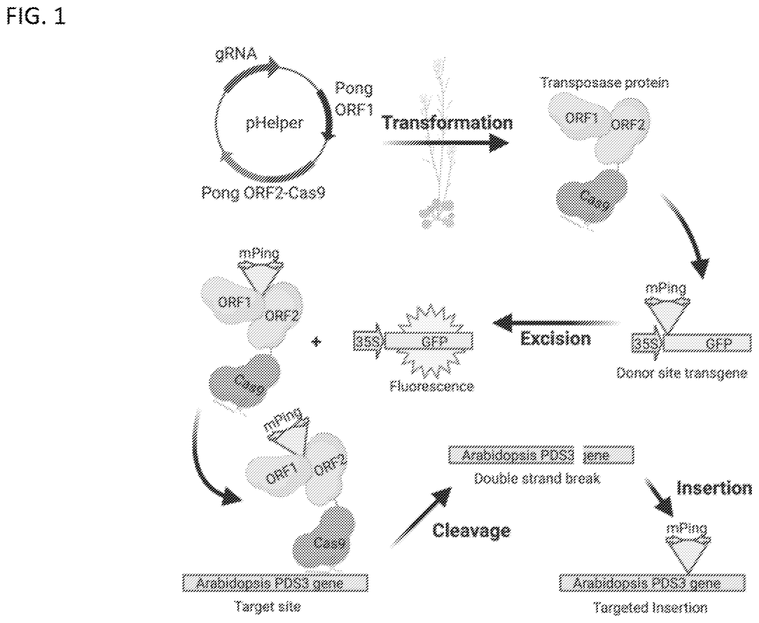

One aspect of the present disclosure encompasses an engineered system for generating a genetically modified cell. The system comprises a targeting nuclease capable of guiding transposition of a donor polynucleotide to a target locus, and a transposase to precisely insert the donor polynucleotide into the target locus. The transposase recognizes and binds transposition sequences flanking the donor polynucleotide, and the targeting nuclease targets the transposase and the donor polynucleotide to a target nucleic acid locus to thereby mediate insertion of the donor polynucleotide into the target nucleic acid locus, and to thereby generate a genetically engineered cell comprising an insertion of the donor polynucleotide into the target nucleic acid locus (FIG. 1). The targeting nuclease, the transposase, and the donor polynucleotide are described in further detail below.

### (a) Transposase

The system comprises a transposase. As used herein, the term “transposase” refers to a protein or a protein fragment derived from any transposable element (TE), wherein the transposase is capable of inserting a polynucleotide at a target locus and/or cutting or copying a donor polynucleotide for inserting the polynucleotide at the target locus. TEs can be assigned to any one of two classes according to their mechanism of transposition, which can be described as either copy and paste (Class I TEs) or cut and paste (Class II TEs).

Class I TEs are retrotransposons that copy and paste themselves into different genomic locations in two stages: first, TE nucleic acid sequences are transcribed from DNA to RNA, and the RNA produced is then reverse transcribed to DNA. This copied DNA is then inserted back into the genome at a new position. The reverse transcription step is catalyzed by a reverse transcriptase activity, which is often encoded by the TE itself. Non-limiting examples of Class I TEs include Tnt1, Opie, Huck, and BARE1.

The transposition mechanism of Class II TEs does not involve an RNA intermediate. The transpositions are catalyzed by a transposase enzyme that cuts the target site, cuts out the transposon or copies the transposon, and positions it for ligation into the target site. Non-limiting examples of Class II TEs include P Instability Factor (PIF), Pong, AciDs, Pong TE or Pong-like TEs, Spm/dSpm, Harbinger, P-elements, Tn5 and Mutator.

Transposases generally recognize and interact with compatible transposition sequences at the ends of the TE to mediate transposition of the TE. For instance, the transposase binds the transposition sequences at the terminal ends of the TE and cleaves the DNA, removing the TE from the excision/donor site, then cleaves the insertion site at a new location in the genome of a cell and integrates the TE at the insertion site. For Class I TEs, the transposases of some TEs recognize the terminal transposition sequences at the ends of an RNA transcript of the TE, reverse transcribe the transcript into DNA, then cleave and integrate the TE at the insertion site. Accordingly, a transposase of the instant disclosure can be any transposase or fragment thereof, provided the transposase recognizes the compatible terminal transposition sequences of the donor polynucleotide and mediates insertion of the polynucleotide at the target locus. Transposition sequences compatible with the transposase can be as described in Section I(b) below.

In an engineered system of the instant disclosure, a transposase recognizes the transposition sequences of the donor polynucleotide. When the transposase is derived from a Class I TE, the transposase first transcribes the donor polynucleotide into an RNA transcript and reverse transcribes the RNA transcript to DNA for insertion at the target locus. When the transposases is derived from a Class II TE, the transposase first cleaves or copies the donor polynucleotide from a source nucleic acid sequence such as a nucleic acid construct encoding the donor polynucleotide for insertion at the target locus. In some aspects, the transposases also cleaves the target locus before inserting the donor polynucleotide. In other aspects, the nucleic acid sequence at the target is cleaved by the targeting nuclease as described further below.

In some aspects, the transposase is derived from a Class II TE. In some aspects, the transposase is derived from the P Instability Factor (PIF) TE or PIF-like TEs. In some aspects, a transposase of the instant disclosure is a split transposase. In some aspects, the transposase is a Pong or Pong-like transposase comprising a Pong ORF1 protein and a Pong ORF2 protein. The transposases of the Pong and Pong-like TEs are split transposases comprising a first protein encoded by open reading frame 1 (ORF1 protein) and a second protein encoded by open reading frame 2 (ORF2 protein) of the TE.

Accordingly, when a transposase of the instant disclosure is a Pong or Pong-like transposase, the system comprises both ORF1 and ORF2 proteins. In some aspects, the Pong ORF1 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 1. In some aspects, the Pong ORF1 protein comprises an amino acid sequence comprising at least about 75% or more, at least about 85% or more, at least about 95% or more, or 100% sequence identity with the nucleic acid sequence of SEQ ID NO: 1. In some aspects, a nucleic acid sequence encoding the Pong ORF1 protein comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 2. In some aspects, a nucleic acid sequence encoding the Pong ORF1 protein comprises at least about 75% or more, at least about 85% or more, at least about 95% or more, or 100% sequence identity with the nucleic acid sequence of SEQ ID NO: 2.

In some aspects, the Pong ORF2 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino sequence of SEQ ID NO: 3. In some aspects, the Pong ORF2 protein comprises an amino acid sequence comprising at least about 75% or more, at least about 85% or more, at least about 95% or more, or 100% sequence identity with the amino acid sequence of SEQ ID NO: 3. In some aspects, a nucleic acid sequence encoding the Pong ORF2 protein comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 4. In some aspects, a nucleic acid sequence encoding the Pong ORF2 protein comprises at least about 75% or more, at least about 85% or more, at least about 95% or more, or 100% sequence identity with the nucleic acid sequence of SEQ ID NO: 4.

### (b) Donor Polynucleotide

Engineered systems of the disclosure also comprise a donor polynucleotide. In the presence of the transposases and the programmable targeting nuclease, the donor polynucleotide is targeted to a target nucleic acid locus by the programmable targeting nuclease to thereby mediate insertion of the donor polynucleotide into the target nucleic acid locus by the transposase. A donor polynucleotide comprises a first transposition sequence at a first end of the donor polynucleotide, and a second transposition sequence at a second end of the donor polynucleotide. The transposition sequences are compatible with the transposase of a system of the instant disclosure. As used herein, the term “compatible” when referring to transposition sequences refers to transposition sequences that can be recognized by a transposase of the instant disclosure for transposition of the donor polynucleotide in the cell.

Generally, the transposition sequences are derived from the TE from which the transposase is derived. However, the transposition sequences can also be derived from TEs other than the TE from which the transposases are derived, provided the transposition sequences are compatible with the transposon of the system. Transposition sequences of the instant disclosure can be derived from autonomous or non-autonomous TEs. Non-autonomous TEs have short internal sequences devoid of open reading frames (ORF) that encode a defective transposase, or do not encode any transposase. Non-autonomous elements transpose through transposases encoded by autonomous TEs. The transposition sequences of the donor polynucleotide can each have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with transposition sequences of the TE from which they are derived.

As explained in Section I(a) above, the transposase recognizes the transposition sequences and mediates the insertion of the donor polynucleotide into the desired target locus. A donor polynucleotide can be an RNA polynucleotide or a DNA polynucleotide. The transposition sequence can flank nucleic acid sequences of interest, and insertion of the donor polynucleotide results in the insertion of the nucleic acid sequences of interest into the desired target locus. Non-limiting examples of nucleic acid sequences that can be of interest for inserting in a target locus can be as described in Section IV herein below.

Further, insertion of the donor polynucleotide in a target locus can alter the function of the target locus. For instance, insertion of a donor polynucleotide in a nucleic acid sequence encoding a reporter can inactivate the reporter, thereby indicating a successful integration event. Conversely, excision of a donor polynucleotide from a nucleic acid sequence encoding a reporter can re-activate the reporter, thereby indicating a successful excision event.

In some aspects, a system of the instant disclosure comprises a donor polynucleotide inserted in a nucleic acid expression construct comprising a promoter operably linked to a polynucleotide sequence encoding a reporter, wherein the reporter is inactivated by the inserted nucleic acid construct comprising the donor polynucleotide, and wherein the reporter is activated by excision of the inserted nucleic acid construct comprising the donor polynucleotide from the expression construct comprising a promoter operably linked to a polynucleotide sequence encoding a reporter by the transposase. The reporter can be a GFP reporter.

In some aspects, the transposase of the instant disclosure is derived from a P/F or P/F-like TE, and the transposition sequences compatible with the transposase are derived from a P/F or a P/F-like TE from which the transposase is derived, or can be derived from a tourist-like miniature inverted-repeat transposable element (MITE). In some aspects, the transposase is derived from a Pong, a Pong-like, Ping, or a Ping-like TE, and the transposition sequences compatible with the transposase can be derived from a stowaway-like MITE. In some aspects, the transposase is derived from a Pong, a Pong-like, a Ping, or a Ping-like TE, and the transposition sequences compatible with the transposase are derived from an mPing or mPing-like MITE.

In some aspects, the transposition sequences are transposition sequences of a miniature inverted-repeat transposable element (MITE). In some aspects, the MITE is an mPing MITE. In some aspects, transposition sequences of the mPing MITE comprise mPing inverted repeat 1 and inverted repeat 2.

In some aspects, mPing inverted repeat 1 comprises a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 7. In some aspects, mPing inverted repeat 1 comprises a nucleotide sequence comprising at least about 75% or more, at least about 85% or more, at least about 95% or more, or 100% sequence identity with the nucleic acid sequence of SEQ ID NO: 7.

In some aspects, mPing inverted repeat 2 comprises a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 8. In some aspects, mPing inverted repeat 2 comprises a nucleotide sequence comprising at least about 75% or more, at least about 85% or more, at least about 95% or more, or 100% sequence identity with the nucleic acid sequence of SEQ ID NO: 8.

In some aspects, the nucleic acid construct comprising the donor polynucleotide comprises a nucleotide sequence comprising heat shock element (HSE) sequences flanked by mPing inverted repeat 1 and inverted repeat 2. In some aspects, the donor polynucleotide comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 69 to base 512 of SEQ ID NO: 81. In some aspects, the donor polynucleotide comprises a nucleic acid sequence comprising at least about 75% or more, at least about 85% or more, at least about 95% or more, or 100% sequence identity with the nucleic acid sequence starting at base 69 to base 512 of SEQ ID NO: 93. In some aspects, the nucleic acid construct comprising the donor polynucleotide comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 69 to base 512 of SEQ ID NO: 93. In some aspects, the nucleic acid construct comprising the donor polynucleotide comprises at least about 75% or more, at least about 85% or more, at least about 95% or more, or 100% sequence identity with the nucleic acid sequence starting at base 69 to base 512 of SEQ ID NO: 93.

The system can further comprise a nucleic acid expression construct comprising a promoter operably linked to a polynucleotide sequence encoding a GFP reporter, wherein the donor polynucleotide is inserted in the nucleic acid expression construct. In some aspects, the nucleic acid expression construct comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at nucleotide 2414 to nucleotide 23460 and nucleotide 1 to nucleotide 42 of SEQ ID NO: 74. In some aspects, the nucleic acid expression construct comprises at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74.

### (c) Programmable Targeting Nuclease

The system comprises a programmable targeting nuclease. A programmable targeting nuclease can be any single or group of components capable of targeting components of the engineered system to a target nucleic acid locus to mediate insertion of the donor polynucleotide into a target locus. The target nucleic acid locus can be in a coding or regulatory region of interest or can be in any other location in a nucleic acid sequence of interest. A gene can be a protein-coding gene, an RNA coding gene, or an intergenic region. The target nucleic acid locus can be in a nuclear, organellar, or extrachromosomal nucleic acid sequence. The cell can be a eukaryotic cell. In some aspects, the cell is a plant cell. In some aspects, the plant is a soybean plant.

As used herein, a “programmable polynucleotide targeting nuclease” generally comprise a programmable, sequence-specific nucleic acid-binding domain and a nuclease domain. Non-limiting examples of programmable polynucleotide targeting nucleases include, without limit, an RNA-guided clustered regularly interspersed short palindromic repeats (CRISPR)/CRISPR-associated (Cas) (CRISPR/Cas) nuclease system, a CRISPR/Cpf1 nuclease system, a zinc finger nuclease (ZFN), a transcription activator-like effector nuclease (TALEN), a meganuclease, a ribozyme, or a programmable DNA binding domain linked to a nuclease domain. Other suitable programmable polynucleotide targeting nucleases will be recognized by individuals skilled in the art.

In some aspects, the programmable polynucleotide targeting nuclease is a programmable nucleic acid editing system. Such editing systems can be engineered to edit specific DNA or RNA sequences to repress transcription or translation of an mRNA encoded by the gene, and/or produce mutant proteins with reduced activity or stability. Non-limiting examples of programmable polynucleotide targeting nucleases include, without limit, an RNA-guided clustered regularly interspersed short palindromic repeats (CRISPR) system, such as a CRISPR-associated (Cas) (CRISPR/Cas) nuclease system, a CRISPR/Cpf1 nuclease system, a zinc finger nuclease (ZFN) system, a transcription activator-like effector nuclease (TALEN) system, a MegaTAL, a homing endonuclease (HE), a meganuclease, a ribozyme, or a programmable DNA binding domain linked to a nuclease domain. Other suitable programmable polynucleotide targeting nucleases will be recognized by individuals skilled in the art. Such systems rely for specificity on the delivery of exogenous protein(s), and/or a guide RNA (gRNA) or single guide RNA (sgRNA) having a sequence which binds specifically to a gene sequence of interest. When the programmable polynucleotide targeting nuclease comprises more than one component, such as a protein and a guide nucleic acid, the multi-component modification system can be modular, in that the different components may optionally be distributed among two or more nucleic acid constructs as described herein. The components can be delivered by a plasmid or viral vector or as a synthetic oligonucleotide. More detailed descriptions of programmable nucleic acid editing system can be as described further below.

The programmable nucleic acid-binding domain may be designed or engineered to recognize and bind different nucleic acid sequences. In some aspects, the nucleic acid-binding domain is mediated by interaction between a protein and the target nucleic acid sequence. Thus, the nucleic acid-binding domain may be programmed to bind a nucleic acid sequence of interest by protein engineering. Methods of programming a nucleic acid domain are well recognized in the art.

In other targeting nucleases, the nucleic acid-binding domain is mediated by a guide nucleic acid that interacts with a protein of the targeting nuclease and the target nucleic acid sequence. In such instances, the programmable nucleic acid-binding domain may be targeted to a nucleic acid sequence of interest by designing the appropriate guide nucleic acid. Methods of designing guide nucleic acids are recognized in the art when provided with a target sequence using available tools that are capable of designing functional guide nucleic acids. It will be recognized that gRNA sequences and design of guide nucleic acids can and will vary at least depending on the particular nuclease used. By way of non-limiting example, guide nucleic acids optimized by sequence for use with a Cas9 nuclease, are likely to differ from guide nucleic acids optimized for use with a CPF1 nuclease, though it is also recognized that the target site location is a key factor in determining guide RNA sequences.

When a targeting nuclease comprises more than one component, such as a protein and a guide nucleic acid, the multi-component targeting nuclease can be modular, in that the different components may optionally be distributed among two or more nucleic acid constructs as described herein.

In some aspects, the programmable targeting nuclease is a CRISPR/Cas nuclease system comprising a nuclease and a guide RNA (gRNA). In some aspects, the targeting nuclease comprises an active nuclease domain. In other aspects, the nuclease activity of the targeting nuclease is altered to only nick or cut a single strand of the double stranded nucleic acid sequence. In some aspects, the programmable targeting nuclease is a CRISPR/Cas system. In some aspects, the CRISPR/Cas system is a CRISPR/Cas9 system and a gRNA.

In some aspects, the Cas9 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 5. In some aspects, the Cas9 protein comprises an amino acid sequence comprising at least about 75% or more, at least about 85% or more, at least about 95% or more, or 100% sequence identity with amino acid sequence of SEQ ID NO: 5.

In some aspects, a nucleic acid sequence encoding the Cas9 protein comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 6. In some aspects, a nucleic acid sequence encoding the Cas9 protein comprises at least about 75% or more, at least about 85% or more, at least about 95% or more, or 100% sequence identity with the nucleic acid sequence of SEQ ID NO: 6.

In some aspects, a nucleic acid sequence encoding the Cas9 nuclease is a deCas9 nickase, and a nucleic acid expression construct for expressing the deCas9 nickase comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 89. In some aspects, a nucleic acid sequence encoding the Cas9 nuclease is a deCas9 nickase, and a nucleic acid expression construct for expressing the deCas9 nickase comprises a nucleic acid sequence comprises at least about 75% or more, at least about 85% or more, at least about 95% or more, or 100% sequence identity with the nucleic acid sequence starting at nucleotide 8218 to nucleotide 13856 of SEQ ID NO: 89.

In some aspects, the gRNA comprises a nucleic acid sequence of SEQ ID NO: 65, SEQ ID NO: 66, SEQ ID NO: 67, SEQ ID NO: 80, or any combination thereof.

In some aspects, the targeting nuclease is not linked to the transposase. In some aspects, the system comprises a nucleic acid nucleic acid expression construct for expressing a Pong ORF1 protein, a nucleic acid nucleic acid expression construct for expressing a Pong ORF2 protein, and a nucleic acid nucleic acid expression construct for expressing a Cas9 nuclease protein.

In other aspects, a transposase of the instant disclosure is linked to the programmable targeting nuclease. In some aspects, the system comprises a nucleic acid nucleic acid expression construct for expressing a Pong ORF1 protein and a nucleic acid nucleic acid expression construct for expressing a Pong ORF2 protein fused to Cas9 nuclease.

Multiple useful methods of linking proteins are known in the art and included herein. For instance, the targeting nuclease can be linked to the transposase by at least one peptide linker. Protein linkers aid fusion protein design by providing appropriate spacing between domains, supporting correct protein folding in the case that N or C termini interactions are crucial to folding. Commonly, protein linkers permit important domain interactions, reinforce stability, and reduce steric hindrance, making them preferred for use in fusion protein design even when N and C termini can be fused. Linkers can be flexible (e.g., comprising small, non-polar (e.g., Gly) or polar (e.g., Ser, Thr) amino acids). Rigid linkers can be formed of large, cyclic proline residues, which can be helpful when highly specific spacing between domains must be maintained. In vivo cleavable linkers are designed to allow the release of one or more fused domains under certain reaction conditions, such as a specific pH gradient, or when coming in contact with another biomolecule in the cell. Examples of suitable linkers are well known in the art, and programs to design linkers are readily available (Crasto et al., Protein Eng., 2000, 13(5):3096-312), the disclosure of which is incorporated herein in its entirety. Non-limiting examples of suitable linkers include GGSGGGSG (SEQ ID NO: 68) and (GGGGS)1-4 (SEQ ID NO: 69). Alternatively, the linker may be rigid, such as AEAAAKEAAAKA (SEQ ID NO: 70), AEAAAKEAAAKEAAAKA (SEQ ID NO: 71), PAPAP (AP)6-8 (SEQ ID NO: 72), GIHGVPAA (SEQ ID NO: 73), EAAAK (SEQ ID NO:76), EAAAKEAAAK (SEQ ID NO: 77), EAAAK EAAAK EAAAK (SEQ ID NO: 78), and EAAAKEAAAKEAAAKEAAAK (SEQ ID NO: 79). Other examples of suitable linkers are well known in the art, and programs to design linkers are readily available (Crasto et al., Protein Eng., 2000, 13(5):3096-312). In alternate aspects, the targeting nuclease and the transposase can be linked directly.

i. CRISPR Nuclease Systems.

The programmable targeting nuclease can be an RNA-guided CRISPR endonuclease system. The CRISPR system comprises a guide RNA or sgRNA to a target sequence at which a protein of the system introduces a double-stranded break in a target nucleic acid sequence, and a CRISPR-associated endonuclease. The gRNA is a short synthetic RNA comprising a sequence necessary for endonuclease binding, and a preselected ˜20 nucleotide spacer sequence targeting the sequence of interest in a genomic target. Non-limiting examples of endonucleases include Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or any combination thereof.

The CRISPR nuclease system may be derived from any type of CRISPR system, including a type I (i.e., IA, IB, IC, ID, IE, or IF), type II (i.e., IIA, IIB, or IIC), type Ill (i.e., IIIA or IIIB), or type V CRISPR system. The CRISPR/Cas system may be from Streptococcus sp. (e.g., Streptococcus pyogenes), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., or Thermosipho sp.

Non-limiting examples of suitable CRISPR systems include CRISPR/Cas systems, CRISPR/Cpf systems, CRISPR/Cmr systems, CRISPR/Csa systems, CRISPR/Csb systems, CRISPR/Csc systems, CRISPR/Cse systems, CRISPR/Csf systems, CRISPR/Csm systems, CRISPR/Csn systems, CRISPR/Csx systems, CRISPR/Csy systems, CRISPR/Csz systems, and derivatives or variants thereof. Preferably, the CRISPR system may be a type II Cas9 protein, a type V Cpf1 protein, or a derivative thereof. In some aspects, the CRISPR/Cas nuclease is Streptococcus pyogenes Cas9 (SpCas9), Streptococcus thermophilus Cas9 (StCas9), Campylobacter jejuni Cas9 (CjCas9), Francisella novicida Cas9 (FnCas9), or Francisella novicida Cpf1 (FnCpf1).

In general, a protein of the CRISPR system comprises a RNA recognition and/or RNA binding domain, which interacts with the guide RNA. A protein of the CRISPR system also comprises at least one nuclease domain having endonuclease activity. For example, a Cas9 protein may comprise a RuvC-like nuclease domain and an HNH-like nuclease domain, and a Cpf1 protein may comprise a RuvC-like domain. A protein of the CRISPR system may also comprise DNA binding domains, helicase domains, RNase domains, protein-protein interaction domains, dimerization domains, as well as other domains.

A protein of the CRISPR system may be associated with guide RNAs (gRNA). The guide RNA may be a single guide RNA (i.e., sgRNA), or may comprise two RNA molecules (i.e., crRNA and tracrRNA). The guide RNA interacts with a protein of the CRISPR system to guide it to a target site in the DNA. The target site has no sequence limitation except that the sequence is bordered by a protospacer adjacent motif (PAM). For example, PAM sequences for Cas9 include 3′-NGG, 3′-NGGNG, 3′-NNAGAAW, and 3′-ACAY, and PAM sequences for Cpf1 include 5′-TTN (wherein N is defined as any nucleotide, W is defined as either A or T, and Y is defined as either C or T). Each gRNA comprises a sequence that is complementary to the target sequence (e.g., a Cas9 gRNA may comprise GN17-20GG). The gRNA may also comprise a scaffold sequence that forms a stem loop structure and a single-stranded region. The scaffold region may be the same in every gRNA. In some aspects, the gRNA may be a single molecule (i.e., sgRNA). In other aspects, the gRNA may be two separate molecules. Those skilled in the art are familiar with gRNA design and construction, e.g., gRNA design tools are available on the internet or from commercial sources.

A CRISPR system may comprise one or more nucleic acid binding domains associated with one or more, or two or more selected guide RNAs used to direct the CRISPR system to one or more, or two or more selected target nucleic acid loci. For instance, a nucleic acid binding domain may be associated with one or more, or two or more selected guide RNAs, each selected guide RNA, when complexed with a nucleic acid binding domain, causing the CRISPR system to localize to the target of the guide RNA.

ii. CRISPR nickase systems.

The programmable targeting nuclease can also be a CRISPR nickase system. CRISPR nickase systems are similar to the CRISPR nuclease systems described above except that a CRISPR nuclease of the system is modified to cleave only one strand of a double-stranded nucleic acid sequence. Thus, a CRISPR nickase, in combination with a guide RNA of the system, may create a single-stranded break or nick in the target nucleic acid sequence. Alternatively, a CRISPR nickase in combination with a pair of offset gRNAs may create a double-stranded break in the nucleic acid sequence.

A CRISPR nuclease of the system may be converted to a nickase by one or more mutations and/or deletions. For example, a Cas9 nickase may comprise one or more mutations in one of the nuclease domains, wherein the one or more mutations may be D10A, E762A, and/or D986A in the RuvC-like domain, or the one or more mutations may be H840A (or H839A), N854A and/or N863A in the HNH-like domain.

iii. ssDNA-Guided Argonaute Systems.

Alternatively, the programmable targeting nuclease may comprise a single-stranded DNA-guided Argonaute endonuclease. Argonautes (Agos) are a family of endonucleases that use 5′-phosphorylated short single-stranded nucleic acids as guides to cleave nucleic acid targets. Some prokaryotic Agos use single-stranded guide DNAs and create double-stranded breaks in nucleic acid sequences. The ssDNA-guided Ago endonuclease may be associated with a single-stranded guide DNA.

The Ago endonuclease may be derived from Alistipes sp., Aquifex sp., Archaeoglobus sp., Bacteroides sp., Bradyrhizobium sp., Burkholderia sp., Cellvibrio sp., Chlorobium sp., Geobacter sp., Mariprofundus sp., Natronobacterium sp., Parabacteriodes sp., Parvularcula sp., Planctomyces sp., Pseudomonas sp., Pyrococcus sp., Thermus sp., or Xanthomonas sp. For instance, the Ago endonuclease may be Natronobacterium gregoryi Ago (NgAgo). Alternatively, the Ago endonuclease may be Thermus thermophilus Ago (TtAgo). The Ago endonuclease may also be Pyrococcus furiosus (PfAgo).

The single-stranded guide DNA (gDNA) of an ssDNA-guided Argonaute system is complementary to the target site in the nucleic acid sequence. The target site has no sequence limitations and does not require a PAM. The gDNA generally ranges in length from about 15-30 nucleotides. The gDNA may comprise a 5′ phosphate group. Those skilled in the art are familiar with ssDNA oligonucleotide design and construction.

iv. Zinc finger nucleases.

The programmable targeting nuclease may be a zinc finger nuclease (ZFN). A ZFN comprises a DNA-binding zinc finger region and a nuclease domain. The zinc finger region may comprise from about two to seven zinc fingers, for example, about four to six zinc fingers, wherein each zinc finger binds three nucleotides. The zinc finger region may be engineered to recognize and bind to any DNA sequence. Zinc finger design tools or algorithms are available on the internet or from commercial sources. The zinc fingers may be linked together using suitable linker sequences.

A ZFN also comprises a nuclease domain, which may be obtained from any endonuclease or exonuclease. Non-limiting examples of endonucleases from which a nuclease domain may be derived include, but are not limited to, restriction endonucleases and homing endonucleases. The nuclease domain may be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MboII, and SapI. The type II-S nuclease domain may be modified to facilitate dimerization of two different nuclease domains. For example, the cleavage domain of FokI may be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI nuclease domains are targets for modification. For example, one modified FokI domain may comprise Q486E, 1499L, and/or N496D mutations, and the other modified FokI domain may comprise E490K, 1538K, and/or H537R mutations.

v. Transcription Activator-Like Effector Nuclease Systems.

The programmable targeting nuclease may also be a transcription activator-like effector nuclease (TALEN) or the like. TALENs comprise a DNA-binding domain composed of highly conserved repeats derived from transcription activator-like effectors (TALEs) that are linked to a nuclease domain. TALEs are proteins secreted by plant pathogen Xanthomonas to alter transcription of genes in host plant cells. TALE repeat arrays may be engineered via modular protein design to target any DNA sequence of interest. Other transcription activator-like effector nuclease systems may comprise, but are not limited to, the repetitive sequence, transcription activator like effector (RipTAL) system from the bacterial plant pathogenic Ralstonia solanacearum species complex (Rssc). The nuclease domain of TALEs may be any nuclease domain as described above in Section (1)(c)(i).

vi. Meganucleases or Rare-Cutting Endonuclease Systems.

The programmable targeting nuclease may also be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG has become a valuable tool for the study of genomes and genome engineering. Non-limiting examples of meganucleases that may be suitable for the instant disclosure include I-SceI, I-CreI, I-DmoI, or variants and combinations thereof. A meganuclease may be targeted to a specific nucleic acid sequence by modifying its recognition sequence using techniques well known to those skilled in the art.

The programmable targeting nuclease can be a rare-cutting endonuclease or derivative thereof. Rare-cutting endonucleases are site-specific endonucleases whose recognition sequence occurs rarely in a genome, such as only once in a genome. The rare-cutting endonuclease may recognize a 7-nucleotide sequence, an 8-nucleotide sequence, or longer recognition sequence. Non-limiting examples of rare-cutting endonucleases include NotI, AscI, Pac, AsiSI, SbfI, and FseI.

vii. Optional Additional Domains.

The programmable targeting nuclease may further comprise at least one nuclear localization signal (NLS), at least one cell-penetrating domain, at least one reporter domain, and/or at least one linker.

In general, an NLS comprises a stretch of basic amino acids. Nuclear localization signals are known in the art (see, e.g., Lange et al., J. Biol. Chem., 2007, 282:5101-5105). The NLS may be located at the N-terminus, the C-terminal, or in an internal location of the fusion protein.

A cell-penetrating domain may be a cell-penetrating peptide sequence derived from the HIV-1 TAT protein. The cell-penetrating domain may be located at the N-terminus, the C-terminal, or in an internal location of the fusion protein.

A programmable targeting nuclease may further comprise at least one linker. For example, the programmable targeting nuclease, the nuclease domain of the targeting nuclease, and other optional domains may be linked via one or more linkers. The linker may be flexible (e.g., comprising small, non-polar (e.g., Gly) or polar (e.g., Ser, Thr) amino acids). Examples of suitable linkers are well known in the art, and programs to design linkers are readily available (Crasto et al., Protein Eng., 2000, 13(5):3096-312). In alternate aspects, the programmable targeting nuclease, the cell cycle regulated protein, and other optional domains may be linked directly.

A programmable targeting nuclease may further comprise an organelle localization or targeting signal that directs a molecule to a specific organelle. A signal may be polynucleotide or polypeptide signal, or may be an organic or inorganic compound sufficient to direct an attached molecule to a desired organelle. Organelle localization signals can be as described in U.S. Patent Publication No. 20070196334, the disclosure of which is incorporated herein in its entirety.

### (d) Engineered System

An engineered system of the instant disclosure generally comprises a nucleic acid expression construct for expressing a tranposase, wherein the expression construct comprises a promoter operably linked to a nucleic acid sequence encoding a transposase. The engineered system also comprises a nucleic acid construct comprising a donor polynucleotide comprising nucleic acid transposition sequences compatible with the transposase and a nucleic acid expression construct for expressing a programmable targeting nuclease, wherein the expression construct comprises a promoter operably linked to a nucleic acid sequence encoding a programmable targeting nuclease. The targeting nuclease is engineered to introduce a cut in a target nucleic acid locus thereby guiding insertion of the donor polynucleotide at the target nucleic acid locus by the transposase to generate a genetically engineered cell comprising the donor polynucleotide inserted at the target nucleic acid locus. The transposase can be linked to the targeting nuclease. Alternatively, the transposase is not linked to the targeting nuclease.

The system can further comprise a nucleic acid expression construct comprising a promoter operably linked to a polynucleotide sequence encoding a reporter, wherein the donor polynucleotide is inserted in the nucleic acid expression construct, wherein the reporter is inactivated by the inserted nucleic acid construct comprising the donor polynucleotide, and wherein the reporter is activated by excision of the inserted nucleic acid construct comprising the donor polynucleotide from the expression construct comprising a promoter operably linked to a polynucleotide sequence encoding a reporter by the transposase. In some aspects, the reporter can be GFP, and the GFP expression construct, wherein the donor polynucleotide is inserted in the nucleic acid expression construct, comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at nucleotide 2414 to nucleotide 23460 and nucleotide 1 to nucleotide 42 of SEQ ID NO: 74. In some aspects, the reporter can be GFP, and the GFP expression construct, wherein the donor polynucleotide is inserted in the nucleic acid expression construct, comprises a nucleic acid sequence comprising at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74.

The transposase can be a split transposase. When the transposase is a split transposase, the transposase can be a Pong or Pong-like transposase comprising a Pong ORF1 protein and a Pong ORF2 protein. In some aspects, the Pong ORF1 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 1. In some aspects, the Pong ORF1 protein comprises an amino acid sequence comprising at least about 75% or more, at least about 85% or more, at least about 95% or more, or 100% sequence identity with the amino acid sequence of SEQ ID NO: 1. A nucleic acid sequence encoding the Pong ORF1 protein can comprise about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 2. A nucleic acid sequence encoding the Pong ORF1 protein can comprise at least about 75% or more, at least about 85% or more, at least about 95% or more, or 100% sequence identity with the nucleic acid sequence of SEQ ID NO: 2.

In some aspects, the Pong ORF2 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 3. In some aspects, the Pong ORF2 protein comprises an amino acid sequence comprising at least about 75% or more, at least about 85% or more, at least about 95% or more, or 100% sequence identity with the amino acid sequence of SEQ ID NO: 3. A nucleic acid sequence encoding the Pong ORF2 protein can comprise about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 4. A nucleic acid sequence encoding the Pong ORF2 protein can comprise at least about 75% or more, at least about 85% or more, at least about 95% or more, or 100% sequence identity with the amino acid sequence of SEQ ID NO: 4.

The transposition sequences can be transposition sequences of a miniature inverted-repeat transposable element (MITE). In some aspects, the MITE is an mPing MITE or a derivative of mPing with sequences added or removed. In some aspects, transposition sequences of the mPing MITE comprise mPing inverted repeat 1 and inverted repeat 2. In some aspects, mPing inverted repeat 1 comprises a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 7. In some aspects, mPing inverted repeat 1 comprises a nucleotide sequence comprising at least about 75% or more, at least about 85% or more, at least about 95% or more, or 100% sequence identity with the nucleic acid sequence of SEQ ID NO: 7. In some aspects, mPing inverted repeat 2 comprises a nucleotide sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 8. In some aspects, mPing inverted repeat 2 comprises a nucleotide sequence comprising at least about 75% or more, at least about 85% or more, at least about 95% or more, or 100% sequence identity with the nucleic acid sequence of SEQ ID NO: 8.

In some aspects, the programmable targeting nuclease comprises a programmable, sequence-specific nucleic acid-binding domain and a nuclease domain. For instance, the programmable targeting nuclease is an RNA-guided clustered regularly interspersed short palindromic repeats (CRISPR)/CRISPR-associated (Cas) (CRISPR/Cas) nuclease system, a zinc finger nuclease (ZFN), a transcription activator-like effector nuclease (TALEN), a meganuclease, a ssDNA-guided Argonaute endonuclease, a meganuclease, a rare-cutting endonuclease, or any combination thereof.

In some aspects, the programmable targeting nuclease is a CRISPR/Cas nuclease system comprising a nuclease and a guide RNA (gRNA). In some aspects, the programmable targeting nuclease is a CRISPR/Cas nuclease system comprising a nuclease and a guide RNA (gRNA). In some aspects, the targeting nuclease comprises an active nuclease domain. In other aspects, the nuclease activity of the targeting nuclease is altered to only nick or cut a single strand of the double stranded nucleic acid sequence. In some aspects, the programmable targeting nuclease is a CRISPR/Cas system. In some aspects, the CRISPR/Cas system is a CRISPR/Cas9 system and a gRNA.

In some aspects, the Cas9 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 5. In some aspects, the Cas9 protein comprises an amino acid sequence comprising at least about 75% or more, at least about 85% or more, at least about 95% or more, or 100% sequence identity with the amino acid sequence of SEQ ID NO: 5. In some aspects, the Cas9 nuclease is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 6. In some aspects, the Cas9 nuclease is encoded by a nucleic acid sequence comprising at least about 75% or more, at least about 85% or more, at least about 95% or more, or 100% sequence identity with the nucleic acid sequence of SEQ ID NO: 6.

In some aspects, the gRNA comprises a nucleic acid sequence of SEQ ID NO: 65, SEQ ID NO: 66, SEQ ID NO: 67, SEQ ID NO: 80, or any combination thereof.

As explained in Section II further below, a system of the instant disclosure can be encoded on one or more nucleic acid constructs encoding the components of the system. Depending on an intended use of the system of the instant disclosure, the number of nucleic acid constructs encoding the components of the system can be on different plasmids based on intended use. For instance, the systems can be a one-component system comprising all the elements of the system. Such a system can provide the convenience and simplicity of introducing a single nucleic acid construct into a cell. Accordingly, in some aspects, a system of the instant disclosure is a one-component system comprising a nucleic acid expression construct for expressing a tranposase, a nucleic acid construct comprising a donor polynucleotide, and a nucleic acid expression construct for expressing a programmable targeting nuclease.

In some aspects, a system of the instant disclosure is a one-component system, wherein the transposase is a Pong transposase, wherein the nucleic acid transposition sequences are mPing inverted repeat 1 and inverted repeat 2, and the programmable targeting nuclease comprises a Cas9 nuclease and a gRNA. In some aspects, the Pong ORF2 protein is fused to the Cas9 nuclease. In some aspects, the Pong ORF2 protein is not fused to the Cas9 nuclease.

In some aspects, a system of the instant disclosure is a one-component system, wherein the Pong ORF2 protein is fused to the Cas9 nuclease and the donor polynucleotide is inserted in a nucleic acid expression construct encoding a GFP reporter, thereby inactivating the reporter. In these aspects, the target nucleic acid locus is in an Arabidopsis PDS3 gene.

In some aspects, a system of the instant disclosure is a one-component system, wherein the Pong ORF2 protein is fused to the Cas9 nuclease and the donor polynucleotide is inserted in a nucleic acid expression construct encoding a GFP reporter, thereby inactivating the reporter. In these aspects, the target nucleic acid locus is in an actin 8 (ACT8) gene.

In other aspects, a system of the instant disclosure is a one-component system, wherein the Pong ORF2 protein fused to a Cas9 nuclease and the target nucleic acid locus is in an Arabidopsis actin 8 (ACT8) gene. In these aspects, the donor polynucleotide can comprise a nucleotide sequence comprising heat shock element (HSE) sequences flanked by mPing inverted repeat 1 and inverted repeat 2.

In some aspects, a system of the instant disclosure is a one-component system, wherein the Cas9 protein is not fused to the Pong ORF2 protein, and the target nucleic acid locus is in a soybean DD20 intergenic region.

In some aspects, a system of the instant disclosure is a one-component system, wherein the Cas9 protein is fused to the Pong ORF2 protein, the donor construct is inserted in an expression construct expressing a GFP reporter, and the target nucleic acid locus is in a soybean DD20 intergenic region.

Alternatively, a system of the instant disclosure can be encoded on more than one nucleic acid construct. In some aspects, a system of the instant disclosure is a two-component system comprising a donor nucleic acid construct comprising the nucleic acid construct comprising a donor polynucleotide of the instant disclosure, and a helper nucleic acid construct comprising a nucleic acid expression construct for expressing a tranposase and the nucleic acid expression construct for expressing the programmable targeting nuclease of the instant disclosure.

In some aspects, a system of the instant disclosure comprises a helper construct and a donor construct, wherein the donor construct comprises the donor polynucleotide, and wherein the helper construct comprises the nucleic acid expression construct for expressing a tranposase and the nucleic acid expression construct for expressing a programmable targeting nuclease. In some aspects, a system of the instant disclosure the transposase is a Pong transposase, the nucleic acid transposition sequences are mPing inverted repeat 1 and inverted repeat 2, and the programmable targeting nuclease comprises a Cas9 nuclease and a gRNA. In some aspects, the Pong ORF2 protein is fused to the Cas9 nuclease. In some aspects, the Pong ORF2 protein is not fused to the Cas9 nuclease, and is expressed from a different expression construct. In some aspects, the Cas9 nuclease is a Cas9 nickase.

In some aspects, the system of the instant disclosure comprises a helper construct and a donor construct, wherein the helper construct comprises a nucleic acid expression construct for expressing Pong ORF1 and a nucleic acid expression construct for expressing Pong ORF2 protein fused to a Cas9 nuclease. In some aspects, the donor polynucleotide is inserted in a nucleic acid expression construct encoding a GFP reporter, thereby inactivating the reporter. In some aspects, the expression construct is inserted in nucleic acid sequence in the genome of the cell. In some aspects, the target nucleic acid locus is in an Arabidopsis PDS3 gene.

In some aspects, the system of the instant disclosure comprises a helper construct and a donor construct, wherein the helper construct comprises a nucleic acid expression construct for expressing Pong ORF1, a nucleic acid expression construct for expressing Pong ORF2 protein, a nucleic acid construct for expressing a deCas9 nickase. In some aspects, the donor construct comprises a nucleic acid expression construct encoding a GFP reporter, wherein the donor nucleic acid construct is inserted into the expression construct thereby inactivating the reporter. In these aspects, the target nucleic acid locus is an Arabidopsis ACT8 gene.

In some aspects, the system of the instant disclosure comprises a helper construct, wherein the helper construct comprises a nucleic acid expression construct for expressing Pong ORF1 and a nucleic acid expression construct for expressing Pong ORF2 protein, wherein the Cas9 nuclease is a deCas9 nickase, wherein the Pong ORF2 protein is not fused to the deCas9 nickase and the target nucleic acid locus is in an Arabidopsis actin 8 (ADH1) gene.

II. Nucleic Acid Constructs

A further aspect of the present disclosure provides one or more nucleic acid constructs encoding the components of the system described above in Section I. In some aspects, the system of nucleic acid constructs encodes the engineered system described in Section I(d).

Any of the multi-component systems described herein are to be considered modular, in that the different components may optionally be distributed among two or more nucleic acid constructs as described herein. The nucleic acid constructs may be DNA or RNA, linear or circular, single-stranded or double-stranded, or any combination thereof. The nucleic acid constructs may be codon optimized for efficient translation into protein, and possibly for transcription into an RNA donor polynucleotide transcript in the cell of interest. Codon optimization programs are available as freeware or from commercial sources.

The nucleic acid constructs can be used to express one or more components of the system for later introduction into a cell to be genetically modified. Alternatively, the nucleic acid constructs can be introduced into the cell to be genetically modified for expression of the components of the system in the cell.

Expression constructs generally comprise DNA coding sequences operably linked to at least one promoter control sequence for expression in a cell of interest. Promoter control sequences may control expression of the transposase, the programmable targeting nuclease, the donor polynucleotide, or combinations thereof in bacterial (e.g., E. coli) cells or eukaryotic (e.g., yeast, insect, mammalian, or plant) cells. Suitable bacterial promoters include, without limit, T7 promoters, lac operon promoters, trp promoters, tac promoters (which are hybrids of trp and lac promoters), variations of any of the foregoing, and combinations of any of the foregoing. Non-limiting examples of suitable eukaryotic promoters include constitutive, regulated, or cell- or tissue-specific promoters. Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-3 promoter, Mb promoter, NphsI promoter, OG-2 promoter, SP-B promoter, SYN1 promoter, and WASP promoter.

Promoters may also be plant-specific promoters, or promoters that may be used in plants. A wide variety of plant promoters are known to those of ordinary skill in the art, as are other regulatory elements that may be used alone or in combination with promoters. Preferably, promoter control sequences control expression in cassava such as promoters disclosed in Wilson et al., 2017, The New Phytologist, 213(4):1632-1641, the disclosure of which is incorporated herein in its entirety.

Promoters may be divided into two types, namely, constitutive promoters and non-constitutive promoters. Constitutive promoters are classified as providing for a range of constitutive expression. Thus, some are weak constitutive promoters, and others are strong constitutive promoters. Non-constitutive promoters include tissue-preferred promoters, tissue-specific promoters, cell-type specific promoters, and inducible-promoters. Suitable plant-specific constitutive promoter control sequences include, but are not limited to, a CaMV35S promoter, CaMV 19S, GOS2, Arabidopsis At6669 promoter, Rice cyclophilin, Maize H3 histone, Synthetic Super MAS, an opine promoter, a plant ubiquitin (Ubi) promoter, an actin 1 (Act-1) promoter, pEMU, Cestrum yellow leaf curling virus promoter (CYMLV promoter), and an alcohol dehydrogenase 1 (Adh-1) promoter. Other constitutive promoters include those in U.S. Pat. Nos. 5,659,026; 5,608,149; 5,608,144; 5,604,121; 5,569,597; 5,466,785; 5,399,680; 5,268,463; and 5,608,142.

Regulated plant promoters respond to various forms of environmental stresses, or other stimuli, including, for example, mechanical shock, heat, cold, flooding, drought, salt, anoxia, pathogens such as bacteria, fungi, and viruses, and nutritional deprivation, including deprivation during times of flowering and/or fruiting, and other forms of plant stress. For example, the promoter may be a promoter which is induced by one or more, but not limited to one of the following: abiotic stresses such as wounding, cold, desiccation, ultraviolet-B, heat shock or other heat stress, drought stress or water stress. The promoter may further be one induced by biotic stresses including pathogen stress, such as stress induced by a virus or fungi, stresses induced as part of the plant defense pathway or by other environmental signals, such as light, carbon dioxide, hormones or other signaling molecules such as auxin, hydrogen peroxide and salicylic acid, sugars and gibberellin or abscisic acid and ethylene. Suitable regulated plant promoter control sequences include, but are not limited to, salt-inducible promoters such as RD29A; drought-inducible promoters such as maize rab17 gene promoter, maize rab28 gene promoter, and maize Ivr2 gene promoter; heat-inducible promoters such as heat tomato hsp80-promoter from tomato.

Tissue-specific promoters may include, but are not limited to, fiber-specific, green tissue-specific, root-specific, stem-specific, flower-specific, callus-specific, pollen-specific, egg-specific, and seed coat-specific. Suitable tissue-specific plant promoter control sequences include, but are not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993], seed-preferred promoters [e.g., from seed-specific genes (Simon et al., Plant Mol. Biol. 5. 191, 1985; Scofield et al., J. Biol. Chem. 262: 12202, 1987; Baszczynski et al., Plant Mol. Biol. 14: 633, 1990), Brazil Nut albumin (Pearson et al., Plant Mol. Biol. 18: 235-245, 1992), legumin (Ellis et al., Plant Mol. Biol. 10: 203-214, 1988), Glutelin (rice) (Takaiwa et al., Mol. Gen. Genet. 208: 15-22, 1986; Takaiwa et al., FEBS Letts. 221: 43-47, 1987), Zein (Matzke et al., Plant Mol Biol, 143: 323-32, 1990), napA (Stalberg et al., Planta 199: 515-519, 1996), Wheat SPA (Albanietal, Plant Cell, 9: 171-184, 1997), sunflower oleosin (Cummins et al., Plant Mol. Biol. 19: 873-876, 1992)], endosperm specific promoters [e.g., wheat LMW and HMW, glutenin-1 (Mol Gen Genet 216:81-90, 1989; NAR 17:461-2), wheat a, b and g gliadins (EMB03:1409-15, 1984), Barley ItrI promoter, barley B1, C, D hordein (Theor Appl Gen 98:1253-62, 1999; Plant J 4:343-55, 1993; Mol Gen Genet 250:750-60, 1996), Barley DOF (Mena et al., The Plant Journal, 116(1): 53-62, 1998), Biz2 (EP99106056.7), Synthetic promoter (Vicente-Carbajosa et al., Plant J. 13: 629-640, 1998), rice prolamin NRP33, rice-globulin Glb-1 (Wu et al., Plant Cell Physiology 39(8) 885-889, 1998), rice alpha-globulin REB/OHP-1 (Nakase et al., Plant Mol. Biol. 33: 513-S22, 1997), rice ADP-glucose PP (Trans Res 6:157-68, 1997), maize ESR gene family (Plant J 12:235-46, 1997), sorgum gamma-kafirin (PMB 32:1029-35, 1996)], embryo-specific promoters [e.g., rice OSH1 (Sato et al., Proc. Natl. Acad. Sci. USA, 93: 8117-8122), KNOX (Postma-Haarsma et al., Plant Mol. Biol. 39:257-71, 1999), rice oleosin (Wu et al., J. Biochem., 123:386, 1998)], and flower-specific promoters [e.g., AtPRP4, chalene synthase (chsA) (Van der Meer et al., Plant Mol. Biol. 15, 95-109, 1990), LAT52 (Twell et al., Mol. Gen Genet. 217:240-245; 1989), apetala-3].

Any of the promoter sequences may be wild type or may be modified for more efficient or efficacious expression. The DNA coding sequence also may be linked to a polyadenylation signal (e.g., SV40 polyA signal, bovine growth hormone (BGH) polyA signal, etc.) and/or at least one transcriptional termination sequence. In some situations, the complex or fusion protein may be purified from the bacterial or eukaryotic cells.

Nucleic acids encoding one or more components of a homologous recombination system and/or transcription activation system may be present in a construct. Suitable constructs include plasmid constructs, viral constructs, and self-replicating RNA (Yoshioka et al., Cell Stem Cell, 2013, 13:246-254). For instance, the nucleic acid encoding one or more components of a homologous recombination system and/or transcription activation system may be present in a plasmid construct.

Non-limiting examples of suitable plasmid constructs include pUC, pBR322, pET, pBluescript, and variants thereof. Alternatively, the nucleic acid encoding one or more components of a homologous recombination system and/or transcription activation system may be part of a viral vector (e.g., lentiviral vectors, adeno-associated viral vectors, adenoviral vectors, and so forth).

The plasmid or viral vector may comprise additional expression control sequences (e.g., enhancer sequences, Kozak sequences, polyadenylation sequences, transcriptional termination sequences, etc.), selectable reporter sequences (e.g., antibiotic resistance genes), origins of replication, T-DNA border sequences, and the like. The plasmid or viral vector may further comprise RNA processing elements such as glycine tRNAs, or Csy4 recognition sites. Such RNA processing elements can, for instance, intersperse polynucleotide sequences encoding multiple gRNAs under the control of a single promoter to produce the multiple gRNAs from a transcript encoding the multiple gRNAs. When a cys4 recognition cite is used, a vector may further comprise sequences for expression of Csy4 RNAse to process the gRNA transcript. Additional information about vectors and use thereof may be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001.

In some aspects, a system of the instant disclosure is a one-component system, wherein the Pong ORF2 protein is fused to the Cas9 nuclease and the donor polynucleotide is inserted in a nucleic acid expression construct encoding a GFP reporter, thereby inactivating the reporter. In these aspects, the target nucleic acid locus is in an Arabidopsis PDS3 gene. The system comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5073 to base 8215 of SEQ ID NO: 89. In some aspects, the nucleic acid expression construct for expressing a Pong ORF1 protein comprises at least about 75% or more, at least about 85% or more, at least about 95% or more, or 100% sequence identity with the nucleic acid sequence starting at base 5073 to base 8215 of SEQ ID NO: 89. The system also comprises a nucleic acid expression construct for expressing a Pong ORF2 protein fused to Cas9 nuclease, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 7451 to base 14807 of SEQ ID NO: 74. In some aspects, the construct for expressing a Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 7451 to base 14807 of SEQ ID NO: 74. The system further comprises a nucleic acid expression construct comprising a promoter operably linked to a polynucleotide sequence encoding GFP, wherein the donor polynucleotide inserted in the nucleic acid expression construct. In some aspects, the GFP expression construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at nucleotide 2414 to nucleotide 23460 and nucleotide 1 to nucleotide 42 of SEQ ID NO: 74. In some aspects, the GFP expression construct comprises a nucleic acid sequence comprising at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74. The system further comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 2632 to base 3343 of SEQ ID NO: 74. In some aspects, the expression construct for expressing the gRNA comprises a nucleic acid sequence comprising at least about 75% or more, at least about 85% or more, at least about 95% or more, or 100% sequence identity with the nucleic acid sequence starting at base 2632 to base 3343 of SEQ ID NO: 74. In some aspects, the system is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 74. In some aspects, the system is encoded on a plasmid comprising a nucleic acid sequence comprising at least about 75% or more, at least about 85% or more, at least about 95% or more, or 100% sequence identity with the nucleic acid sequence of SEQ ID NO: 74.

In some aspects, a system of the instant disclosure is a one-component system, wherein the Pong ORF2 protein is fused to the Cas9 nuclease and the donor polynucleotide is inserted in a nucleic acid expression construct encoding a GFP reporter, thereby inactivating the reporter. In these aspects, the target nucleic acid locus is in an actin 8 (ACT8) gene. The system comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 1456 to base 5362 of SEQ ID NO: 92. In some aspects, the nucleic acid expression construct for expressing a Pong ORF1 protein comprises at least about 75% or more, at least about 85% or more, at least about 95% or more, or 100% sequence identity with the nucleic acid sequence starting at base 1456 to base 5362 of SEQ ID NO: 92. The system also comprises a nucleic acid nucleic acid expression construct for expressing a Pong ORF2 protein fused to Cas9 nuclease, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5548 to base 12904 of SEQ ID NO: 92. In some aspects, the construct for expressing a Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 5548 to base 12904 of SEQ ID NO: 92. The system further comprises a nucleic acid construct comprising the donor polynucleotide, wherein the nucleic acid construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 69 to base 498 of SEQ ID NO: 92. In some aspects, the nucleic acid construct comprising the donor polynucleotide comprises a nucleic acid sequence comprising at least about 75% or more, at least about 85% or more, at least about 95% or more, or 100% sequence identity with the nucleic acid sequence starting at base 69 to base 498 of SEQ ID NO: 92. The system comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 729 to base 1440 of SEQ ID NO: 92. In some aspects, the expression construct for expressing the gRNA comprises a nucleic acid sequence comprising at least about 75% or more, at least about 85% or more, at least about 95% or more, or 100% sequence identity with the nucleic acid sequence starting at base 729 to base 1440 of SEQ ID NO: 92. In some aspects, the system is encoded on a plasmid comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 92. In some aspects, the system is encoded on a plasmid comprising at least about 75% or more, at least about 85% or more, at least about 95% or more, or 100% sequence identity with SEQ ID NO: 92.

In other aspects, a system of the instant disclosure is a one-component system, wherein the Pong ORF2 protein fused to a Cas9 nuclease and the target nucleic acid locus is in an Arabidopsis actin 8 (ACT8) gene. In these aspects, the donor polynucleotide comprises a nucleotide sequence comprising heat shock element (HSE) sequences flanked by mPing inverted repeat 1 and inverted repeat 2. The system comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 1481 to base 5390 of SEQ ID NO: 93. In some aspects, the nucleic acid expression construct for expressing a Pong ORF1 protein comprises at least about 75% or more, at least about 85% or more, at least about 95% or more, or 100% sequence identity with the nucleic acid sequence starting at base 1481 to base 5390 of SEQ ID NO: 93. The system also comprises a nucleic acid expression construct for expressing a Pong ORF2 protein fused to Cas9 nuclease, wherein the expression construct for expressing the Pong ORF2 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 1481 to base 5390 of SEQ ID NO: 93. In some aspects, the expression construct for expressing the Pong ORF2 protein comprises a nucleic acid sequence comprising at least about 75% or more, at least about 85% or more, at least about 95% or more, or 100% sequence identity with the nucleic acid sequence starting at base 1481 to base 5390 of SEQ ID NO: 93. The system further comprises a nucleic acid construct comprising the donor polynucleotide, wherein the donor polynucleotide comprises a nucleotide sequence comprising HSE sequences flanked by mPing inverted repeat 1 and inverted repeat 2, and wherein the donor polynucleotide comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 69 to base 512 of SEQ ID NO: 93. In some aspects, the donor polynucleotide comprises at least about 75% or more, at least about 85% or more, at least about 95% or more, or 100% sequence identity with the nucleic acid sequence starting at base 69 to base 512 of SEQ ID NO: 93. The system comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 754 to base 1465 of SEQ ID NO: 93. In some aspects, the expression construct for expressing the gRNA comprises a nucleic acid sequence comprising at least about 75% or more, at least about 85% or more, at least about 95% or more, or 100% sequence identity with the nucleic acid sequence starting at base 754 to base 1465 of SEQ ID NO: 93. In some aspects, the system is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 93. In some aspects, the system is encoded on a plasmid comprising a nucleic acid sequence comprising at least about 75% or more, at least about 85% or more, at least about 95% or more, or 100% sequence identity with the nucleic acid sequence of SEQ ID NO: 93.

In some aspects, a system of the instant disclosure is a one-component system, wherein the Cas9 protein is not fused to the Pong ORF2 protein, and the target nucleic acid locus is in a soybean DD20 intergenic region. The system comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with nucleic acid sequence starting at base 3593 to base 7502 of SEQ ID NO: 94. In some aspects, the nucleic acid expression construct for expressing a Pong ORF1 protein comprises at least about 75% or more, at least about 85% or more, at least about 95% or more, or 100% sequence identity with the nucleic acid sequence starting at base 3593 to base 7502 of SEQ ID NO: 94. The system also comprises a nucleic acid nucleic acid expression construct for expressing a Pong ORF2 protein, wherein the expression construct for expressing the Pong ORF2 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 7685 to base 10827 of SEQ ID NO: 94. In some aspects, the expression construct for expressing the Pong ORF2 protein comprises a nucleic acid sequence comprising at least about 75% or more, at least about 85% or more, at least about 95% or more, or 100% sequence identity with the nucleic acid sequence starting at base 7685 to base 10827 of SEQ ID NO: 94. The system also comprises a nucleic acid expression construct for expressing a Cas9 nuclease, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 10857 to base 16495 of SEQ ID NO: 94. In some aspects, the construct for expressing the Cas9 nuclease comprises a nucleic acid sequence comprising at least about 75% or more, at least about 85% or more, at least about 95% or more, or 100% sequence identity with the nucleic acid sequence starting at base 10857 to base 16495 of SEQ ID NO: 94. The system comprises a nucleic acid construct comprising the donor polynucleotide, wherein the nucleic acid construct comprising the donor polynucleotide comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or comprising at least about 75% or more, at least about 85% or more, at least about 95% or more, or 100% sequence identity with the nucleic acid sequence starting at base 2201 to base 2630 of SEQ ID NO: 94. The system also comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or comprising at least about 75% or more, at least about 85% or more, at least about 95% or more, or 100% sequence identity with the nucleic acid sequence starting at base 2861 to base 3572 of SEQ ID NO: 94. In some aspects, the system is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 2861 to base 3572 of SEQ ID NO: 94. In some aspects, the system is encoded on a plasmid comprising a nucleic acid sequence comprising at least about 75% or more, at least about 85% or more, at least about 95% or more, or 100% sequence identity with the nucleic acid sequence of SEQ ID NO: 94.

In some aspects, a system of the instant disclosure is a one-component system, wherein the Cas9 protein is fused to the Pong ORF2 protein, the donor construct is inserted in an expression construct expressing a GFP reporter, and the target nucleic acid locus is in a soybean DD20 intergenic region. The system comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5490 to base 9399 of SEQ ID NO: 95. In some aspects, the nucleic acid expression construct for expressing a Pong ORF1 protein comprises at least about 75% or more, at least about 85% or more, at least about 95% or more, or 100% sequence identity with the nucleic acid sequence starting at base 5490 to base 9399 of SEQ ID NO: 95. The system also comprises a nucleic acid nucleic acid expression construct for expressing a Pong ORF2 protein fused to a Cas9 nuclease, wherein the expression construct for expressing the Pong ORF2 protein fused to a Cas9 nuclease comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 9582 to base 16938 of SEQ ID NO: 95. In some aspects, the expression construct for expressing the Pong ORF2 protein fused to a Cas9 nuclease comprises a nucleic acid sequence comprising at least about 75% or more, at least about 85% or more, at least about 95% or more, or 100% sequence identity with the nucleic acid sequence starting at base 9582 to base 16938 of SEQ ID NO: 95. The system comprises a nucleic acid construct comprising the donor polynucleotide, wherein the nucleic acid construct comprising the donor polynucleotide comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or comprising at least about 75% or more, at least about 85% or more, at least about 95% or more, or 100% sequence identity with the nucleic acid sequence starting at base 4545 to base 2173 of SEQ ID NO: 95. The system also comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or comprising at least about 75% or more, at least about 85% or more, at least about 95% or more, or 100% sequence identity with the nucleic acid sequence starting at base 4763 to base 5474 of SEQ ID NO: 95. In some aspects, the system is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 95. In some aspects, the system is encoded on a plasmid comprising a nucleic acid sequence comprising at least about 75% or more, at least about 85% or more, at least about 95% or more, or 100% sequence identity with the nucleic acid sequence of SEQ ID NO: 95.

In some aspects, the system of the instant disclosure comprises a helper construct and a donor construct, wherein the helper construct comprises a nucleic acid expression construct for expressing Pong ORF1 and a nucleic acid expression construct for expressing Pong ORF2 protein fused to a Cas9 nuclease. The system comprises a nucleic acid expression construct for expressing a Pong ORF1 protein, wherein the expression construct for expressing the Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 981 to base 4890 of SEQ ID NO: 75. In some aspects, the nucleic acid expression construct for expressing a Pong ORF1 protein comprises at least about 75% or more, at least about 85% or more, at least about 95% or more, or 100% sequence identity with the nucleic acid sequence starting at base 981 to base 4890 of SEQ ID NO: 75. The system also comprises a nucleic acid expression construct for expressing a Pong ORF2 protein fused to Cas9 nuclease, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5073 to base 12429 of SEQ ID NO: 75. In some aspects, the construct for expressing a Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 5073 to base 12429 of SEQ ID NO: 75. The system further comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 254 to base 965 of SEQ ID NO: 75. In some aspects, the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 75. In some aspects, the system is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 75. In some aspects, the system is encoded on a plasmid comprising a nucleic acid sequence comprising at least about 75% or more, at least about 85% or more, at least about 95% or more, or 100% sequence identity with the nucleic acid sequence of SEQ ID NO: 75.

In some aspects, the donor polynucleotide is inserted in a nucleic acid expression construct encoding a GFP reporter, thereby inactivating the reporter. In some aspects, the expression construct is inserted in nucleic acid sequence in the genome of the cell. In some aspects, the target nucleic acid locus is in an Arabidopsis PDS3 gene.

In some aspects, the system of the instant disclosure comprises a helper construct and a donor construct. In some aspects, the donor construct comprises a nucleic acid expression construct encoding a GFP reporter. The donor nucleic acid construct is inserted into the expression construct thereby inactivating the reporter. In these aspects, the target nucleic acid locus is an Arabidopsis ADH1 gene. The helper construct comprises a nucleic acid expression construct for expressing Pong ORF1, a nucleic acid expression construct for expressing Pong ORF2 protein, and a nucleic acid construct for expressing a deCas9 nickase. The expression construct for expressing a Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 981 to base 4890 of SEQ ID NO: 89. In some aspects, the nucleic acid expression construct for expressing a Pong ORF1 protein comprises at least about 75% or more, at least about 85% or more, at least about 95% or more, or 100% sequence identity with the nucleic acid sequence starting at base 981 to base 4890 of SEQ ID NO: 89. The system also comprises a nucleic acid expression construct for expressing a Pong ORF2 protein, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5073 to base 8215 of SEQ ID NO: 89. In some aspects, the construct for expressing a Pong ORF2 protein comprises a nucleic acid sequence comprising at least about 75% or more, at least about 85% or more, at least about 95% or more, or 100% sequence identity with the nucleic acid sequence starting at base 5073 to base 8215 of SEQ ID NO: 89. The system also comprises a nucleic acid expression construct for expressing a deCas9 nickase, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at nucleotide 8218 to nucleotide 13856 of SEQ ID NO: 89. In some aspects, the construct for expressing a deCas9 nickase protein comprises a nucleic acid sequence comprising at least about 75% or more, at least about 85% or more, at least about 95% or more, or 100% sequence identity with the nucleic acid sequence starting at nucleotide 8218 to nucleotide 13856 of SEQ ID NO: 89. The system further comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 254 to base 965 of SEQ ID NO: 89. In some aspects, the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 89. In some aspects, the helper construct is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 89. In some aspects, the helper construct is encoded on a plasmid comprising a nucleic acid sequence comprising at least about 75% or more, at least about 85% or more, at least about 95% or more, or 100% sequence identity with the nucleic acid sequence of SEQ ID NO: 89.

In some aspects, the system of the instant disclosure comprises a helper construct and a donor construct. In some aspects, the donor construct comprises a nucleic acid expression construct encoding a GFP reporter, wherein the donor nucleic acid construct is inserted into the expression construct thereby inactivating the reporter. In these aspects, the target nucleic acid locus is an Arabidopsis ACT8 gene. The helper construct comprises a nucleic acid expression construct for expressing Pong ORF1 and a nucleic acid expression construct for expressing Pong ORF2 protein fused to a Cas9 nuclease. The expression construct for expressing a Pong ORF1 protein comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 981 to base 4890 of SEQ ID NO: 91. In some aspects, the nucleic acid expression construct for expressing a Pong ORF1 protein comprises at least about 75% or more, at least about 85% or more, at least about 95% or more, or 100% sequence identity with the nucleic acid sequence starting at base 981 to base 4890 of SEQ ID NO: 91. The system also comprises a nucleic acid expression construct for expressing a Pong ORF2 protein fused to Cas9 nuclease, wherein the construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 5073 to base 12429 of SEQ ID NO: 91. In some aspects, the construct for expressing a Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 5073 to base 12429 of SEQ ID NO: 91. The system further comprises an expression construct for expressing a gRNA, wherein the expression construct for expressing the gRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 254 to base 965 of SEQ ID NO: 91. In some aspects, the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 91. In some aspects, the helper construct is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 91. In some aspects, the helper construct is encoded on a plasmid comprising a nucleic acid sequence comprising at least about 75% or more, at least about 85% or more, at least about 95% or more, or 100% sequence identity with the nucleic acid sequence of SEQ ID NO: 91.

The donor construct comprises a nucleic acid expression construct comprising a promoter operably linked to a polynucleotide sequence encoding GFP, wherein the donor polynucleotide inserted in the nucleic acid expression construct. In some aspects, the GFP expression construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence starting at base 3037 clockwise to base 665 of SEQ ID NO: 90. In some aspects, the GFP expression construct comprises a nucleic acid sequence comprising at least about 75% or more, at least about 85% or more, at least about 95% or more, or 100% sequence identity with the nucleic acid sequence starting at base 3037 clockwise to base 665 of SEQ ID NO: 90. In some aspects, the donor construct is encoded on a plasmid comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 90. In some aspects, the donor construct is encoded on a plasmid comprising a nucleic acid sequence comprising at least about 75% or more, at least about 85% or more, at least about 95% or more, or 100% sequence identity with the nucleic acid sequence of SEQ ID NO: 90.

III. Cells

In another aspect, the present disclosure provides a cell, a tissue, or an organism comprising an engineered system described in Section I above. One or more components of the engineered system in the cell may be encoded by one or more nucleic acid constructs of a system of nucleic acid constructs as described in Section II above.

A variety of cells are suitable for use in the methods disclosed herein. The cell may be a prokaryotic cell. Alternatively, the cell is a eukaryotic cell. For example, the cell may be a prokaryotic cell, a human mammalian cell, a non-human mammalian cell, a non-mammalian vertebrate cell, an invertebrate cell, an insect cell, a plant cell, a yeast cell, or a single cell eukaryotic organism. The cell may also be a one-cell embryo. For example, a non-human mammalian embryo including rat, hamster, rodent, rabbit, feline, canine, ovine, porcine, bovine, equine, plant, and primate embryos. The cell may also be a stem cell such as embryonic stem cells, ES-like stem cells, fetal stem cells, adult stem cells, and the like. The cell may be in vitro, ex vivo, or in vivo (i.e., within an organism or within a tissue of an organism).

Non-limiting examples of suitable mammalian cells or cell lines include human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells; Chinese hamster ovary (CHO) cells; baby hamster kidney (BHK) cells; mouse myeloma NS0 cells; mouse embryonic fibroblast 3T3 cells (NIH3T3); mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells; mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; Afrimay green monkey kidney (VERO-76) cells. An extensive list of mammalian cell lines may be found in the Amerimay Type Culture Collection catalog (ATCC, Manassas, VA).

The cell may be a plant cell, a plant part, or a plant. Plant cells include germ cells and somatic cells. Non-limiting examples of plant cells include parenchyma cells, sclerenchyma cells, collenchyma cells, xylem cells, and phloem cells. Plant parts include, but are not limited to, stems, roots, ovules, stamens, leaves, embryos, meristematic regions, callus tissue, gametophytes, sporophytes, pollen, microspores, and the like. The plant can be a monocot plant or a dicot plant. For instance, the plant can be soybean; maize; sugar cane; beet; tobacco; wheat; barley; poppy; rape; sunflower; alfalfa; sorghum; rose; carnation; gerbera; carrot; tomato; lettuce; chicory; pepper; melon; cabbage; oat; rye; cotton; millet; flax; potato; pine; walnut; citrus (including oranges, grapefruit etc.); hemp; oak; rice; petunia; orchids; Arabidopsis; broccoli; cauliflower; brussels sprouts; onion; garlic; leek; squash; pumpkin; celery; pea; bean (including various legumes); strawberries; grapes; apples; cherries; pears; peaches; banana; palm; cocoa; cucumber; pineapple; apricot; plum; sugar beet; lawn grasses; maple; teosinte; Tripsacum; Coix; triticale; safflower; peanut; cassava, and olive.

The invention also provides an agricultural product produced by any of the described transgenic plants, plant parts, and plant seeds. Agricultural products include, but are not limited to, plant extracts, proteins, amino acids, carbohydrates, fats, oils, polymers, vitamins, and the like.

IV. Methods

A further aspect of the present disclosure provides a method of inserting a donor polynucleotide into a target nucleic acid locus in a cell. In a method of the instant disclosure, the cell can be ex vivo or in vivo. The locus can be in a chromosomal DNA, organellar DNA, or extrachromosomal DNA. The method can be used to insert a single donor polynucleotide or more than one donor polynucleotide at one or more target loci.

The method comprises providing or having provided an engineered system for generating a genetically modified cell, and introducing the system into the cell. The method further comprises maintaining the cell under appropriate conditions such that the donor polynucleotide is inserted in the target locus. Optionally, the method further comprises identifying an accurate insertion of the donor polynucleotide in the nucleic acid locus. The engineered system can be as described in Section I; nucleic acid constructs encoding one or more components of the homologous recombination compositions can be as described in Section II; and the cells can be as described in Section III.

Insertion of the donor polynucleotide into a target nucleic acid locus in a cell can have a number of uses known to individuals of skill in the art. For instance, insertion of the donor polynucleotide can introduce cargo nucleic acid sequences of interest into nucleic acid sequences in a cell, including genes of interest or regulatory nucleic acid sequences of interest. Alternatively, insertion of a donor polynucleotide can be used to introduce nucleic acid modifications in nucleic acid sequences in the cell. The system can be used to modulate transcriptional or post-transcriptional expression of an endogenous nucleic acid sequence in the cell, to investigate RNA-protein interactions, or to determine the function of a protein or RNA, or investigate RNA-protein interactions, or to alter the stability, accumulation, and protein production from the RNA.

In general, nucleic acid sequences can be introduced into a nucleic acid sequence of a cell by flanking the nucleic acid sequence to be introduced with the transposition sequences compatible with the transposase. Introduced nucleic acid sequences can include, without limitation, genes of interest, such as genes encoding disease resistance or short RNAs, reporters, programmable nucleic acid-modification systems, epigenetic modification systems, and any combination thereof.

In some aspects, a system of the instant disclosure is used to alter expression of a gene of interest. The method comprises introducing an array of six heat-shock enhancer elements flanked by the mPing transposition sequences for insertion into the promoter of the Arabidopsis ACT8 gene. These enhancers have a short size and regulate expression of the gene irrespective of the orientation of the introduced sequences.

(a) Introduction into the Cell

The method comprises introducing the engineered system into a cell of interest. The engineered system may be introduced into the cell as a purified isolated composition, purified isolated components of a composition, as one or more nucleic acid constructs encoding the engineered system, or combinations thereof. Further, components of the engineered system can be separately introduced into a cell. For example, a transposase, a donor polynucleotide, and a programmable targeting nuclease can be introduced into a cell sequentially or simultaneously.

The engineered system described above may be introduced into the cell by a variety of means. Suitable delivery means include microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, implantable devices, magnetofection, lipofection, impalefection, optical transfection, proprietary agent-enhanced uptake of nucleic acids, Agrobacterium tumefaciens mediated foreign gene transformation, proprietary agent-enhanced uptake of nucleic acids, and delivery via liposomes, immunoliposomes, virosomes, or artificial virions. The choice of means of introducing the system into a cell can and will vary depending on the cell, or the system or nucleic acid nucleic acid constructs encoding the system, among other variables.

### (b) Culturing a Cell

The method further comprises maintaining the cell under appropriate conditions such that the donor polynucleotide is inserted in the target locus. When the cell is in tissue ex vivo, or in vivo within an organism or within a tissue of an organism, the tissue and/or organism may also be maintained under appropriate conditions for insertion of the donor polynucleotide. In general, the cell is maintained under conditions appropriate for cell growth and/or maintenance. Those of skill in the art appreciate that methods for culturing cells are known in the art and may and will vary depending on the cell type. Routine optimization may be used, in all cases, to determine the best techniques for a particular cell type. See for example, in Santiago et al. (2008) PNAS 105:5809-5814; Moehle et al. (2007) PNAS 104:3055-3060; Urnov et al. (2005) Nature 435:646-651; and Lombardo et al. (2007) Nat. Biotechnology 25:1298-1306; Taylor et al., (2012) Tropical Plant Biology 5: 127-139.

In some aspects, the method further comprises identifying an accurate insertion of the donor polynucleotide using methods known in the art. Upon confirmation that an accurate insertion has occurred, single cell clones may be isolated. Additionally, cells comprising one accurate insertion may undergo one or more additional rounds of targeted insertions of additional polynucleotides.

V. Kits

A further aspect of the present disclosure provides kits for generating a genetically modified cell. The kit comprises one or more engineered systems detailed above in Section I. The engineered systems can be encoded by a system of one or more nucleic acid constructs encoding the components of the system as described above described above in Section II. Alternatively, the kit may comprise one or more cells comprising one or more engineered systems, one or more nucleic acid constructs, or combinations thereof.

A further aspect of the present disclosure provides a system of one or more nucleic acid constructs encoding the components of the system described above

The kits may further comprise transfection reagents, cell growth media, selection media, in-vitro transcription reagents, nucleic acid purification reagents, protein purification reagents, buffers, and the like. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), an internet address that provides the instructions, and the like. As used herein, the term “instructions” may include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “gene” refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

A “genetically modified” cell refers to a cell in which the nuclear, organellar or extrachromosomal nucleic acid sequences of a cell has been modified, i.e., the cell contains at least one nucleic acid sequence that has been engineered to contain an insertion of at least one nucleotide, a deletion of at least one nucleotide, and/or a substitution of at least one nucleotide.

The terms “genome modification” and “genome editing” refer to processes by which a specific nucleic acid sequence in a genome is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

As used herein, the term “compatible transposition sequences” refers to any transposition sequences recognized by the transposase for transposition. For instance, the transposition sequences can be transposition sequences of the TE from which the transposase is derived, or from another autonomous or non-autonomous TE recognized by the transposase for transposition.

As used herein, the term “engineered” when applied to a targeting protein refers to targeting proteins modified to specifically recognize and bind to a nucleic acid sequence at or near a target nucleic acid locus. A “genetically modified” plant refers to a cell in which the nuclear, organellar or extrachromosomal nucleic acid sequences of a cell have been modified, i.e., the cell contains at least one nucleic acid sequence that has been engineered to contain an insertion of at least one nucleotide, a deletion of at least one nucleotide, and/or a substitution of at least one nucleotide.

The term “nucleic acid modification” refers to processes by which a specific nucleic acid sequence in a polynucleotide is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

As used herein, “protein expression” includes but is not limited to one or more of the following: transcription of a gene into precursor mRNA; splicing and other processing of the precursor mRNA to produce mature mRNA; mRNA stability; translation of the mature mRNA into protein (including codon usage and tRNA availability); production of a mutant protein comprising a mutation that modifies the activity of the protein, including the calcium channel activity; and glycosylation and/or other modifications of the translation product, if required for proper expression and function. The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site”, “target sequence”, or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

As used herein, the term “encode” is understood to have its plain and ordinary meaning as used in the biological fields, i.e., specifying a biological sequence. For instance, when a construct is encoding a protein of the system, the term is understood to mean that the construct further comprises nucleic acid sequences required for expressing the components of the system.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Targeted Integration of a Transposable Element

Transgenesis in plants is accomplished via bombardment or Agrobacterium-mediated transformation and results in the integration of foreign DNA into a plant's genome. During this process, the transgene integration site within the plant DNA is not controlled, and follow-up experiments must be performed to determine where in the genome the transgene integrated. En mass transformation experiments have demonstrated that the integration typically occurs at sites of open chromatin configuration, such as actively transcribing genes, however integration into heterochromatic closed chromatin can also occur. Transgene integration into or near genes can generate new mutations or alter the regulation of nearby genes, while insertions into heterochromatic regions are often not permissive to the desired high levels of transgene expression or do not provide stable expression over multiple generations. Insertion of transgenes is also associated with mutations (deletions and rearrangements) of the target region and transferred DNA. In addition, to study or create a product from a gene of interest, it needs to be taken out of its native context and added back to the plant as a transgene, and key distal regulatory enhancers or repressor elements can be missed or rearranged during this process. The lack of user-defined control of transgene integration site generates variability and inconsistency in experiments and products.

The control of transgene integration site is desired to direct transgenes to the same expression-permissive regions of the genome (to reduce variability), to add sequences to genes at their native locations, and/or to maintain gene order on the chromosome. Multiple attempts have been made to overcome these issues and perform target site-directed integration. The FLP-FRT recombination system has been used to reproducibly target transgene insertion into one location in plant genomes. However, this insertion site must also be transgenic to carry the correct targeting sequences. Current methods to insert DNA into any user-defined targeted region of a plant genome involve homology-directed repair (HDR) off a provided DNA template after a double-strand DNA break induced by a Meganuclease, Zinc Finger Nuclease, TALEN or CRISPR/Cas9 (or related) system. In plants, currently available tools using targeted insertion of a transgene via HDR are inefficient for two reasons. First, the complementary repair template and nuclease system must be added to the cell via traditional transgenesis, which particularly in crop plants is laborious. Second, plant cells favor the resolution of double-strand DNA breaks by the non-homology end joining (NHEJ) pathway, which bypasses the integration of new DNA.

Recently, research has uncovered naturally-occurring fusions between transposase proteins and the CRISPR/Cas system in prokaryotes. The CRISPR/Cas system provides sequence specificity to the transposase for selection of the integration site, and was proven to be programmable by altering the sequence of the CRISPR guide RNA (gRNA). However, none of the systems currently available that use CRISPR-targeting of a transposase protein were successful in targeting to a specific gene location in eukaryotic cells. To date, the programmability of transposase-mediated integration of DNA has not been accomplished in a eukaryote.

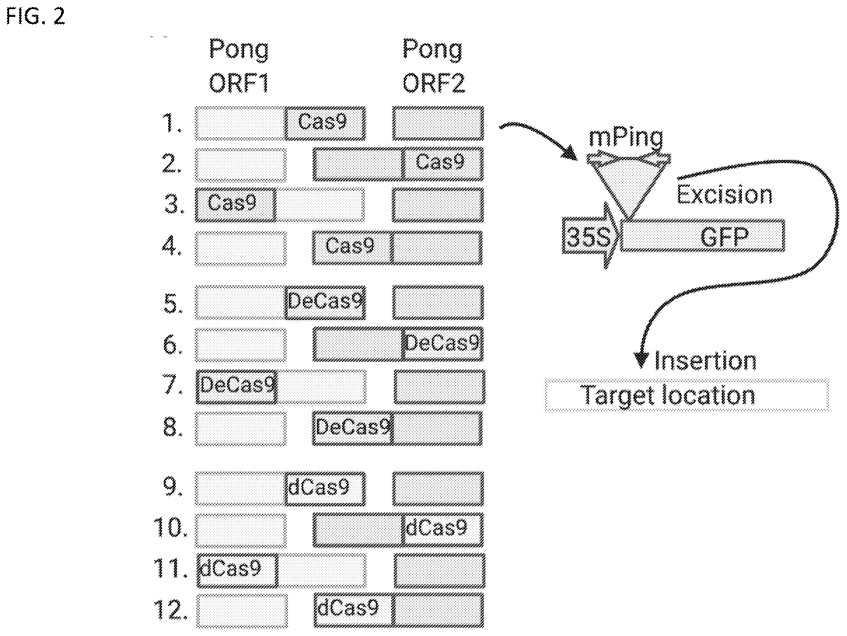

In an attempt to overcome the difficulties in guiding insertion of a transgene into a target locus, the inventors fused a TE-encoded transposase protein to the CRISPR/Cas9 system to achieve targeted integration of DNA in plants. The inventors reasoned that the transposase protein would need to have two features to broadly function in this system. First, a wide host-range of functionality in plants was desired to create a universal tool for plant biology. Second, using split-transposase proteins (where the single transposase was encoded by two proteins that function together to achieve excision and insertion) would have a lower probability of disturbing protein function. It was reasoned that the rice mPing/Pong system would provide the highest probably of functioning when fused to Cas9, as the Pong transposase is split into two proteins (ORF1 and ORF2) and can mobilize the mPing non-autonomous (non-protein coding) TE in a range of plant species. An mPing/Pong engineered system was used that had the Pong transposase ORF1 and ORF2 immobilized by the removal of the Pong TIRs. In this system, mPing excision can be visualized by its removal from a constitutively expressed GFP gene (FIG. 1). The Pong ORF1/ORF2 system was engineered with the G4S (GSSSS) flexible protein linker to allow efficient fusions to Cas9 proteins on either the N- or C-terminus of ORF1 or ORF2, and an SV40 nuclear localization signal (NLS) was added to these protein fusions. Three versions of the Cas9 protein were used, the catalytically active Cas9, the single-stranded nickase deCas9, and the catalytically inactive dCas9. A total of 12 constructs were generated (3 Cas9 proteins×4 ORF1/ORF2 positions; FIG. 2) with a gRNA known to target the Arabidopsis PDS3 gene.

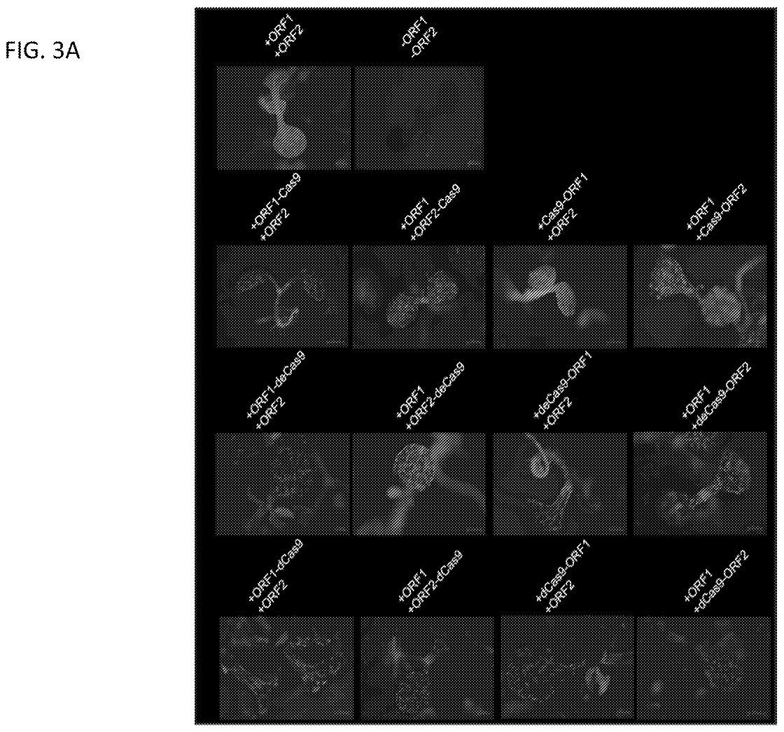

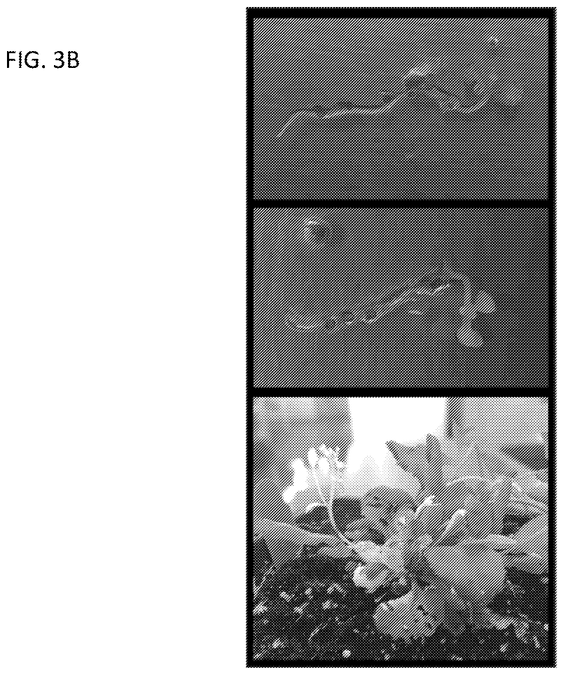

To determine if the Pong transposase was functional when fused to Cas9 derivatives, GFP fluorescence was visualized in seedlings. GFP fluorescence is a marker of mPing excision from the GFP donor site, and this fluorescence was detected for all 12 fusion proteins, but not the negative control without ORF1/ORF2 (FIG. 3A), verifying that ORF1 and ORF2 are co-creating a functional transposase protein even while fused to Cas9. A functional CRISPR/Cas9 system was verified through the observation of white seedlings and sectors in plants with the Cas9 and deCas9 proteins (in this experiment, dCas9 plants did not display white plants or sectors) (FIG. 3B). Overall, the results demonstrate that fusion of the Cas9 and transposase proteins does not stop their function.

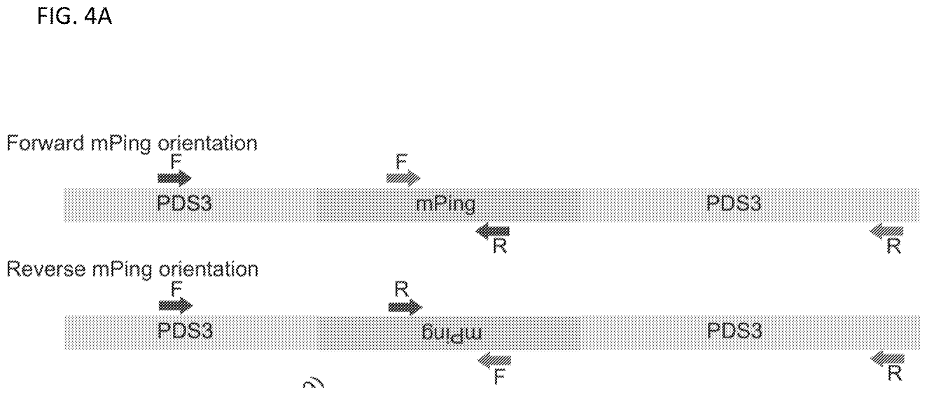

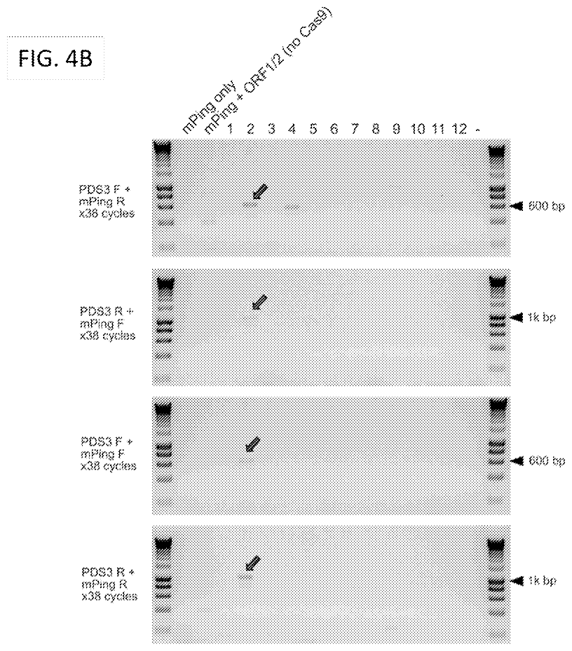

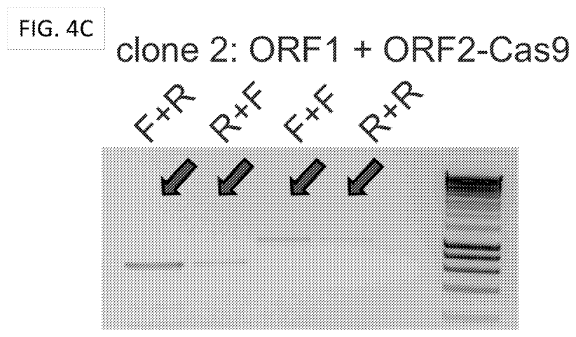

A PCR amplification strategy was used to detect targeted mPing insertions into the Arabidopsis PDS3 gene (FIG. 4A). T2 seedling pools were screened using negative control lines that either lack ORF1/ORF2, or that lack the Cas9 fusion (FIG. 4B). It was found that clone #2 displayed the correct size PCR band in all PCR assays (FIG. 4B). The PCR can identify mPing insertions in the forward or reverse orientation (FIG. 4A), and the fact that clone #2 amplified for both suggests that there is more than one mPing insertion in this pool of plants. Clone #2 encodes for ORF1+ORF2-Cas9, where ORF2 has a C-terminal fusion to the Cas9 protein. This data demonstrates targeted insertion of mPing into the PDS3 gene using a targeting nuclease having full double stranded cleavage activity of Cas9.

### Example 2. Characterization of Target Site Insertions

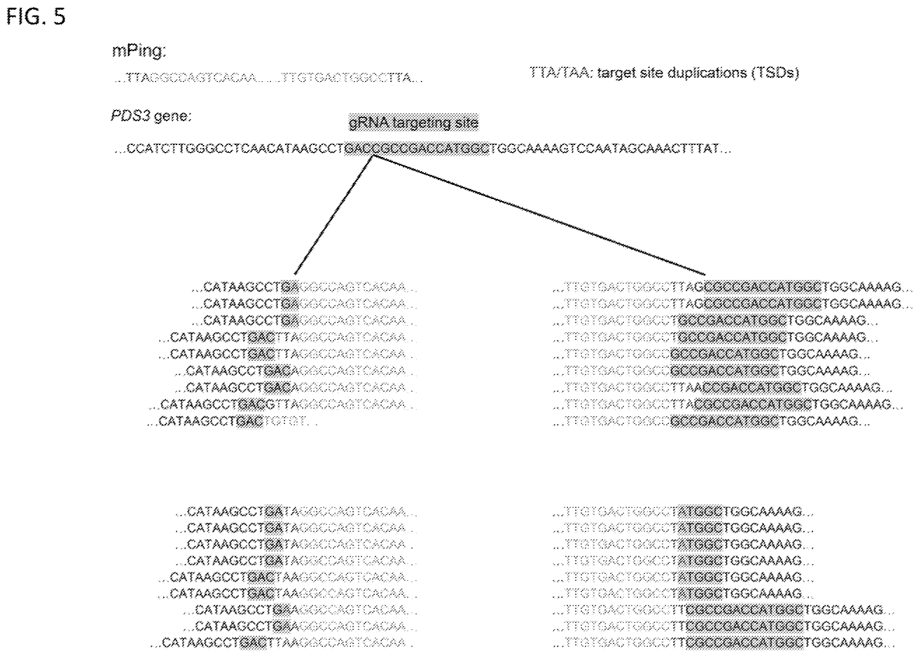

The target-site PCR assay was replicated (FIG. 4C), and PCR products cloned and sequenced. In all, 36 clones were sequenced. The sequenced clones represent at least nine (9) unique targeted transposition events (FIG. 5). Both mPing forward and reverse orientation insertions were identified, demonstrating the random directionality of the targeted insertion event.

The targeted insertion occurred between the third and fourth base of the gRNA target sequence, as expected based on the known cleavage activity of Cas9 (FIG. 5). The results show that mPing is intact in each sequenced clone except one. In each case there is one target site duplication, on either the 5′ or 3′ of mPing. Additional single-base insertions are found in some clones. The sequencing represents at least nine distinct events, meaning that mPing inserted into the PDS3 gene in the line with clone #2 at least nine different times. Most insertions have either intact or partial TTA/TAA sequence on only one end of the insertion. This sequence originates from the donor site and is part of the known target site duplication (TSD) of the Pong/mPing TE system. The presence of only one TSD, rather than one on either side of the TE insertion, signifies that Cas9 created a blunt cut at the insertion site, but the transposase protein made a staggered cut at the donor site before the integration event. This demonstrates that both the Cas9 and transposase proteins are functional for generating this set of insertions.

For each insertion, the gRNA target sequence was preserved and mPing had inserted at the expected Cas9 cleavage point between the third and fourth nucleotide. In all but one sequence read the mPing element is complete, with only single base insertions. The lack of deletions or other insertions at these insertion sites demonstrates the seamless repair of the insertion events by the transposase protein compared to typical sites of blunt-end DNA breaks.

### Example 3. Integration into any DNA Break

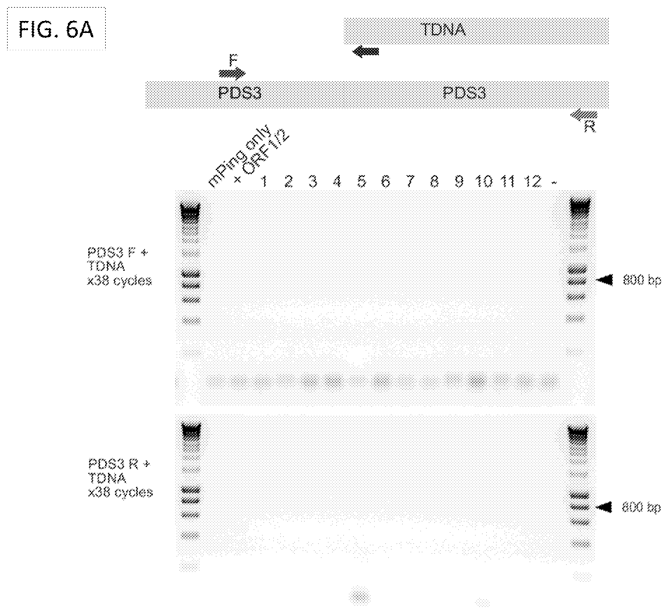

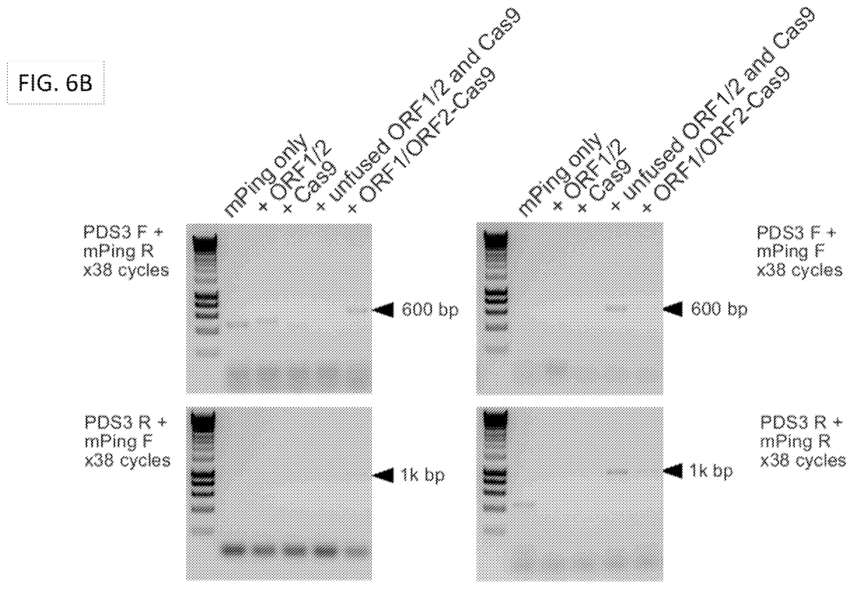

Several previous reports have demonstrated that transgenes will insert at a low frequency into any site of double-strand break. To determine if the mPing targeted insertion detected in Examples 1 and 2 requires the transposase protein, a PCR assay was performed for the integration of the transgene backbone encoding the ORF2-Cas9 protein into the DNA break generated at PDS3. It was reasoned that if the mPing insertion into PDS3 was a product of transgene insertion, rather than transposition, it would be equally likely to detect other parts of the transgene at this insertion site location. However, transgene was detected at PDS3 (FIG. 6A), demonstrating that mPing insertion requires the transposase to excise the mPing element from the donor position.

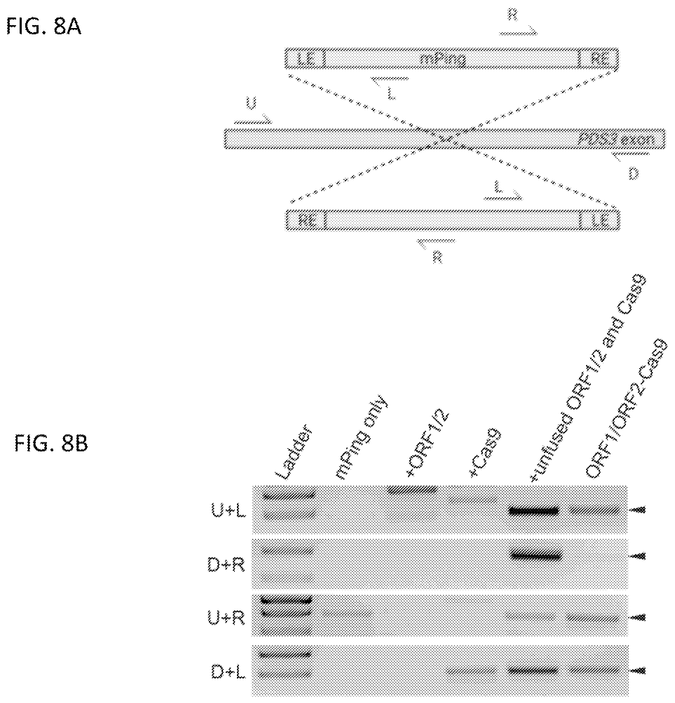

Next, it was assayed whether it was essential that the transposase protein and Cas9 were directly fused, or if both proteins unfused in the same cell could perform targeted insertion. It was discovered that in some cases, the two proteins could be unfused and targeted insertion would take place (FIG. 6B). At the same time, it was demonstrated that both proteins are functional and that in this instance, the catalytic activity of Cas9 is used (FIG. 6B). Together, this data demonstrates that to obtain targeted insertion, it is essential that the transposase excise the element out of the donor position, and that Cas9 cleave the insertion site, but the two proteins do not necessarily need to be fused together (see FIGS. 8A and 8B and Example 5).

### Example 4. Programmability of Target Sites

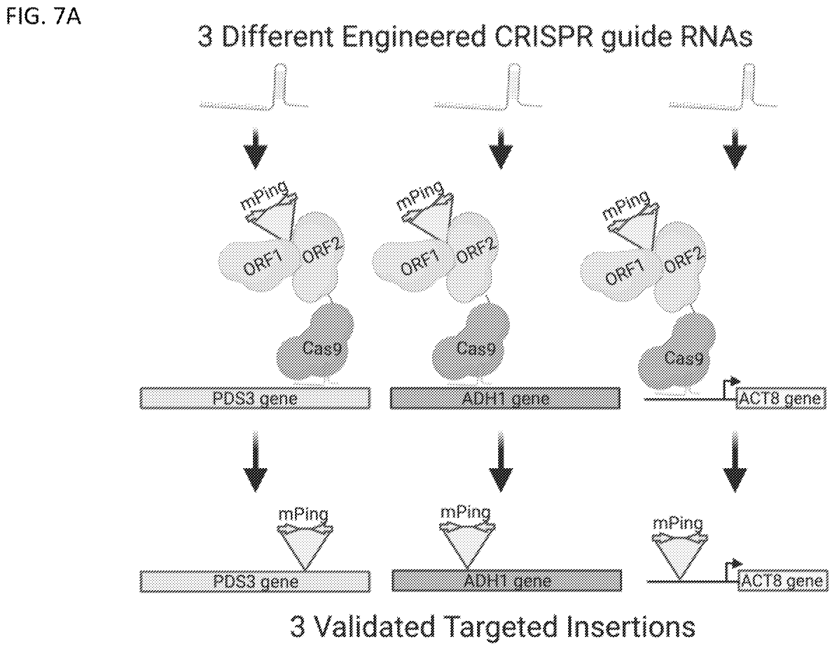

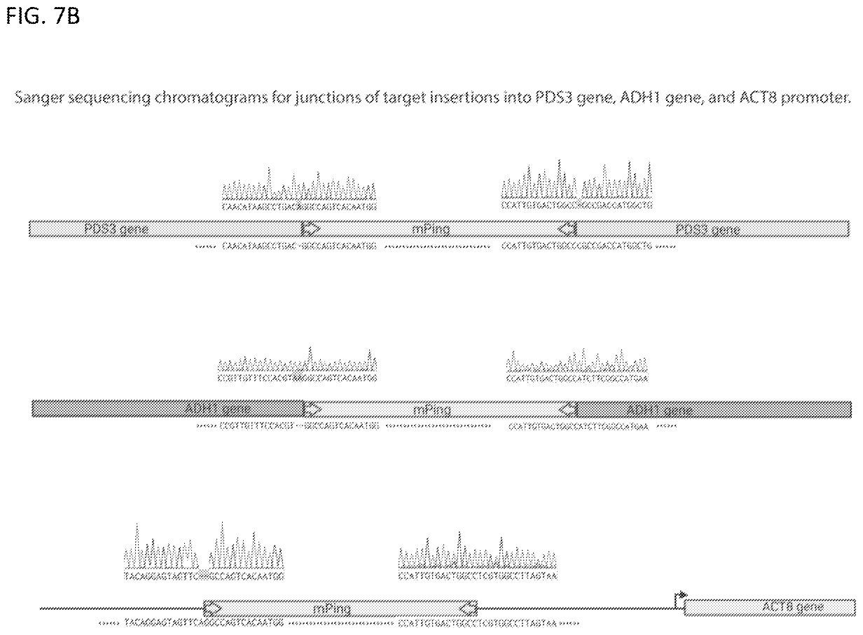

Multiple sites in the Arabidopsis genome were targeted using the system of the instant disclosure. Two additional gRNAs were designed for integration into two additional target loci; the ADH1 gene and a non-coding region upstream of the ACT8 gene of Arabidopsis. The gRNAs were used in a system described herein to integrate mPing into the two target loci (FIG. 7A). FIG. 7B shows the Sanger sequencing results of junctions of each identified target insertion into the PDS3 gene, the ADH1 gene, and the promoter of ACT8 gene. The chromatograms above the sequence show the sequences at the insertion sites. The sequences below mPing are the expected sequence if a perfect “seamless” insertion is obtained. These results clearly confirm that the insertion of a donor polynucleotide is surprisingly and unexpectedly inserted on target and unexpectedly accurate and seamless.

### Example 5. Direct Fusion of the Transposase Proteins ORF1 and ORF2 to the Nuclease is not Required for Targeted Insertions

Using methods described in Example 3, whether a system wherein the transposase proteins ORF1 and ORF2 are not directly fused to the Cas9 nuclease was tested. FIG. 8A shows that mPing can be targeted to the Arabidopsis PDS3 gene by the CRISPR gRNA and can insert in either the forward direction (above the PDS3 region) or reverse direction (below the PDS3 region). A combination of 2 out of 4 PCR primers corresponding to the PDS3 exon (U,D) and the mPing gene (R, L) were used. FIG. 8A shows the location of these 4 PCR primers (R,L,U,D) for orientation.

The mPing targeted insertion was detected with PCR using the primer sets from part A. FIG. 8B shows a representative agarose gel with PCR products observed. Arrowheads denote the correct size of the PCR products for each set of primers. “mPing only”, “+ORF1/2” and “+Cas9” are negative controls. Any bands from these lanes near the correct size were sequenced and shown not to be specific targeted insertions of mPing. The bands shown in the “+unfused ORF1/2 and Cas9” lane show that using unfused constructs can generate real targeted insertions, as does the biological replicate of ORF2 fused to Cas9 in the “ORF1/ORF2-Cas9” lane. All PCR products from this assay were also verified by Sanger sequencing. These data confirm the results from FIG. 6B and demonstrate that direct fusion of the transposase proteins to the nuclease is not required for targeted insertions.

### Example 6: Targeted Insertion Driven by Single Transgene Vector

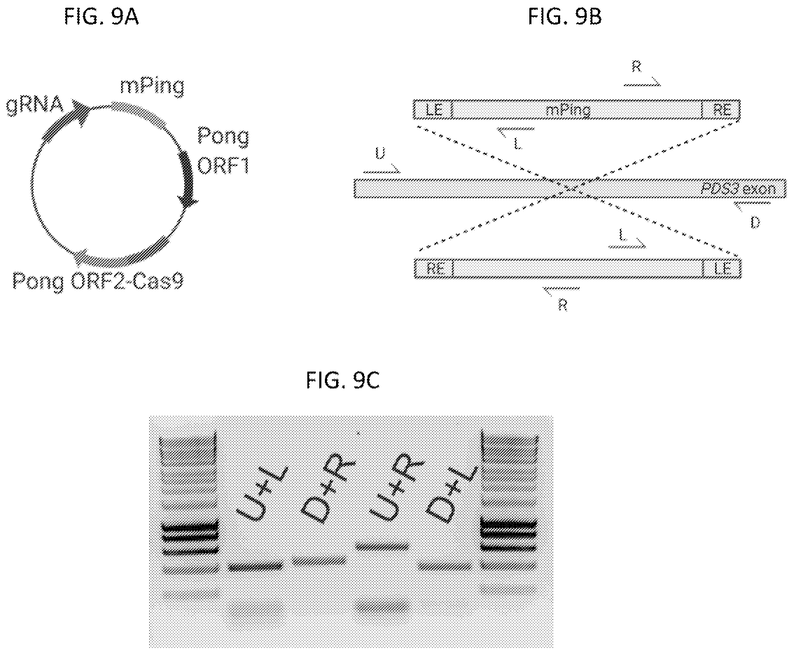

In the previously described experiments, the system comprised a donor construct and a helper construct. Here, a single transgene vector was developed containing all the elements required for targeted insertion in a plant cell. The vector is diagrammed in FIG. 9A and contains the CRISPR/Cas9 system (including gRNA), the mPing donor element, and ORF1 and ORF2 transposase proteins.

Using methods described in the examples above, mPing was targeted to the Arabidopsis PDS3 gene by the CRISPR gRNA. As shown in FIG. 9B, mPing can insert in either the forward direction (above the PDS3 region) or reverse direction (below the PSD3 region). The location of 4 PCR primers (R, L, U, D) are shown for orientation. FIG. 9C shows a representative agarose gel with PCR detection of mPing targeted insertion in the Arabidopsis genome using the primer sets from part B. The largest PCR fragment for each primer set is the correct size and was Sanger sequenced to ensure that it is a bonafide targeted insertion of mPing into the PDS3 gene.

### Example 7: Targeted and Seamless Integration in Plant Genomes Using CRISPR-Transposases

Introduction

Transgenesis in plants is accomplished via bombardment or agrobacterium-mediated transformation and results in the integration of foreign DNA into a plant's genome. During this process, the transgene integration site within the plant DNA is not controlled, and follow-up experiments must be performed to determine where in the genome the transgene integrated. En mass transformation experiments have demonstrated that the integration typically occurs at sites of open chromatin configuration, such as actively transcribing genes, however integration into heterochromatic closed chromatin can also occur. Transgene integration into or near genes can generate new mutations or alter the regulation of nearby genes, while insertions into heterochromatic regions are often not permissive to the desired high levels of transgene expression or do not provide stable expression over multiple generations. Insertion of transgenes is also associated with mutations (deletions and rearrangements) of the target region and transferred DNA. In addition, to study or create a product from a gene of interest, it needs to be taken out of its native context and added back to the plant as a transgene, and key distal regulatory enhancers or repressor elements can be missed or rearranged during this process. The lack of user-defined control of transgene integration site generates variability and inconsistency in experiments and products.

The control of transgene integration site is desired to direct transgenes to the same expression-permissive regions of the genome (to reduce variability), to add sequences to genes at their native locations, and/or to maintain gene order on the chromosome. Multiple attempts have been made to overcome these issues and perform targeted site-directed integration. Recombination systems have been used to reproducibly target transgene insertion into one location in plant genomes, however, this insertion site must also be transgenic to carry the correct targeting sequences. Current methods to insert DNA into any user-defined targeted region of a plant genome involve homology-directed repair (HDR) off a provided DNA template after a double-strand DNA break induced by a Meganuclease, Zinc Finger Nuclease, TALEN or CRISPR/Cas9 (or related) system. In plants, targeting insertion of a transgene via HDR is inefficient for two reasons. First, the complementary repair template and nuclease system must be added to the cell via traditional transgenesis, which particularly in crop plants is laborious. Second, plant cells favor the resolution of double-strand DNA breaks by the non-homology end joining (NHEJ) pathway, which bypasses the integration of new DNA. Therefore, addition of custom sequences to a targeted location in a plant genome is laborious, requiring screening for a low-frequency event. In addition, because free ends of DNA are exposed during this process, the ends of the inserted fragment of DNA or the native DNA at the insertion site is often subject to degradation, creating deletions and unintended base changes at the HDR site.

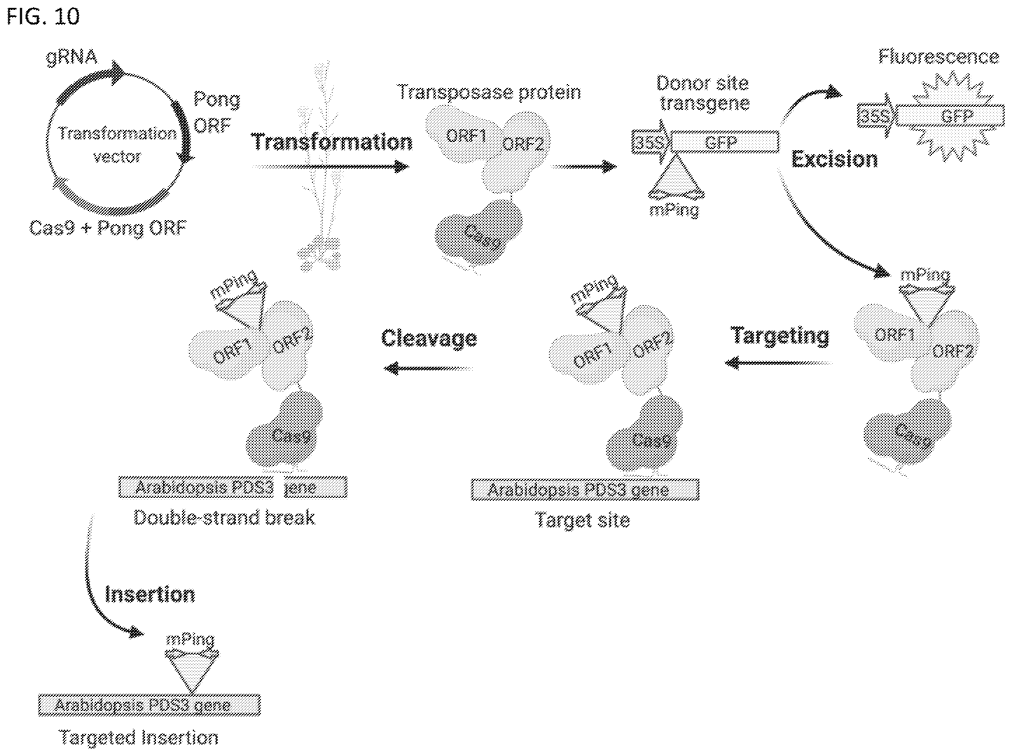

Transposases are transposable element (TE)-derived proteins that naturally mobilize pieces of DNA from one location in the genome to another. Transposases function by binding the repeated ends of a TE called the terminal inverted repeats (TIRs) within the same TE family. The transposase cleaves the DNA, removing the TE from the excision/donor site, then cleaves and integrates the TE at the insertion site. Plant transposases select their insertion site by chromatin context and DNA accessibility but are not targeted to individual regions or specific sequences of plant genomes. Recently, research has uncovered naturally-occurring fusions between transposase proteins and the CRISPR/Cas system in prokaryotes. The CRISPR/Cas system provides sequence specificity to the transposase for selection of the integration site, and was proven to be programmable by altering the sequence of the CRISPR guide RNA (gRNA). Several laboratories have taken the approach to identify natural Cas protein fusions to transposable elements in prokaryotic genomes, with the intent of moving these fusion proteins into eukaryotes. In human cell culture, CRISPR-targeting of a transposase protein has been attempted but failed to target to a specific gene location, although the integration into targeted repetitive retrotransposon sites were enriched. The inventors took the approach of starting with a transposase protein known to work in a wide variety of plants, and Cas9 and CFP1, which have also been shown to work in plants. Rather than identifying a natural fusion in a prokaryotic genome, both of these proteins were artificially used at the same time, including fusing these proteins together, to accomplish targeted insertion in a plant genome. An overview of this process is shown in FIG. 10.

Results

Targeted Integration of a Transposable Element

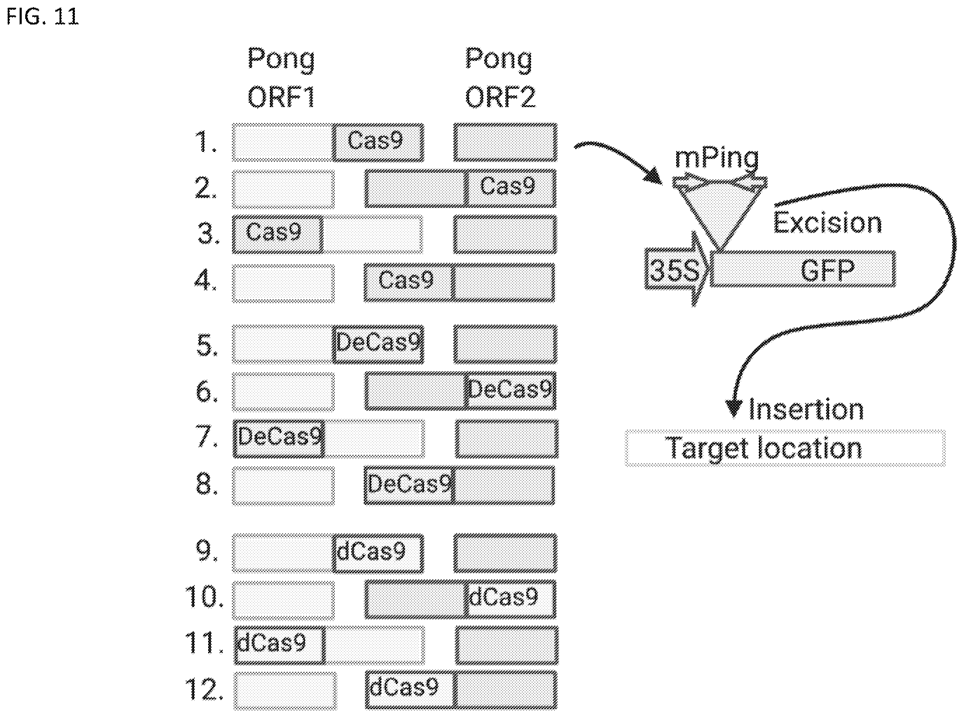

The goal was to fuse a TE-encoded transposase protein to the CRISPR/Cas9 system to achieve targeted integration of DNA in plants. The reason lies in that the transposase protein would need to have two features to broadly function in this system. First, a wide host-range of functionality in plants was desired to create a universal tool for plant biology. Second, using split-transposase proteins (where the single transposase was encoded by two proteins that function together to achieve excision and insertion) would have a lower probability of disturbing protein function. It was reasoned that the rice mPing/Pong system would provide the highest probably of functioning when fused to Cas9, as the Pong transposase is split into two proteins (ORF1 and ORF2) and can mobilize the mPing non-autonomous (non-protein coding) TE in a range of plant species. mPing/Pong engineered system was obtained where the Pong transposase ORF1 and ORF2 were immobilized by the removal of the Pong TIRs, and mPing excision can be visualized by its removal from a constitutively expressed GFP gene (cartoons in FIG. 11). The Pong ORF1/ORF2 system was engineered with the G4S (GSSSS, SEQ ID NO: 64) flexible protein linker to allow efficient fusions to Cas9 proteins on either the N- or C-terminus of ORF1 or ORF2 and added an SV40 nuclear localization signal (NLS) to these protein fusions. Three versions of the Cas9 protein where used, the catalytically active Cas9, the single-stranded nickase deCas9, and the catalytically inactive dCas9. A total of 12 constructs were generated (3 Cas9 proteins×4 ORF1/ORF2 positions) (FIG. 11) with a gRNA known to target the Arabidopsis PDS3 gene (https://doi.org/10.1038/nbt.2655).

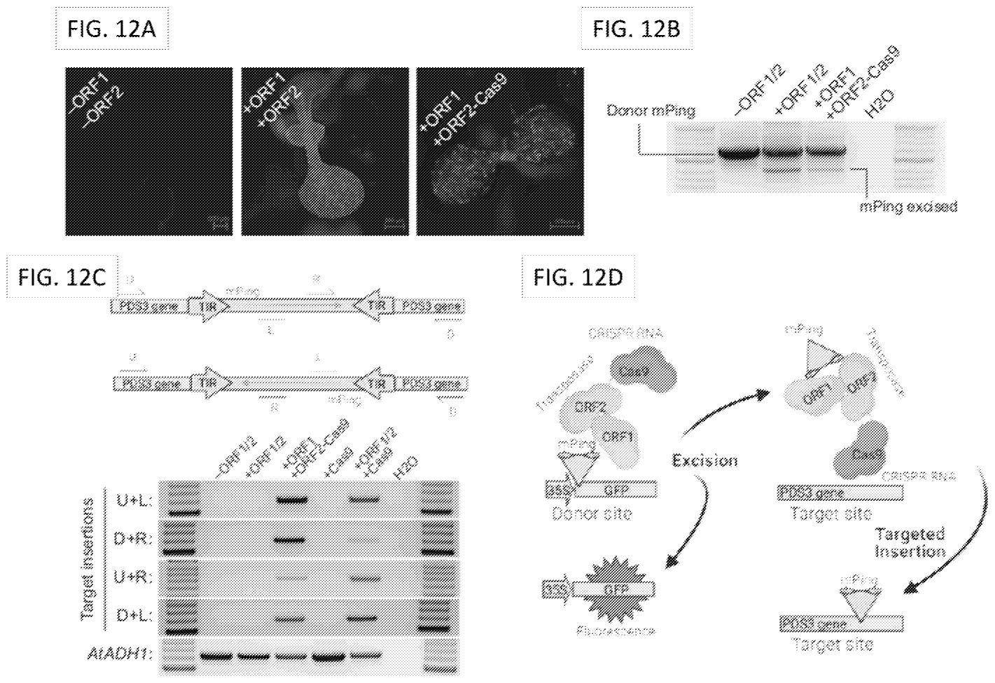

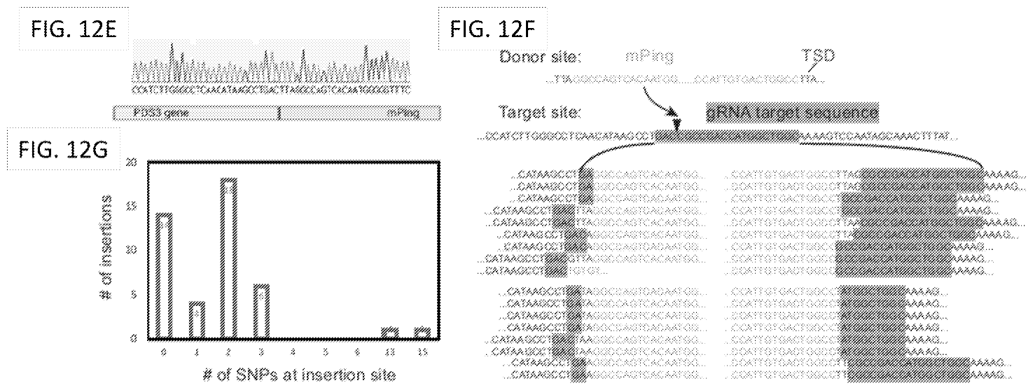

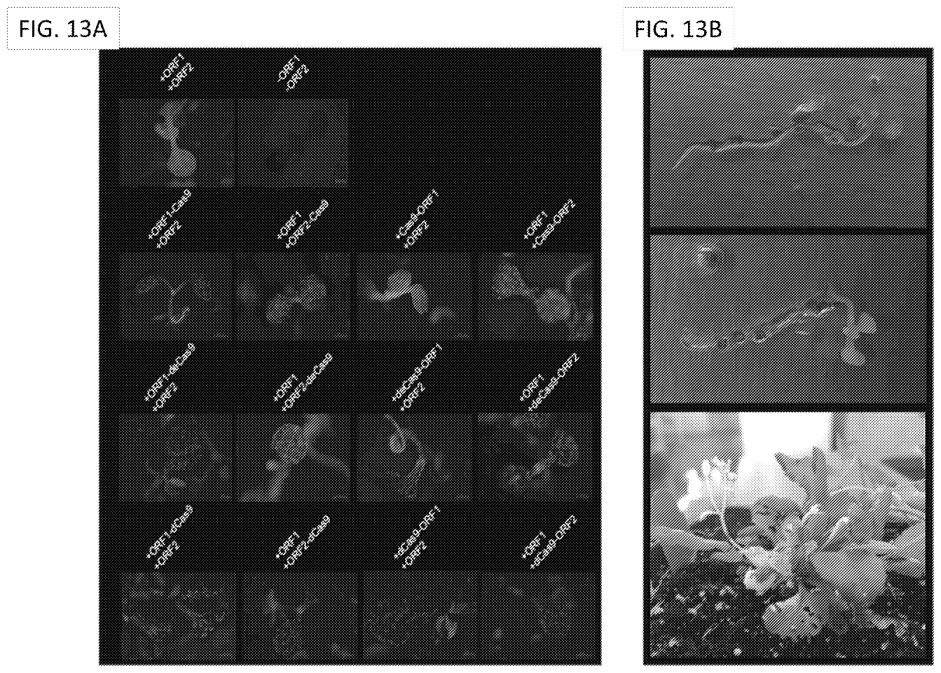

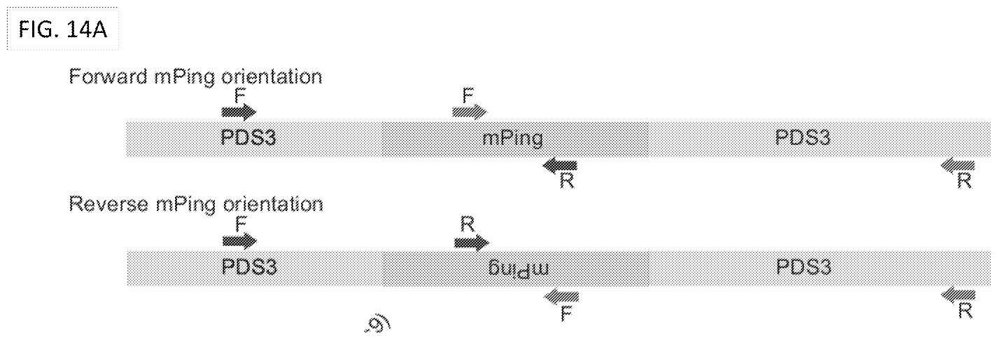

To determine if the Pong transposase was functional when fused to Cas9 derivatives, mPing excision from the donor site within GFP was assayed by visualizing the GFP fluorescence of seedlings (FIG. 12A and FIG. 13A). GFP fluorescence is a marker of mPing excision from the GFP donor site, and this fluorescence was detected for all 12 fusion proteins, but not the negative control without ORF1/ORF2 (summarized in FIG. 12A, full data in FIG. 13A), verifying that ORF1 and ORF2 are co-creating a functional transposase protein even while fused to Cas9. The function of the transposase was additionally verified using a PCR assay to detect mPing excision from the donor site. mPing excises out of its donor position when the transposase is fused to Cas9 (FIG. 12B), although the frequency may be decreased compared to transposase proteins with no fusion (FIG. 12B). A functional CRISPR/Cas9 system was verified through the observation of white seedlings and sectors in plants with the Cas9 proteins (dCas9 plants did not display white plants or sectors) (FIG. 13B). These white sectors and plants are generated by CRISPR/Cas9 targeted mutation of the PDS3 target region. Overall, these results demonstrate that fusion of the Cas9 and transposase proteins does not stop either the function of Cas9 nor the transposase.

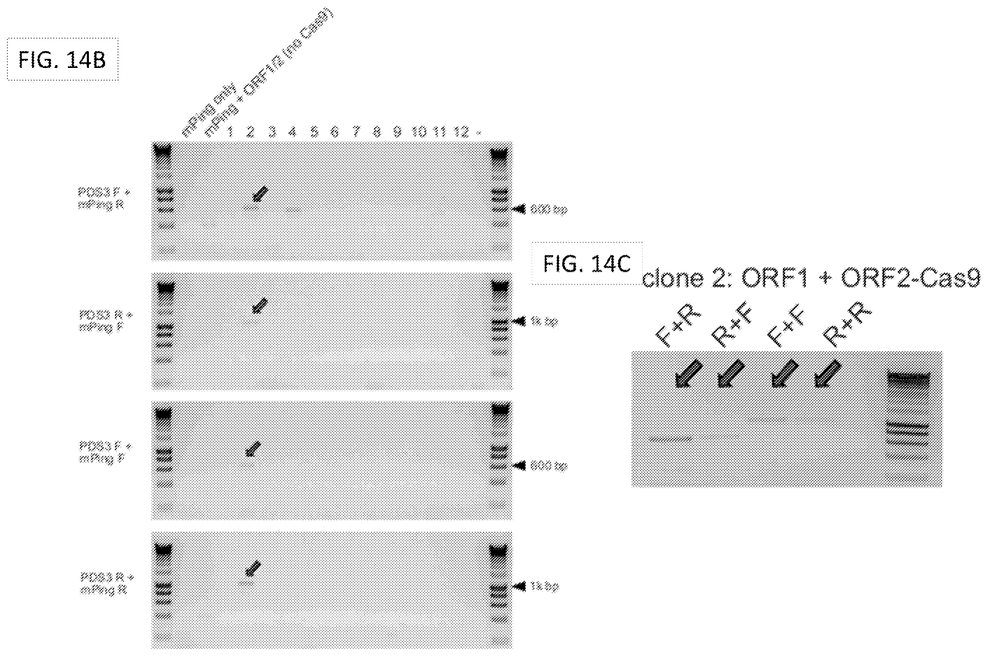

A PCR amplification strategy was employed to detect targeted mPing insertions into the Arabidopsis PDS3 gene (summarized in FIG. 12C, full data in FIGS. 14A-14B). As controls, T2 seedling pools were screened using negative control lines that either lack ORF1/ORF2, or that lack the Cas9 protein. Based on the strict expectations regarding the size of the PCR product that corresponds to the precise insertion of mPing into PDS3 (black arrowheads, FIG. 14B), it was found that clone #2 displayed the correct size PCR band in all PCR assays (FIG. 14B, FIG. 14C). This targeted insertion was only detected if both the transposase proteins (ORF1/ORF2) and Cas9 were in the same plants (FIG. 12C and FIG. 14B). The PCR can identify mPing insertions in the forward or reverse orientation (FIG. 14A), and the fact that clone #2 amplified for both suggested that there is more than one mPing insertion in this pool of plants. Clone #2 encodes for ORF1+ORF2-Cas9, where ORF2 has a C-terminal fusion to the Cas9 protein. This data demonstrated targeted insertion of mPing into the PDS3 gene (summarized in FIG. 12D), and since the catalytically-dead dCas9 version tested does not show targeted insertion, this demonstrated that the cleavage activity of Cas9 is required for targeted insertion of mPing.

Characterization of Target Site Insertions

To characterize the sequence at the junction of the targeted insertion site, the target-site PCR assay was biologically replicated (FIG. 14C), these PCR products were cloned and sequenced using Sanger sequencing. An example of the Sanger sequencing junction of mPing and PDS3 at a targeted integration event is shown in FIG. 12E. A total of 96 clones was sequenced and found that they represented at least 44 unique targeted transposition events. Both mPing forward and reverse orientation insertions were identified, demonstrating the random directionality of the targeted insertion event (FIG. 12F). Most insertions have either intact or partial TTA/TAA sequence on one end of the insertion (FIG. 12F). This sequence came from the donor site and is part of the known target site duplication (TSD) of the Pong/mPing TE system. The presence of only one TSD, rather than one on either side of the TE insertion, as usual for a transposable element duplication event, signifies that Cas9 created a blunt cut at the insertion site, but the transposase protein made a staggered (sticky-end) cut at the donor site, before the integration event. This demonstrates that both the Cas9 and transposase proteins are functional and necessary for generating this targeted insertion: the transposase cuts mPing out from the donor site using a staggered cut with a TTA/TAA overhang on one side, and Cas9 cuts the insertion site guided by the gRNA sequence.

For each insertion, the gRNA target sequence was preserved and mPing had inserted at the expected Cas9 cleavage point between the third and fourth nucleotide (FIG. 12F). In all but one sequence read the mPing element is complete, with only small base insertions or deletions found at the target site. Of the 44 distinct insertion events, most (95%) had 0-3 nucleotide changes compared to the expected insertion junction (FIG. 12G), and 32% had perfect seamless junctions without any SNPs (FIG. 12G). The lack of deletions or other insertions at these insertion sites demonstrated the seamless or near-seamless repair of the insertion events by the transposase protein compared to typical sites of blunt-end DNA breaks.

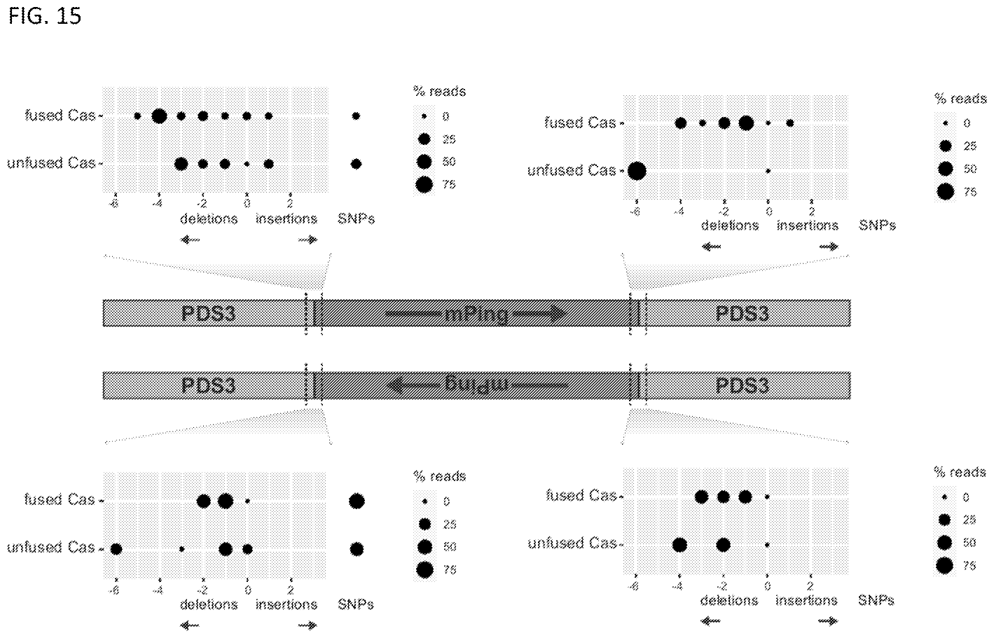

To better characterize the insertion site junctions upon targeted integration of mPing, mPing targeted integration events were deep sequenced. As shown in FIG. 15, nearly all insertions had between 0-3 nucleotide changes compared to the predicted insertion configuration. The number of base deletions and insertions at the 5′ and 3′ junctions of mPing inserted into PDS3 was assayed, and since mPing can insert in either orientation, this provided four junctions for analysis (FIG. 15). When the transposase ORF2 was translationally fused to Cas9 (as in FIG. 11), it was found 0-1 base insertions, and 0-5 base deletions, however, the majority of the deletions are 0-3 bases (FIG. 15). Together, this data demonstrated that upon targeted integration of mPing, the junctions were either seamless (zero base insertions or deletions) or just a few nucleotide bases away (near-seamless). This low rate of change during targeted insertion was likely due to the transposase protein stabilizing and protecting the cleaved ends of mPing DNA and the insertion site DNA from nucleases during the integration event.

Not Random Integration

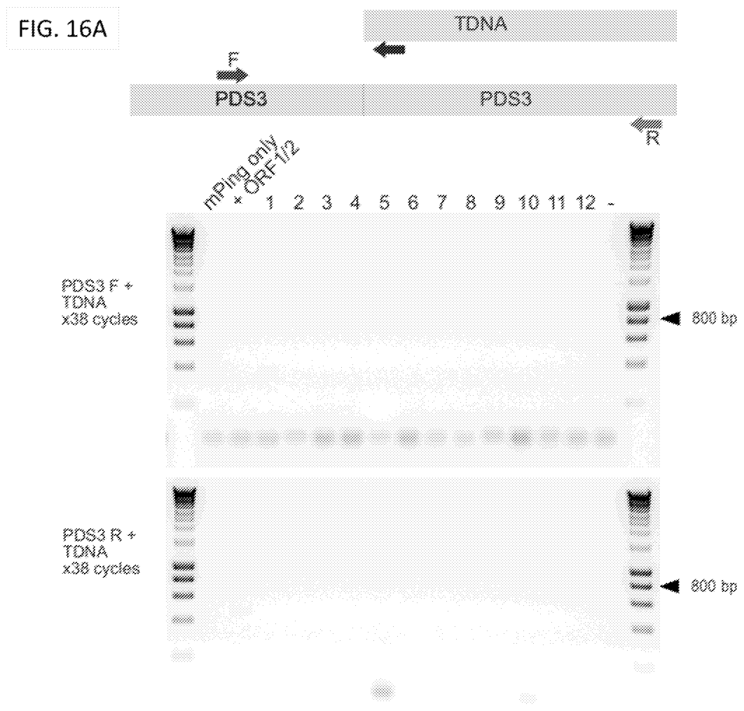

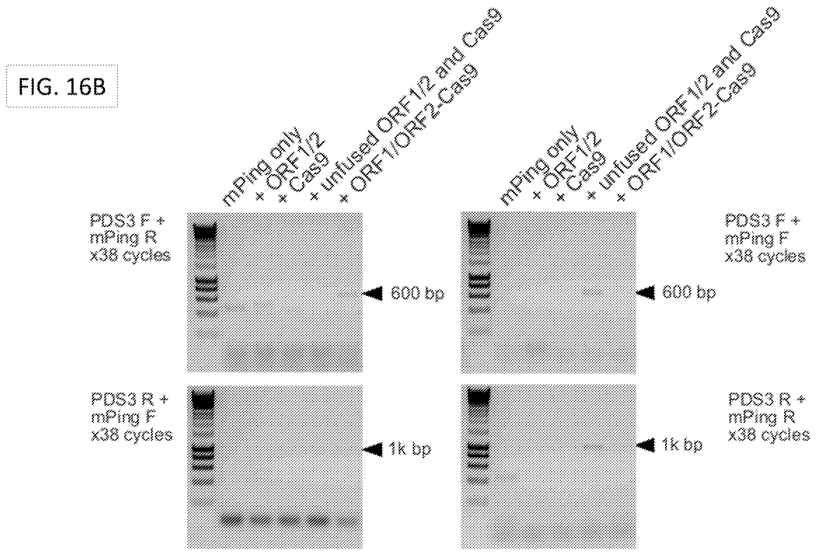

Several previous reports have demonstrated that transgenes will insert at a low frequency into any site of double-strand break. This is likely due to the transgene being extra-chromosomal DNA at the time of repair of a double-strand DNA break caused by Cas9. To determine if the mPing targeted insertion detected in FIGS. 12-14 requires the transposase protein, a PCR assay was performed for the integration of the transgene backbone encoding the ORF2-Cas9 protein into the DNA break generated at PDS3. It was reasoned that if the mPing insertion into PDS3 was a product of transgene insertion, rather than specifically transposition, it would be equally likely to detect other parts of our transgene at this insertion site location. However, the transgene sequences at PDS3 was not detected (FIG. 16A), demonstrating that mPing insertion required the transposase to excise the mPing element from the donor position to participate in targeted integration.

Next it was determined whether it was essential that the transposase protein and Cas9 were directly fused, or if both proteins unfused in the same cell could perform targeted insertion. The findings were that in some cases the two proteins could be unfused and targeted insertion would take place (FIG. 16B and FIG. 12C). At the same time, both transposase proteins (ORF1 and ORF2) were required and that the catalytic activity of Cas9 was necessary (FIG. 16B and FIG. 12C). Together, this data demonstrated that to obtain targeted insertion, it was essential that the transposase excise the element out of the donor position, and that Cas9 cleave the insertion site, but the two proteins do not necessarily need to be fused together. The success of the unfused configuration of Cas9 and ORF2 suggested that any extra-chromosomal DNA can be used by the cell to repair a double-stranded break caused by Cas9, and the transposase provided this available extra-chromosomal DNA by excising mPing out of the chromosome.

The accuracy of the integration events was compared when Cas9 was fused to ORF2 compared to when the two proteins where unfused and in the same cell (FIG. 15). In three of the four mPing junctions analyzed by deep sequencing, the unfused ORF2/Cas9 configuration had larger 4-6 base deletions compared to the fused ORF2-Cas9 (FIG. 15). This was likely due to the more rapid binding of the transposase protein to the site that just underwent Cas9 cleavage when the two proteins are physically fused. This more rapid binding will protect free ends of DNA from degradation by nucleases. This data also suggested a key advantage of fusing Cas9 to ORF2: more accurate insertions at the single base pair resolution.

Programmability of Target Sites

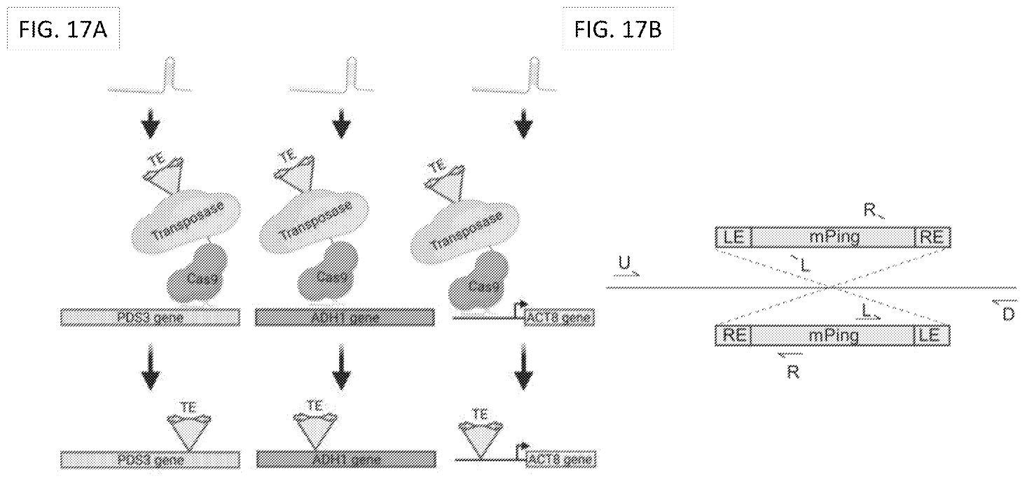

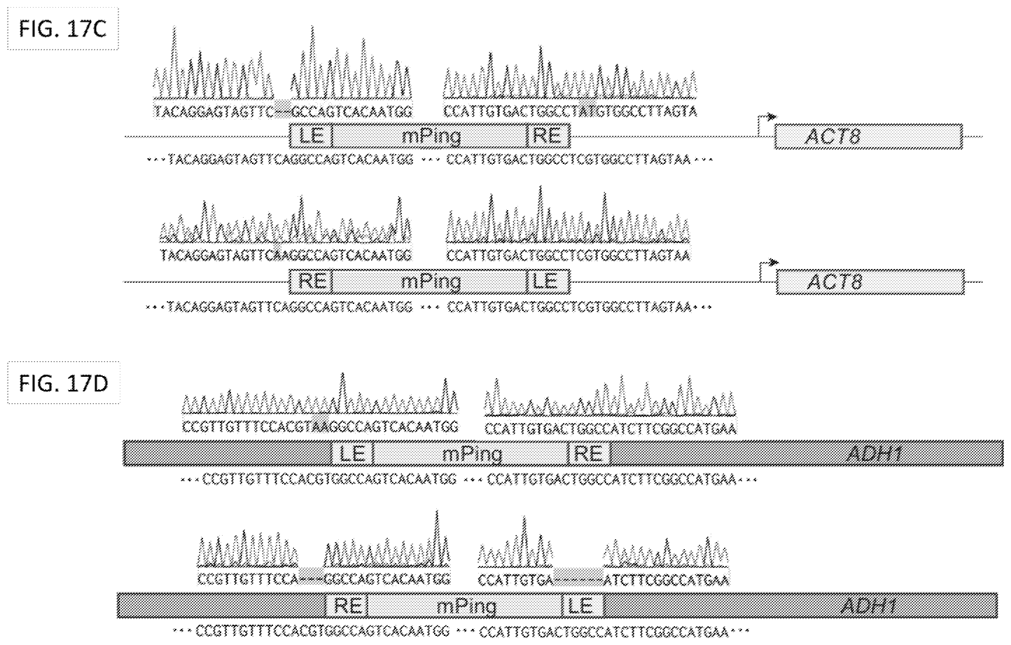

Multiple sites in the Arabidopsis genome have been successfully targeted where the inventors or others from the literature have demonstrated functional gRNAs (summarized in FIG. 17A). In addition to using gRNAs that target the gene body of PDS3 (FIGS. 12-16), the ADH1 gene and the region upstream of the ACT8 gene were successfully targeted. The PCR strategy to detect these insertions is shown in FIG. 17B. These were either within genes (PDS3 and ADH1) (ADH1 insertion shown in FIG. 17D), or in non-coding promoter regions of the ACT8 gene (shown in FIG. 17C). This data demonstrated the programmability of the targeted insertion system (summarized in FIG. 17A), as all needs to do to target a different region of the genome was to change the CRISPR gRNA sequence.

Measurement of Frequency of Targeted Insertion

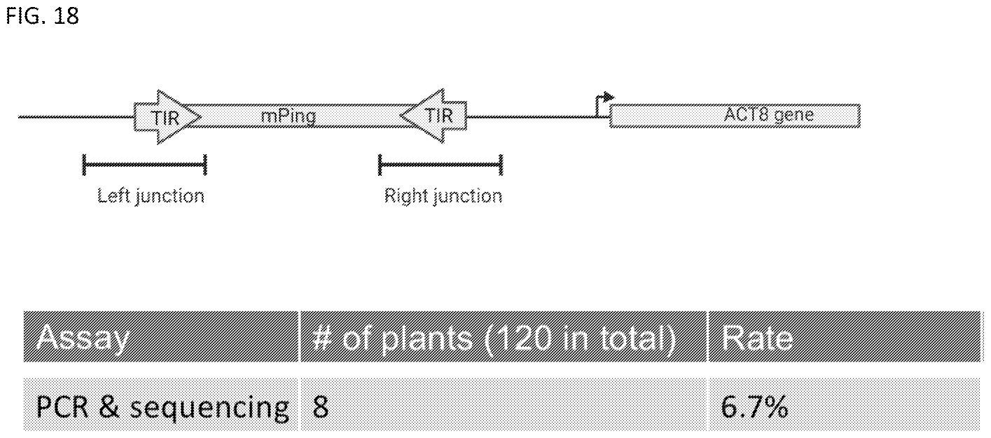

Since insertions into PDS3 generate albino plants and are lethal, insertions into the ACT8 promoter were used to measure the frequency of insertion (since the insertion will not create a gene knock-out mutation that may be selected against). Both ends of the mPing element were inserted into the ACT8 in 6.7% of T2 progeny plants (FIG. 18). This rate of more than 1 successful targeted insertion in 15 plants screened is a high rate that was easily screened for during transgenesis.

Alteration of Cargo DNA

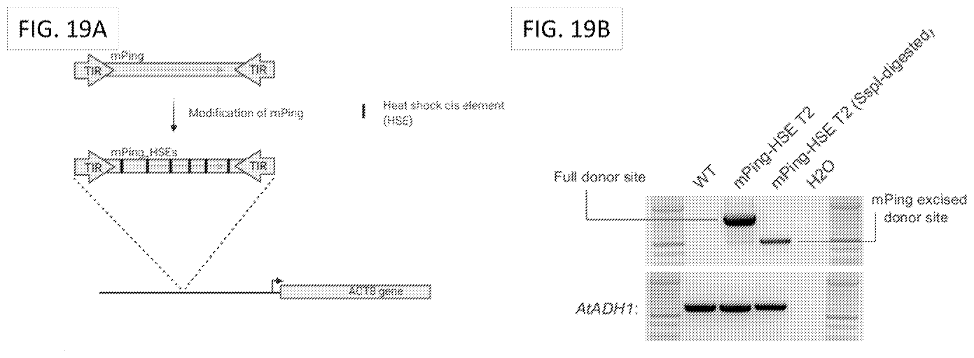

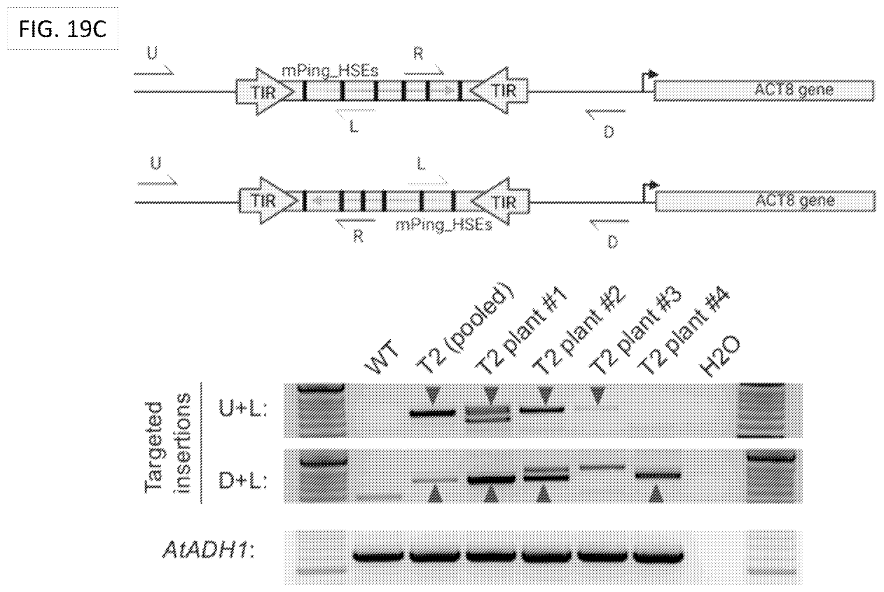

The mPing transposon is composed of terminal inverted repeats (TIRs) with DNA between them. The sequence of the TIRs is essential for transposition (as binding sites for the ORF1- and ORF2-encoded transposase proteins), but the sequence of the DNA between them (cargo) is not essential. To determine if different engineered DNA could be delivered to the target site, the cargo DNA was altered in the donor plasmid. An mPing element was engineered to carry an array of six heat-shock enhancer elements (FIG. 19A), with the goal of transposing these into a gene's promoter. A well-characterized Arabidopsis heat shock enhancer sequence was used, which is known to occur in arrays of more than one element. These enhancers were chosen because their short size and the fact that their direction upstream of a promoter did not matter, as the orientation of mPing insertion cannot be controlled. It was found that this new heat shock element-loaded mPing element (mPing-HSE) could perform the operation of a TE, as it could be excised by the transposase proteins (FIG. 19B). It was found upon transposition, mPing-HSE could successfully undergo targeted insertion similar to mPing, guided by Cas9 and the gRNA into the promoter region of the ACT8 gene (FIG. 19C), demonstrating the targeted delivery of engineered cargo DNA to a gene in its native context on the chromosome.

Use of Other Nucleases

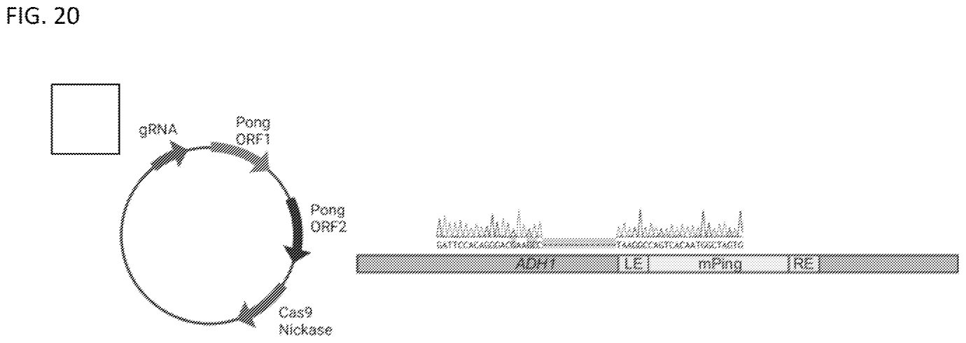

In order to determine if the system of the instant disclosure would only work with the Cas9 nuclease, or could use any sequence-specific programmable nuclease, as it was unable to detect targeted insertion with the Cas9 nickase fusion proteins created in FIG. 11. A further attempt was to detect targeted insertion with an unfused nickase Cas9 protein in the same vector as the ORF1 and ORF2 transposase proteins (FIG. 20). This Cas9 derivative has a mutation that results in it only cutting one strand of DNA (nicking), not both strands as the canonical Cas9. A low frequency of targeted insertion was detected using the Cas9 nickase protein. Upon Sanger sequencing this insertion displayed a 14 nucleotide deletion (FIG. 20). This data demonstrated that other derivative versions of Cas9 can be used with transposase ORFs for targeted insertion, but since the integration site was less precise compared to Cas9, targeted insertion with the Cas9 nickase was not being pursued further.

Second, Cas9 was replaced with CFP1 nuclease, belonging to a different class of targeting nucleases, and a gRNA specific for use with CPF1 nucleases was designed. CPF1 was fused to the ORF2 transposase protein and again demonstrated successful targeted integration of mPing. This data demonstrates that the system of the instant disclosure is not specific to Cas9, and any targeted nuclease can be used. In addition, in this experiment, two gRNAs were simultaneously used in one vector and plants that had insertions in both ADH1 and the ACT8 promoter were identified. This demonstrated that two or more regions of the genome can be targeted simultaneously and efficiently. This was important for downstream multiplex engineering of more than one genome locus at a time.

One-Component Vs. Two-Component Systems

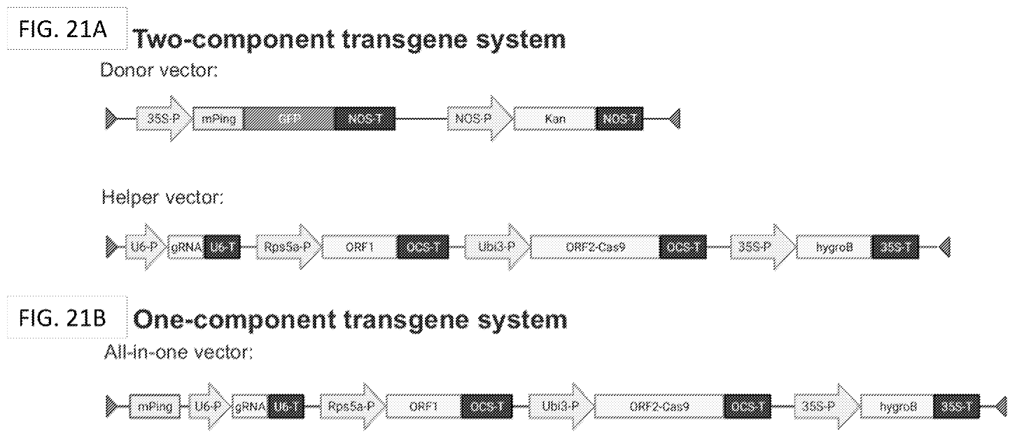

It was discovered that mPing excision and targeted insertion could take place from either the same transgene as ORF1, ORF2, Cas9 and the gRNA were encoded from (one-component system, FIG. 21B), or if the mPing donor site was already integrated into the Arabidopsis genome (two-component system) (FIG. 21A). Previous targeted insertions (FIGS. 11-16) used a 35S promoter-mPing-GFP donor site that had been previously integrated into the Arabidopsis genome (see cartoons in FIG. 10-11 and donor vector in FIG. 21A). In contrast, the mPing-HSE donor site was present on the same transgene as ORF1, ORF2, Cas9 and the gRNA are encoded from (FIG. 21B) and can still excise and undergo targeted insertion (FIG. 19). This is important because attempts to target mPing and derivative elements in other plants or with different cargo will want to use only the one-component transgene and the one cycle of transgenesis to accomplish targeted insertion. Of note, the one-component mPing donor site was not in the 35S-GFP sequence, but rather in different sequence that was used to cut down on the size of the transgene and does not provide the excision reporter of GFP fluorescence (FIG. 21). Instead, when using the one-component system, excision is monitored by PCR only (FIG. 18B), and this demonstrated that the surrounding DNA sequence around mPing at the donor site was not important in this system.

### Example 8: Measuring Specificity/Off-Target Integration Rate

The rate of off-target mPing insertion into the genome is tested. This is important because it is reasoned that the direct fusion between Cas9 and ORF2 has fewer off-targets compared to having the two proteins present but unfused. Therefore, fusing the two proteins can be important to limit the activity of the transposase protein so it does not integrate mPing all over the genome.

Approaches to detect mPing insertion sites include Southern blot, PCR ‘transposable-element display’ and long-read sequencing to sequence the full genome and detect other full or partial integration events of mPing.

To improve propagation of the insertion events into the next generation and limit the off-target effect, the promoter of the Cas9-transposase fusion protein is altered to only expressed in the egg cell. Accordingly, all cells of the plant will have the same insertion that occurred in the egg cell, while the insertions will not continue to accumulate during plant development.

### Example 9: Testing Other Uses of Targeted Insertion

Repeated delivery of different transgene cargos to the same permissive location in the genome is tested. The results demonstrate the reduced variability and improved experimental/product reproducibility when transgenes are targeted to the same region of the genome using systems of the instant disclosure.

Targeted delivery of a protein tag to a coding region using systems of the instant disclosure is also tested. The protein tag can be used to epitope tag a protein at its native location and within its native regulatory context.

Targeted addition of a strong promoter to drive constitutive expression of a gene at its native position for either over-expression of the sense mRNA or antisense expression for gene silencing is also tested.

### Example 10: Rewiring Gene Regulation Based on Targeted Insertion

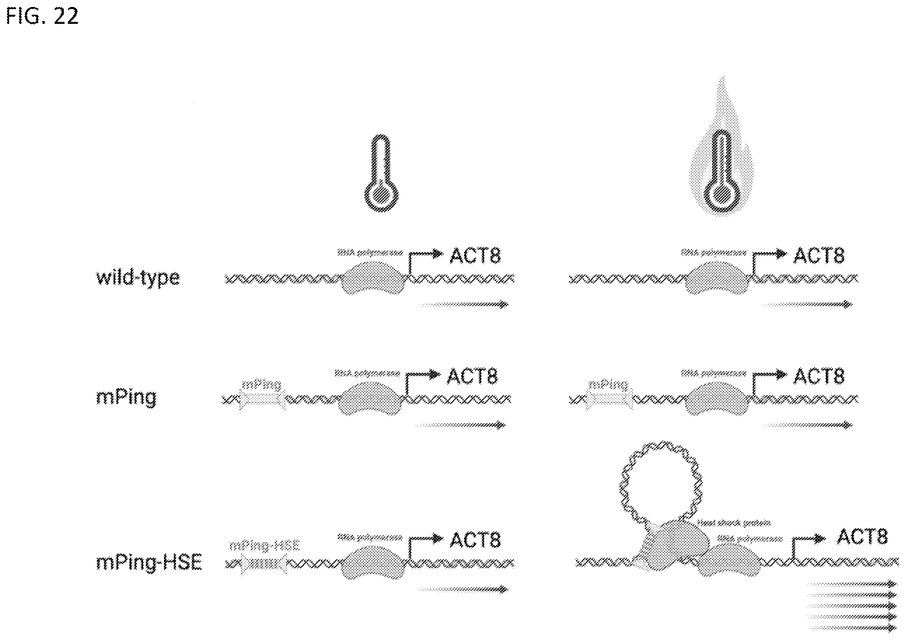

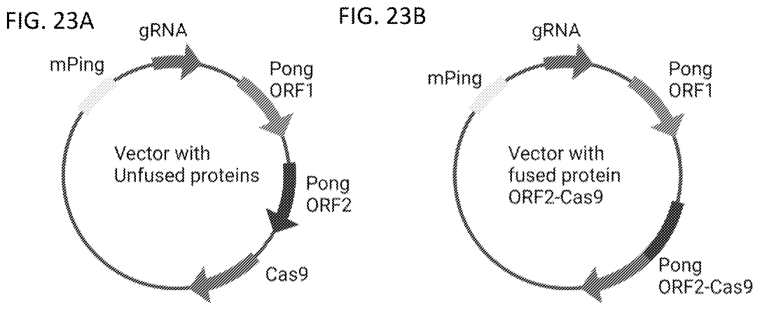

The mPing-HSE element was previously generated, in which the cargo DNA has an array of six heat-shock cis-regulatory enhancer elements (FIG. 19A). During the heat shock response, these enhancer elements are bound by a heat shock protein and enhance the transcription of a nearby gene. The one-component transgene system (FIG. 21B) is used to target the distal promoter region of the ACT8 gene (FIG. 19C). The ACT8 gene is chosen because it is not regulated by heat and is often used as a control gene because of its steady transcription into mRNA even during heat stress (FIG. 22). The goal is to demonstrate the utility of the targeted insertion technology by rewiring the ACT8 gene in its native chromosomal context, providing this gene the new programmed ability to increase expression as a response to heat stress. Lines with the original mPing (no heat-shock elements) inserted at the same location are used as controls (insertion in FIG. 17, experimental design in FIG. 22). An additional control is wild-type plants without any insertion upstream of ACT8. Both of these controls do not to provide ACT8 with higher expression during heat shock (FIG. 22).

### Example 12: Targeted Insertion in a Crop

A variation of the systems of the instant disclosure was transformed into soybean plants (Glycine max). Soybean is annually one of the top three crops grown in the United States, and the #1 oil crop. Transformation was performed by the Danforth Center's Plant Transformation Facility (PTF). Soybean explants were transformed using Agrobacterium, cultured, and selected for the integration of the transgene. Next, roots and shoots were regenerated and the plants transplanted to soil and sampled.

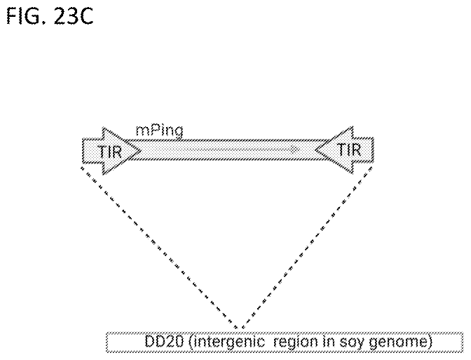

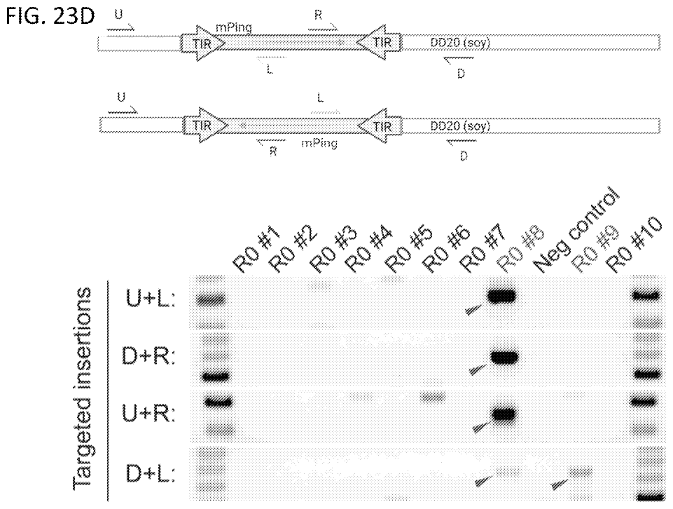

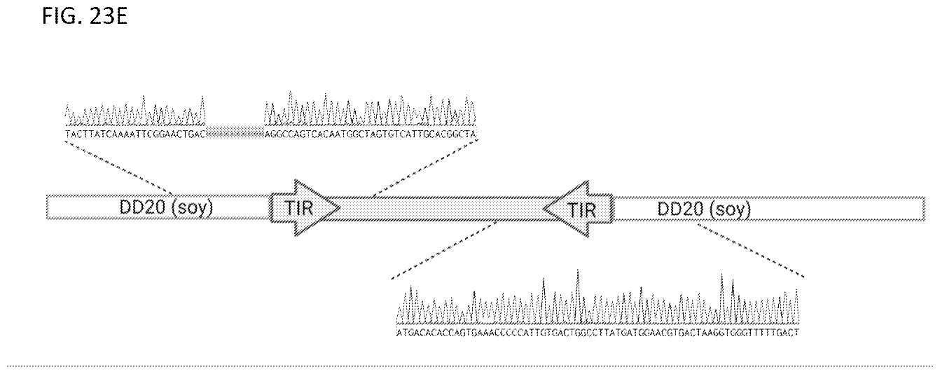

To transfer the system to soybeans, a binary vector that is proven to function in soybean transformation was used. The transgenes all have the same mPing and ORF1 sequences, and a different gRNA that has been previously demonstrated to function in the soybean genome, which targets an intergenic region called “DD20” (PMID 26294043). Two configurations of the transgene system were used in soybean: 1) ORF2 unfused to Cas9 (FIG. 23A), and 2) ORF2 fused to Cas9 (FIG. 23B).

RO plants that have been regenerated from the transformation process were screened and confirmed via PCR to have the entire transgene integrated into the genome. Plants were assayed for mPing excision which demonstrates the successful transposition of the donor polynucleotide, Cas9 cleavage and mutation of the target locus (demonstrates that the CRISPR/Cas parts of the system are working), and for targeted insertion of mPing (see below). Screening for targeted insertion was performed using four PCR reactions that target each end of the mPing insertion, in either direction of potential insertion (FIG. 23D).

Of the 10 transgenic RO plants produced from the unfused transgene configuration in FIG. 23A, two amplified in our assays for targeted insertion of mPing (Plant #8 and #9, FIG. 23D). These PCR products were sequenced and confirmed to be targeted integrations of mPing at the DD20 intergenic target locus (FIG. 23E). This rate of 20% of RO plants is very high compared to other methods of crop genome targeted integration or HDR. Of note, since plant #8 amplifies in all four PCR reactions (FIG. 23E), it represents more than one insertion event.

The identified targeted insertion event of mPing that is a near-seamless insertion on the 3′ side, and has a 10 base pair deletion on the 5′ end. This deletion is all of soybean DD20 DNA, while the mPing insertion is identical to mPing at the donor site. This again demonstrates that the mutations, if they do occur, are in the target site DNA, and not in the newly transposed element.

A total of 61 RO plants were investigated with the ORF2-Cas9 fused protein in FIG. 23B. Even with considerable effort, a targeted insertion in these plants was not identified. It was found that ˜28% of these plants have mPing excision, demonstrating that the transposase aspect of our system is working, but none of these plants showed mutation accumulation at the target site, which demonstrates that Cas9 was not functional when fused to ORF2 in soybean plants. Different linker sequences are to improve the fusion of Cas9 to ORF2 towards a functional CRISPR/Cas9 system in these plants.

SEQUENCES

| SEQ. | | | | |
|---|---|---|---|---|
| ID | | Sequence | | |
| NO. | Source | type | Sequence | Name |
| 1 | Oryza | Protein | MDPSPAVDPSPAVDPSPAAETRRRATGK | Pong ORF1 |
| | sativa | | GGKQRGGKQLGLKRPPPISVPATPPPAA | protein |
| | | | TSSSPAAPTAIPPRPPQSSPIFVPDSPN | |
| | | | PSPAAPTSSLASGTSTARPPQPQGGGWG | |
| | | | PTSTISPNFASFFGNQQDPNSCLVRGYP | |
| | | | PGGFVNFIQQNCPPQPQQQGENFHFVGH | |
| | | | NMGFNPISPQPPSAYGTPTPQATNQGTS | |
| | | | TNIMIDEEDNNDDSRAAKKRWTHEEEER | |
| | | | LASAWLNASKDSIHGNDKKGDTFWKEVT | |
| | | | DEFNKKGNGKRRREINQLKVHWSRLKSA | |
| | | | ISEFNDYWSTVTQMHTSGYSDDMLEKEA | |
| | | | QRLYANRFGKPFALVHWWKILKREPKWC | |
| | | | AQFEKRKRKSEMDAVPEQQKRPIGREAA | |
| | | | KSERKRKRKKENVMEGIVLLGDNVQKII | |
| | | | KVTQDRKLEREKVTEAQIHISNVNLKAA | |
| | | | EQQKEAKMFEVYNSLLTQDTSNMSEEQK | |
| | | | ARRDKALQKLEEKLFAD* | |
| 2 | Oryza | DNA | atggatccgtcgccggccgtggatccgt | DNA |
| | sativa | | cgccggccgtggatccgtcgccggctgc | sequence |
| | | | tgaaacccggcggcgtgcaaccgggaaa | encoding |
| | | | ggaggcaaacagcgcgggggcaagcaac | Pong ORF1 |
| | | | taggattgaagaggccgccgccgatttc | protein |
| | | | tgtcccggccaccccgcctcctgctgcg | |
| | | | acgtcttcatcccctgctgcgccgacgg | |
| | | | ccatcccaccacgaccaccgcaatcttc | |
| | | | gccgattttcgtccccgattcgccgaat | |
| | | | ccgtcaccggctgcgccgacctcctctc | |
| | | | ttgcttcggggacatcgacggcaaggcc | |
| | | | accgcaaccacaaggaggaggatgggga | |
| | | | ccaacatcgaccatttccccaaactttg | |
| | | | catctttctttggaaaccaacaagaccc | |
| | | | aaattcatgtttggtcaggggttatcct | |
| | | | ccaggagggtttgtcaattttattcaac | |
| | | | aaaattgtccgccgcagccacaacagca | |
| | | | aggtgaaaattttcatttcgttggtcac | |
| | | | aatatggggttcaacccaatatctccac | |
| | | | agccaccaagtgcctacggaacaccaac | |
| | | | accccaagctacgaaccaaggcacttca | |
| | | | acaaacattatgattgatgaagaggaca | |
| | | | acaatgatgacagtagggcagcaaagaa | |
| | | | aagatggactcatgaagaggaagagaga | |
| | | | ctggccagtgcttggttgaatgcttcta | |
| | | | aagactcaattcatgggaatgataagaa | |
| | | | aggtgatacattttggaaggaagtcact | |
| | | | gatgaatttaacaagaaagggaatggaa | |
| | | | aacgtaggagggaaattaaccaactgaa | |
| | | | ggttcactggtcaaggttgaagtcagcg | |
| | | | atctctgagttcaatgactattggagta | |
| | | | cggttactcaaatgcatacaagcggata | |
| | | | ctcagacgacatgcttgagaaagaggca | |
| | | | cagaggctgtatgcaaacaggtttggaa | |
| | | | aaccttttgcgttggtccattggtggaa | |
| | | | gatactcaaaagagagcccaaatggtgt | |
| | | | gctcagtttgaaaagaggaaaaggaaga | |
| | | | gcgaaatggatgctgttccagaacagca | |
| | | | gaaacgtcctattggtagagaagcagca | |
| | | | aagtctgagcgcaaaagaaagcgcaaga | |
| | | | aagaaaatgttatggaaggcattgtcct | |
| | | | cctaggggacaatgtccagaaaattatc | |
| | | | aaagtgacgcaagatcggaagctggagc | |
| | | | gtgagaaggtcactgaagcacagattca | |
| | | | catttcaaacgtaaatttgaaggcagca | |
| | | | gaacagcaaaaagaagcaaagatgtttg | |
| | | | aggtatacaattccctgctcactcaaga | |
| | | | tacaagtaacatgtctgaagaacagaag | |
| | | | gctcgccgagacaaggcattacaaaagc | |
| | | | tggaggaaaagttatttgctgactag | |
| 3 | Oryza | Protein | MQSLAISLLLSETHSLFSHTKTSSLLSL | Pong ORF2 |
| | sativa | | LFLSSSKMSEQNTDGSQVPVNLLDEFLA | protein |
| | | | EDEIIDDLLTEATVVVQSTIEGLQNEAS | |
| | | | DHRHHPRKHIKRPREEAHQQLVNDYFSE | |
| | | | NPLYPSKIFRRRFRMSRPLFLRIVEALG | |
| | | | QWSVYFTQRVDAVNRKGLSPLQKCTAAI | |
| | | | RQLATGSGADELDEYLKIGETTAMEAMK | |
| | | | NFVKGLQDVFGERYLRRPTMEDTERLLQ | |
| | | | LGEKRGFPGMFGSIDCMHWHWERCPVAW | |
| | | | KGQFTRGDQKVPTLILEAVASHDLWIWH | |
| | | | AFFGAAGSNNDINVLNQSTVFIKELKGQ | |
| | | | APRVQYMVNGNQYNTGYFLADGIYPEWA | |
| | | | VFVKSIRLPNTEKEKLYADMQEGARKDI | |
| | | | ERAFGVLQRRFCILKRPARLYDRGVLRD | |
| | | | VVLACIILHNMIVEDEKETRIIEEDADA | |
| | | | NVPPSSSTVQEPEFSPEQNTPFDRVLEK | |
| | | | DISIRDRAAHNRLKKDLVEHIWNKFGGA | |
| | | | AHRTGN | |
| 4 | Oryza | DNA | atgcagagtttagccatctctctactcc | DNA |
| | sativa | | tctcagaaactcattccctcttttctca | sequence |
| | | | tacgaagacctcctcccttttatcttta | encoding |
| | | | ctgtttctctcttcttcaaagatgtctg | Pong ORF2 |
| | | | agcaaaatactgatggaagtcaagttcc | protein |
| | | | agtgaacttgttggatgagttcctggct | |
| | | | gaggatgagatcatagatgatcttctca | |
| | | | ctgaagccacggtggtagtacagtccac | |
| | | | tatagaaggtcttcaaaacgaggcttct | |
| | | | gaccatcgacatcatccgaggaagcaca | |
| | | | tcaagaggccacgagaggaagcacatca | |
| | | | gcaactGgtgaatgattacttttcagaa | |
| | | | aatcctctttacccttccaaaatttttc | |
| | | | gtcgaagatttcgtatgtctaggccact | |
| | | | ttttcttcgcatcgttgaggcattaggc | |
| | | | cagtggtcagtgtatttcacacaaaggg | |
| | | | tggatgctgttaatcggaaaggactcag | |
| | | | tccactgcaaaagtgtactgcagctatt | |
| | | | cgccagttggctactggtagtggcgcag | |
| | | | atgaactagatgaatatctgaagatagg | |
| | | | agagactacagcaatggaggcaatgaag | |
| | | | aattttgtcaaaggtcttcaagatgtgt | |
| | | | ttggtgagaggtatcttaggcgccccac | |
| | | | tatggaagataccgaacggcttctccaa | |
| | | | cttggtgagaaacgtggttttcctggaa | |
| | | | tgttcggcagcattgactgcatgcactg | |
| | | | gcattgggaaagatgcccagtagcatgg | |
| | | | aagggtcagttcactcgtggagatcaga | |
| | | | aagtgccaaccctgattcttgaggctgt | |
| | | | ggcatcgcatgatctttggatttggcat | |
| | | | gcattttttggagcagcgggttccaaca | |
| | | | atgatatcaatgtattgaaccaatctac | |
| | | | tgtatttatcaaggagctcaaaggacaa | |
| | | | gctcctagagtccagtacatggtaaatg | |
| | | | ggaatcaatacaatactgggtattttct | |
| | | | tgctgatggaatctaccctgaatgggca | |
| | | | gtgtttgttaagtcaatacgactcccaa | |
| | | | acactgaaaaggagaaattgtatgcaga | |
| | | | tatgcaagaaggggcaagaaaagatatc | |
| | | | gagagagcctttggtgtattgcagcgaa | |
| | | | gattttgcatcttaaaacgaccagctcg | |
| | | | tctatatgatcgaggtgtactgcgagat | |
| | | | gttgttctagcttgcatcatacttcaca | |
| | | | atatgatagttgaagatgagaaggaaac | |
| | | | cagaattattgaagaagatgcagatgca | |
| | | | aatgtgcctcctagttcatcaaccgttc | |
| | | | aggaacctgagttctctcctgaacagaa | |
| | | | cacaccatttgatagagttttagaaaaa | |
| | | | gatatttctatccgagatcgagcggctc | |
| | | | ataaccgacttaagaaagatttggtgga | |
| | | | acacatttggaataagtttggtggtgct | |
| | | | gcacatagaactggaaat | |
| 5 | Streptococcus | Protein | APKKKRKVGIHGVPAADKKYSIGLDIGT | Cas 9 |
| | pyogenes | | NSVGWAVITDEYKVPSKKFKVLGNTDRH | protein |
| | | | SIKKNLIGALLFDSGETAEATRLKRTAR | |
| | | | RRYTRRKNRICYLQEIFSNEMAKVDDSF | |
| | | | FHRLEESFLVEEDKKHERHPIFGNIVDE | |
| | | | VAYHEKYPTIYHLRKKLVDSTDKADLRL | |
| | | | IYLALAHMIKFRGHFLIEGDLNPDNSDV | |
| | | | DKLFIQLVQTYNQLFEENPINASGVDAK | |
| | | | AILSARLSKSRRLENLIAQLPGEKKNGL | |
| | | | FGNLIALSLGLTPNFKSNFDLAEDAKLQ | |
| | | | LSKDTYDDDLDNLLAQIGDQYADLFLAA | |
| | | | KNLSDAILLSDILRVNTEITKAPLSASM | |
| | | | IKRYDEHHQDLTLLKALVRQQLPEKYKE | |
| | | | IFFDQSKNGYAGYIDGGASQEEFYKFIK | |
| | | | PILEKMDGTEELLVKLNREDLLRKQRTE | |
| | | | DNGSIPHQIHLGELHAILRRQEDFYPEL | |
| | | | KDNREKIEKILTFRIPYYVGPLARGNSR | |
| | | | FAWMTRKSEETITPWNFEEVVDKGASAQ | |
| | | | SFIERMTNFDKNLPNEKVLPKHSLLYEY | |
| | | | FTVYNELTKVKYVTEGMRKPAFLSGEQK | |
| | | | KAIVDLLFKTNRKVTVKQLKEDYFKKIE | |
| | | | CFDSVEISGVEDRFNASLGTYHDLLKII | |
| | | | KDKDFLDNEENEDILEDIVLTLTLFEDR | |
| | | | EMIEERLKTYAHLFDDKVMKQLKRRRYT | |
| | | | GWGRLSRKLINGIRDKQSGKTILDFLKS | |
| | | | DGFANRNFMQLIHDDSLTFKEDIQKAQV | |
| | | | SGQGDSLHEHIANLAGSPAIKKGILQTV | |
| | | | KVVDELVKVMGRHKPENIVIEMARENQT | |
| | | | TQKGQKNSRERMKRIEEGIKELGSQILK | |
| | | | EHPVENTQLQNEKLYLYYLQNGRDMYVD | |
| | | | QELDINRLSDYDVDHIVPQSFLKDDSID | |
| | | | NKVLTRSDKNRGKSDNVPSEEVVKKMKN | |
| | | | YWRQLLNAKLITQRKFDNLTKAERGGLS | |
| | | | ELDKAGFIKRQLVETRQITKHVAQILDS | |
| | | | RMNTKYDENDKLIREVKVITLKSKLVSD | |
| | | | FRKDFQFYKVREINNYHHAHDAYLNAVV | |
| | | | GTALIKKYPKLESEFVYGDYKVYDVRKM | |
| | | | IAKSEQEIGKATAKYFFYSNIMNFFKTE | |
| | | | ITLANGEIRKRPLIETNGETGEIVWDKG | |
| | | | RDFATVRKVLSMPQVNIVKKTEVQTGGF | |
| | | | SKESILPKRNSDKLIARKKDWDPKKYGG | |
| | | | FDSPTVAYSVLVVAKVEKGKSKKLKSVK | |
| | | | ELLGITIMERSSFEKNPIDFLEAKGYKE | |
| | | | VKKDLIIKLPKYSLFELENGRKRMLASA | |
| | | | GELQKGNELALPSKYVNFLYLASHYEKL | |
| | | | KGSPEDNEQKOLFVEQHKHYLDEIIEQI | |
| | | | SEFSKRVILADANLDKVLSAYNKHRDKP | |
| | | | IREQAENIIHLFTLTNLGAPAAFKYFDT | |
| | | | TIDRKRYTSTKEVLDATLIHQSITGLYE | |
| | | | TRIDLSQLGGDKRPAATKKAGQAKKKK* | |
| 6 | Streptococcus | DNA | gctccgaagaagaagaggaaggttggca | Cas 9 DNA |
| | pyogenes | | tccacggggtgccagctgctgacaagaa | |
| | | | gtactcgatcggcctcgatattgggact | |
| | | | aactctgttggctgggccgtgatcaccg | |
| | | | acgagtacaaggtgccctcaaagaagtt | |
| | | | caaggtcctgggcaacaccgatcggcat | |
| | | | tccatcaagaagaatctcattggcgctc | |
| | | | tcctgttcgacagcggcgagacggctga | |
| | | | ggctacgcggctcaagcgcaccgcccgc | |
| | | | aggcggtacacgcgcaggaagaatcgca | |
| | | | tctgctacctgcaggagattttctccaa | |
| | | | cgagatggcgaaggttgacgattctttc | |
| | | | ttccacaggctggaggagtcattcctcg | |
| | | | tggaggaggataagaagcacgagcggca | |
| | | | tccaatcttcggcaacattgtcgacgag | |
| | | | gttgcctaccacgagaagtaccctacga | |
| | | | tctaccatctgcggaagaagctcgtgga | |
| | | | ctccacagataaggcggacctccgcctg | |
| | | | atctacctcgctctggcccacatgatta | |
| | | | agttcaggggccatttcctgatcgaggg | |
| | | | ggatctcaacccggacaatagcgatgtt | |
| | | | gacaagctgttcatccagctcgtgcaga | |
| | | | cgtacaaccagctcttcgaggagaaccc | |
| | | | cattaatgcgtcaggcgtcgacgcgaag | |
| | | | gctatcctgtccgctaggctctcgaagt | |
| | | | ctcggcgcctcgagaacctgatcgccca | |
| | | | gctgccgggcgagaagaagaacggcctg | |
| | | | ttcgggaatctcattgcgctcagcctgg | |
| | | | ggctcacgcccaacttcaagtcgaattt | |
| | | | cgatctcgctgaggacgccaagctgcag | |
| | | | ctctccaaggacacatacgacgatgacc | |
| | | | tggataacctcctggcccagatcggcga | |
| | | | tcagtacgcggacctgttcctcgctgcc | |
| | | | aagaatctgtcggacgccatcctcctgt | |
| | | | ctgatattctcagggtgaacaccgagat | |
| | | | tacgaaggctccgctctcagcctccatg | |
| | | | atcaagcgctacgacgagcaccatcagg | |
| | | | atctgaccctcctgaaggcgctggtcag | |
| | | | gcagcagctccccgagaagtacaaggag | |
| | | | atcttcttcgatcagtcgaagaacggct | |
| | | | acgctgggtacattgacggcggggcctc | |
| | | | tcaggaggagttctacaagttcatcaag | |
| | | | ccgattctggagaagatggacggcacgg | |
| | | | aggagctgctggtgaagctcaatcgcga | |
| | | | ggacctcctgaggaagcagcggacattc | |
| | | | gataacggcagcatcccacaccagattc | |
| | | | atctcggggagctgcacgctatcctgag | |
| | | | gaggcaggaggacttctaccctttcctc | |
| | | | aaggataaccgcgagaagatcgagaaga | |
| | | | ttctgactttcaggatcccgtactacgt | |
| | | | cggcccactcgctaggggcaactcccgc | |
| | | | ttcgcttggatgacccgcaagtcagagg | |
| | | | agacgatcacgccgtggaacttcgagga | |
| | | | ggtggtcgacaagggcgctagcgctcag | |
| | | | tcgttcatcgagaggatgacgaatttcg | |
| | | | acaagaacctgccaaatgagaaggtgct | |
| | | | ccctaagcactcgctcctgtacgagtac | |
| | | | ttcacagtctacaacgagctgactaagg | |
| | | | tgaagtatgtgaccgagggcatgaggaa | |
| | | | gccggctttcctgtctggggagcagaag | |
| | | | aaggccatcgtggacctcctgttcaaga | |
| | | | ccaaccggaaggtcacggttaagcagct | |
| | | | caaggaggactacttcaagaagattgag | |
| | | | tgcttcgattcggtcgagatctctggcg | |
| | | | ttgaggaccgcttcaacgcctccctggg | |
| | | | gacctaccacgatctcctgaagatcatt | |
| | | | aaggataaggacttcctggacaacgagg | |
| | | | agaatgaggatatcctcgaggacattgt | |
| | | | gctgacactcactctgttcgaggaccgg | |
| | | | gagatgatcgaggagcgcctgaagactt | |
| | | | acgcccatctcttcgatgacaaggtcat | |
| | | | gaagcagctcaagaggaggaggtacacc | |
| | | | ggctgggggaggctgagcaggaagctca | |
| | | | tcaacggcattcgggacaagcagtccgg | |
| | | | gaagacgatcctcgacttcctgaagagc | |
| | | | gatggcttcgcgaaccgcaatttcatgc | |
| | | | agctgattcacgatgacagcctcacatt | |
| | | | caaggaggatatccagaaggctcaggtg | |
| | | | agcggccagggggactcgctgcacgagc | |
| | | | atatcgcgaacctcgctggctcgccagc | |
| | | | tatcaagaaggggattctgcagaccgtg | |
| | | | aaggttgtggacgagctggtgaaggtca | |
| | | | tgggcaggcacaagcctgagaacatcgt | |
| | | | cattgagatggcccgggagaatcagacc | |
| | | | acgcagaagggccagaagaactcacgcg | |
| | | | agaggatgaagaggatcgaggagggcat | |
| | | | taaggagctggggtcccagatcctcaag | |
| | | | gagcacccggtggagaacacgcagctgc | |
| | | | agaatgagaagctctacctgtactacct | |
| | | | ccagaatggccgcgatatgtatgtggac | |
| | | | caggagctggatattaacaggctcagcg | |
| | | | attacgacgtcgatcatatcgttccaca | |
| | | | gtcattcctgaaggatgactccattgac | |
| | | | aacaaggtcctcaccaggtcggacaaga | |
| | | | accggggcaagtctgataatgttccttc | |
| | | | agaggaggtcgttaagaagatgaagaac | |
| | | | tactggcgccagctcctgaatgccaagc | |
| | | | tgatcacgcagcggaagttcgataacct | |
| | | | cacaaaggctgagaggggcgggctctct | |
| | | | gagctggacaaggcgggcttcatcaaga | |
| | | | ggcagctggtcgagacacggcagatcac | |
| | | | taagcacgttgcgcagattctcgactca | |
| | | | cggatgaacactaagtacgatgagaatg | |
| | | | acaagctgatccgcgaggtgaaggtcat | |
| | | | caccctgaagtcaaagctcgtctccgac | |
| | | | ttcaggaaggatttccagttctacaagg | |
| | | | ttcgggagatcaacaattaccaccatgc | |
| | | | ccatgacgcgtacctgaacgcggtggtc | |
| | | | ggcacagctctgatcaagaagtacccaa | |
| | | | agctcgagagcgagttcgtgtacgggga | |
| | | | ctacaaggtttacgatgtgaggaagatg | |
| | | | atcgccaagtcggagcaggagattggca | |
| | | | aggctaccgccaagtacttcttctactc | |
| | | | taacattatgaatttcttcaagacagag | |
| | | | atcactctggccaatggcgagatccgga | |
| | | | agcgccccctcatcgagacgaacggcga | |
| | | | gacgggggagatcgtgtgggacaagggc | |
| | | | agggatttcgcgaccgtcaggaaggttc | |
| | | | tctccatgccacaagtgaatatcgtcaa | |
| | | | gaagacagaggtccagactggcgggttc | |
| | | | tctaaggagtcaattctgcctaagcgga | |
| | | | acagcgacaagctcatcgcccgcaagaa | |
| | | | ggactgggatccgaagaagtacggcggg | |
| | | | ttcgacagccccactgtggcctactcgg | |
| | | | tcctggttgtggcgaaggttgagaaggg | |
| | | | caagtccaagaagctcaagagcgtgaag | |
| | | | gagctgctggggatcacgattatggagc | |
| | | | gctccagcttcgagaagaacccgatcga | |
| | | | tttcctggaggcgaagggctacaaggag | |
| | | | gtgaagaaggacctgatcattaagctcc | |
| | | | ccaagtactcactcttcgagctggagaa | |
| | | | cggcaggaagcggatgctggcttccgct | |
| | | | ggcgagctgcagaaggggaacgagctgg | |
| | | | ctctgccgtccaagtatgtgaacttcct | |
| | | | ctacctggcctcccactacgagaagctc | |
| | | | aagggcagccccgaggacaacgagcaga | |
| | | | agcagctgttcgtcgagcagcacaagca | |
| | | | ttacctcgacgagatcattgagcagatt | |
| | | | tccgagttctccaagcgcgtgatcctgg | |
| | | | ccgacgcgaatctggataaggtcctctc | |
| | | | cgcgtacaacaagcaccgcgacaagcca | |
| | | | atcagggagcaggctgagaatatcattc | |
| | | | atctcttcaccctgacgaacctcggcgc | |
| | | | ccctgctgctttcaagtacttcgacaca | |
| | | | actatcgatcgcaagaggtacacaagca | |
| | | | ctaaggaggtcctggacgcgaccctcat | |
| | | | ccaccagtcgattaccggcctctacgag | |
| | | | acgcgcatcgacctgtctcagctcgggg | |
| | | | gcgacaagcggccagcggcgacgaagaa | |
| | | | ggcggggcaggcgaagaagaagaagtga | |
| 7 | Oryza | DNA | GGCCAGTCACAA | mPing |
| | sativa | | | inverted |
| | | | | repeat 1 |
| 8 | Oryza | DNA | TTGTGACTGGCC | mPing |
| | sativa | | | inverted |
| | | | | repeat 2 |
| 9 | Artificial / | DNA | TTAGGCCAGTCACAA | Sequence |
| | synthetic | | | at |
| | | | | insertion |
| | | | | site |
| 10 | Artificial / | DNA | TTGTGACTGGCCTTA | Sequence |
| | synthetic | | | at |
| | | | | insertion |
| | | | | site |
| 11 | Arabidopsis | DNA | CCATCTTGGGCCTCAACATAAGCCTGAC | gRNA |
| | benthamiana | | CGCCGACCATGGCTGGCAAAAGTCCAAT | targeting |
| | | | AGCAAACTTTAT | site in |
| | | | | PDS 3 and |
| | | | | surrounding |
| | | | | sequence. |
| 12 | Artificial / | DNA | CATAAGCCTGAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 13 | Artificial / | DNA | TTGTGACTGGCCTTAGCGCCGACCATGG | Nucleic |
| | synthetic | | CTGGCAAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 14 | Artificial / | DNA | CATAAGCCTGAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 15 | Artificial / | DNA | TTGTGACTGGCCTTAGCGCCGACCATGG | Nucleic |
| | synthetic | | CTGGCAAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 16 | Artificial / | DNA | CATAAGCCTGAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 17 | Artificial / | DNA | TTGTGACTGGCCTGCCGACCATGGCTGG | Nucleic |
| | synthetic | | CAAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 18 | Artificial / | DNA | CATAAGCCTGACTTAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 19 | Artificial / | DNA | TTGTGACTGGCCTGCCGACCATGGCTGG | Nucleic |
| | synthetic | | CAAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 20 | Artificial / | DNA | CATAAGCCTGACTTAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 21 | Artificial / | DNA | TTGTGACTGGCCGCCGACCATGGCTGGC | Nucleic |
| | synthetic | | AAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 22 | Artificial / | DNA | CATAAGCCTGACAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 23 | Artificial / | DNA | TTGTGACTGGCCGCCGACCATGGCTGGC | Nucleic |
| | synthetic | | AAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 24 | Artificial / | DNA | CATAAGCCTGACAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 25 | Artificial / | DNA | TTGTGACTGGCCTTAACCGACCATGGCT | Nucleic |
| | synthetic | | GGCAAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 26 | Artificial / | DNA | CATAAGCCTGACGTTAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 27 | Artificial / | DNA | TTGTGACTGGCCTTACGCCGACCATGGC | Nucleic |
| | synthetic | | TGGCAAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 28 | Artificial / | DNA | CATAAGCCTGACTGTGT | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 29 | Artificial / | DNA | TTGTGACTGGCCGCCGACCATGGCTGGC | Nucleic |
| | synthetic | | AAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 30 | Artificial / | DNA | CATAAGCCTGATAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 31 | Artificial / | DNA | TTGTGACTGGCCTATGGCTGGCAAAAG | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 32 | Artificial / | DNA | CATAAGCCTGATAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 33 | Artificial / | DNA | TTGTGACTGGCCTATGGCTGGCAAAAG | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 34 | Artificial / | DNA | CATAAGCCTGATAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 35 | Artificial / | DNA | TTGTGACTGGCCTATGGCTGGCAAAAG | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 36 | Artificial / | DNA | CATAAGCCTGATAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 37 | Artificial / | DNA | TTGTGACTGGCCTATGGCTGGCAAAAG | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 38 | Artificial / | DNA | CATAAGCCTGACTAAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 39 | Artificial / | DNA | TTGTGACTGGCCTATGGCTGGCAAAAG | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 40 | Artificial / | DNA | CATAAGCCTGACTAAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 41 | Artificial / | DNA | TTGTGACTGGCCTTCGCCGACCATGGCT | Nucleic |
| | synthetic | | GGCAAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 42 | Artificial / | DNA | CATAAGCCTGAAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 43 | Artificial / | DNA | TTGTGACTGGCCTTCGCCGACCATGGCT | Nucleic |
| | synthetic | | GGCAAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 44 | Artificial / | DNA | CATAAGCCTGAAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 45 | Artificial / | DNA | TTGTGACTGGCCTTCGCCGACCATGGCT | Nucleic |
| | synthetic | | GGCAAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 46 | Artificial / | DNA | CATAAGCCTGACTTAAGGCCAGTCACAA | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 47 | Artificial / | DNA | TTGTGACTGGCCTTCGCCGACCATGGCT | Nucleic |
| | synthetic | | GGCAAAAG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 5 |
| 48 | Artificial / | DNA | CAACATAAGCCTGACAGGCCAGTCACAA | Nucleic |
| | synthetic | | TGG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 49 | Artificial / | DNA | CCATTGTGACTGGCC | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 50 | Artificial / | DNA | GCCGACCATGGCTG | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 51 | Artificial / | DNA | CAACATAAGCCTGAC | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 52 | Artificial / | DNA | GGCCAGTCACAATGG | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 53 | Artificial / | DNA | CCATTGTGACTGGCCCGCCGACCATGGC | Nucleic |
| | synthetic | | TG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 54 | Artificial / | DNA | CCGTTGTTTCCACGTAAGGCCAGTCACA | Nucleic |
| | synthetic | | ATGG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 55 | Artificial / | DNA | CCATTGTGACTGGCCATCTTCGGCCATG | Nucleic |
| | synthetic | | AA | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 56 | Artificial / | DNA | CCGTTGTTTCCACGT | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 57 | Artificial / | DNA | GGCCAGTCACAATGG | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 58 | Artificial / | DNA | CCATGTGACTGGCCATCTTCGGCCATGA | Nucleic |
| | synthetic | | A | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 59 | Artificial / | DNA | TACAGGAGTAGTTC | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 60 | Artificial / | DNA | GCCAGTCACAATGG | Nucleic |
| | synthetic | | | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 61 | Artificial / | DNA | CCATTGTGACTGGCCTCGTGGCCTTAGT | Nucleic |
| | synthetic | | AA | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 62 | Artificial / | DNAa | TACAGGAGTAGTTCAGGCCAGTCACAAT | Nucleic |
| | synthetic | | GG | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 63 | Artificial / | DNA | CCATTGTGACTGGCCTCGTGGCCTTAGT | Nucleic |
| | synthetic | | AA | acid |
| | | | | sequence |
| | | | | at |
| | | | | insertion |
| | | | | sites of |
| | | | | a unique |
| | | | | transposi- |
| | | | | tion |
| | | | | event of |
| | | | | FIG. 7B |
| 64 | Artificial / | Protein | GSSSS | Flexible |
| | synthetic | | | protein |
| | | | | linker |
| 65 | Artificial / | DNA | GCCAGCCATGGTCGGCGGTC | DNA |
| | synthetic | | | encoding |
| | | | | gRNA |
| | | | | targeting |
| | | | | Arabidopsis |
| | | | | PDS3 |
| 66 | Artificial / | DNA | GCTTCATGGCCGAAGATACG | DNA |
| | synthetic | | | encoding |
| | | | | gRNA |
| | | | | targeting |
| | | | | Arabidopsis |
| | | | | ADH1 |
| 67 | Artificial / | DNA | GTTACAGGAGTAGTTCATCG | DNA |
| | synthetic | | | encoding |
| | | | | gRNA |
| | | | | targeting |
| | | | | Arabidopsis |
| | | | | ACT8 |
| 68 | Artificial / | Protein | GGSGGGSG | Linker |
| | synthetic | | | |
| 69 | Artificial / | Protein | (GGGGS)1- 4 | Linker |
| | synthetic | | | |
| 70 | Artificial / | Protein | AEAAAKEAAAKA | Linker |
| | synthetic | | | |
| 71 | Artificial / | Protein | AEAAAKEAAAKEAAAKA | Linker |
| | synthetic | | | |
| 72 | Artificial / | Protein | PAPAP (AP)6-8 | Linker |
| | synthetic | | | |
| 73 | Artificial / | Protein | GIHGVPAA | Linker |
| | synthetic | | | |
| 76 | | | EAAAK | |
| 77 | | | EAAAK EAAAK | |
| 78 | | | EAAAK EAAAK EAAAK | |
| 79 | | | EAAAK EAAAK EAAAK EAAAK | |
| 80 | Artificial / | DNA | GGAACTGACACACGACATGA | DNA |
| | synthetic | | | encoding |
| | | | | gRNA |
| | | | | targeting |
| | | | | Soybean |
| | | | | DD20 |
| 81 | Artificial / | DNA | ggccagtcacaatggctagtgtcattgcacggct | mPing |
| | synthetic | | acccaaaatattataccatcttctctcaaatgaa | modified |
| | | | atcttttatgaaacaatccccacagtggaggggt | with HSEs |
| | | | ttcttgaAcgttccaagactaagcaaagcattta | |
| | | | attgatacaagttCgcgAAgaTtcatttgtaccc | |
| | | | aaaatccggcgcggcgcgggagaatgTTcTggAa | |
| | | | ggtcgcacggcggaggcggacgcaagagatccgg | |
| | | | tgaatgTTCaagaatcggcctcaacgggggtttc | |
| | | | actctgttaccgaggAacttTCTggaaacgacgc | |
| | | | tgacgagtttcaccaggatgaaactctttccAGA | |
| | | | AAGttctctctcatccccatttcatgcaaataat | |
| | | | cattttttattcagtcttacccctattaaatgtg | |
| | | | catgacacaccagtgaaacccccattgtgactgg | |
| | | | cc | |
| 82 | Artificial / | DNA | ttcttgaAcgttc | HSE1 |
| | synthetic | | | |
| 83 | Artificial / | DNA | ttCgcgAAgaTtc | HSE2 |
| | synthetic | | | |
| 84 | Artificial / | DNA | tTccAgAAcattc | HSE3 |
| | synthetic | | | |
| 85 | Artificial / | DNA | ttcttGAAcattc | HSE4 |
| | synthetic | | | |
| 86 | Artificial / | DNA | ttccAGAaagtTc | HSE5 |
| | synthetic | | | |
| 87 | Artificial / | DNA | ttccAGAAAGttc | HSE6 |
| | synthetic | | | |
| 88 | Artificial / | DNA | GGSGGSGGS | Linker |
| | synthetic | | | |

SEQ ID NO: 74. All_in_one_vector: mPING in GFP, gRNA, Pong CRF1 and ORF2 fused
to Cas9 23463 bp dse-DNA circular 28-MAY-2021

| | |
|---|---|
| DEFINITION | . ORF1, the ORF2 protein fused to the Cas9 protein, and the gRNA. |
| ACCESSION | pVec1 |
| VERSION | pVec1.1 |
| FEATURES | Location/Qualifiers |
| Agro tDNA cut site | 1 . . . 25 |
| | /label = “RB″ |
| regulatory | complement (42 . . . 297) |
| | /label = “NOS Terminator″ |
| misc_feature | complement (317 . . . 1105) |
| | /label = “eGFP5-ere″ |
| misc_feature | 1132 . . . 1134 |
| | /label = “TSD″ |
| Transposon | 1135 . . . 1564 |
| | /label = “mPing″ |
| misc_feature | 1565 . . . 1567 |
| | /label = “TSD″ |
| promoter | complement (1581 . . . 2414) |
| | /label = “CaMV Promoter″ |
| misc_feature | 2632 . . . 3055 |
| | /label = “U6-26promoter″ |
| misc_feature | 3056 . . . 3075 |
| | /label = “gRNA to PDS3 exon″ |
| misc_feature | 3076 . . . 3151 |
| | /label = “gRNA scaffold″ |
| misc_feature | 3152 . . . 3343 |
| | /label = “U6-26 terminator″ |
| promoter | 3359 . . . 5045 |
| | /label = “Rps5a″ |
| misc_feature | 5082 . . . 6479 |
| | /label = “ORF1″ |
| terminator | 6543 . . . 7268 |
| | /label = “OCS terminator″ |
| promoter | 7451 . . . 8370 |
| | /label = “GmUbi3 Promoter″ |
| misc_feature | 8392 . . . 9837 |
| | /label = “Pong TPase LA″ |
| misc_feature | 9841 . . . 9855 |
| | /label = “G4S linker″ |
| feature | 9859 . . . 9879 |
| | /label = “SV40 NLS″ |
| misc_feature | 9883 . . . 14052 |
| | /label = “Cas9″ |
| misc_feature | 14005 . . . 14052 |
| | /label = “N_S″ |
| terminator | 14080 . . . 14807 |
| | /label = “OCS Terminator″ |
| promoter | 15058 . . . 15799 |
| | /label = “CaMVd35S promoter″ |
| gene | 15890 . . . 16885 |
| | /label = “hygroB (variant) ″ |
| misc_feature | complement (17503 . . . 17525) |
| | /label = “LB″ |
| gene | 17641 . . . 18435 |
| | /label = “KanR1″ |
| origin | 18506 . . . 19118 |
| | /label = “pBR322 origin″ |

ORIGIN

| | |
|---|---|
| 1 | gtttacccgc caatatatcc tgtcaaacac tgatagtttt tcccgatcta gtaacataga |
| 61 | tgacaccgcg cgcgataatt tatcctagtt tgcgcgctat attttgtttt ctatcgcgta |
| 121 | ttaaatgtat aattgcggga ctctaatcat aaaaacccat ctcataaata acgtcatgca |
| 181 | ttacatgtta attattacat gcttaacgta attcaacaga aattatatga taatcatcgc |
| 241 | aagaccggca acaggattca atcttaagaa actttattgc caaatgtttg aacgatcggg |
| 301 | gaaattcgag ctcttaaagc tcatcatgtt tgtatagttc atccatgcca tgtgtaatcc |
| 361 | cagcagctgt tacaaactca agaaggacca tgtggtctct cttttcgttg ggatctttcg |
| 421 | aaagggcaga ttgtgtggac aggtaatggt tgtctggtaa aaggacaggg ccatcgccaa |
| 481 | ttggagtatt ttgttgataa tgatcagcga gttgcacgcc gccgtcttcg atgttgtggc |
| 541 | gggtcttgaa gttggctttg atgccgttct tttgcttgtc ggccatgatg tatacgttgt |
| 601 | gggagttgta gttgtattcc aacttgtggc cgaggatgtt tccgtcctcc ttgaaatcga |
| 661 | ttcccttaag ctcgatcctg ttgacgaggg tgtctccctc aaacttgact tcagcacgtg |
| 721 | tcttgtagtt cccgtcgtcc ttgaagaaga tggtcctctc ctgcacgtat ccctcaggca |
| 781 | tggcgctctt gaagaagtcg tgccgcttca tatgatctgg gtatcttgaa aagcattgaa |
| 841 | caccataaga gaaagtagtg acaagtgttg gccatggaac aggtagtttt ccagtagtgc |
| 901 | aaataaattt aagggtaagt tttccgtatg ttgcatcacc ttcaccctct ccactgacag |
| 961 | aaaatttgtg cccattaaca tcaccatcta attcaacaag aattgggaca actccagtga |
| 1021 | aaagttcttc tcctttactg aattcggccg aggataatga taggagaagt gaaaagatga |
| 1081 | gaaagagaaa aagattagtc ttcattgtta tatctccttg gatcctctag attaggccag |
| 1141 | tcacaatggc tagtgtcatt gcacggctac ccaaaatatt ataccatctt ctctcaaatg |
| 1201 | aaatctttta tgaaacaatc cccacagtgg aggggtttca ctttgacgtt tccaagacta |
| 1261 | agcaaagcat ttaattgata caagttgctg ggatcatttg tacccaaaat ccggcgcggc |
| 1321 | gcgggagaat gcggaggtcg cacggcggag gcggacgcaa gagatccggt gaatgaaacg |
| 1381 | aatcggcctc aacgggggtt tcactctgtt accgaggact tggaaacgac gctgacgagt |
| 1441 | ttcaccagga tgaaactctt tccttctctc tcatccccat ttcatgcaaa taatcatttt |
| 1501 | ttattcagtc ttacccctat taaatgtgca tgacacacca gtgaaacccc cattgtgact |
| 1561 | ggccttatct agagtccccc gtgttctctc caaatgaaat gaacttcctt atatagagga |
| 1621 | agggtcttgc gaaggatagt gggattgtgc gtcatccctt acgtcagtgg agatatcaca |
| 1681 | tcaatccact tgctttgaag acgtggttgg aacgtcttct ttttccacga tgctcctcgt |
| 1741 | gggtgggggt ccatctttgg gaccactgtc ggcagaggca tcttcaacga tggcctttcc |
| 1801 | tttatcgcaa tgatggcatt tgtaggagcc accttccttt tccactatct tcacaataaa |
| 1861 | gtgacagata gctgggcaat ggaatccgag gaggtttccg gatattaccc tttgttgaaa |
| 1921 | agtctcaatt gccctttggt cttctgagac tgtatctttg atatttttgg agtagacaag |
| 1981 | tgtgtcgtgc tccaccatgt tgacgaagat tttcttcttg tcattgagtc gtaagagact |
| 2041 | ctgtatgaac tgttcgccag tctttacggc gagttctgtt aggtcctcta tttgaatctt |
| 2101 | tgactccatg gcctttgatt cagtgggaac taccttttta gagactccaa tctctattac |
| 2161 | ttgccttggt ttgtgaagca agccttgaat cgtccatact ggaatagtac ttctgatctt |
| 2221 | gagaaatata tctttctctg tgttcttgat gcagttagtc ctgaatcttt tgactgcatc |
| 2281 | tttaaccttc ttgggaaggt atttgatttc ctggagatta ttgctcgggt agatcgtctt |
| 2341 | gatgagacct gctgcgtaag cctctctaac catctgtggg ttagcattct ttctgaaatt |
| 2401 | gaaaaggcta atctgggaaa ctgaaggcgg gaaacgacaa tctgatccaa gctcaagctg |
| 2461 | ctctagcatt cgccattcag gctgcgcaac tgttgggaag ggcgatcggt gcgggcctct |
| 2521 | tcgctattac gccagctggc gaaaggggga tgtgctgcaa ggcgattaag ttgggtaacg |
| 2581 | ccagggtttt cccagtcacg acgttgtaaa acgacggcca gtgccaagct tcgacttgcc |
| 2641 | ttccgcacaa tacatcattt cttcttagct ttttttcttc ttcttcgttc atacagtttt |
| 2701 | tttttgttta tcagcttaca ttttcttgaa ccgtagcttt cgttttcttc tttttaactt |
| 2761 | tccattcgga gtttttgtat cttgtttcat agtttgtccc aggattagaa tgattaggca |
| 2821 | tcgaaccttc aagaatttga ttgaataaaa catcttcatt cttaagatat gaagataatc |
| 2881 | ttcaaaaggc ccctgggaat ctgaaagaag agaagcaggc ccatttatat gggaaagaac |
| 2941 | aatagtattt cttatatagg cccatttaag ttgaaaacaa tcttcaaaag tcccacatcg |
| 3001 | cttagataag aaaacgaagc tgagtttata tacagctaga gtcgaagtag tgattGCCAG |
| 3061 | CCATGGTCGG CGGTCgtttt agagctagaa atagcaagtt aaaataaggc tagtccgtta |
| 3121 | tcaacttgaa aaagtggcac cgagtcggtg cttttttttg caaaattttc cagatcgatt |
| 3181 | tcttcttcct ctgttcttcg gcgttcaatt tctggggttt tctcttcgtt ttctgtaact |
| 3241 | gaaacctaaa atttgaccta aaaaaaatct caaataatat gattcagtgg ttttgtactt |
| 3301 | ttcagttagt tgagttttgc agttccgatg agataaacca ataccatgtt agagagcgct |
| 3361 | agttcgtgag tagatatatt actcaacttt tgattcgcta tttgcagtgc acctgtggcg |
| 3421 | ttcatcacat cttttgtgac actgtttgca ctggtcattg ctattacaaa ggaccttcct |
| 3481 | gatgttgaag gagatcgaaa gtaagtaact gcacgcataa ccattttctt tccgctcttt |
| 3541 | ggctcaatcc atttgacagt caaagacaat gtttaaccag ctccgtttga tatattgtct |
| 3601 | ttatgtgttt gttcaagcat gtttagttaa tcatgccttt gattgatctt gaataggttc |
| 3661 | caaatatcaa ccctggcaac aaaacttgga gtgagaaaca ttgcattcct cggttctgga |
| 3721 | cttctgctag taaattatgt ttcagccata tcactagctt tctacatgcc tcaggtgaat |
| 3781 | tcatctattt ccgtcttaac tatttcggtt aatcaaagca cgaacaccat tactgcatgt |
| 3841 | agaagcttga taaactatcg ccaccaattt atttttgttg cgatattgtt actttcctca |
| 3901 | gtatgcagct ttgaaaagac caaccctctt atcctttaac aatgaacagg tttttagagg |
| 3961 | tagcttgatg attcctgcac atgtgatctt ggcttcaggc ttaattttcc aggtaaagca |
| 4021 | ttatgagata ctcttatatc tcttacatac ttttgagata atgcacaaga acttcataac |
| 4081 | tatatgcttt agtttctgca tttgacactg ccaaattcat taatctctaa tatctttgtt |
| 4141 | gttgatcttt ggtagacatg ggtactagaa aaagcaaact acaccaaggt aaaatacttt |
| 4201 | tgtacaaaca taaactcgtt atcacggaac atcaatggag tgtatatcta acggagtgta |
| 4261 | gaaacatttg attattgcag gaagctatct caggatatta tcggtttata tggaatctct |
| 4321 | tctacgcaga gtatctgtta ttccccttcc tctagctttc aatttcatgg tgaggatatg |
| 4381 | cagttttctt tgtatatcat tcttcttctt ctttgtagct tggagtcaaa atcggttcct |
| 4441 | tcatgtacat acatcaagga tatgtccttc tgaattttta tatcttgcaa taaaaatgct |
| 4501 | tgtaccaatt gaaacaccag ctttttgagt tctatgatca ctgacttggt tctaaccaaa |
| 4561 | aaaaaaaaaa tgtttaattt acatatctaa aagtaggttt agggaaacct aaacagtaaa |
| 4621 | atatttgtat attattcgaa tttcactcat cataaaaact taaattgcac cataaaattt |
| 4681 | tgttttacta ttaatgatgt aatttgtgta acttaagata aaaataatat tccgtaagtt |
| 4741 | aaccggctaa aaccacgtat aaaccaggga acctgttaaa ccggttcttt actggataaa |
| 4801 | gaaatgaaag cccatgtaga cagctccatt agagcccaaa ccctaaattt ctcatctata |
| 4861 | taaaaggagt gacattaggg tttttgttcg tcctcttaaa gcttctcgtt ttctctgccg |
| 4921 | tctctctcat tcgcgcgacg caaacgatct tcaggtgatc ttctttctcc aaatcctctc |
| 4981 | tcataactct gatttcgtac ttgtgtattt gagctcacgc tctgtttctc tcaccacagc |
| 5041 | cggattcgag atcacaagtt tgtacaaaaa agcaggcttc catggatccg tcgccggccg |
| 5101 | tggatccgtc gccggccgtg gatccgtcgc cggctgctga aacccggcgg cgtgcaaccg |
| 5161 | ggaaaggagg caaacagcgc gggggcaagc aactaggatt gaagaggccg ccgccgattt |
| 5221 | ctgtcccggc caccccgcct cctgctgcga cgtcttcatc ccctgctgcg ccgacggcca |
| 5281 | tcccaccacg accaccgcaa tcttcgccga ttttcgtccc cgattcgccg aatccgtcac |
| 5341 | cggctgcgcc gacctcctct cttgcttcgg ggacatcgac ggcaaggcca ccgcaaccac |
| 5401 | aaggaggagg atggggacca acatcgacca tttccccaaa ctttgcatct ttctttggaa |
| 5461 | accaacaaga cccaaattca tgtttggtca ggggttatcc tccaggaggg tttgtcaatt |
| 5521 | ttattcaaca aaattgtccg ccgcagccac aacagcaagg tgaaaatttt catttcgttg |
| 5581 | gtcacaatat ggggttcaac ccaatatctc cacagccacc aagtgcctac ggaacaccaa |
| 5641 | caccccaagc tacgaaccaa ggcacttcaa caaacattat gattgatgaa gaggacaaca |
| 5701 | atgatgacag tagggcagca aagaaaagat ggactcatga agaggaagag agactggcca |
| 5761 | gtgcttggtt gaatgcttct aaagactcaa ttcatgggaa tgataagaaa ggtgatacat |
| 5821 | tttggaagga agtcactgat gaatttaaca agaaagggaa tggaaaacgt aggagggaaa |
| 5881 | ttaaccaact gaaggttcac tggtcaaggt tgaagtcagc gatctctgag ttcaatgact |
| 5941 | attggagtac ggttactcaa atgcatacaa gcggatactc agacgacatg cttgagaaag |
| 6001 | aggcacagag gctgtatgca aacaggtttg gaaaaccttt tgcgttggtc cattggtgga |
| 6061 | agatactcaa aagagagccc aaatggtgtg ctcagtttga aaagaggaaa aggaagagcg |
| 6121 | aaatggatgc tgttccagaa cagcagaaac gtcctattgg tagagaagca gcaaagtctg |
| 6181 | agcgcaaaag aaagcgcaag aaagaaaatg ttatggaagg cattgtcctc ctaggggaca |
| 6241 | atgtccagaa aattatcaaa gtgacgcaag atcggaagct ggagcgtgag aaggtcactg |
| 6301 | aagcacagat tcacatttca aacgtaaatt tgaaggcagc agaacagcaa aaagaagcaa |
| 6361 | agatgtttga ggtatacaat tccctgctca ctcaagatac aagtaacatg tctgaagaac |
| 6421 | agaaggctcg ccgagacaag gcattacaaa agctggagga aaagttattt gctgactagt |
| 6481 | gacccagctt tcttgtacaa agtggtgcct aggtgagtct agagagttga ttaagacccg |
| 6541 | ggactggtcc ctagagtcct gctttaatga gatatgcgag acgcctatga tcgcatgata |
| 6601 | tttgctttca attctgttgt gcacgttgta aaaaacctga gcatgtgtag ctcagatcct |
| 6661 | taccgccggt ttcggttcat tctaatgaat atatcacccg ttactatcgt atttttatga |
| 6721 | ataatattct ccgttcaatt tactgattgt accctactac ttatatgtac aatattaaaa |
| 6781 | tgaaaacaat atattgtgct gaataggttt atagcgacat ctatgataga gcgccacaat |
| 6841 | aacaaacaat tgcgttttat tattacaaat ccaattttaa aaaaagcggc agaaccggtc |
| 6901 | aaacctaaaa gactgattac ataaatctta ttcaaatttc aaaagtgccc caggggctag |
| 6961 | tatctacgac acaccgagcg gcgaactaat aacgctcact gaagggaact ccggttcccc |
| 7021 | gccggcgcgc atgggtgaga ttccttgaag ttgagtattg gccgtccgct ctaccgaaag |
| 7081 | ttacgggcac cattcaaccc ggtccagcac ggcggccggg taaccgactt gctgccccga |
| 7141 | gaattatgca gcattttttt ggtgtatgtg ggccccaaat gaagtgcagg tcaaaccttg |
| 7201 | acagtgacga caaatcgttg ggcgggtcca gggcgaattt tgcgacaaca tgtcgaggct |
| 7261 | cagcaggacc tgcaggcatg caagcttggc actggccgtc gttttacaac gtcgtgactg |
| 7321 | ggaaaaccct ggcgttaccc aacttaatcg ccttgcagca catccccctt tcgccagctg |
| 7381 | gcgtaatagc gaagaggccc gcaccgatcg cccttcccaa cagttgcgca gcctgaatgg |
| 7441 | cgaatgctag agcagcttga gcttggatca gattgtcgtt tcccgccttc agtttcttga |
| 7501 | aggtgcatgt gactccgtca agattacgaa accgccaact accacgcaaa ttgcaattct |
| 7561 | caatttccta gaaggactct ccgaaaatgc atccaatacc aaatattacc cgtgtcatag |
| 7621 | gcaccaagtg acaccataca tgaacacgcg tcacaatatg actggagaag ggttccacac |
| 7681 | cttatgctat aaaacgcccc acacccctcc tccttccttc gcagttcaat tccaatatat |
| 7741 | tccattctct ctgtgtattt ccctacctct cccttcaagg ttagtcgatt tcttctgttt |
| 7801 | ttcttcttcg ttctttccat gaattgtgta tgttctttga tcaatacgat gttgatttga |
| 7861 | ttgtgttttg tttggtttca tcgatcttca attttcataa tcagattcag cttttattat |
| 7921 | ctttacaaca acgtccttaa tttgatgatt ctttaatcgt agatttgctc taattagagc |
| 7981 | tttttcatgt cagatccctt tacaacaagc cttaattgtt gattcattaa tcgtagatta |
| 8041 | gggctttttt cattgattac ttcagatccg ttaaacgtaa ccatagatca gggctttttc |
| 8101 | atgaattact tcagatccgt taaacaacag ccttattttt tatacttctg tggtttttca |
| 8161 | agaaattgtt cagatccgtt gacaaaaagc cttattcgtt gattctatat cgtttttcga |
| 8221 | gagatattgc tcagatctgt tagcaactgc cttgtttgtt gattctattg ccgtggatta |
| 8281 | gggttttttt tcacgagatt gcttcagatc cgtacttaag attacgtaat ggattttgat |
| 8341 | tctgatttat ctgtgattgt tgactcgaca ggtaccttca aacggcgcgc catgcagagt |
| 8401 | ttagccatct ctctactcct ctcagaaact cattccctct tttctcatac gaagacctcc |
| 8461 | tcccttttat ctttactgtt tctctcttct tcaaagatgt ctgagcaaaa tactgatgga |
| 8521 | agtcaagttc cagtgaactt gttggatgag ttcctggctg aggatgagat catagatgat |
| 8581 | cttctcactg aagccacggt ggtagtacag tccactatag aaggtcttca aaacgaggct |
| 8641 | tctgaccatc gacatcatcc gaggaagcac atcaagaggc cacgagagga agcacatcag |
| 8701 | caactggtga atgattactt ttcagaaaat cctctttacc cttccaaaat ttttcgtcga |
| 8761 | agatttcgta tgtctaggcc actttttctt cgcatcgttg aggcattagg ccagtggtca |
| 8821 | gtgtatttca cacaaagggt ggatgctgtt aatcggaaag gactcagtcc actgcaaaag |
| 8881 | tgtactgcag ctattcgcca gttggctact ggtagtggcg cagatgaact agatgaatat |
| 8941 | ctgaagatag gagagactac agcaatggag gcaatgaaga attttgtcaa aggtcttcaa |
| 9001 | gatgtgtttg gtgagaggta tcttaggcgc cccactatgg aagataccga acggcttctc |
| 9061 | caacttggtg agaaacgtgg ttttcctgga atgttcggca gcattgactg catgcactgg |
| 9121 | cattgggaaa gatgcccagt agcatggaag ggtcagttca ctcgtggaga tcagaaagtg |
| 9181 | ccaaccctga ttcttgaggc tgtggcatcg catgatcttt ggatttggca tgcatttttt |
| 9241 | ggagcagcgg gttccaacaa tgatatcaat gtattgaacc aatctactgt atttatcaag |
| 9301 | gagctcaaag gacaagctcc tagagtccag tacatggtaa atgggaatca atacaatact |
| 9361 | gggtattttc ttgctgatgg aatctaccct gaatgggcag tgtttgttaa gtcaatacga |
| 9421 | ctcccaaaca ctgaaaagga gaaattgtat gcagatatgc aagaaggggc aagaaaagat |
| 9481 | atcgagagag cctttggtgt attgcagcga agattttgca tcttaaaacg accagctcgt |
| 9541 | ctatatgatc gaggtgtact gcgagatgtt gttctagctt gcatcatact tcacaatatg |
| 9601 | atagttgaag atgagaagga aaccagaatt attgaagaag atgcagatgc aaatgtgcct |
| 9661 | cctagttcat caaccgttca ggaacctgag ttctctcctg aacagaacac accatttgat |
| 9721 | agagttttag aaaaagatat ttctatccga gatcgagcgg ctcataaccg acttaagaaa |
| 9781 | gatttggtgg aacacatttg gaataagttt ggtggtgctg cacatagaac tggaaattat |
| 9841 | ggcgggggag gtagcgctcc gaagaagaag aggaaggttg gcatccacgg ggtgccagct |
| 9901 | gctgacaaga agtactcgat cggcctcgat attgggacta actctgttgg ctgggccgtg |
| 9961 | atcaccgacg agtacaaggt gccctcaaag aagttcaagg tcctgggcaa caccgatcgg |
| 10021 | cattccatca agaagaatct cattggcgct ctcctgttcg acagcggcga gacggctgag |
| 10081 | gctacgcggc tcaagcgcac cgcccgcagg cggtacacgc gcaggaagaa tcgcatctgc |
| 10141 | tacctgcagg agattttctc caacgagatg gcgaaggttg acgattcttt cttccacagg |
| 10201 | ctggaggagt cattcctcgt ggaggaggat aagaagcacg agcggcatcc aatcttcggc |
| 10261 | aacattgtcg acgaggttgc ctaccacgag aagtacccta cgatctacca tctgcggaag |
| 10321 | aagctcgtgg actccacaga taaggcggac ctccgcctga tctacctcgc tctggcccac |
| 10381 | atgattaagt tcaggggcca tttcctgatc gagggggatc tcaacccgga caatagcgat |
| 10441 | gttgacaagc tgttcatcca gctcgtgcag acgtacaacc agctcttcga ggagaacccc |
| 10501 | attaatgcgt caggcgtcga cgcgaaggct atcctgtccg ctaggctctc gaagtctcgg |
| 10561 | cgcctcgaga acctgatcgc ccagctgccg ggcgagaaga agaacggcct gttcgggaat |
| 10621 | ctcattgcgc tcagcctggg gctcacgccc aacttcaagt cgaatttcga tctcgctgag |
| 10681 | gacgccaagc tgcagctctc caaggacaca tacgacgatg acctggataa cctcctggcc |
| 10741 | cagatcggcg atcagtacgc ggacctgttc ctcgctgcca agaatctgtc ggacgccatc |
| 10801 | ctcctgtctg atattctcag ggtgaacacc gagattacga aggctccgct ctcagcctcc |
| 10861 | atgatcaagc gctacgacga gcaccatcag gatctgaccc tcctgaaggc gctggtcagg |
| 10921 | cagcagctcc ccgagaagta caaggagatc ttcttcgatc agtcgaagaa cggctacgct |
| 10981 | gggtacattg acggcggggc ctctcaggag gagttctaca agttcatcaa gccgattctg |
| 11041 | gagaagatgg acggcacgga ggagctgctg gtgaagctca atcgcgagga cctcctgagg |
| 11101 | aagcagcgga cattcgataa cggcagcatc ccacaccaga ttcatctcgg ggagctgcac |
| 11161 | gctatcctga ggaggcagga ggacttctac cctttcctca aggataaccg cgagaagatc |
| 11221 | gagaagattc tgactttcag gatcccgtac tacgtcggcc cactcgctag gggcaactcc |
| 11281 | cgcttcgctt ggatgacccg caagtcagag gagacgatca cgccgtggaa cttcgaggag |
| 11341 | gtggtcgaca agggcgctag cgctcagtcg ttcatcgaga ggatgacgaa tttcgacaag |
| 11401 | aacctgccaa atgagaaggt gctccctaag cactcgctcc tgtacgagta cttcacagtc |
| 11461 | tacaacgagc tgactaaggt gaagtatgtg accgagggca tgaggaagcc ggctttcctg |
| 11521 | tctggggagc agaagaaggc catcgtggac ctcctgttca agaccaaccg gaaggtcacg |
| 11581 | gttaagcagc tcaaggagga ctacttcaag aagattgagt gcttcgattc ggtcgagatc |
| 11641 | tctggcgttg aggaccgctt caacgcctcc ctggggacct accacgatct cctgaagatc |
| 11701 | attaaggata aggacttcct ggacaacgag gagaatgagg atatcctcga ggacattgtg |
| 11761 | ctgacactca ctctgttcga ggaccgggag atgatcgagg agcgcctgaa gacttacgcc |
| 11821 | catctcttcg atgacaaggt catgaagcag ctcaagagga ggaggtacac cggctggggg |
| 11881 | aggctgagca ggaagctcat caacggcatt cgggacaagc agtccgggaa gacgatcctc |
| 11941 | gacttcctga agagcgatgg cttcgcgaac cgcaatttca tgcagctgat tcacgatgac |
| 12001 | agcctcacat tcaaggagga tatccagaag gctcaggtga gcggccaggg ggactcgctg |
| 12061 | cacgagcata tcgcgaacct cgctggctcg ccagctatca agaaggggat tctgcagacc |
| 12121 | gtgaaggttg tggacgagct ggtgaaggtc atgggcaggc acaagcctga gaacatcgtc |
| 12181 | attgagatgg cccgggagaa tcagaccacg cagaagggcc agaagaactc acgcgagagg |
| 12241 | atgaagagga tcgaggaggg cattaaggag ctggggtccc agatcctcaa ggagcacccg |
| 12301 | gtggagaaca cgcagctgca gaatgagaag ctctacctgt actacctcca gaatggccgc |
| 12361 | gatatgtatg tggaccagga gctggatatt aacaggctca gcgattacga cgtcgatcat |
| 12421 | atcgttccac agtcattcct gaaggatgac tccattgaca acaaggtcct caccaggtcg |
| 12481 | gacaagaacc ggggcaagtc tgataatgtt ccttcagagg aggtcgttaa gaagatgaag |
| 12541 | aactactggc gccagctcct gaatgccaag ctgatcacgc agcggaagtt cgataacctc |
| 12601 | acaaaggctg agaggggcgg gctctctgag ctggacaagg cgggcttcat caagaggcag |
| 12661 | ctggtcgaga cacggcagat cactaagcac gttgcgcaga ttctcgactc acggatgaac |
| 12721 | actaagtacg atgagaatga caagctgatc cgcgaggtga aggtcatcac cctgaagtca |
| 12781 | aagctcgtct ccgacttcag gaaggatttc cagttctaca aggttcggga gatcaacaat |
| 12841 | taccaccatg cccatgacgc gtacctgaac gcggtggtcg gcacagctct gatcaagaag |
| 12901 | tacccaaagc tcgagagcga gttcgtgtac ggggactaca aggtttacga tgtgaggaag |
| 12961 | atgatcgcca agtcggagca ggagattggc aaggctaccg ccaagtactt cttctactct |
| 13021 | aacattatga atttcttcaa gacagagatc actctggcca atggcgagat ccggaagcgc |
| 13081 | cccctcatcg agacgaacgg cgagacgggg gagatcgtgt gggacaaggg cagggatttc |
| 13141 | gcgaccgtca ggaaggttct ctccatgcca caagtgaata tcgtcaagaa gacagaggtc |
| 13201 | cagactggcg ggttctctaa ggagtcaatt ctgcctaagc ggaacagcga caagctcatc |
| 13261 | gcccgcaaga aggactggga tccgaagaag tacggcgggt tcgacagccc cactgtggcc |
| 13321 | tactcggtcc tggttgtggc gaaggttgag aagggcaagt ccaagaagct caagagcgtg |
| 13381 | aaggagctgc tggggatcac gattatggag cgctccagct tcgagaagaa cccgatcgat |
| 13441 | ttcctggagg cgaagggcta caaggaggtg aagaaggacc tgatcattaa gctccccaag |
| 13501 | tactcactct tcgagctgga gaacggcagg aagcggatgc tggcttccgc tggcgagctg |
| 13561 | cagaagggga acgagctggc tctgccgtcc aagtatgtga acttcctcta cctggcctcc |
| 13621 | cactacgaga agctcaaggg cagccccgag gacaacgagc agaagcagct gttcgtcgag |
| 13681 | cagcacaagc attacctcga cgagatcatt gagcagattt ccgagttctc caagcgcgtg |
| 13741 | atcctggccg acgcgaatct ggataaggtc ctctccgcgt acaacaagca ccgcgacaag |
| 13801 | ccaatcaggg agcaggctga gaatatcatt catctcttca ccctgacgaa cctcggcgcc |
| 13861 | cctgctgctt tcaagtactt cgacacaact atcgatcgca agaggtacac aagcactaag |
| 13921 | gaggtcctgg acgcgaccct catccaccag tcgattaccg gcctctacga gacgcgcatc |
| 13981 | gacctgtctc agctcggggg cgacaagcgg ccagcggcga cgaagaaggc ggggcaggcg |
| 14041 | aagaagaaga agtgataatt gacattctaa tctagagtcc tgctttaatg agatatgcga |
| 14101 | gacgcctatg atcgcatgat atttgctttc aattctgttg tgcacgttgt aaaaaacctg |
| 14161 | agcatgtgta gctcagatcc ttaccgccgg tttcggttca ttctaatgaa tatatcaccc |
| 14221 | gttactatcg tatttttatg aataatattc tccgttcaat ttactgattg taccctacta |
| 14281 | cttatatgta caatattaaa atgaaaacaa tatattgtgc tgaataggtt tatagcgaca |
| 14341 | tctatgatag agcgccacaa taacaaacaa ttgcgtttta ttattacaaa tccaatttta |
| 14401 | aaaaaagcgg cagaaccggt caaacctaaa agactgatta cataaatctt attcaaattt |
| 14461 | caaaagtgcc ccaggggcta gtatctacga cacaccgagc ggcgaactaa taacgttcac |
| 14521 | tgaagggaac tccggttccc cgccggcgcg catgggtgag attccttgaa gttgagtatt |
| 14581 | ggccgtccgc tctaccgaaa gttacgggca ccattcaacc cggtccagca cggcggccgg |
| 14641 | gtaaccgact tgctgccccg agaattatgc agcatttttt tggtgtatgt gggccccaaa |
| 14701 | tgaagtgcag gtcaaacctt gacagtgacg acaaatcgtt gggcgggtcc agggcgaatt |
| 14761 | ttgcgacaac atgtcgaggc tcagcaggac ctgcaggcat gcaagatcgc gaattcgtaa |
| 14821 | tcatgtcata gctgtttcct gtgtgaaatt gttatccgct cacaattcca cacaacatac |
| 14881 | gagccggaag cataaagtgt aaagcctggg gtgcctaatg agtgagctaa ctcacattaa |
| 14941 | ttgcgttgcg ctcactgccc gctttccagt cgggaaacct gtcgtgccag ctgcattaat |
| 15001 | gaatcggcca acgcgcgggg agaggcggtt tgcgtattgg ctagagcagc ttgccaacat |
| 15061 | ggtggagcac gacactctcg tctactccaa gaatatcaaa gatacagtct cagaagacca |
| 15121 | aagggctatt gagacttttc aacaaagggt aatatcggga aacctcctcg gattccattg |
| 15181 | cccagctatc tgtcacttca tcaaaaggac agtagaaaag gaaggtggca cctacaaatg |
| 15241 | ccatcattgc gataaaggaa aggctatcgt tcaagatgcc tctgccgaca gtggtcccaa |
| 15301 | agatggaccc ccacccacga ggagcatcgt ggaaaaagaa gacgttccaa ccacgtcttc |
| 15361 | aaagcaagtg gattgatgtg ataacatggt ggagcacgac actctcgtct actccaagaa |
| 15421 | tatcaaagat acagtctcag aagaccaaag ggctattgag acttttcaac aaagggtaat |
| 15481 | atcgggaaac ctcctcggat tccattgccc agctatctgt cacttcatca aaaggacagt |
| 15541 | agaaaaggaa ggtggcacct acaaatgcca tcattgcgat aaaggaaagg ctatcgttca |
| 15601 | agatgcctct gccgacagtg gtcccaaaga tggaccccca cccacgagga gcatcgtgga |
| 15661 | aaaagaagac gttccaacca cgtcttcaaa gcaagtggat tgatgtgata tctccactga |
| 15721 | cgtaagggat gacgcacaat cccactatcc ttcgcaagac cttcctctat ataaggaagt |
| 15781 | tcatttcatt tggagaggac acgctgaaat caccagtctc tctctacaaa tctatctctc |
| 15841 | tcgagctttc gcagatcccg gggggcaatg agatatgaaa aagcctgaac tcaccgcgac |
| 15901 | gtctgtcgag aagtttctga tcgaaaagtt cgacagcgtc tccgacctga tgcagctctc |
| 15961 | ggagggcgaa gaatctcgtg ctttcagctt cgatgtagga gggcgtggat atgtcctgcg |
| 16021 | ggtaaatagc tgcgccgatg gtttctacaa agatcgttat gtttatcggc actttgcatc |
| 16081 | ggccgcgctc ccgattccgg aagtgcttga cattggggag tttagcgaga gcctgaccta |
| 16141 | ttgcatctcc cgccgtgcac agggtgtcac gttgcaagac ctgcctgaaa ccgaactgcc |
| 16201 | cgctgttcta caaccggtcg cggaggctat ggatgcgatc gctgcggccg atcttagcca |
| 16261 | gacgagcggg ttcggcccat tcggaccgca aggaatcggt caatacacta catggcgtga |
| 16321 | tttcatatgc gcgattgctg atccccatgt gtatcactgg caaactgtga tggacgacac |
| 16381 | cgtcagtgcg tccgtcgcgc aggctctcga tgagctgatg ctttgggccg aggactgccc |
| 16441 | cgaagtccgg cacctcgtgc acgcggattt cggctccaac aatgtcctga cggacaatgg |
| 16501 | ccgcataaca gcggtcattg actggagcga ggcgatgttc ggggattccc aatacgaggt |
| 16561 | cgccaacatc ttcttctgga ggccgtggtt ggcttgtatg gagcagcaga cgcgctactt |
| 16621 | cgagcggagg catccggagc ttgcaggatc gccacgactc cgggcgtata tgctccgcat |
| 16681 | tggtcttgac caactctatc agagcttggt tgacggcaat ttcgatgatg cagcttgggc |
| 16741 | gcagggtcga tgcgacgcaa tcgtccgatc cggagccggg actgtcgggc gtacacaaat |
| 16801 | cgcccgcaga agcgcggccg tctggaccga tggctgtgta gaagtactcg ccgatagtgg |
| 16861 | aaaccgacgc cccagcactc gtccgagggc aaagaaatag agtagatgcc gaccggatct |
| 16921 | gtcgatcgac aagctcgagt ttctccataa taatgtgtga gtagttccca gataagggaa |
| 16981 | ttagggttcc tatagggttt cgctcatgtg ttgagcatat aagaaaccct tagtatgtat |
| 17041 | ttgtatttgt aaaatacttc tatcaataaa atttctaatt cctaaaacca aaatccagta |
| 17101 | ctaaaatcca gatcccccga attaattcgg cgttaattca gtacattaaa aacgtccgca |
| 17161 | atgtgttatt aagttgtcta agcgtcaatt tgtttacacc acaatatatc ctgccaccag |
| 17221 | ccagccaaca gctccccgac cggcagctcg gcacaaaatc accactcgat acaggcagcc |
| 17281 | catcagtccg ggacggcgtc agcgggagag ccgttgtaag gcggcagact ttgctcatgt |
| 17341 | taccgatgct attcggaaga acggcaacta agctgccggg tttgaaacac ggatgatctc |
| 17401 | gcggagggta gcatgttgat tgtaacgatg acagagcgtt gctgcctgtg atcaccgcgg |
| 17461 | tttcaaaatc ggctccgtcg atactatgtt atacgccaac tttgaaaaca actttgaaaa |
| 17521 | agctgttttc tggtatttaa ggttttagaa tgcaaggaac agtgaattgg agttcgtctt |
| 17581 | gttataatta gcttcttggg gtatctttaa atactgtaga aaagaggaag gaaataataa |
| 17641 | atggctaaaa tgagaatatc accggaattg aaaaaactga tcgaaaaata ccgctgcgta |
| 17701 | aaagatacgg aaggaatgtc tcctgctaag gtatataagc tggtgggaga aaatgaaaac |
| 17761 | ctatatttaa aaatgacgga cagccggtat aaagggacca cctatgatgt ggaacgggaa |
| 17821 | aaggacatga tgctatggct ggaaggaaag ctgcctgttc caaaggtcct gcactttgaa |
| 17881 | cggcatgatg gctggagcaa tctgctcatg agtgaggccg atggcgtcct ttgctcggaa |
| 17941 | gagtatgaag atgaacaaag ccctgaaaag attatcgagc tgtatgcgga gtgcatcagg |
| 18001 | ctctttcact ccatcgacat atcggattgt ccctatacga atagcttaga cagccgctta |
| 18061 | gccgaattgg attacttact gaataacgat ctggccgatg tggattgcga aaactgggaa |
| 18121 | gaagacactc catttaaaga tccgcgcgag ctgtatgatt ttttaaagac ggaaaagccc |
| 18181 | gaagaggaac ttgtcttttc ccacggcgac ctgggagaca gcaacatctt tgtgaaagat |
| 18241 | ggcaaagtaa gtggctttat tgatcttggg agaagcggca gggcggacaa gtggtatgac |
| 18301 | attgccttct gcgtccggtc gatcagggag gatatcgggg aagaacagta tgtcgagcta |
| 18361 | ttttttgact tactggggat caagcctgat tgggagaaaa taaaatatta tattttactg |
| 18421 | gatgaattgt tttagtacct agaatgcatg accaaaatcc cttaacgtga gttttcgttc |
| 18481 | cactgagcgt cagaccccgt agaaaagatc aaaggatctt cttgagatcc tttttttctg |
| 18541 | cgcgtaatct gctgcttgca aacaaaaaaa ccaccgctac cagcggtggt ttgtttgccg |
| 18601 | gatcaagagc taccaactct ttttccgaag gtaactggct tcagcagagc gcagatacca |
| 18661 | aatactgtcc ttctagtgta gccgtagtta ggccaccact tcaagaactc tgtagcaccg |
| 18721 | cctacatacc tcgctctgct aatcctgtta ccagtggctg ctgccagtgg cggtgtctta |
| 18781 | ccgggttgga ctcaagacga tagttaccgg ataaggcgca gcggtcgggc tgaacggggg |
| 18841 | gttcgtgcac acagcccagc ttggagcgaa cgacctacac cgaactgaga tacctacagc |
| 18901 | gtgagctatg agaaagcgcc acgcttcccg aagggagaaa ggcggacagg tatccggtaa |
| 18961 | gcggcagggt cggaacagga gagcgcacga gggagcttcc agggggaaac gcctggtatc |
| 19021 | tttatagtcc tgtcgggttt cgccacctct gacttgagcg tcgatttttg tgatgctcgt |
| 19081 | caggggggcg gagcctatgg aaaaacgcca gcaacgcggc ctttttacgg ttcctggcct |
| 19141 | tttgctggcc ttttgctcac atgttctttc ctgcgttatc ccctgattct gtggataacc |
| 19201 | gtattaccgc ctttgagtga gctgataccg ctcgccgcag ccgaacgacc gagcgcagcg |
| 19261 | agtcagtgag cgaggaagcg gaagagcgcc tgatgcggta ttttctcctt acgcatctgt |
| 19321 | gcggtatttc acaccgcata tggtgcactc tcagtacaat ctgctctgat gccgcatagt |
| 19381 | taagccagta tacactccgc tatcgctacg tgactgggtc atggctgcgc cccgacaccc |
| 19441 | gccaacaccc gctgacgcgc cctgacgggc ttgtctgctc ccggcatccg cttacagaca |
| 19501 | agctgtgacc gtctccggga gctgcatgtg tcagaggttt tcaccgtcat caccgaaacg |
| 19561 | cgcgaggcag ggtgccttga tgtgggcgcc ggcggtcgag tggcgacggc gcggcttgtc |
| 19621 | cgcgccctgg tagattgcct ggccgtaggc cagccatttt tgagcggcca gcggccgcga |
| 19681 | taggccgacg cgaagcggcg gggcgtaggg agcgcagcga ccgaagggta ggcgcttttt |
| 19741 | gcagctcttc ggctgtgcgc tggccagaca gttatgcaca ggccaggcgg gttttaagag |
| 19801 | ttttaataag ttttaaagag ttttaggcgg aaaaatcgcc ttttttctct tttatatcag |
| 19861 | tcacttacat gtgtgaccgg ttcccaatgt acggctttgg gttcccaatg tacgggttcc |
| 19921 | ggttcccaat gtacggcttt gggttcccaa tgtacgtgct atccacagga aacagacctt |
| 19981 | ttcgaccttt ttcccctgct agggcaattt gccctagcat ctgctccgta cattaggaac |
| 20041 | cggcggatgc ttcgccctcg atcaggttgc ggtagcgcat gactaggatc gggccagcct |
| 20101 | gccccgcctc ctccttcaaa tcgtactccg gcaggtcatt tgacccgatc agcttgcgca |
| 20161 | cggtgaaaca gaacttcttg aactctccgg cgctgccact gcgttcgtag atcgtcttga |
| 20221 | acaaccatct ggcttctgcc ttgcctgcgg cgcggcgtgc caggcggtag agaaaacggc |
| 20281 | cgatgccggg atcgatcaaa aagtaatcgg ggtgaaccgt cagcacgtcc gggttcttgc |
| 20341 | cttctgtgat ctcgcggtac atccaatcag ctagctcgat ctcgatgtac tccggccgcc |
| 20401 | cggtttcgct ctttacgatc ttgtagcggc taatcaaggc ttcaccctcg gataccgtca |
| 20461 | ccaggcggcc gttcttggcc ttcttcgtac gctgcatggc aacgtgcgtg gtgtttaacc |
| 20521 | gaatgcaggt ttctaccagg tcgtctttct gctttccgcc atcggctcgc cggcagaact |
| 20581 | tgagtacgtc cgcaacgtgt ggacggaaca cgcggccggg cttgtctccc ttcccttccc |
| 20641 | ggtatcggtt catggattcg gttagatggg aaaccgccat cagtaccagg tcgtaatccc |
| 20701 | acacactggc catgccggcc ggccctgcgg aaacctctac gtgcccgtct ggaagctcgt |
| 20761 | agcggatcac ctcgccagct cgtcggtcac gcttcgacag acggaaaacg gccacgtcca |
| 20821 | tgatgctgcg actatcgcgg gtgcccacgt catagagcat cggaacgaaa aaatctggtt |
| 20881 | gctcgtcgcc cttgggcggc ttcctaatcg acggcgcacc ggctgccggc ggttgccggg |
| 20941 | attctttgcg gattcgatca gcggccgctt gccacgattc accggggcgt gcttctgcct |
| 21001 | cgatgcgttg ccgctgggcg gcctgcgcgg ccttcaactt ctccaccagg tcatcaccca |
| 21061 | gcgccgcgcc gatttgtacc gggccggatg gtttgcgacc gctcacgccg attcctcggg |
| 21121 | cttgggggtt ccagtgccat tgcagggccg gcagacaacc cagccgctta cgcctggcca |
| 21181 | accgcccgtt cctccacaca tggggcattc cacggcgtcg gtgcctggtt gttcttgatt |
| 21241 | ttccatgccg cctcctttag ccgctaaaat tcatctactc atttattcat ttgctcattt |
| 21301 | actctggtag ctgcgcgatg tattcagata gcagctcggt aatggtcttg ccttggcgta |
| 21361 | ccgcgtacat cttcagcttg gtgtgatcct ccgccggcaa ctgaaagttg acccgcttca |
| 21421 | tggctggcgt gtctgccagg ctggccaacg ttgcagcctt gctgctgcgt gcgctcggac |
| 21481 | ggccggcact tagcgtgttt gtgcttttgc tcattttctc tttacctcat taactcaaat |
| 21541 | gagttttgat ttaatttcag cggccagcgc ctggacctcg cgggcagcgt cgccctcggg |
| 21601 | ttctgattca agaacggttg tgccggcggc ggcagtgcct gggtagctca cgcgctgcgt |
| 21661 | gatacgggac tcaagaatgg gcagctcgta cccggccagc gcctcggcaa cctcaccgcc |
| 21721 | gatgcgcgtg cctttgatcg cccgcgacac gacaaaggcc gcttgtagcc ttccatccgt |
| 21781 | gacctcaatg cgctgcttaa ccagctccac caggtcggcg gtggcccata tgtcgtaagg |
| 21841 | gcttggctgc accggaatca gcacgaagtc ggctgccttg atcgcggaca cagccaagtc |
| 21901 | cgccgcctgg ggcgctccgt cgatcactac gaagtcgcgc cggccgatgg ccttcacgtc |
| 21961 | gcggtcaatc gtcgggcggt cgatgccgac aacggttagc ggttgatctt cccgcacggc |
| 22021 | cgcccaatcg cgggcactgc cctggggatc ggaatcgact aacagaacat cggccccggc |
| 22081 | gagttgcagg gcgcgggcta gatgggttgc gatggtcgtc ttgcctgacc cgcctttctg |
| 22141 | gttaagtaca gcgataacct tcatggttc cccttgcgta tttgtttatt tactcatcgc |
| 22201 | atcatatacg cagcgaccgc atgacgcaag ctgttttact caaatacaca tcaccttttt |
| 22261 | agacggcggc gctcggtttc ttcagcggcc aagctggccg gccaggccgc cagcttggca |
| 22321 | tcagacaaac cggccaggat ttcatgcagc cgcacggttg agacgtgcgc gggcggctcg |
| 22381 | aacacgtacc cggccgcgat catctccgcc tcgatctctt cggtaatgaa aaacggttcg |
| 22441 | tcctggccgt cctggtgcgg tttcatgctt gttcctcttg gcgttcattc tcggcggccg |
| 22501 | ccagggcgtc ggcctcggtc aatgcgtcct cacggaaggc accgcgccgc ctggcctcgg |
| 22561 | tgggcgtcac ttcctcgctg cgctcaagtg cgcggtacag ggtcgagcga tgcacgccaa |
| 22621 | gcagtgcagc cgcctctttc acggtgcggc cttcctggtc gatcagctcg cgggcgtgcg |
| 22681 | cgatctgtgc cggggtgagg gtagggcggg ggccaaactt cacgcctcgg gccttggcgg |
| 22741 | cctcgcgccc gctccgggtg cggtcgatga ttagggaacg ctcgaactcg gcaatgccgg |
| 22801 | cgaacacggt caacaccatg cggccggccg gcgtggtggt gtcggcccac ggctctgcca |
| 22861 | ggctacgcag gcccgcgccg gcctcctgga tgcgctcggc aatgtccagt aggtcgcggg |
| 22921 | tgctgcgggc caggcggtct agcctggtca ctgtcacaac gtcgccaggg cgtaggtggt |
| 22981 | caagcatcct ggccagctcc gggcggtcgc gcctggtgcc ggtgatcttc tcggaaaaca |
| 23041 | gcttggtgca gccggccgcg tgcagttcgg cccgttggtt ggtcaagtcc tggtcgtcgg |
| 23101 | tgctgacgcg ggcatagccc agcaggccag cggcggcgct cttgttcatg gcgtaatgtc |
| 23161 | tccggttcta gtcgcaagta ttctacttta tgcgactaaa acacgcgaca agaaaacgcc |
| 23221 | aggaaaaggg cagggcggca gcctgtcgcg taacttagga cttgtgcgac atgtcgtttt |
| 23281 | cagaagacgg ctgcactgaa cgtcagaagc cgactgcact atagcagcgg aggggttgga |
| 23341 | tcaaagtact ttgatcccga ggggaaccct gtggttggca tgcacataca aatggacgaa |
| 23401 | cggataaacc ttttcacgcc cttttaaata tccgttattc taataaacgc tcttttctct |
| 23461 | tag |

//
SEQ ID NO: 75.

| | |
|---|---|
| LOCUS | pHelper_in_fig._1; gRNA, Pong ORF1 and ORF2 fused |
| | to Cas9 21092 bp ds-DNA circular 02-JUN.-2021. ORF1 protein, the |
| | ORF2 protein, the Cas9 protein, and the gRNA |
| DEFINITION | . |
| ACCESSION | pVec1 |
| VERSION | pVec1 .1 |
| FEATURES | Location/Qualifiers |
| Agro tDNA cut site | 1 . . . 25 |
| | /label = “RB″ |
| misc_feature | 254 . . . 677 |
| | /label = “U6-26 promoter″ |
| misc_feature | 678 . . . 697 |
| | /label = “gRNA″ |
| misc_feature | 698 . . . 773 |
| | /label = “gRNA scaffold″ |
| misc_feature | 774 . . . 965 |
| | /label = “U6-26 terminator″ |
| promoter | 981 . . . 2667 |
| | /label = “Rps5a promoter″ |
| misc_feature | 2704 . . . 4101 |
| | /label = “Pong ORF1″ |
| CDS | 2704 . . . 4101 |
| | /label = “Translation 2704-4101″ |
| terminator | 4165 . . . 4890 |
| | /label = “OCS terminator″ |
| promoter | 5073 . . . 5992 |
| | /label = “GmUbi3 promoter″ |
| misc_feature | 6014 . . . 7459 |
| | /label = “Pong ORF2″ |
| CDS | 6014 . . . 11677 |
| | /label = “Translation 6014-11677″ |
| misc_feature | 7463 . . . 7477 |
| | /label = “G4S linker″ |
| feature | 7481 . . . 7501 |
| | /label = “NLS″ |
| misc_feature | 7505 . . . 11626 |
| | /label = “Cas9″ |
| misc_feature | 11627 . . . 11674 |
| | /label = “NLS″ |
| terminator | 11702 . . . 12429 |
| | /label = “OCS terminator″ |
| promoter | 12680 . . . 13420 |
| | /label = “CaMV 35S promoter″ |
| gene | 13510 . . . 14505 |
| | /label = “HygR″ |
| CDS | 13510 . . . 14505 |
| | /label = “Translation 13510-14505″ |
| misc_feature | complement (15124 . . . 15146) |
| | /label = “LB″ |
| gene | 15262 . . . 16056 |
| | /label = “KanR″ |
| origin | 16127 . . . 16746 |
| | /label = “pBR322_origin″ |
| ORIGIN | |
| 1 | gtttacccgc caatatatcc tgtcaaacac tgatagttta aactgaaggc gggaaacgac |
| 61 | aatctgatcc aagctcaagc tgctctagca ttcgccattc aggctgcgca actgttggga |
| 121 | agggcgatcg gtgcgggcct cttcgctatt acgccagctg gcgaaagggg gatgtgctgc |
| 181 | aaggcgatta agttgggtaa cgccagggtt ttcccagtca cgacgttgta aaacgacggc |
| 241 | cagtgccaag cttcgacttg ccttccgcac aatacatcat ttcttcttag ctttttttct |
| 301 | tcttcttcgt tcatacagtt tttttttgtt tatcagctta cattttcttg aaccgtagct |
| 361 | ttcgttttct tctttttaac tttccattcg gagtttttgt atcttgtttc atagtttgtc |
| 421 | ccaggattag aatgattagg catcgaacct tcaagaattt gattgaataa aacatcttca |
| 481 | ttcttaagat atgaagataa tcttcaaaag gcccctggga atctgaaaga agagaagcag |
| 541 | gcccatttat atgggaaaga acaatagtat ttcttatata ggcccattta agttgaaaac |
| 601 | aatcttcaaa agtcccacat cgcttagata agaaaacgaa gctgagttta tatacagcta |
| 661 | gagtcgaagt agtgattGCC AGCCATGGTC GGCGGTCgtt ttagagctag aaatagcaag |
| 721 | ttaaaataag gctagtccgt tatcaacttg aaaaagtggc accgagtcgg tgcttttttt |
| 781 | tgcaaaattt tccagatcga tttcttcttc ctctgttctt cggcgttcaa tttctggggt |
| 841 | tttctcttcg ttttctgtaa ctgaaaccta aaatttgacc taaaaaaaat ctcaaataat |
| 901 | atgattcagt ggttttgtac ttttcagtta gttgagtttt gcagttccga tgagataaac |
| 961 | caataccatg ttagagagcg ctagttcgtg agtagatata ttactcaact tttgattcgc |
| 1021 | tatttgcagt gcacctgtgg cgttcatcac atcttttgtg acactgtttg cactggtcat |
| 1081 | tgctattaca aaggaccttc ctgatgttga aggagatcga aagtaagtaa ctgcacgcat |
| 1141 | aaccattttc tttccgctct ttggctcaat ccatttgaca gtcaaagaca atgtttaacc |
| 1201 | agctccgttt gatatattgt ctttatgtgt ttgttcaagc atgtttagtt aatcatgcct |
| 1261 | ttgattgatc ttgaataggt tccaaatatc aaccctggca acaaaacttg gagtgagaaa |
| 1321 | cattgcattc ctcggttctg gacttctgct agtaaattat gtttcagcca tatcactagc |
| 1381 | tttctacatg cctcaggtga attcatctat ttccgtctta actatttcgg ttaatcaaag |
| 1441 | cacgaacacc attactgcat gtagaagctt gataaactat cgccaccaat ttatttttgt |
| 1501 | tgcgatattg ttactttcct cagtatgcag ctttgaaaag accaaccctc ttatccttta |
| 1561 | acaatgaaca ggtttttaga ggtagcttga tgattcctgc acatgtgatc ttggcttcag |
| 1621 | gcttaatttt ccaggtaaag cattatgaga tactcttata tctcttacat acttttgaga |
| 1681 | taatgcacaa gaacttcata actatatgct ttagtttctg catttgacac tgccaaattc |
| 1741 | attaatctct aatatctttg ttgttgatct ttggtagaca tgggtactag aaaaagcaaa |
| 1801 | ctacaccaag gtaaaatact tttgtacaaa cataaactcg ttatcacgga acatcaatgg |
| 1861 | agtgtatatc taacggagtg tagaaacatt tgattattgc aggaagctat ctcaggatat |
| 1921 | tatcggttta tatggaatct cttctacgca gagtatctgt tattcccctt cctctagctt |
| 1981 | tcaatttcat ggtgaggata tgcagttttc tttgtatatc attcttcttc ttctttgtag |
| 2041 | cttggagtca aaatcggttc cttcatgtac atacatcaag gatatgtcct tctgaatttt |
| 2101 | tatatcttgc aataaaaatg cttgtaccaa ttgaaacacc agctttttga gttctatgat |
| 2161 | cactgacttg gttctaacca aaaaaaaaaa aatgtttaat ttacatatct aaaagtaggt |
| 2221 | ttagggaaac ctaaacagta aaatatttgt atattattcg aatttcactc atcataaaaa |
| 2281 | cttaaattgc accataaaat tttgttttac tattaatgat gtaatttgtg taacttaaga |
| 2341 | taaaaataat attccgtaag ttaaccggct aaaaccacgt ataaaccagg gaacctgtta |
| 2401 | aaccggttct ttactggata aagaaatgaa agcccatgta gacagctcca ttagagccca |
| 2461 | aaccctaaat ttctcatcta tataaaagga gtgacattag ggtttttgtt cgtcctctta |
| 2521 | aagcttctcg ttttctctgc cgtctctctc attcgcgcga cgcaaacgat cttcaggtga |
| 2581 | tcttctttct ccaaatcctc tctcataact ctgatttcgt acttgtgtat ttgagctcac |
| 2641 | gctctgtttc tctcaccaca gccggattcg agatcacaag tttgtacaaa aaagcaggct |
| 2701 | tccatggatc cgtcgccggc cgtggatccg tcgccggccg tggatccgtc gccggctgct |
| 2761 | gaaacccggc ggcgtgcaac cgggaaagga ggcaaacagc gcgggggcaa gcaactagga |
| 2821 | ttgaagaggc cgccgccgat ttctgtcccg gccaccccgc ctcctgctgc gacgtcttca |
| 2881 | tcccctgctg cgccgacggc catcccacca cgaccaccgc aatcttcgcc gattttcgtc |
| 2941 | cccgattcgc cgaatccgtc accggctgcg ccgacctcct ctcttgcttc ggggacatcg |
| 3001 | acggcaaggc caccgcaacc acaaggagga ggatggggac caacatcgac catttcccca |
| 3061 | aactttgcat ctttctttgg aaaccaacaa gacccaaatt catgtttggt caggggttat |
| 3121 | cctccaggag ggtttgtcaa ttttattcaa caaaattgtc cgccgcagcc acaacagcaa |
| 3181 | ggtgaaaatt ttcatttcgt tggtcacaat atggggttca acccaatatc tccacagcca |
| 3241 | ccaagtgcct acggaacacc aacaccccaa gctacgaacc aaggcacttc aacaaacatt |
| 3301 | atgattgatg aagaggacaa caatgatgac agtagggcag caaagaaaag atggactcat |
| 3361 | gaagaggaag agagactggc cagtgcttgg ttgaatgctt ctaaagactc aattcatggg |
| 3421 | aatgataaga aaggtgatac attttggaag gaagtcactg atgaatttaa caagaaaggg |
| 3481 | aatggaaaac gtaggaggga aattaaccaa ctgaaggttc actggtcaag gttgaagtca |
| 3541 | gcgatctctg agttcaatga ctattggagt acggttactc aaatgcatac aagcggatac |
| 3601 | tcagacgaca tgcttgagaa agaggcacag aggctgtatg caaacaggtt tggaaaacct |
| 3661 | tttgcgttgg tccattggtg gaagatactc aaaagagagc ccaaatggtg tgctcagttt |
| 3721 | gaaaagagga aaaggaagag cgaaatggat gctgttccag aacagcagaa acgtcctatt |
| 3781 | ggtagagaag cagcaaagtc tgagcgcaaa agaaagcgca agaaagaaaa tgttatggaa |
| 3841 | ggcattgtcc tcctagggga caatgtccag aaaattatca aagtgacgca agatcggaag |
| 3901 | ctggagcgtg agaaggtcac tgaagcacag attcacattt caaacgtaaa tttgaaggca |
| 3961 | gcagaacagc aaaaagaagc aaagatgttt gaggtataca attccctgct cactcaagat |
| 4021 | acaagtaaca tgtctgaaga acagaaggct cgccgagaca aggcattaca aaagctggag |
| 4081 | gaaaagttat ttgctgacta gtgacccagc tttcttgtac aaagtggtgc ctaggtgagt |
| 4141 | ctagagagtt gattaagacc cgggactggt ccctagagtc ctgctttaat gagatatgcg |
| 4201 | agacgcctat gatcgcatga tatttgcttt caattctgtt gtgcacgttg taaaaaacct |
| 4261 | gagcatgtgt agctcagatc cttaccgccg gtttcggttc attctaatga atatatcacc |
| 4321 | cgttactatc gtatttttat gaataatatt ctccgttcaa tttactgatt gtaccctact |
| 4381 | acttatatgt acaatattaa aatgaaaaca atatattgtg ctgaataggt ttatagcgac |
| 4441 | atctatgata gagcgccaca ataacaaaca attgcgtttt attattacaa atccaatttt |
| 4501 | aaaaaaagcg gcagaaccgg tcaaacctaa aagactgatt acataaatct tattcaaatt |
| 4561 | tcaaaagtgc cccaggggct agtatctacg acacaccgag cggcgaacta ataacgctca |
| 4621 | ctgaagggaa ctccggttcc ccgccggcgc gcatgggtga gattccttga agttgagtat |
| 4681 | tggccgtccg ctctaccgaa agttacgggc accattcaac ccggtccagc acggcggccg |
| 4741 | ggtaaccgac ttgctgcccc gagaattatg cagcattttt ttggtgtatg tgggccccaa |
| 4801 | atgaagtgca ggtcaaacct tgacagtgac gacaaatcgt tgggcgggtc cagggcgaat |
| 4861 | tttgcgacaa catgtcgagg ctcagcagga cctgcaggca tgcaagcttg gcactggccg |
| 4921 | tcgttttaca acgtcgtgac tgggaaaacc ctggcgttac ccaacttaat cgccttgcag |
| 4981 | cacatccccc tttcgccagc tggcgtaata gcgaagaggc ccgcaccgat cgcccttccc |
| 5041 | aacagttgcg cagcctgaat ggcgaatgct agagcagctt gagcttggat cagattgtcg |
| 5101 | tttcccgcct tcagtttctt gaaggtgcat gtgactccgt caagattacg aaaccgccaa |
| 5161 | ctaccacgca aattgcaatt ctcaatttcc tagaaggact ctccgaaaat gcatccaata |
| 5221 | ccaaatatta cccgtgtcat aggcaccaag tgacaccata catgaacacg cgtcacaata |
| 5281 | tgactggaga agggttccac accttatgct ataaaacgcc ccacacccct cctccttcct |
| 5341 | tcgcagttca attccaatat attccattct ctctgtgtat ttccctacct ctcccttcaa |
| 5401 | ggttagtcga tttcttctgt ttttcttctt cgttctttcc atgaattgtg tatgttcttt |
| 5461 | gatcaatacg atgttgattt gattgtgttt tgtttggttt catcgatctt caattttcat |
| 5521 | aatcagattc agcttttatt atctttacaa caacgtcctt aatttgatga ttctttaatc |
| 5581 | gtagatttgc tctaattaga gctttttcat gtcagatccc tttacaacaa gccttaattg |
| 5641 | ttgattcatt aatcgtagat tagggctttt ttcattgatt acttcagatc cgttaaacgt |
| 5701 | aaccatagat cagggctttt tcatgaatta cttcagatcc gttaaacaac agccttattt |
| 5761 | tttatacttc tgtggttttt caagaaattg ttcagatccg ttgacaaaaa gccttattcg |
| 5821 | ttgattctat atcgtttttc gagagatatt gctcagatct gttagcaact gccttgtttg |
| 5881 | ttgattctat tgccgtggat tagggttttt tttcacgaga ttgcttcaga tccgtactta |
| 5941 | agattacgta atggattttg attctgattt atctgtgatt gttgactcga caggtacctt |
| 6001 | caaacggcgc gccatgcaga gtttagccat ctctctactc ctctcagaaa ctcattccct |
| 6061 | cttttctcat acgaagacct cctccctttt atctttactg tttctctctt cttcaaagat |
| 6121 | gtctgagcaa aatactgatg gaagtcaagt tccagtgaac ttgttggatg agttcctggc |
| 6181 | tgaggatgag atcatagatg atcttctcac tgaagccacg gtggtagtac agtccactat |
| 6241 | agaaggtctt caaaacgagg cttctgacca tcgacatcat ccgaggaagc acatcaagag |
| 6301 | gccacgagag gaagcacatc agcaactggt gaatgattac ttttcagaaa atcctcttta |
| 6361 | cccttccaaa atttttcgtc gaagatttcg tatgtctagg ccactttttc ttcgcatcgt |
| 6421 | tgaggcatta ggccagtggt cagtgtattt cacacaaagg gtggatgctg ttaatcggaa |
| 6481 | aggactcagt ccactgcaaa agtgtactgc agctattcgc cagttggcta ctggtagtgg |
| 6541 | cgcagatgaa ctagatgaat atctgaagat aggagagact acagcaatgg aggcaatgaa |
| 6601 | gaattttgtc aaaggtcttc aagatgtgtt tggtgagagg tatcttaggc gccccactat |
| 6661 | ggaagatacc gaacggcttc tccaacttgg tgagaaacgt ggttttcctg gaatgttcgg |
| 6721 | cagcattgac tgcatgcact ggcattggga aagatgccca gtagcatgga agggtcagtt |
| 6781 | cactcgtgga gatcagaaag tgccaaccct gattcttgag gctgtggcat cgcatgatct |
| 6841 | ttggatttgg catgcatttt ttggagcagc gggttccaac aatgatatca atgtattgaa |
| 6901 | ccaatctact gtatttatca aggagctcaa aggacaagct cctagagtcc agtacatggt |
| 6961 | aaatgggaat caatacaata ctgggtattt tcttgctgat ggaatctacc ctgaatgggc |
| 7021 | agtgtttgtt aagtcaatac gactcccaaa cactgaaaag gagaaattgt atgcagatat |
| 7081 | gcaagaaggg gcaagaaaag atatcgagag agcctttggt gtattgcagc gaagattttg |
| 7141 | catcttaaaa cgaccagctc gtctatatga tcgaggtgta ctgcgagatg ttgttctagc |
| 7201 | ttgcatcata cttcacaata tgatagttga agatgagaag gaaaccagaa ttattgaaga |
| 7261 | agatgcagat gcaaatgtgc ctcctagttc atcaaccgtt caggaacctg agttctctcc |
| 7321 | tgaacagaac acaccatttg atagagtttt agaaaaagat atttctatcc gagatcgagc |
| 7381 | ggctcataac cgacttaaga aagatttggt ggaacacatt tggaataagt ttggtggtgc |
| 7441 | tgcacataga actggaaatt atggcggggg aggtagcgct ccgaagaaga agaggaaggt |
| 7501 | tggcatccac ggggtgccag ctgctgacaa gaagtactcg atcggcctcg atattgggac |
| 7561 | taactctgtt ggctgggccg tgatcaccga cgagtacaag gtgccctcaa agaagttcaa |
| 7621 | ggtcctgggc aacaccgatc ggcattccat caagaagaat ctcattggcg ctctcctgtt |
| 7681 | cgacagcggc gagacggctg aggctacgcg gctcaagcgc accgcccgca ggcggtacac |
| 7741 | gcgcaggaag aatcgcatct gctacctgca ggagattttc tccaacgaga tggcgaaggt |
| 7801 | tgacgattct ttcttccaca ggctggagga gtcattcctc gtggaggagg ataagaagca |
| 7861 | cgagcggcat ccaatcttcg gcaacattgt cgacgaggtt gcctaccacg agaagtaccc |
| 7921 | tacgatctac catctgcgga agaagctcgt ggactccaca gataaggcgg acctccgcct |
| 7981 | gatctacctc gctctggccc acatgattaa gttcaggggc catttcctga tcgaggggga |
| 8041 | tctcaacccg gacaatagcg atgttgacaa gctgttcatc cagctcgtgc agacgtacaa |
| 8101 | ccagctcttc gaggagaacc ccattaatgc gtcaggcgtc gacgcgaagg ctatcctgtc |
| 8161 | cgctaggctc tcgaagtctc ggcgcctcga gaacctgatc gcccagctgc cgggcgagaa |
| 8221 | gaagaacggc ctgttcggga atctcattgc gctcagcctg gggctcacgc ccaacttcaa |
| 8281 | gtcgaatttc gatctcgctg aggacgccaa gctgcagctc tccaaggaca catacgacga |
| 8341 | tgacctggat aacctcctgg cccagatcgg cgatcagtac gcggacctgt tcctcgctgc |
| 8401 | caagaatctg tcggacgcca tcctcctgtc tgatattctc agggtgaaca ccgagattac |
| 8461 | gaaggctccg ctctcagcct ccatgatcaa gcgctacgac gagcaccatc aggatctgac |
| 8521 | cctcctgaag gcgctggtca ggcagcagct ccccgagaag tacaaggaga tcttcttcga |
| 8581 | tcagtcgaag aacggctacg ctgggtacat tgacggcggg gcctctcagg aggagttcta |
| 8641 | caagttcatc aagccgattc tggagaagat ggacggcacg gaggagctgc tggtgaagct |
| 8701 | caatcgcgag gacctcctga ggaagcagcg gacattcgat aacggcagca tcccacacca |
| 8761 | gattcatctc ggggagctgc acgctatcct gaggaggcag gaggacttct accctttcct |
| 8821 | caaggataac cgcgagaaga tcgagaagat tctgactttc aggatcccgt actacgtcgg |
| 8881 | cccactcgct aggggcaact cccgcttcgc ttggatgacc cgcaagtcag aggagacgat |
| 8941 | cacgccgtgg aacttcgagg aggtggtcga caagggcgct agcgctcagt cgttcatcga |
| 9001 | gaggatgacg aatttcgaca agaacctgcc aaatgagaag gtgctcccta agcactcgct |
| 9061 | cctgtacgag tacttcacag tctacaacga gctgactaag gtgaagtatg tgaccgaggg |
| 9121 | catgaggaag ccggctttcc tgtctgggga gcagaagaag gccatcgtgg acctcctgtt |
| 9181 | caagaccaac cggaaggtca cggttaagca gctcaaggag gactacttca agaagattga |
| 9241 | gtgcttcgat tcggtcgaga tctctggcgt tgaggaccgc ttcaacgcct ccctggggac |
| 9301 | ctaccacgat ctcctgaaga tcattaagga taaggacttc ctggacaacg aggagaatga |
| 9361 | ggatatcctc gaggacattg tgctgacact cactctgttc gaggaccggg agatgatcga |
| 9421 | ggagcgcctg aagacttacg cccatctctt cgatgacaag gtcatgaagc agctcaagag |
| 9481 | gaggaggtac accggctggg ggaggctgag caggaagctc atcaacggca ttcgggacaa |
| 9541 | gcagtccggg aagacgatcc tcgacttcct gaagagcgat ggcttcgcga accgcaattt |
| 9601 | catgcagctg attcacgatg acagcctcac attcaaggag gatatccaga aggctcaggt |
| 9661 | gagcggccag ggggactcgc tgcacgagca tatcgcgaac ctcgctggct cgccagctat |
| 9721 | caagaagggg attctgcaga ccgtgaaggt tgtggacgag ctggtgaagg tcatgggcag |
| 9781 | gcacaagcct gagaacatcg tcattgagat ggcccgggag aatcagacca cgcagaaggg |
| 9841 | ccagaagaac tcacgcgaga ggatgaagag gatcgaggag ggcattaagg agctggggtc |
| 9901 | ccagatcctc aaggagcacc cggtggagaa cacgcagctg cagaatgaga agctctacct |
| 9961 | gtactacctc cagaatggcc gcgatatgta tgtggaccag gagctggata ttaacaggct |
| 10021 | cagcgattac gacgtcgatc atatcgttcc acagtcattc ctgaaggatg actccattga |
| 10081 | caacaaggtc ctcaccaggt cggacaagaa ccggggcaag tctgataatg ttccttcaga |
| 10141 | ggaggtcgtt aagaagatga agaactactg gcgccagctc ctgaatgcca agctgatcac |
| 10201 | gcagcggaag ttcgataacc tcacaaaggc tgagaggggc gggctctctg agctggacaa |
| 10261 | ggcgggcttc atcaagaggc agctggtcga gacacggcag atcactaagc acgttgcgca |
| 10321 | gattctcgac tcacggatga acactaagta cgatgagaat gacaagctga tccgcgaggt |
| 10381 | gaaggtcatc accctgaagt caaagctcgt ctccgacttc aggaaggatt tccagttcta |
| 10441 | caaggttcgg gagatcaaca attaccacca tgcccatgac gcgtacctga acgcggtggt |
| 10501 | cggcacagct ctgatcaaga agtacccaaa gctcgagagc gagttcgtgt acggggacta |
| 10561 | caaggtttac gatgtgagga agatgatcgc caagtcggag caggagattg gcaaggctac |
| 10621 | cgccaagtac ttcttctact ctaacattat gaatttcttc aagacagaga tcactctggc |
| 10681 | caatggcgag atccggaagc gccccctcat cgagacgaac ggcgagacgg gggagatcgt |
| 10741 | gtgggacaag ggcagggatt tcgcgaccgt caggaaggtt ctctccatgc cacaagtgaa |
| 10801 | tatcgtcaag aagacagagg tccagactgg cgggttctct aaggagtcaa ttctgcctaa |
| 10861 | gcggaacagc gacaagctca tcgcccgcaa gaaggactgg gatccgaaga agtacggcgg |
| 10921 | gttcgacagc cccactgtgg cctactcggt cctggttgtg gcgaaggttg agaagggcaa |
| 10981 | gtccaagaag ctcaagagcg tgaaggagct gctggggatc acgattatgg agcgctccag |
| 11041 | cttcgagaag aacccgatcg atttcctgga ggcgaagggc tacaaggagg tgaagaagga |
| 11101 | cctgatcatt aagctcccca agtactcact cttcgagctg gagaacggca ggaagcggat |
| 11161 | gctggcttcc gctggcgagc tgcagaaggg gaacgagctg gctctgccgt ccaagtatgt |
| 11221 | gaacttcctc tacctggcct cccactacga gaagctcaag ggcagccccg aggacaacga |
| 11281 | gcagaagcag ctgttcgtcg agcagcacaa gcattacctc gacgagatca ttgagcagat |
| 11341 | ttccgagttc tccaagcgcg tgatcctggc cgacgcgaat ctggataagg tcctctccgc |
| 11401 | gtacaacaag caccgcgaca agccaatcag ggagcaggct gagaatatca ttcatctctt |
| 11461 | caccctgacg aacctcggcg cccctgctgc tttcaagtac ttcgacacaa ctatcgatcg |
| 11521 | caagaggtac acaagcacta aggaggtcct ggacgcgacc ctcatccacc agtcgattac |
| 11581 | cggcctctac gagacgcgca tcgacctgtc tcagctcggg ggcgacaagc ggccagcggc |
| 11641 | gacgaagaag gcggggcagg cgaagaagaa gaagtgataa ttgacattct aatctagagt |
| 11701 | cctgctttaa tgagatatgc gagacgccta tgatcgcatg atatttgctt tcaattctgt |
| 11761 | tgtgcacgtt gtaaaaaacc tgagcatgtg tagctcagat ccttaccgcc ggtttcggtt |
| 11821 | cattctaatg aatatatcac ccgttactat cgtattttta tgaataatat tctccgttca |
| 11881 | atttactgat tgtaccctac tacttatatg tacaatatta aaatgaaaac aatatattgt |
| 11941 | gctgaatagg tttatagcga catctatgat agagcgccac aataacaaac aattgcgttt |
| 12001 | tattattaca aatccaattt taaaaaaagc ggcagaaccg gtcaaaccta aaagactgat |
| 12061 | tacataaatc ttattcaaat ttcaaaagtg ccccaggggc tagtatctac gacacaccga |
| 12121 | gcggcgaact aataacgttc actgaaggga actccggttc cccgccggcg cgcatgggtg |
| 12181 | agattccttg aagttgagta ttggccgtcc gctctaccga aagttacggg caccattcaa |
| 12241 | cccggtccag cacggcggcc gggtaaccga cttgctgccc cgagaattat gcagcatttt |
| 12301 | tttggtgtat gtgggcccca aatgaagtgc aggtcaaacc ttgacagtga cgacaaatcg |
| 12361 | ttgggcgggt ccagggcgaa ttttgcgaca acatgtcgag gctcagcagg acctgcaggc |
| 12421 | atgcaagatc gcgaattcgt aatcatgtca tagctgtttc ctgtgtgaaa ttgttatccg |
| 12481 | ctcacaattc cacacaacat acgagccgga agcataaagt gtaaagcctg gggtgcctaa |
| 12541 | tgagtgagct aactcacatt aattgcgttg cgctcactgc ccgctttcca gtcgggaaac |
| 12601 | ctgtcgtgcc agctgcatta atgaatcggc caacgcgcgg ggagaggcgg tttgcgtatt |
| 12661 | ggctagagca gcttgccaac atggtggagc acgacactct cgtctactcc aagaatatca |
| 12721 | aagatacagt ctcagaagac caaagggcta ttgagacttt tcaacaaagg gtaatatcgg |
| 12781 | gaaacctcct cggattccat tgcccagcta tctgtcactt catcaaaagg acagtagaaa |
| 12841 | aggaaggtgg cacctacaaa tgccatcatt gcgataaagg aaaggctatc gttcaagatg |
| 12901 | cctctgccga cagtggtccc aaagatggac ccccacccac gaggagcatc gtggaaaaag |
| 12961 | aagacgttcc aaccacgtct tcaaagcaag tggattgatg tgaacatggt ggagcacgac |
| 13021 | actctcgtct actccaagaa tatcaaagat acagtctcag aagaccaaag ggctattgag |
| 13081 | acttttcaac aaagggtaat atcgggaaac ctcctcggat tccattgccc agctatctgt |
| 13141 | cacttcatca aaaggacagt agaaaaggaa ggtggcacct acaaatgcca tcattgcgat |
| 13201 | aaaggaaagg ctatcgttca agatgcctct gccgacagtg gtcccaaaga tggaccccca |
| 13261 | cccacgagga gcatcgtgga aaaagaagac gttccaacca cgtcttcaaa gcaagtggat |
| 13321 | tgatgtgata tctccactga cgtaagggat gacgcacaat cccactatcc ttcgcaagaC |
| 13381 | ccttcctcta tataaggaag ttcatttcat ttggagagga cacgctgaaa tcaccagtct |
| 13441 | ctctctacaa atctatctct ctcgagcttt cgcagatccg gggggcaatg agatatgaaa |
| 13501 | aagcctgaac tcaccgcgac gtctgtcgag aagtttctga tcgaaaagtt cgacagcgtc |
| 13561 | tccgacctga tgcagctctc ggagggcgaa gaatctcgtg ctttcagctt cgatgtagga |
| 13621 | gggcgtggat atgtcctgcg ggtaaatagc tgcgccgatg gtttctacaa agatcgttat |
| 13681 | gtttatcggc actttgcatc ggccgcgctc ccgattccgg aagtgcttga cattggggag |
| 13741 | tttagcgaga gcctgaccta ttgcatctcc cgccgtTcac agggtgtcac gttgcaagac |
| 13801 | ctgcctgaaa ccgaactgcc cgctgttcta caaccggtcg cggaggctat ggatgcgatc |
| 13861 | gctgcggccg atcttagcca gacgagcggg ttcggcccat tcggaccgca aggaatcggt |
| 13921 | caatacacta catggcgtga tttcatatgc gcgattgctg atccccatgt gtatcactgg |
| 13981 | caaactgtga tggacgacac cgtcagtgcg tccgtcgcgc aggctctcga tgagctgatg |
| 14041 | ctttgggccg aggactgccc cgaagtccgg cacctcgtgc acgcggattt cggctccaac |
| 14101 | aatgtcctga cggacaatgg ccgcataaca gcggtcattg actggagcga ggcgatgttc |
| 14161 | ggggattccc aatacgaggt cgccaacatc ttcttctgga ggccgtggtt ggcttgtatg |
| 14221 | gagcagcaga cgcgctactt cgagcggagg catccggagc ttgcaggatc gccacgactc |
| 14281 | cgggcgtata tgctccgcat tggtcttgac caactctatc agagcttggt tgacggcaat |
| 14341 | ttcgatgatg cagcttgggc gcagggtcga tgcgacgcaa tcgtccgatc cggagccggg |
| 14401 | actgtcgggc gtacacaaat cgcccgcaga agcgcggccg tctggaccga tggctgtgta |
| 14461 | gaagtactcg ccgatagtgg aaaccgacgc cccagcactc gtccgagggc aaagaaatag |
| 14521 | agtagatgcc gaccGggatc tgtcgatcga caagctcgag tttctccata ataatgtgtg |
| 14581 | agtagttccc agataaggga attagggttc ctatagggtt tcgctcatgt gttgagcata |
| 14641 | taagaaaccc ttagtatgta tttgtatttg taaaatactt ctatcaataa aatttctaat |
| 14701 | tcctaaaacc aaaatccagt actaaaatcc agatcccccg aattaattcg gcgttaattc |
| 14761 | agtacattaa aaacgtccgc aatgtgttat taagttgtct aagcgtcaat ttgtttacac |
| 14821 | cacaatatat cctgccacca gccagccaac agctccccga ccggcagctc ggcacaaaat |
| 14881 | caccactcga tacaggcagc ccatcagtcc gggacggcgt cagcgggaga gccgttgtaa |
| 14941 | ggcggcagac tttgctcatg ttaccgatgc tattcggaag aacggcaact aagctgccgg |
| 15001 | gtttgaaaca cggatgatct cgcggagggt agcatgttga ttgtaacgat gacagagcgt |
| 15061 | tgctgcctgt gatcaccgcg gtttcaaaat cggctccgtc gatactatgt tatacgccaa |
| 15121 | ctttgaaaac aactttgaaa aagctgtttt ctggtattta aggttttaga atgcaaggaa |
| 15181 | cagtgaattg gagttcgtct tgttataatt agcttcttgg ggtatcttta aatactgtag |
| 15241 | aaaagaggaa ggaaataata aatggctaaa atgagaatat caccggaatt gaaaaaactg |
| 15301 | atcgaaaaat accgctgcgt aaaagatacg gaaggaatgt ctcctgctaa ggtatataag |
| 15361 | ctggtgggag aaaatgaaaa cctatattta aaaatgacgg acagccggta taaagggacc |
| 15421 | acctatgatg tggaacggga aaaggacatg atgctatggc tggaaggaaa gctgcctgtt |
| 15481 | ccaaaggtcc tgcactttga acggcatgat ggctggagca atctgctcat gagtgaggcc |
| 15541 | gatggcgtcc tttgctcgga agagtatgaa gatgaacaaa gccctgaaaa gattatcgag |
| 15601 | ctgtatgcgg agtgcatcag gctctttcac tccatcgaca tatcggattg tccctatacg |
| 15661 | aatagcttag acagccgctt agccgaattg gattacttac tgaataacga tctggccgat |
| 15721 | gtggattgcg aaaactggga agaagacact ccatttaaag atccgcgcga gctgtatgat |
| 15781 | tttttaaaga cggaaaagcc cgaagaggaa cttgtctttt cccacggcga cctgggagac |
| 15841 | agcaacatct ttgtgaaaga tggcaaagta agtggcttta ttgatcttgg gagaagcggc |
| 15901 | agggcggaca agtggtatga cattgccttc tgcgtccggt cgatcaggga ggatatcggg |
| 15961 | gaagaacagt atgtcgagct attttttgac ttactgggga tcaagcctga ttgggagaaa |
| 16021 | ataaaatatt atattttact ggatgaattg ttttagtacc tagaatgcat gaccaaaatc |
| 16081 | ccttaacgtg agttttcgtt ccactgagcg tcagaccccg tagaaaagat caaaggatct |
| 16141 | tcttgagatc ctttttttct gcgcgtaatc tgctgcttgc aaacaaaaaa accaccgcta |
| 16201 | ccagcggtgg tttgtttgcc ggatcaagag ctaccaactc tttttccgaa ggtaactggc |
| 16261 | ttcagcagag cgcagatacc aaatactgtc cttctagtgt agccgtagtt aggccaccac |
| 16321 | ttcaagaact ctgtagcacc gcctacatac ctcgctctgc taatcctgtt accagtggct |
| 16381 | gctgccagtg gcgATAAGTC gtgtcttacc gggttggact caagacgata gttaccggat |
| 16441 | aaggcgcagc ggtcgggctg aacggggggt tcgtgcacac agcccagctt ggagcgaacg |
| 16501 | acctacaccg aactgagata cctacagcgt gagctatgag aaagcgccac gcttcccgaa |
| 16561 | gggagaaagg cggacaggta tccggtaagc ggcagggtcg gaacaggaga gcgcacgagg |
| 16621 | gagcttccag ggggaaacgc ctggtatctt tatagtcctg tcgggtttcg ccacctctga |
| 16681 | cttgagcgtc gatttttgtg atgctcgtca ggggggcgga gcctatggaa aaacgccagc |
| 16741 | aacgcggcct ttttacggtt cctggccttt tgctggcctt ttgctcacat gttctttcct |
| 16801 | gcgttatccc ctgattctgt ggataaccgt attaccgcct ttgagtgagc tgataccgct |
| 16861 | cgccgcagcc gaacgaccga gcgcagcgag tcagtgagcg aggaagcgga agagcgcctg |
| 16921 | atgcggtatt ttctccttac gcatctgtgc ggtatttcac accgcatatg gtgcactctc |
| 16981 | agtacaatct gctctgatgc cgcatagtta agccagtata cactccgcta tcgctacgtg |
| 17041 | actgggtcat ggctgcgccc cgacacccgc caacacccgc tgacgcgccc tgacgggctt |
| 17101 | gtctgctccc ggcatccgct tacagacaag ctgtgaccgt ctccgggagc tgcatgtgtc |
| 17161 | agaggttttc accgtcatca ccgaaacgcg cgaggcaggg tgccttgatg tgggcgccgg |
| 17221 | cggtcgagtg gcgacggcgc ggcttgtccg cgccctggta gattgcctgg ccgtaggcca |
| 17281 | gccatttttg agcggccagc ggccgcgata ggccgacgcg aagcggcggg gcgtagggag |
| 17341 | cgcagcgacc gaagggtagg cgctttttgc agctcttcgg ctgtgcgctg gccagacagt |
| 17401 | tatgcacagg ccaggcgggt tttaagagtt ttaataagtt ttaaagagtt ttaggcggaa |
| 17461 | aaatcgcctt ttttctcttt tatatcagtc acttacatgt gtgaccggtt cccaatgtac |
| 17521 | ggctttgggt tcccaatgta cgggttccgg ttcccaatgt acggctttgg gttcccaatg |
| 17581 | tacgtgctat ccacaggaaa cagacctttt cgaccttttt cccctgctag ggcaatttgc |
| 17641 | cctagcatct gctccgtaca ttaggaaccg gcggatgctt cgccctcgat caggttgcgg |
| 17701 | tagcgcatga ctaggatcgg gccagcctgc cccgcctcct ccttcaaatc gtactccggc |
| 17761 | aggtcatttg acccgatcag cttgcgcacg gtgaaacaga acttcttgaa ctctccggcg |
| 17821 | ctgccactgc gttcgtagat cgtcttgaac aaccatctgg cttctgcctt gcctgcggcg |
| 17881 | cggcgtgcca ggcggtagag aaaacggccg atgccgggat cgatcaaaaa gtaatcgggg |
| 17941 | tgaaccgtca gcacgtccgg gttcttgcct tctgtgatct cgcggtacat ccaatcagct |
| 18001 | agctcgatct cgatgtactc cggccgcccg gtttcgctct ttacgatctt gtagcggcta |
| 18061 | atcaaggctt caccctcgga taccgtcacc aggcggccgt tcttggcctt cttcgtacgc |
| 18121 | tgcatggcaa cgtgcgtggt gtttaaccga atgcaggttt ctaccaggtc gtctttctgc |
| 18181 | tttccgccat cggctcgccg gcagaacttg agtacgtccg caacgtgtgg acggaacacg |
| 18241 | cggccgggct tgtctccctt cccttcccgg tatcggttca tggattcggt tagatgggaa |
| 18301 | accgccatca gtaccaggtc gtaatcccac acactggcca tgccggccgg ccctgcggaa |
| 18361 | acctctacgt gcccgtctgg aagctcgtag cggatcacct cgccagctcg tcggtcacgc |
| 18421 | ttcgacagac ggaaaacggc cacgtccatg atgctgcgac tatcgcgggt gcccacgtca |
| 18481 | tagagcatcg gaacgaaaaa atctggttgc tcgtcgccct tgggcggctt cctaatcgac |
| 18541 | ggcgcaccgg ctgccggcgg ttgccgggat tctttgcgga ttcgatcagc ggccgcttgc |
| 18601 | cacgattcac cggggcgtgc ttctgcctcg atgcgttgcc gctgggcggc ctgcgcggcc |
| 18661 | ttcaacttct ccaccaggtc atcacccagc gccgcgccga tttgtaccgg gccggatggt |
| 18721 | ttgcgaccgc tcacgccgat tcctcgggct tgggggttcc agtgccattg cagggccggc |
| 18781 | agGcaaccca gccgcttacg cctggccaac cgcccgttcc tccacacatg gggcattcca |
| 18841 | cggcgtcggt gcctggttgt tcttgatttt ccatgccgcc tcctttagcc gctaaaattc |
| 18901 | atctactcat ttattcattt gctcatttac tctggtagct gcgcgatgta ttcagatagc |
| 18961 | agctcggtaa tggtcttgcc ttggcgtacc gcgtacatct tcagcttggt gtgatcctcc |
| 19021 | gccggcaact gaaagttgac ccgcttcatg gctggcgtgt ctgccaggct ggccaacgtt |
| 19081 | gcagccttgc tgctgcgtgc gctcggacgg ccggcactta gcgtgtttgt gcttttgctc |
| 19141 | attttctctt tacctcatta actcaaatga gttttgattt aatttcagcg gccagcgcct |
| 19201 | ggacctcgcg ggcagcgtcg ccctcgggtt ctgattcaag aacggttgtg ccggcggcgg |
| 19261 | cagtgcctgg gtagctcacg cgctgcgtga tacgggactc aagaatgggc agctcgtacc |
| 19321 | cggccagcgc ctcggcaacc tcaccgccga tgcgcgtgcc tttgatcgcc cgcgacacga |
| 19381 | caaaggccgc ttgtagcctt ccatccgtga cctcaatgcg ctgcttaacc agctccacca |
| 19441 | ggtcggcggt ggcccatatg tcgtaagggc ttggctgcac cggaatcagc acgaagtcgg |
| 19501 | ctgccttgat cgcggacaca gccaagtccg ccgcctgggg cgctccgtcg atcactacga |
| 19561 | agtcgcgccg gccgatggcc ttcacgtcgc ggtcaatcgt cgggcggtcg atgccgacaa |
| 19621 | cggttagcgg ttgatcttcc cgcacggccg cccaatcgcg ggcactgccc tggggatcgg |
| 19681 | aatcgactaa cagaacatcg gccccggcga gttgcagggc gcgggctaga tgggttgcga |
| 19741 | tggtcgtctt gcctgacccg cctttctggt taagtacagc gataaccttc atgcgttccc |
| 19801 | cttgcgtatt tgtttattta ctcatcgcat catatacgca gcgaccgcat gacgcaagct |
| 19861 | gttttactca aatacacatc acctttttag acggcggcgc tcggtttctt cagcggccaa |
| 19921 | gctggccggc caggccgcca gcttggcatc agacaaaccg gccaggattt catgcagccg |
| 19981 | cacggttgag acgtgcgcgg gcggctcgaa cacgtacccg gccgcgatca tctccgcctc |
| 20041 | gatctcttcg gtaatgaaaa acggttcgtc ctggccgtcc tggtgcggtt tcatgcttgt |
| 20101 | tcctcttggc gttcattctc ggcggccgcc agggcgtcgg cctcggtcaa tgcgtcctca |
| 20161 | cggaaggcac cgcgccgcct ggcctcggtg ggcgtcactt cctcgctgcg ctcaagtgcg |
| 20221 | cggtacaggg tcgagcgatg cacgccaagc agtgcagccg cctctttcac ggtgcggcct |
| 20281 | tcctggtcga tcagctcgcg ggcgtgcgcg atctgtgccg gggtgagggt agggcggggg |
| 20341 | ccaaacttca cgcctcgggc cttggcggcc tcgcgcccgc tccgggtgcg gtcgatgatt |
| 20401 | agggaacgct cgaactcggc aatgccggcg aacacggtca acaccatgcg gccggccggc |
| 20461 | gtggtggtgt cggcccacgg ctctgccagg ctacgcaggc ccgcgccggc ctcctggatg |
| 20521 | cgctcggcaa tgtccagtag gtcgcgggtg ctgcgggcca ggcggtctag cctggtcact |
| 20581 | gtcacaacgt cgccagggcg taggtggtca agcatcctgg ccagctccgg gcggtcgcgc |
| 20641 | ctggtgccgg tgatcttctc ggaaaacagc ttggtgcagc cggccgcgtg cagttcggcc |
| 20701 | cgttggttgg tcaagtcctg gtcgtcggtg ctgacgcggg catagcccag caggccagcg |
| 20761 | gcggcgctct tgttcatggc gtaatgtctc cggttctagt cgcaagtatt ctactttatg |
| 20821 | cgactaaaac acgcgacaag aaaacgccag gaaaagggca gggcggcagc ctgtcgcgta |
| 20881 | acttaggact tgtgcgacat gtcgttttca gaagacggct gcactgaacg tcagaagccg |
| 20941 | actgcactat agcagcggag gggttggatc aaagtacttt gatcccgagg ggaaccctgt |
| 21001 | ggttggcatg cacatacaaa tggacgaacg gataaacctt ttcacgccct tttaaatatc |
| 21061 | cgAttattct aataaacgct cttttctctt ag |

//
SEQ ID NO: 89. Unfused nickase, Pong ORF1and ORF2, gRNA

| | |
|---|---|
| LOCUS | Vector_comprising_unfu 22510 bp ds-DNA circular 09-MAR.-2022 |
| DEFINITION | . |
| ACCESSION | pVec1 |
| VERSION | pVec1 .1 |
| FEATURES | Location/Qualifiers |
| Agro | tDNA cut site 1 . . . 25 |
| | /label = “RB″ |
| misc | feature 254 . . . 677 |
| | /label = “U6-26promoter″ |
| misc | feature 678 . . . 697 |
| | /label = “gRNA to ADH1″ |
| misc | feature 698 . . . 773 |
| | /label = “gRNA scaffold″ |
| misc | feature 774 . . . 965 |
| | /label = “U6-26 terminator″ |
| promoter | 981 . . . 2667 |
| | /label = “Rps5a″ |
| gene | 2683 . . . 4121 |
| | /label = “ORF1SC1″ |
| terminator | 4165 . . . 4890 |
| | /label = “OCS terminator″ |
| promoter | 5073 . . . 5992 |
| | /label = “GmUbi3 Promoter″ |
| gene | 6014 . . . 7462 |
| | /label = “Pong TPase LA″ |
| terminator | 7488 . . . 8215 |
| | /label = “OCS Terminator″ |
| promoter | 8218 . . . 8942 |
| | /label = “AtUBQ10 promoter″ |
| CDS | 8955 . . . 13226 |
| | /label = “Translation 8955-13226″ |
| feature | 8958 . . . 8978 |
| | /label = “FLAG″ |
| feature | 8979 . . . 8999 |
| | /label = “FLAG″ |
| feature | 9000 . . . 9023 |
| | /label = “FLAG″ |
| feature | 9030 . . . 9050 |
| | /label = “SV40 NLS″ |
| misc_feature | 9075 . . . 13226 |
| | /label = “Cas9 Nickase (D10A)″ |
| misc_feature | 9099 . . . 9101 |
| | /label = “D10A″ |
| misc_feature | 13176 . . . 13223 |
| | /label = “NLS″ |
| misc_feature | 13232 . . . 13856 |
| | /label = “Rbs Term″ |
| promoter | 14105 . . . 14846 |
| | /label = “CaMVd35S_promoter″ |
| gene | 14937 . . . 15932 |
| | /label = “hygroB (variant) ″ |
| misc_feature | complement (16550 . . . 16572) |
| | /label = “LB R″ |
| gene | 16688 . . . 17482 |
| | /label = “KanR1″ |
| origin | 17553 . . . 18165 |
| | /label = “pBR322_origin″ |
| ORIGIN | |
| 1 | gtttacccgc caatatatcc tgtcaaacac tgatagttta aactgaaggc gggaaacgac |
| 61 | aatctgatcc aagctcaagc tgctctagca ttcgccattc aggctgcgca actgttggga |
| 121 | agggcgatcg gtgcgggcct cttcgctatt acgccagctg gcgaaagggg gatgtgctgc |
| 181 | aaggcgatta agttgggtaa cgccagggtt ttcccagtca cgacgttgta aaacgacggc |
| 241 | cagtgccaag cttcgacttg ccttccgcac aatacatcat ttcttcttag ctttttttct |
| 301 | tcttcttcgt tcatacagtt tttttttgtt tatcagctta cattttcttg aaccgtagct |
| 361 | ttcgttttct tctttttaac tttccattcg gagtttttgt atcttgtttc atagtttgtc |
| 421 | ccaggattag aatgattagg catcgaacct tcaagaattt gattgaataa aacatcttca |
| 481 | ttcttaagat atgaagataa tcttcaaaag gcccctggga atctgaaaga agagaagcag |
| 541 | gcccatttat atgggaaaga acaatagtat ttcttatata ggcccattta agttgaaaac |
| 601 | aatcttcaaa agtcccacat cgcttagata agaaaacgaa gctgagttta tatacagcta |
| 661 | gagtcgaagt agtgattGCT TCATGGCCGA AGATACGgtt ttagagctag aaatagcaag |
| 721 | ttaaaataag gctagtccgt tatcaacttg aaaaagtggc accgagtcgg tgcttttttt |
| 781 | tgcaaaattt tccagatcga tttcttcttc ctctgttctt cggcgttcaa tttctggggt |
| 841 | tttctcttcg ttttctgtaa ctgaaaccta aaatttgacc taaaaaaaat ctcaaataat |
| 901 | atgattcagt ggttttgtac ttttcagtta gttgagtttt gcagttccga tgagataaac |
| 961 | caataccatg ttagagagcg ctagttcgtg agtagatata ttactcaact tttgattcgc |
| 1021 | tatttgcagt gcacctgtgg cgttcatcac atcttttgtg acactgtttg cactggtcat |
| 1081 | tgctattaca aaggaccttc ctgatgttga aggagatcga aagtaagtaa ctgcacgcat |
| 1141 | aaccattttc tttccgctct ttggctcaat ccatttgaca gtcaaagaca atgtttaacc |
| 1201 | agctccgttt gatatattgt ctttatgtgt ttgttcaagc atgtttagtt aatcatgcct |
| 1261 | ttgattgatc ttgaataggt tccaaatatc aaccctggca acaaaacttg gagtgagaaa |
| 1321 | cattgcattc ctcggttctg gacttctgct agtaaattat gtttcagcca tatcactagc |
| 1381 | tttctacatg cctcaggtga attcatctat ttccgtctta actatttcgg ttaatcaaag |
| 1441 | cacgaacacc attactgcat gtagaagctt gataaactat cgccaccaat ttatttttgt |
| 1501 | tgcgatattg ttactttcct cagtatgcag ctttgaaaag accaaccctc ttatccttta |
| 1561 | acaatgaaca ggtttttaga ggtagcttga tgattcctgc acatgtgatc ttggcttcag |
| 1621 | gcttaatttt ccaggtaaag cattatgaga tactcttata tctcttacat acttttgaga |
| 1681 | taatgcacaa gaacttcata actatatgct ttagtttctg catttgacac tgccaaattc |
| 1741 | attaatctct aatatctttg ttgttgatct ttggtagaca tgggtactag aaaaagcaaa |
| 1801 | ctacaccaag gtaaaatact tttgtacaaa cataaactcg ttatcacgga acatcaatgg |
| 1861 | agtgtatatc taacggagtg tagaaacatt tgattattgc aggaagctat ctcaggatat |
| 1921 | tatcggttta tatggaatct cttctacgca gagtatctgt tattcccctt cctctagctt |
| 1981 | tcaatttcat ggtgaggata tgcagttttc tttgtatatc attcttcttc ttctttgtag |
| 2041 | cttggagtca aaatcggttc cttcatgtac atacatcaag gatatgtcct tctgaatttt |
| 2101 | tatatcttgc aataaaaatg cttgtaccaa ttgaaacacc agctttttga gttctatgat |
| 2161 | cactgacttg gttctaacca aaaaaaaaaa aatgtttaat ttacatatct aaaagtaggt |
| 2221 | ttagggaaac ctaaacagta aaatatttgt atattattcg aatttcactc atcataaaaa |
| 2281 | cttaaattgc accataaaat tttgttttac tattaatgat gtaatttgtg taacttaaga |
| 2341 | taaaaataat attccgtaag ttaaccggct aaaaccacgt ataaaccagg gaacctgtta |
| 2401 | aaccggttct ttactggata aagaaatgaa agcccatgta gacagctcca ttagagccca |
| 2461 | aaccctaaat ttctcatcta tataaaagga gtgacattag ggtttttgtt cgtcctctta |
| 2521 | aagcttctcg ttttctctgc cgtctctctc attcgcgcga cgcaaacgat cttcaggtga |
| 2581 | tcttctttct ccaaatcctc tctcataact ctgatttcgt acttgtgtat ttgagctcac |
| 2641 | gctctgtttc tctcaccaca gccggattcg agatcacaag tttgtacaaa aaagcaggct |
| 2701 | tccatggatc cgtcgccggc cgtggatccg tcgccggccg tggatccgtc gccggctgct |
| 2761 | gaaacccggc ggcgtgcaac cgggaaagga ggcaaacagc gcgggggcaa gcaactagga |
| 2821 | ttgaagaggc cgccgccgat ttctgtcccg gccaccccgc ctcctgctgc gacgtcttca |
| 2881 | tcccctgctg cgccgacggc catcccacca cgaccaccgc aatcttcgcc gattttcgtc |
| 2941 | cccgattcgc cgaatccgtc accggctgcg ccgacctcct ctcttgcttc ggggacatcg |
| 3001 | acggcaaggc caccgcaacc acaaggagga ggatggggac caacatcgac catttcccca |
| 3061 | aactttgcat ctttctttgg aaaccaacaa gacccaaatt catgtttggt caggggttat |
| 3121 | cctccaggag ggtttgtcaa ttttattcaa caaaattgtc cgccgcagcc acaacagcaa |
| 3181 | ggtgaaaatt ttcatttcgt tggtcacaat atggggttca acccaatatc tccacagcca |
| 3241 | ccaagtgcct acggaacacc aacaccccaa gctacgaacc aaggcacttc aacaaacatt |
| 3301 | atgattgatg aagaggacaa caatgatgac agtagggcag caaagaaaag atggactcat |
| 3361 | gaagaggaag agagactggc cagtgcttgg ttgaatgctt ctaaagactc aattcatggg |
| 3421 | aatgataaga aaggtgatac attttggaag gaagtcactg atgaatttaa caagaaaggg |
| 3481 | aatggaaaac gtaggaggga aattaaccaa ctgaaggttc actggtcaag gttgaagtca |
| 3541 | gcgatctctg agttcaatga ctattggagt acggttactc aaatgcatac aagcggatac |
| 3601 | tcagacgaca tgcttgagaa agaggcacag aggctgtatg caaacaggtt tggaaaacct |
| 3661 | tttgcgttgg tccattggtg gaagatactc aaaagagagc ccaaatggtg tgctcagttt |
| 3721 | gaaaagagga aaaggaagag cgaaatggat gctgttccag aacagcagaa acgtcctatt |
| 3781 | ggtagagaag cagcaaagtc tgagcgcaaa agaaagcgca agaaagaaaa tgttatggaa |
| 3841 | ggcattgtcc tcctagggga caatgtccag aaaattatca aagtgacgca agatcggaag |
| 3901 | ctggagcgtg agaaggtcac tgaagcacag attcacattt caaacgtaaa tttgaaggca |
| 3961 | gcagaacagc aaaaagaagc aaagatgttt gaggtataca attccctgct cactcaagat |
| 4021 | acaagtaaca tgtctgaaga acagaaggct cgccgagaca aggcattaca aaagctggag |
| 4081 | gaaaagttat ttgctgacta gtgacccagc tttcttgtac aaagtggtgc ctaggtgagt |
| 4141 | ctagagagtt gattaagacc cgggactggt ccctagagtc ctgctttaat gagatatgcg |
| 4201 | agacgcctat gatcgcatga tatttgcttt caattctgtt gtgcacgttg taaaaaacct |
| 4261 | gagcatgtgt agctcagatc cttaccgccg gtttcggttc attctaatga atatatcacc |
| 4321 | cgttactatc gtatttttat gaataatatt ctccgttcaa tttactgatt gtaccctact |
| 4381 | acttatatgt acaatattaa aatgaaaaca atatattgtg ctgaataggt ttatagcgac |
| 4441 | atctatgata gagcgccaca ataacaaaca attgcgtttt attattacaa atccaatttt |
| 4501 | aaaaaaagcg gcagaaccgg tcaaacctaa aagactgatt acataaatct tattcaaatt |
| 4561 | tcaaaagtgc cccaggggct agtatctacg acacaccgag cggcgaacta ataacgctca |
| 4621 | ctgaagggaa ctccggttcc ccgccggcgc gcatgggtga gattccttga agttgagtat |
| 4681 | tggccgtccg ctctaccgaa agttacgggc accattcaac ccggtccagc acggcggccg |
| 4741 | ggtaaccgac ttgctgcccc gagaattatg cagcattttt ttggtgtatg tgggccccaa |
| 4801 | atgaagtgca ggtcaaacct tgacagtgac gacaaatcgt tgggcgggtc cagggcgaat |
| 4861 | tttgcgacaa catgtcgagg ctcagcagga cctgcaggca tgcaagcttg gcactggccg |
| 4921 | tcgttttaca acgtcgtgac tgggaaaacc ctggcgttac ccaacttaat cgccttgcag |
| 4981 | cacatccccc tttcgccagc tggcgtaata gcgaagaggc ccgcaccgat cgcccttccc |
| 5041 | aacagttgcg cagcctgaat ggcgaatgct agagcagctt gagcttggat cagattgtcg |
| 5101 | tttcccgcct tcagtttctt gaaggtgcat gtgactccgt caagattacg aaaccgccaa |
| 5161 | ctaccacgca aattgcaatt ctcaatttcc tagaaggact ctccgaaaat gcatccaata |
| 5221 | ccaaatatta cccgtgtcat aggcaccaag tgacaccata catgaacacg cgtcacaata |
| 5281 | tgactggaga agggttccac accttatgct ataaaacgcc ccacacccct cctccttcct |
| 5341 | tcgcagttca attccaatat attccattct ctctgtgtat ttccctacct ctcccttcaa |
| 5401 | ggttagtcga tttcttctgt ttttcttctt cgttctttcc atgaattgtg tatgttcttt |
| 5461 | gatcaatacg atgttgattt gattgtgttt tgtttggttt catcgatctt caattttcat |
| 5521 | aatcagattc agcttttatt atctttacaa caacgtcctt aatttgatga ttctttaatc |
| 5581 | gtagatttgc tctaattaga gctttttcat gtcagatccc tttacaacaa gccttaattg |
| 5641 | ttgattcatt aatcgtagat tagggctttt ttcattgatt acttcagatc cgttaaacgt |
| 5701 | aaccatagat cagggctttt tcatgaatta cttcagatcc gttaaacaac agccttattt |
| 5761 | tttatacttc tgtggttttt caagaaattg ttcagatccg ttgacaaaaa gccttattcg |
| 5821 | ttgattctat atcgtttttc gagagatatt gctcagatct gttagcaact gccttgtttg |
| 5881 | ttgattctat tgccgtggat tagggttttt tttcacgaga ttgcttcaga tccgtactta |
| 5941 | agattacgta atggattttg attctgattt atctgtgatt gttgactcga caggtacctt |
| 6001 | caaacggcgc gccatgcaga gtttagccat ctctctactc ctctcagaaa ctcattccct |
| 6061 | cttttctcat acgaagacct cctccctttt atctttactg tttctctctt cttcaaagat |
| 6121 | gtctgagcaa aatactgatg gaagtcaagt tccagtgaac ttgttggatg agttcctggc |
| 6181 | tgaggatgag atcatagatg atcttctcac tgaagccacg gtggtagtac agtccactat |
| 6241 | agaaggtctt caaaacgagg cttctgacca tcgacatcat ccgaggaagc acatcaagag |
| 6301 | gccacgagag gaagcacatc agcaactggt gaatgattac ttttcagaaa atcctcttta |
| 6361 | cccttccaaa atttttcgtc gaagatttcg tatgtctagg ccactttttc ttcgcatcgt |
| 6421 | tgaggcatta ggccagtggt cagtgtattt cacacaaagg gtggatgctg ttaatcggaa |
| 6481 | aggactcagt ccactgcaaa agtgtactgc agctattcgc cagttggcta ctggtagtgg |
| 6541 | cgcagatgaa ctagatgaat atctgaagat aggagagact acagcaatgg aggcaatgaa |
| 6601 | gaattttgtc aaaggtcttc aagatgtgtt tggtgagagg tatcttaggc gccccactat |
| 6661 | ggaagatacc gaacggcttc tccaacttgg tgagaaacgt ggttttcctg gaatgttcgg |
| 6721 | cagcattgac tgcatgcact ggcattggga aagatgccca gtagcatgga agggtcagtt |
| 6781 | cactcgtgga gatcagaaag tgccaaccct gattcttgag gctgtggcat cgcatgatct |
| 6841 | ttggatttgg catgcatttt ttggagcagc gggttccaac aatgatatca atgtattgaa |
| 6901 | ccaatctact gtatttatca aggagctcaa aggacaagct cctagagtcc agtacatggt |
| 6961 | aaatgggaat caatacaata ctgggtattt tcttgctgat ggaatctacc ctgaatgggc |
| 7021 | agtgtttgtt aagtcaatac gactcccaaa cactgaaaag gagaaattgt atgcagatat |
| 7081 | gcaagaaggg gcaagaaaag atatcgagag agcctttggt gtattgcagc gaagattttg |
| 7141 | catcttaaaa cgaccagctc gtctatatga tcgaggtgta ctgcgagatg ttgttctagc |
| 7201 | ttgcatcata cttcacaata tgatagttga agatgagaag gaaaccagaa ttattgaaga |
| 7261 | agatgcagat gcaaatgtgc ctcctagttc atcaaccgtt caggaacctg agttctctcc |
| 7321 | tgaacagaac acaccatttg atagagtttt agaaaaagat atttctatcc gagatcgagc |
| 7381 | ggctcataac cgacttaaga aagatttggt ggaacacatt tggaataagt ttggtggtgc |
| 7441 | tgcacataga actggaaatt aattaattga cattctaatc tagagtcctg ctttaatgag |
| 7501 | atatgcgaga cgcctatgat cgcatgatat ttgctttcaa ttctgttgtg cacgttgtaa |
| 7561 | aaaacctgag catgtgtagc tcagatcctt accgccggtt tcggttcatt ctaatgaata |
| 7621 | tatcacccgt tactatcgta tttttatgaa taatattctc cgttcaattt actgattgta |
| 7681 | ccctactact tatatgtaca atattaaaat gaaaacaata tattgtgctg aataggttta |
| 7741 | tagcgacatc tatgatagag cgccacaata acaaacaatt gcgttttatt attacaaatc |
| 7801 | caattttaaa aaaagcggca gaaccggtca aacctaaaag actgattaca taaatcttat |
| 7861 | tcaaatttca aaagtgcccc aggggctagt atctacgaca caccgagcgg cgaactaata |
| 7921 | acgttcactg aagggaactc cggttccccg ccggcgcgca tgggtgagat tccttgaagt |
| 7981 | tgagtattgg ccgtccgctc taccgaaagt tacgggcacc attcaacccg gtccagcacg |
| 8041 | gcggccgggt aaccgacttg ctgccccgag aattatgcag catttttttg gtgtatgtgg |
| 8101 | gccccaaatg aagtgcaggt caaaccttga cagtgacgac aaatcgttgg gcgggtccag |
| 8161 | ggcgaatttt gcgacaacat gtcgaggctc agcaggacct gcaggcatgc aagatcggat |
| 8221 | caggatattc ttgtttaaga tgttgaactc tatggaggtt tgtatgaact gatgatctag |
| 8281 | gaccggataa gttcccttct tcatagcgaa cttattcaaa gaatgttttg tgtatcattc |
| 8341 | ttgttacatt gttattaatg aaaaaatatt attggtcatt ggactgaaca cgagtgttaa |
| 8401 | atatggacca ggccccaaat aagatccatt gatatatgaa ttaaataaca agaataaatc |
| 8461 | gagtcaccaa accacttgcc ttttttaacg agacttgttc accaacttga tacaaaagtc |
| 8521 | attatcctat gcaaatcaat aatcatacaa aaatatccaa taacactaaa aaattaaaag |
| 8581 | aaatggataa tttcacaata tgttatacga taaagaagtt acttttccaa gaaattcact |
| 8641 | gattttataa gcccacttgc attagataaa tggcaaaaaa aaacaaaaag gaaaagaaat |
| 8701 | aaagcacgaa gaattctaga aaatacgaaa tacgcttcaa tgcagtggga cccacggttc |
| 8761 | aattattgcc aattttcagc tccaccgtat atttaaaaaa taaaacgata atgctaaaaa |
| 8821 | aatataaatc gtaacgatcg ttaaatctca acggctggat cttatgacga ccgttagaaa |
| 8881 | ttgtggttgt cgacgagtca gtaataaacg gcgtcaaagt ggttgcagcc ggcacacacg |
| 8941 | aggcgcgcct ctagatggat tacaaggacc acgacgggga ttacaaggac cacgacattg |
| 9001 | attacaagga tgatgatgac aagatggctc cgaagaagaa gaggaaggtt ggcatccacg |
| 9061 | gggtgccagc tgctgacaag aagtactcga tcggcctcgc tattgggact aactctgttg |
| 9121 | gctgggccgt gatcaccgac gagtacaagg tgccctcaaa gaagttcaag gtcctgggca |
| 9181 | acaccgatcg gcattccatc aagaagaatc tcattggcgc tctcctgttc gacagcggcg |
| 9241 | agacggctga ggctacgcgg ctcaagcgca ccgcccgcag gcggtacacg cgcaggaaga |
| 9301 | atcgcatctg ctacctgcag gagattttct ccaacgagat ggcgaaggtt gacgattctt |
| 9361 | tcttccacag gctggaggag tcattcctcg tggaggagga taagaagcac gagcggcatc |
| 9421 | caatcttcgg caacattgtc gacgaggttg cctaccacga gaagtaccct acgatctacc |
| 9481 | atctgcggaa gaagctcgtg gactccacag ataaggcgga cctccgcctg atctacctcg |
| 9541 | ctctggccca catgattaag ttcaggggcc atttcctgat cgagggggat ctcaacccgg |
| 9601 | acaatagcga tgttgacaag ctgttcatcc agctcgtgca gacgtacaac cagctcttcg |
| 9661 | aggagaaccc cattaatgcg tcaggcgtcg acgcgaaggc tatcctgtcc gctaggctct |
| 9721 | cgaagtctcg gcgcctcgag aacctgatcg cccagctgcc gggcgagaag aagaacggcc |
| 9781 | tgttcgggaa tctcattgcg ctcagcctgg ggctcacgcc caacttcaag tcgaatttcg |
| 9841 | atctcgctga ggacgccaag ctgcagctct ccaaggacac atacgacgat gacctggata |
| 9901 | acctcctggc ccagatcggc gatcagtacg cggacctgtt cctcgctgcc aagaatctgt |
| 9961 | cggacgccat cctcctgtct gatattctca gggtgaacac cgagattacg aaggctccgc |
| 10021 | tctcagcctc catgatcaag cgctacgacg agcaccatca ggatctgacc ctcctgaagg |
| 10081 | cgctggtcag gcagcagctc cccgagaagt acaaggagat cttcttcgat cagtcgaaga |
| 10141 | acggctacgc tgggtacatt gacggcgggg cctctcagga ggagttctac aagttcatca |
| 10201 | agccgattct ggagaagatg gacggcacgg aggagctgct ggtgaagctc aatcgcgagg |
| 10261 | acctcctgag gaagcagcgg acattcgata acggcagcat cccacaccag attcatctcg |
| 10321 | gggagctgca cgctatcctg aggaggcagg aggacttcta ccctttcctc aaggataacc |
| 10381 | gcgagaagat cgagaagatt ctgactttca ggatcccgta ctacgtcggc ccactcgcta |
| 10441 | ggggcaactc ccgcttcgct tcgatjaccc gcaagtcaga ggagacgatc acgccgtgga |
| 10501 | acttcgagga ggtggtcgac aagggcgcta gcgctcagtc gttcatcgag aggatgacga |
| 10561 | atttcgacaa gaacctgcca aatgagaagg tgctccctaa gcactcgctc ctgtacgagt |
| 10621 | acttcacagt ctacaacgag ctgactaagg tgaagtatgt gaccgagggc atgaggaagc |
| 10681 | cggctttcct gtctggggag cagaagaagg ccatcgtgga cctcctgttc aagaccaacc |
| 10741 | ggaaggtcac ggttaagcag ctcaaggagg actacttcaa gaagattgag tgcttcgatt |
| 10801 | cggtcgagat ctctggcgtt gaggaccgct tcaacgcctc cctggggacc taccacgatc |
| 10861 | tcctgaagat cattaaggat aaggacttcc tggacaacga ggagaatgag gatatcctcg |
| 10921 | aggacattgt gctgacactc actctgttcg aggaccggga gatgatcgag gagcgcctga |
| 10981 | agacttacgc ccatctcttc gatgacaagg tcatgaagca gctcaagagg aggaggtaca |
| 11041 | ccggctgggg gaggctgagc aggaagctca tcaacggcat tcgggacaag cagtccggga |
| 11101 | agacgatcct cgacttcctg aagagcgatg gcttcgcgaa ccgcaatttc atgcagctga |
| 11161 | ttcacgatga cagcctcaca ttcaaggagg atatccagaa ggctcaggtg agcggccagg |
| 11221 | gggactcgct gcacgagcat atcgcgaacc tcgctggctc gccagctatc aagaagggga |
| 11281 | ttctgcagac cgtgaaggtt gtggacgagc tggtgaaggt catgggcagg cacaagcctg |
| 11341 | agaacatcgt cattgagatg gcccgggaga atcagaccac gcagaagggc cagaagaact |
| 11401 | cacgcgagag gatgaagagg atcgaggagg gcattaagga gctggggtcc cagatcctca |
| 11461 | aggagcaccc ggtggagaac acgcagctgc agaatgagaa gctctacctg tactacctcc |
| 11521 | agaatggccg cgatatgtat gtggaccagg agctggatat taacaggctc agcgattacg |
| 11581 | acgtcgatca tatcgttcca cagtcattcc tgaaggatga ctccattgac aacaaggtcc |
| 11641 | tcaccaggtc ggacaagaac cggggcaagt ctgataatgt tccttcagag gaggtcgtta |
| 11701 | agaagatgaa gaactactgg cgccagctcc tgaatgccaa gctgatcacg cagcggaagt |
| 11761 | tcgataacct cacaaaggct gagaggggcg ggctctctga gctggacaag gcgggcttca |
| 11821 | tcaagaggca gctggtcgag acacggcaga tcactaagca cgttgcgcag attctcgact |
| 11881 | cacggatgaa cactaagtac gatgagaatg acaagctgat ccgcgaggtg aaggtcatca |
| 11941 | ccctgaagtc aaagctcgtc tccgacttca ggaaggattt ccagttctac aaggttcggg |
| 12001 | agatcaacaa ttaccaccat gcccatgacg cgtacctgaa cgcggtggtc ggcacagctc |
| 12061 | tgatcaagaa gtacccaaag ctcgagagcg agttcgtgta cggggactac aaggtttacg |
| 12121 | atgtgaggaa gatgatcgcc aagtcggagc aggagattgg caaggctacc gccaagtact |
| 12181 | tcttctactc taacattatg aatttcttca agacagagat cactctggcc aatggcgaga |
| 12241 | tccggaagcg ccccctcatc gagacgaacg gcgagacggg ggagatcgtg tgggacaagg |
| 12301 | gcagggattt cgcgaccgtc aggaaggttc tctccatgcc acaagtgaat atcgtcaaga |
| 12361 | agacagaggt ccagactggc gggttctcta aggagtcaat tctgcctaag cggaacagcg |
| 12421 | acaagctcat cgcccgcaag aaggactggg atccgaagaa gtacggcggg ttcgacagcc |
| 12481 | ccactgtggc ctactcggtc ctggttgtgg cgaaggttga gaagggcaag tccaagaagc |
| 12541 | tcaagagcgt gaaggagctg ctggggatca cgattatgga gcgctccagc ttcgagaaga |
| 12601 | acccgatcga tttcctggag gcgaagggct acaaggaggt gaagaaggac ctgatcatta |
| 12661 | agctccccaa gtactcactc ttcgagctgg agaacggcag gaagcggatg ctggcttccg |
| 12721 | ctggcgagct gcagaagggg aacgagctgg ctctgccgtc caagtatgtg aacttcctct |
| 12781 | acctggcctc ccactacgag aagctcaagg gcagccccga ggacaacgag cagaagcagc |
| 12841 | tgttcgtcga gcagcacaag cattacctcg acgagatcat tgagcagatt tccgagttct |
| 12901 | ccaagcgcgt gatcctggcc gacgcgaatc tggataaggt cctctccgcg tacaacaagc |
| 12961 | accgcgacaa gccaatcagg gagcaggctg agaatatcat tcatctcttc accctgacga |
| 13021 | acctcggcgc ccctgctgct ttcaagtact tcgacacaac tatcgatcgc aagaggtaca |
| 13081 | caagcactaa ggaggtcctg gacgcgaccc tcatccacca gtcgattacc ggcctctacg |
| 13141 | agacgcgcat cgacctgtct cagctcgggg gcgacaagcg gccagcggcg acgaagaagg |
| 13201 | cggggcaggc gaagaagaag aagtgagctc agagctttcg ttcgtatcat cggtttcgac |
| 13261 | aacgttcgtc aagttcaatg catcagtttc attgcgcaca caccagaatc ctactgagtt |
| 13321 | tgagtattat ggcattggga aaactgtttt tcttgtacca tttgttgtgc ttgtaattta |
| 13381 | ctgtgttttt tattcggttt tcgctatcga actgtgaaat ggaaatggat ggagaagagt |
| 13441 | taatgaatga tatggtcctt ttgttcattc tcaaattaat attatttgtt ttttctctta |
| 13501 | tttgttgtgt gttgaatttg aaattataag agatatgcaa acattttgtt ttgagtaaaa |
| 13561 | atgtgtcaaa tcgtggcctc taatgaccga agttaatatg aggagtaaaa cacttgtagt |
| 13621 | tgtaccatta tgcttattca ctaggcaaca aatatatttt cagacctaga aaagctgcaa |
| 13681 | atgttactga atacaagtat gtcctcttgt gttttagaca tttatgaact ttcctttatg |
| 13741 | taattttcca gaatccttgt cagattctaa tcattgcttt ataattatag ttatactcat |
| 13801 | ggatttgtag ttgagtatga aaatattttt taatgcattt tatgacttgc caattgcgaa |
| 13861 | ttcgtaatca tgtcatagct gtttcctgtg tgaaattgtt atccgctcac aattccacac |
| 13921 | aacatacgag ccggaagcat aaagtgtaaa gcctggggtg cctaatgagt gagctaactc |
| 13981 | acattaattg cgttgcgctc actgcccgct ttccagtcgg gaaacctgtc gtgccagctg |
| 14041 | cattaatgaa tcggccaacg cgcggggaga ggcggtttgc gtattggcta gagcagcttg |
| 14101 | ccaacatggt ggagcacgac actctcgtct actccaagaa tatcaaagat acagtctcag |
| 14161 | aagaccaaag ggctattgag acttttcaac aaagggtaat atcgggaaac ctcctcggat |
| 14221 | tccattgccc agctatctgt cacttcatca aaaggacagt agaaaaggaa ggtggcacct |
| 14281 | acaaatgcca tcattgcgat aaaggaaagg ctatcgttca agatgcctct gccgacagtg |
| 14341 | gtcccaaaga tggaccccca cccacgagga gcatcgtgga aaaagaagac gttccaacca |
| 14401 | cgtcttcaaa gcaagtggat tgatgtgata acatggtgga gcacgacact ctcgtctact |
| 14461 | ccaagaatat caaagataca gtctcagaag accaaagggc tattgagact tttcaacaaa |
| 14521 | gggtaatatc gggaaacctc ctcggattcc attgcccagc tatctgtcac ttcatcaaaa |
| 14581 | ggacagtaga aaaggaaggt ggcacctaca aatgccatca ttgcgataaa ggaaaggcta |
| 14641 | tcgttcaaga tgcctctgcc gacagtggtc ccaaagatgg acccccaccc acgaggagca |
| 14701 | tcgtggaaaa agaagacgtt ccaaccacgt cttcaaagca agtggattga tgtgatatct |
| 14761 | ccactgacgt aagggatgac gcacaatccc actatccttc gcaagacctt cctctatata |
| 14821 | aggaagttca tttcatttgg agaggacacg ctgaaatcac cagtctctct ctacaaatct |
| 14881 | atctctctcg agctttcgca gatcccgggg ggcaatgaga tatgaaaaag cctgaactca |
| 14941 | ccgcgacgtc tgtcgagaag tttctgatcg aaaagttcga cagcgtctcc gacctgatgc |
| 15001 | agctctcgga gggcgaagaa tctcgtgctt tcagcttcga tgtaggaggg cgtggatatg |
| 15061 | tcctgcgggt aaatagctgc gccgatggtt tctacaaaga tcgttatgtt tatcggcact |
| 15121 | ttgcatcggc cgcgctcccg attccggaag tgcttgacat tggggagttt agcgagagcc |
| 15181 | tgacctattg catctcccgc cgtgcacagg gtgtcacgtt gcaagacctg cctgaaaccg |
| 15241 | aactgcccgc tgttctacaa ccggtcgcgg aggctatgga tgcgatcgct gcggccgatc |
| 15301 | ttagccagac gagcgggttc ggcccattcg gaccgcaagg aatcggtcaa tacactacat |
| 15361 | ggcgtgattt catatgcgcg attgctgatc cccatgtgta tcactggcaa actgtgatgg |
| 15421 | acgacaccgt cagtgcgtcc gtcgcgcagg ctctcgatga gctgatgctt tgggccgagg |
| 15481 | actgccccga agtccggcac ctcgtgcacg cggatttcgg ctccaacaat gtcctgacgg |
| 15541 | acaatggccg cataacagcg gtcattgact ggagcgaggc gatgttcggg gattcccaat |
| 15601 | acgaggtcgc caacatcttc ttctggaggc cgtggttggc ttgtatggag cagcagacgc |
| 15661 | gctacttcga gcggaggcat ccggagcttg caggatcgcc acgactccgg gcgtatatgc |
| 15721 | tccgcattgg tcttgaccaa ctctatcaga gcttggttga cggcaatttc gatgatgcag |
| 15781 | cttgggcgca gggtcgatgc gacgcaatcg tccgatccgg agccgggact gtcgggcgta |
| 15841 | cacaaatcgc ccgcagaagc gcggccgtct ggaccgatgg ctgtgtagaa gtactcgccg |
| 15901 | atagtggaaa ccgacgcccc agcactcgtc cgagggcaaa gaaatagagt agatgccgac |
| 15961 | cggatctgtc gatcgacaag ctcgagtttc tccataataa tgtgtgagta gttcccagat |
| 16021 | aagggaatta gggttcctat agggtttcgc tcatgtgttg agcatataag aaacccttag |
| 16081 | tatgtatttg tatttgtaaa atacttctat caataaaatt tctaattcct aaaaccaaaa |
| 16141 | tccagtacta aaatccagat cccccgaatt aattcggcgt taattcagta cattaaaaac |
| 16201 | gtccgcaatg tgttattaag ttgtctaagc gtcaatttgt ttacaccaca atatatcctg |
| 16261 | ccaccagcca gccaacagct ccccgaccgg cagctcggca caaaatcacc actcgataca |
| 16321 | ggcagcccat cagtccggga cggcgtcagc gggagagccg ttgtaaggcg gcagactttg |
| 16381 | ctcatgttac cgatgctatt cggaagaacg gcaactaagc tgccgggttt gaaacacgga |
| 16441 | tgatctcgcg gagggtagca tgttgattgt aacgatgaca gagcgttgct gcctgtgatc |
| 16501 | accgcggttt caaaatcggc tccgtcgata ctatgttata cgccaacttt gaaaacaact |
| 16561 | ttgaaaaagc tgttttctgg tatttaaggt tttagaatgc aaggaacagt gaattggagt |
| 16621 | tcgtcttgtt ataattagct tcttggggta tctttaaata ctgtagaaaa gaggaaggaa |
| 16681 | ataataaatg gctaaaatga gaatatcacc ggaattgaaa aaactgatcg aaaaataccg |
| 16741 | ctgcgtaaaa gatacggaag gaatgtctcc tgctaaggta tataagctgg tgggagaaaa |
| 16801 | tgaaaaccta tatttaaaaa tgacggacag ccggtataaa gggaccacct atgatgtgga |
| 16861 | acgggaaaag gacatgatgc tatggctgga aggaaagctg cctgttccaa aggtcctgca |
| 16921 | ctttgaacgg catgatggct ggagcaatct gctcatgagt gaggccgatg gcgtcctttg |
| 16981 | ctcggaagag tatgaagatg aacaaagccc tgaaaagatt atcgagctgt atgcggagtg |
| 17041 | catcaggctc tttcactcca tcgacatatc ggattgtccc tatacgaata gcttagacag |
| 17101 | ccgcttagcc gaattggatt acttactgaa taacgatctg gccgatgtgg attgcgaaaa |
| 17161 | ctgggaagaa gacactccat ttaaagatcc gcgcgagctg tatgattttt taaagacgga |
| 17221 | aaagcccgaa gaggaacttg tcttttccca cggcgacctg ggagacagca acatctttgt |
| 17281 | gaaagatggc aaagtaagtg gctttattga tcttgggaga agcggcaggg cggacaagtg |
| 17341 | gtatgacatt gccttctgcg tccggtcgat cagggaggat atcggggaag aacagtatgt |
| 17401 | cgagctattt tttgacttac tggggatcaa gcctgattgg gagaaaataa aatattatat |
| 17461 | tttactggat gaattgtttt agtacctaga atgcatgacc aaaatccctt aacgtgagtt |
| 17521 | ttcgttccac tgagcgtcag accccgtaga aaagatcaaa ggatcttctt gagatccttt |
| 17581 | ttttctgcgc gtaatctgct gcttgcaaac aaaaaaacca ccgctaccag cggtggtttg |
| 17641 | tttgccggat caagagctac caactctttt tccgaaggta actggcttca gcagagcgca |
| 17701 | gataccaaat actgtccttc tagtgtagcc gtagttaggc caccacttca agaactctgt |
| 17761 | agcaccgcct acatacctcg ctctgctaat cctgttacca gtggctgctg ccagtggcgg |
| 17821 | tgtcttaccg ggttggactc aagacgatag ttaccggata aggcgcagcg gtcgggctga |
| 17881 | acggggggtt cgtgcacaca gcccagcttg gagcgaacga cctacaccga actgagatac |
| 17941 | ctacagcgtg agctatgaga aagcgccacg cttcccgaag ggagaaaggc ggacaggtat |
| 18001 | ccggtaagcg gcagggtcgg aacaggagag cgcacgaggg agcttccagg gggaaacgcc |
| 18061 | tggtatcttt atagtcctgt cgggtttcgc cacctctgac ttgagcgtcg atttttgtga |
| 18121 | tgctcgtcag gggggcggag cctatggaaa aacgccagca acgcggcctt tttacggttc |
| 18181 | ctggcctttt gctggccttt tgctcacatg ttctttcctg cgttatcccc tgattctgtg |
| 18241 | gataaccgta ttaccgcctt tgagtgagct gataccgctc gccgcagccg aacgaccgag |
| 18301 | cgcagcgagt cagtgagcga ggaagcggaa gagcgcctga tgcggtattt tctccttacg |
| 18361 | catctgtgcg gtatttcaca ccgcatatgg tgcactctca gtacaatctg ctctgatgcc |
| 18421 | gcatagttaa gccagtatac actccgctat cgctacgtga ctgggtcatg gctgcgcccc |
| 18481 | gacacccgcc aacacccgct gacgcgccct gacgggcttg tctgctcccg gcatccgctt |
| 18541 | acagacaagc tgtgaccgtc tccgggagct gcatgtgtca gaggttttca ccgtcatcac |
| 18601 | cgaaacgcgc gaggcagggt gccttgatgt gggcgccggc ggtcgagtgg cgacggcgcg |
| 18661 | gcttgtccgc gccctggtag attgcctggc cgtaggccag ccatttttga gcggccagcg |
| 18721 | gccgcgatag gccgacgcga agcggcgggg cgtagggagc gcagcgaccg aagggtaggc |
| 18781 | gctttttgca gctcttcggc tgtgcgctgg ccagacagtt atgcacaggc caggcgggtt |
| 18841 | ttaagagttt taataagttt taaagagttt taggcggaaa aatcgccttt tttctctttt |
| 18901 | atatcagtca cttacatgtg tgaccggttc ccaatgtacg gctttgggtt cccaatgtac |
| 18961 | gggttccggt tcccaatgta cggctttggg ttcccaatgt acgtgctatc cacaggaaac |
| 19021 | agaccttttc gacctttttc ccctgctagg gcaatttgcc ctagcatctg ctccgtacat |
| 19081 | taggaaccgg cggatgcttc gccctcgatc aggttgcggt agcgcatgac taggatcggg |
| 19141 | ccagcctgcc ccgcctcctc cttcaaatcg tactccggca ggtcatttga cccgatcagc |
| 19201 | ttgcgcacgg tgaaacagaa cttcttgaac tctccggcgc tgccactgcg ttcgtagatc |
| 19261 | gtcttgaaca accatctggc ttctgccttg cctgcggcgc ggcgtgccag gcggtagaga |
| 19321 | aaacggccga tgccgggatc gatcaaaaag taatcggggt gaaccgtcag cacgtccggg |
| 19381 | ttcttgcctt ctgtgatctc gcggtacatc caatcagcta gctcgatctc gatgtactcc |
| 19441 | ggccgcccgg tttcgctctt tacgatcttg tagcggctaa tcaaggcttc accctcggat |
| 19501 | accgtcacca ggcggccgtt cttggccttc ttcgtacgct gcatggcaac gtgcgtggtg |
| 19561 | tttaaccgaa tgcaggtttc taccaggtcg tctttctgct ttccgccatc ggctcgccgg |
| 19621 | cagaacttga gtacgtccgc aacgtgtgga cggaacacgc ggccgggctt gtctcccttc |
| 19681 | ccttcccggt atcggttcat ggattcggtt agatgggaaa ccgccatcag taccaggtcg |
| 19741 | taatcccaca cactggccat gccggccggc cctgcggaaa cctctacgtg cccgtctgga |
| 19801 | agctcgtagc ggatcacctc gccagctcgt cggtcacgct tcgacagacg gaaaacggcc |
| 19861 | acgtccatga tgctgcgact atcgcgggtg cccacgtcat agagcatcgg aacgaaaaaa |
| 19921 | tctggttgct cgtcgccctt gggcggcttc ctaatcgacg gcgcaccggc tgccggcggt |
| 19981 | tgccgggatt ctttgcggat tcgatcagcg gccgcttgcc acgattcacc ggggcgtgct |
| 20041 | tctgcctcga tgcgttgccg ctgggcggcc tgcgcggcct tcaacttctc caccaggtca |
| 20101 | tcacccagcg ccgcgccgat ttgtaccggg ccggatggtt tgcgaccgct cacgccgatt |
| 20161 | cctcgggctt gggggttcca gtgccattgc agggccggca gacaacccag ccgcttacgc |
| 20221 | ctggccaacc gcccgttcct ccacacatgg ggcattccac ggcgtcggtg cctggttgtt |
| 20281 | cttgattttc catgccgcct cctttagccg ctaaaattca tctactcatt tattcatttg |
| 20341 | ctcatttact ctggtagctg cgcgatgtat tcagatagca gctcggtaat ggtcttgcct |
| 20401 | tggcgtaccg cgtacatctt cagcttggtg tgatcctccg ccggcaactg aaagttgacc |
| 20461 | cgcttcatgg ctggcgtgtc tgccaggctg gccaacgttg cagccttgct gctgcgtgcg |
| 20521 | ctcggacggc cggcacttag cgtgtttgtg cttttgctca ttttctcttt acctcattaa |
| 20581 | ctcaaatgag ttttgattta atttcagcgg ccagcgcctg gacctcgcgg gcagcgtcgc |
| 20641 | cctcgggttc tgattcaaga acggttgtgc cggcggcggc agtgcctggg tagctcacgc |
| 20701 | gctgcgtgat acgggactca agaatgggca gctcgtaccc ggccagcgcc tcggcaacct |
| 20761 | caccgccgat gcgcgtgcct ttgatcgccc gcgacacgac aaaggccgct tgtagccttc |
| 20821 | catccgtgac ctcaatgcgc tgcttaacca gctccaccag gtcggcggtg gcccatatgt |
| 20881 | cgtaagggct tggctgcacc ggaatcagca cgaagtcggc tgccttgatc gcggacacag |
| 20941 | ccaagtccgc cgcctggggc gctccgtcga tcactacgaa gtcgcgccgg ccgatggcct |
| 21001 | tcacgtcgcg gtcaatcgtc gggcggtcga tgccgacaac ggttagcggt tgatcttccc |
| 21061 | gcacggccgc ccaatcgcgg gcactgccct ggggatcgga atcgactaac agaacatcgg |
| 21121 | ccccggcgag ttgcagggcg cgggctagat gggttgcgat ggtcgtcttg cctgacccgc |
| 21181 | ctttctggtt aagtacagcg ataaccttca tgcgttcccc ttgcgtattt gtttatttac |
| 21241 | tcatcgcatc atatacgcag cgaccgcatg acgcaagctg ttttactcaa atacacatca |
| 21301 | cctttttaga cggcggcgct cggtttcttc agcggccaag ctggccggcc aggccgccag |
| 21361 | cttggcatca gacaaaccgg ccaggatttc atgcagccgc acggttgaga cgtgcgcggg |
| 21421 | cggctcgaac acgtacccgg ccgcgatcat ctccgcctcg atctcttcgg taatgaaaaa |
| 21481 | cggttcgtcc tggccgtcct ggtgcggttt catgcttgtt cctcttggcg ttcattctcg |
| 21541 | gcggccgcca gggcgtcggc ctcggtcaat gcgtcctcac ggaaggcacc gcgccgcctg |
| 21601 | gcctcggtgg gcgtcacttc ctcgctgcgc tcaagtgcgc ggtacagggt cgagcgatgc |
| 21661 | acgccaagca gtgcagccgc ctctttcacg gtgcggcctt cctggtcgat cagctcgcgg |
| 21721 | gcgtgcgcga tctgtgccgg ggtgagggta gggcgggggc caaacttcac gcctcgggcc |
| 21781 | ttggcggcct cgcgcccgct ccgggtgcgg tcgatgatta gggaacgctc gaactcggca |
| 21841 | atgccggcga acacggtcaa caccatgcgg ccggccggcg tggtggtgtc ggcccacggc |
| 21901 | tctgccaggc tacgcaggcc cgcgccggcc tcctggatgc gctcggcaat gtccagtagg |
| 21961 | tcgcgggtgc tgcgggccag gcggtctagc ctggtcactg tcacaacgtc gccagggcgt |
| 22021 | aggtggtcaa gcatcctggc cagctccggg cggtcgcgcc tggtgccggt gatcttctcg |
| 22081 | gaaaacagct tggtgcagcc ggccgcgtgc agttcggccc gttggttggt caagtcctgg |
| 22141 | tcgtcggtgc tgacgcgggc atagcccagc aggccagcgg cggcgctctt gttcatggcg |
| 22201 | taatgtctcc ggttctagtc gcaagtattc tactttatgc gactaaaaca cgcgacaaga |
| 22261 | aaacgccagg aaaagggcag ggcggcagcc tgtcgcgtaa cttaggactt gtgcgacatg |
| 22321 | tcgttttcag aagacggctg cactgaacgt cagaagccga ctgcactata gcagcggagg |
| 22381 | ggttggatca aagtactttg atcccgaggg gaaccctgtg gttggcatgc acatacaaat |
| 22441 | ggacgaacgg ataaaccttt tcacgccctt ttaaatatcc gttattctaa taaacgctct |
| 22501 | TTTCTCTTAG |

SEQ ID NO: 90.

| | |
|---|---|
| LOCUS | donor_vector_mPing in GFP ds-DNA circular |
| | 09-MAR.-2022 |
| DEFINITION | . |
| ACCESSION | urn.local . . . .16-av3vsf2 |
| VERSION | urn.local . . . .16-av3vsf2 |
| FEATURES | Location/Qualifiers |
| misc_feature | 1 . . . 26 |
| | /label = “LB″ |
| regulatory | complement (665 . . . 920) |
| | /label = “NOS Terminator″ |
| misc_feature | complement (940 . . . 1728) |
| | /label = “eGFP5-er″ |
| Transposon | 1758 . . . 2187 |
| | /label = “mPing″ |
| promoter | complement (2204 . . . 3037) |
| | /label = “CaMV Promoter″ |
| regulatory | complement (3734 . . . 3989) |
| | / label = “NOS Terminator″ |
| misc_feature | complement (4379 . . . 5176) |
| | /label = “Kan Resistance″ |
| regulatory | complement (5186 . . . 5492) |
| | /label = “NOS Promoter″ |
| Agro tDNA cut site | complement (5533 . . . 5557) |
| | /label = “RB″ |

ORIGIN

| | |
|---|---|
| 1 | tggcaggata tattgtggtg taaacaaatt gacgcttaga caacttaata acacattgcg |
| 61 | gacgttttta atgtactggg gtggtttttc ttttcaccag tgagacgggc aacagctgat |
| 121 | tgcccttcac cgcctggccc tgagagagtt gcagcaagcg gtccacgctg gtttgcccca |
| 181 | gcaggcgaaa atcctgtttg atggtggttc cgaaatcggc aaaatccctt ataaatcaaa |
| 241 | agaatagccc gagatagggt tgagtgttgt tccagtttgg aacaagagtc cactattaaa |
| 301 | gaacgtggac tccaacgtca aagggcgaaa aaccgtctat cagggcgatg gcccactacg |
| 361 | tgaaccatca cccaaatcaa gttttttggg gtcgaggtgc cgtaaagcac taaatcggaa |
| 421 | ccctaaaggg agcccccgat ttagagcttg acggggaaag ccggcgaacg tggcgagaaa |
| 481 | ggaagggaag aaagcgaaag gagcgggcgc cattcaggct gcgcaactgt tgggaagggc |
| 541 | gatcggtgcg ggcctcttcg ctattacgcc agctggcgaa agggggatgt gctgcaaggc |
| 601 | gattaagttg ggtaacgcca gggttttccc agtcacgacg ttgtaaaacg acggccagtg |
| 661 | aattcccgat ctagtaacat agatgacacc gcgcgcgata atttatccta gtttgcgcgc |
| 721 | tatattttgt tttctatcgc gtattaaatg tataattgcg ggactctaat cataaaaacc |
| 781 | catctcataa ataacgtcat gcattacatg ttaattatta catgcttaac gtaattcaac |
| 841 | agaaattata tgataatcat cgcaagaccg gcaacaggat tcaatcttaa gaaactttat |
| 901 | tgccaaatgt ttgaacgatc ggggaaattc gagctcttaa agctcatcat gtttgtatag |
| 961 | ttcatccatg ccatgtgtaa tcccagcagc tgttacaaac tcaagaagga ccatgtggtc |
| 1021 | tctcttttcg ttgggatctt tcgaaagggc agattgtgtg gacaggtaat ggttgtctgg |
| 1081 | taaaaggaca gggccatcgc caattggagt attttgttga taatgatcag cgagttgcac |
| 1141 | gccgccgtct tcgatgttgt ggcgggtctt gaagttggct ttgatgccgt tcttttgctt |
| 1201 | gtcggccatg atgtatacgt tgtgggagtt gtagttgtat tccaacttgt ggccgaggat |
| 1261 | gtttccgtcc tccttgaaat cgattccctt aagctcgatc ctgttgacga gggtgtctcc |
| 1321 | ctcaaacttg acttcagcac gtgtcttgta gttcccgtcg tccttgaaga agatggtcct |
| 1381 | ctcctgcacg tatccctcag gcatggcgct cttgaagaag tcgtgccgct tcatatgatc |
| 1441 | tgggtatctt gaaaagcatt gaacaccata agagaaagta gtgacaagtg ttggccatgg |
| 1501 | aacaggtagt tttccagtag tgcaaataaa tttaagggta agttttccgt atgttgcatc |
| 1561 | accttcaccc tctccactga cagaaaattt gtgcccatta acatcaccat ctaattcaac |
| 1621 | aagaattggg acaactccag tgaaaagttc ttctccttta ctgaattcgg ccgaggataa |
| 1681 | tgataggaga agtgaaaaga tgagaaagag aaaaagatta gtcttcattg ttatatctcc |
| 1741 | ttggatcctc tagattaggc cagtcacaat ggctagtgtc attgcacggc tacccaaaat |
| 1801 | attataccat cttctctcaa atgaaatctt ttatgaaaca atccccacag tggaggggtt |
| 1861 | tcactttgac gtttccaaga ctaagcaaag catttaattg atacaagttg ctgggatcat |
| 1921 | ttgtacccaa aatccggcgc ggcgcgggag aatgcggagg tcgcacggcg gaggcggacg |
| 1981 | caagagatcc ggtgaatgaa acgaatcggc ctcaacgggg gtttcactct gttaccgagg |
| 2041 | acttggaaac gacgctgacg agtttcacca ggatgaaact ctttccttct ctctcatccc |
| 2101 | catttcatgc aaataatcat tttttattca gtcttacccc tattaaatgt gcatgacaca |
| 2161 | ccagtgaaac ccccattgtg actggcctta tctagagtcc cccgtgttct ctccaaatga |
| 2221 | aatgaacttc cttatataga ggaagggtct tgcgaaggat agtgggattg tgcgtcatcc |
| 2281 | cttacgtcag tggagatatc acatcaatcc acttgctttg aagacgtggt tggaacgtct |
| 2341 | tctttttcca cgatgctcct cgtgggtggg ggtccatctt tgggaccact gtcggcagag |
| 2401 | gcatcttcaa cgatggcctt tcctttatcg caatgatggc atttgtagga gccaccttcc |
| 2461 | ttttccacta tcttcacaat aaagtgacag atagctgggc aatggaatcc gaggaggttt |
| 2521 | ccggatatta ccctttgttg aaaagtctca attgcccttt ggtcttctga gactgtatct |
| 2581 | ttgatatttt tggagtagac aagtgtgtcg tgctccacca tgttgacgaa gattttcttc |
| 2641 | ttgtcattga gtcgtaagag actctgtatg aactgttcgc cagtctttac ggcgagttct |
| 2701 | gttaggtcct ctatttgaat ctttgactcc atggcctttg attcagtggg aactaccttt |
| 2761 | ttagagactc caatctctat tacttgcctt ggtttgtgaa gcaagccttg aatcgtccat |
| 2821 | actggaatag tacttctgat cttgagaaat atatctttct ctgtgttctt gatgcagtta |
| 2881 | gtcctgaatc ttttgactgc atctttaacc ttcttgggaa ggtatttgat ttcctggaga |
| 2941 | ttattgctcg ggtagatcgt cttgatgaga cctgctgcgt aagcctctct aaccatctgt |
| 3001 | gggttagcat tctttctgaa attgaaaagg ctaatctggg gacctgcagg catgcaagct |
| 3061 | tggcgtaatc atggtcatag ctgtttcctg tgtgaaattg ttatccgctc acaattccac |
| 3121 | acaacatacg agccggaagc ataaagtgta aagcctgggg tgcctaatga gtgagctaac |
| 3181 | tcacattaat tgcgttgcgc tcactgcccg ctttccagtc gggaaacctg tcgtgccagc |
| 3241 | tgcattaatg aatcggccaa cgcgcgggga gaggcggttt gcgtattggg ccaaagacaa |
| 3301 | aagggcgaca ttcaaccgat tgagggaggg aaggtaaata ttgacggaaa ttattcatta |
| 3361 | aaggtgaatt atcaccgtca ccgacttgag ccatttggga attagagcca gcaaaatcac |
| 3421 | cagtagcacc attaccatta gcaaggccgg aaacgtcacc aatgaaacca tcgatagcag |
| 3481 | caccgtaatc agtagcgaca gaatcaagtt tgcctttagc gtcagactgt agcgcgtttt |
| 3541 | catcggcatt ttcggtcata gcccccttat tagcgtttgc catcttttca taatcaaaat |
| 3601 | caccggaacc agagccacca ccggaaccgc ctccctcaga gccgccaccc tcagaaccgc |
| 3661 | caccctcaga gccaccaccc tcagagccgc caccagaacc accaccagag ccgccgccag |
| 3721 | cattgacagg aggcccgatc tagtaacata gatgacaccg cgcgcgataa tttatcctag |
| 3781 | tttgcgcgct atattttgtt ttctatcgcg tattaaatgt ataattgcgg gactctaatc |
| 3841 | ataaaaaccc atctcataaa taacgtcatg cattacatgt taattattac atgcttaacg |
| 3901 | taattcaaca gaaattatat gataatcatc gcaagaccgg caacaggatt caatcttaag |
| 3961 | aaactttatt gccaaatgtt tgaacgatcg gggatcatcc gggtctgtgg cgggaactcc |
| 4021 | acgaaaatat ccgaacgcag caagatatcg cggtgcatct cggtcttgcc tgggcagtcg |
| 4081 | ccgccgacgc cgttgatgtg gacgccgggc ccgatcatat tgtcgctcag gatcgtggcg |
| 4141 | ttgtgcttgt cggccgttgc tgtcgtaatg atatcggcac cttcgaccgc ctgttccgca |
| 4201 | gagatcccgt gggcgaagaa ctccagcatg agatccccgc gctggaggat catccagccg |
| 4261 | gcgtcccgga aaacgattcc gaagcccaac ctttcataga aggcggcggt ggaatcgaaa |
| 4321 | tctcgtgatg gcaggttggg cgtcgcttgg tcggtcattt cgaaccccag agtcccgctc |
| 4381 | agaagaactc gtcaagaagg cgatagaagg cgatgcgctg cgaatcggga gcggcgatac |
| 4441 | cgtaaagcac gaggaagcgg tcagcccatt cgccgccaag ctcttcagca atatcacggg |
| 4501 | tagccaacgc tatgtcctga tagcggtccg ccacacccag ccggccacag tcgatgaatc |
| 4561 | cagaaaagcg gccattttcc accatgatat tcggcaagca ggcatcgcca tgggtcacga |
| 4621 | cgagatcatc gccgtcgggc atgcgcgcct tgagcctggc gaacagttcg gctggcgcga |
| 4681 | gcccctgatg ctcttcgtcc agatcatcct gatcgacaag accggcttcc atccgagtac |
| 4741 | gtgctcgctc gatgcgatgt ttcgcttggt ggtcgaatgg gcaggtagcc ggatcaagcg |
| 4801 | tatgcagccg ccgcattgca tcagccatga tggatacttt ctcggcagga gcaaggtgag |
| 4861 | atgacaggag atcctgcccc ggcacttcgc ccaatagcag ccagtccctt cccgcttcag |
| 4921 | tgacaacgtc gagcacagct gcgcaaggaa cgcccgtcgt ggccagccac gatagccgcg |
| 4981 | ctgcctcgtc ctgcagttca ttcagggcac cggacaggtc ggtcttgaca aaaagaaccg |
| 5041 | ggcgcccctg cgctgacagc cggaacacgg cggcatcaga gcagccgatt gtctgttgtg |
| 5101 | cccagtcata gccgaatagc ctctccaccc aagcggccgg agaacctgcg tgcaatccat |
| 5161 | cttgttcaat catgcgaaac gatccagatc cggtgcagat tatttggatt gagagtgaat |
| 5221 | atgagactct aattggatac cgaggggaat ttatggaacg tcagtggagc atttttgaca |
| 5281 | agaaatattt gctagctgat agtgacctta ggcgactttt gaacgcgcaa taatggtttc |
| 5341 | tgacgtatgt gcttagctca ttaaactcca gaaacccgcg gctgagtggc tccttcaacg |
| 5401 | ttgcggttct gtcagttcca aacgtaaaac ggcttgtccc gcgtcatcgg cgggggtcat |
| 5461 | aacgtgactc ccttaattct ccgctcatga tcagattgtc gtttcccgcc ttcagtttaa |
| 5521 | actatcagtg tttgacagga tatattggcg ggtaaaccta agagaaaaga gcgtttatta |
| 5581 | gaataatcgg atatttaaaa gggcgtgaaa aggtttatcc gttcgtccat ttgtatgtgc |
| 5641 | atgccaacca cagggttccc cagatctggc gccggccagc gagacgagca agattggccg |
| 5701 | ccgcccgaaa cgatccgaca gcgcgcccag cacaggtgcg caggcaaatt gcaccaacgc |
| 5761 | atacagcgcc agcagaatgc catagtgggc ggtgacgtcg ttcgagtgaa ccagatcgcg |
| 5821 | caggaggccc ggcagcaccg gcataatcag gccgatgccg acagcgtcga gcgcgacagt |
| 5881 | gctcagaatt acgatcaggg gtatgttggg tttcacgtct ggcctccgga ccagcctccg |
| 5941 | ctggtccgat tgaacgcgcg gattctttat cactgataag ttggtggaca tattatgttt |
| 6001 | atcagtgata aagtgtcaag catgacaaag ttgcagccga atacagtgat ccgtgccgcc |
| 6061 | ctggacctgt tgaacgaggt cggcgtagac ggtctgacga cacgcaaact ggcggaacgg |
| 6121 | ttgggggttc agcagccggc gctttactgg cacttcagga acaagcgggc gctgctcgac |
| 6181 | gcactggccg aagccatgct ggcggagaat catacgcatt cggtgccgag agccgacgac |
| 6241 | gactggcgct catttctgat cgggaatgcc cgcagcttca ggcaggcgct gctcgcctac |
| 6301 | cgcgatggcg cgcgcatcca tgccggcacg cgaccgggcg caccgcagat ggaaacggcc |
| 6361 | gacgcgcagc ttcgcttcct ctgcgaggcg ggtttttcgg ccggggacgc cgtcaatgcg |
| 6421 | ctgatgacaa tcagctactt cactgttggg gccgtgcttg aggagcaggc cggcgacagc |
| 6481 | gatgccggcg agcgcggcgg caccgttgaa caggctccgc tctcgccgct gttgcgggcc |
| 6541 | gcgatagacg ccttcgacga agccggtccg gacgcagcgt tcgagcaggg actcgcggtg |
| 6601 | attgtcgatg gattggcgaa aaggaggctc gttgtcagga acgttgaagg accgagaaag |
| 6661 | ggtgacgatt gatcaggacc gctgccggag cgcaacccac tcactacagc agagccatgt |
| 6721 | agacaacatc ccctccccct ttccaccgcg tcagacgccc gtagcagccc gctacgggct |
| 6781 | ttttcatgcc ctgccctagc gtccaagcct cacggccgcg ctcggcctct ctggcggcct |
| 6841 | tctggcgctc ttccgcttcc tcgctcactg actcgctgcg ctcggtcgtt cggctgcggc |
| 6901 | gagcggtatc agctcactca aaggcggtaa tacggttatc cacagaatca ggggataacg |
| 6961 | caggaaagaa catgtgagca aaaggccagc aaaaggccag gaaccgtaaa aaggccgcgt |
| 7021 | tgctggcgtt tttccatagg ctccgccccc ctgacgagca tcacaaaaat cgacgctcaa |
| 7081 | gtcagaggtg gcgaaacccg acaggactat aaagatacca ggcgtttccc cctggaagct |
| 7141 | ccctcgtgcg ctctcctgtt ccgaccctgc cgcttaccgg atacctgtcc gcctttctcc |
| 7201 | cttcgggaag cgtggcgctt ttccgctgca taaccctgct tcggggtcat tatagcgatt |
| 7261 | ttttcggtat atccatcctt tttcgcacga tatacaggat tttgccaaag ggttcgtgta |
| 7321 | gactttcctt ggtgtatcca acggcgtcag ccgggcagga taggtgaagt aggcccaccc |
| 7381 | gcgagcgggt gttccttctt cactgtccct tattcgcacc tggcggtgct caacgggaat |
| 7441 | cctgctctgc gaggctggcc ggctaccgcc ggcgtaacag atgagggcaa gcggatggct |
| 7501 | gatgaaacca agccaaccag gaagggcagc ccacctatca aggtgtactg ccttccagac |
| 7561 | gaacgaagag cgattgagga aaaggcggcg gcggccggca tgagcctgtc ggcctacctg |
| 7621 | ctggccgtcg gccagggcta caaaatcacg ggcgtcgtgg actatgagca cgtccgcgag |
| 7681 | ctggcccgca tcaatggcga cctgggccgc ctgggcggcc tgctgaaact ctggctcacc |
| 7741 | gacgacccgc gcacggcgcg gttcggtgat gccacgatcc tcgccctgct ggcgaagatc |
| 7801 | gaagagaagc aggacgagct tggcaaggtc atgatgggcg tggtccgccc gagggcagag |
| 7861 | ccatgacttt tttagccgct aaaacggccg gggggtgcgc gtgattgcca agcacgtccc |
| 7921 | catgcgctcc atcaagaaga gcgacttcgc ggagctggtg aagtacatca ccgacgagca |
| 7981 | aggcaagacc gagcgccttt gcgacgctca ccgggctggt tgccctcgcc gctgggctgg |
| 8041 | cggccgtcta tggccctgca aacgcgccag aaacgccgtc gaagccgtgt gcgagacacc |
| 8101 | gcggccgccg gcgttgtgga tacctcgcgg aaaacttggc cctcactgac agatgagggg |
| 8161 | cggacgttga cacttgaggg gccgactcac ccggcgcggc gttgacagat gaggggcagg |
| 8221 | ctcgatttcg gccggcgacg tggagctggc cagcctcgca aatcggcgaa aacgcctgat |
| 8281 | tttacgcgag tttcccacag atgatgtgga caagcctggg gataagtgcc ctgcggtatt |
| 8341 | gacacttgag gggcgcgact actgacagat gaggggcgcg atccttgaca cttgaggggc |
| 8401 | agagtgctga cagatgaggg gcgcacctat tgacatttga ggggctgtcc acaggcagaa |
| 8461 | aatccagcat ttgcaagggt ttccgcccgt ttttcggcca ccgctaacct gtcttttaac |
| 8521 | ctgcttttaa accaatattt ataaaccttg tttttaacca gggctgcgcc ctgtgcgcgt |
| 8581 | gaccgcgcac gccgaagggg ggtgcccccc cttctcgaac cctcccggcc cgctaacgcg |
| 8641 | ggcctcccat ccccccaggg gctgcgcccc tcggccgcga acggcctcac cccaaaaatg |
| 8701 | gcagcgctgg cagtccttgc cattgccggg atcggggcag taacgggatg ggcgatcagc |
| 8761 | ccgagcgcga cgcccggaag cattgacgtg ccgcaggtgc tggcatcgac attcagcgac |
| 8821 | caggtgccgg gcagtgaggg cggcggcctg ggtggcggcc tgcccttcac ttcggccgtc |
| 8881 | ggggcattca cggacttcat ggcggggccg gcaattttta ccttgggcat tcttggcata |
| 8941 | gtggtcgcgg gtgccgtgct cgtgttcggg ggtgcgataa acccagcgaa ccatttgagg |
| 9001 | tgataggtaa gattataccg aggtatgaaa acgagaattg gacctttaca gaattactct |
| 9061 | atgaagcgcc atatttaaaa agctaccaag acgaagagga tgaagaggat gaggaggcag |
| 9121 | attgccttga atatattgac aatactgata agataatata tcttttatat agaagatatc |
| 9181 | gccgtatgta aggatttcag ggggcaaggc ataggcagcg cgcttatcaa tatatctata |
| 9241 | gaatgggcaa agcataaaaa cttgcatgga ctaatgcttg aaacccagga caataacctt |
| 9301 | atagcttgta aattctatca taattgggta atgactccaa cttattgata gtgttttatg |
| 9361 | ttcagataat gcccgatgac tttgtcatgc agctccaccg attttgagaa cgacagcgac |
| 9421 | ttccgtccca gccgtgccag gtgctgcctc agattcaggt tatgccgctc aattcgctgc |
| 9481 | gtatatcgct tgctgattac gtgcagcttt cccttcaggc gggattcata cagcggccag |
| 9541 | ccatccgtca tccatatcac cacgtcaaag ggtgacagca ggctcataag acgccccagc |
| 9601 | gtcgccatag tgcgttcacc gaatacgtgc gcaacaaccg tcttccggag actgtcatac |
| 9661 | gcgtaaaaca gccagcgctg gcgcgattta gccccgacat agccccactg ttcgtccatt |
| 9721 | tccgcgcaga cgatgacgtc actgcccggc tgtatgcgcg aggttaccga ctgcggcctg |
| 9781 | agttttttaa gtgacgtaaa atcgtgttga ggccaacgcc cataatgcgg gctgttgccc |
| 9841 | ggcatccaac gccattcatg gccatatcaa tgattttctg gtgcgtaccg ggttgagaag |
| 9901 | cggtgtaagt gaactgcagt tgccatgttt tacggcagtg agagcagaga tagcgctgat |
| 9961 | gtccggcggt gcttttgccg ttacgcacca ccccgtcagt agctgaacag gagggacagc |
| 10021 | tgatagacac agaagccact ggagcacctc aaaaacacca tcatacacta aatcagtaag |
| 10081 | ttggcagcat cacccataat tgtggtttca aaatcggctc cgtcgatact atgttatacg |
| 10141 | ccaactttga aaacaacttt gaaaaagctg ttttctggta tttaaggttt tagaatgcaa |
| 10201 | ggaacagtga attggagttc gtcttgttat aattagcttc ttggggtatc tttaaatact |
| 10261 | gtagaaaaga ggaaggaaat aataaatggc taaaatgaga atatcaccgg aattgaaaaa |
| 10321 | actgatcgaa aaataccgct gcgtaaaaga tacggaagga atgtctcctg ctaaggtata |
| 10381 | taagctggtg ggagaaaatg aaaacctata tttaaaaatg acggacagcc ggtataaagg |
| 10441 | gaccacctat gatgtggaac gggaaaagga catgatgcta tggctggaag gaaagctgcc |
| 10501 | tgttccaaag gtcctgcact ttgaacggca tgatggctgg agcaatctgc tcatgagtga |
| 10561 | ggccgatggc gtcctttgct cggaagagta tgaagatgaa caaagccctg aaaagattat |
| 10621 | cgagctgtat gcggagtgca tcaggctctt tcactccatc gacatatcgg attgtcccta |
| 10681 | tacgaatagc ttagacagcc gcttagccga attggattac ttactgaata acgatctggc |
| 10741 | cgatgtggat tgcgaaaact gggaagaaga cactccattt aaagatccgc gcgagctgta |
| 10801 | tgatttttta aagacggaaa agcccgaaga ggaacttgtc ttttcccacg gcgacctggg |
| 10861 | agacagcaac atctttgtga aagatggcaa agtaagtggc tttattgatc ttgggagaag |
| 10921 | cggcagggcg gacaagtggt atgacattgc cttctgcgtc cggtcgatca gggaggatat |
| 10981 | cggggaagaa cagtatgtcg agctattttt tgacttactg gggatcaagc ctgattggga |
| 11041 | gaaaataaaa tattatattt tactggatga attgttttag tacctagatg tggcgcaacg |
| 11101 | atgccggcga caagcaggag cgcaccgact tcttccgcat caagtgtttt ggctctcagg |
| 11161 | ccgaggccca cggcaagtat ttgggcaagg ggtcgctggt attcgtgcag ggcaagattc |
| 11221 | ggaataccaa gtacgagaag gacggccaga cggtctacgg gaccgacttc attgccgata |
| 11281 | aggtggatta tctggacacc aaggcaccag gcgggtcaaa tcaggaataa gggcacattg |
| 11341 | ccccggcgtg agtcggggca atcccgcaag gagggtgaat gaatcggacg tttgaccgga |
| 11401 | aggcatacag gcaagaactg atcgacgcgg ggttttccgc cgaggatgcc gaaaccatcg |
| 11461 | caagccgcac cgtcatgcgt gcgccccgcg aaaccttcca gtccgtcggc tcgatggtcc |
| 11521 | agcaagctac ggccaagatc gagcgcgaca gcgtgcaact ggctccccct gccctgcccg |
| 11581 | cgccatcggc cgccgtggag cgttcgcgtc gtctcgaaca ggaggcggca ggtttggcga |
| 11641 | agtcgatgac catcgacacg cgaggaacta tgacgaccaa gaagcgaaaa accgccggcg |
| 11701 | aggacctggc aaaacaggtc agcgaggcca agcaggccgc gttgctgaaa cacacgaagc |
| 11761 | agcagatcaa ggaaatgcag ctttccttgt tcgatattgc gccgtggccg gacacgatgc |
| 11821 | gagcgatgcc aaacgacacg gcccgctctg ccctgttcac cacgcgcaac aagaaaatcc |
| 11881 | cgcgcgaggc gctgcaaaac aaggtcattt tccacgtcaa caaggacgtg aagatcacct |
| 11941 | acaccggcgt cgagctgcgg gccgacgatg acgaactggt gtggcagcag gtgttggagt |
| 12001 | acgcgaagcg cacccctatc ggcgagccga tcaccttcac gttctacgag ctttgccagg |
| 12061 | acctgggctg gtcgatcaat ggccggtatt acacgaaggc cgaggaatgc ctgtcgcgcc |
| 12121 | tacaggcgac ggcgatgggc ttcacgtccg accgcgttgg gcacctggaa tcggtgtcgc |
| 12181 | tgctgcaccg cttccgcgtc ctggaccgtg gcaagaaaac gtcccgttgc caggtcctga |
| 12241 | tcgacgagga aatcgtcgtg ctgtttgctg gcgaccacta cacgaaattc atatgggaga |
| 12301 | agtaccgcaa gctgtcgccg acggcccgac ggatgttcga ctatttcagc tcgcaccggg |
| 12361 | agccgtaccc gctcaagctg gaaaccttcc gcctcatgtg cggatcggat tccacccgcg |
| 12421 | tgaagaagtg gcgcgagcag gtcggcgaag cctgcgaaga gttgcgaggc agcggcctgg |
| 12481 | tggaacacgc ctgggtcaat gatgacctgg tgcattgcaa acgctagggc cttgtggggt |
| 12541 | cagttccggc tgggggttca gcagccagcg ctttactggc atttcaggaa caagcgggca |
| 12601 | ctgctcgacg cacttgcttc gctcagtatc gctcgggacg cacggcgcgc tctacgaact |
| 12661 | gccgataaac agaggattaa aattgacaat tgtgattaag gctcagattc gacggcttgg |
| 12721 | agcggccgac gtgcaggatt tccgcgagat ccgattgtcg gccctgaaga aagctccaga |
| 12781 | gatgttcggg tccgtttacg agcacgagga gaaaaagccc atggaggcgt tcgctgaacg |
| 12841 | gttgcgagat gccgtggcat tcggcgccta catcgacggc gagatcattg ggctgtcggt |
| 12901 | cttcaaacag gaggacggcc ccaaggacgc tcacaaggcg catctgtccg gcgttttcgt |
| 12961 | ggagcccgaa cagcgaggcc gaggggtcgc cggtatgctg ctgcgggcgt tgccggcggg |
| 13021 | tttattgctc gtgatgatcg tccgacagat tccaacggga atctggtgga tgcgcatctt |
| 13081 | catcctcggc gcacttaata tttcgctatt ctggagcttg ttgtttattt cggtctaccg |
| 13141 | cctgccgggc ggggtcgcgg cgacggtagg cgctgtgcag ccgctgatgg tcgtgttcat |
| 13201 | ctctgccgct ctgctaggta gcccgatacg attgatggcg gtcctggggg ctatttgcgg |
| 13261 | aactgcgggc gtggcgctgt tggtgttgac accaaacgca gcgctagatc ctgtcggcgt |
| 13321 | cgcagcgggc ctggcggggg cggtttccat ggcgttcgga accgtgctga cccgcaagtg |
| 13381 | gcaacctccc gtgcctctgc tcacctttac cgcctggcaa ctggcggccg gaggacttct |
| 13441 | gctcgttcca gtagctttag tgtttgatcc gccaatcccg atgcctacag gaaccaatgt |
| 13501 | tctcggcctg gcgtggctcg gcctgatcgg agcgggttta acctacttcc tttggttccg |
| 13561 | ggggatctcg cgactcgaac ctacagttgt ttccttactg ggctttctca gccccagatc |
| 13621 | tggggtcgat cagccgggga tgcatcaggc cgacagtcgg aacttcgggt ccccgacctg |
| 13681 | taccattcgg tgagcaatgg ataggggagt tgatatcgtc aacgttcact tctaaagaaa |
| 13741 | tagcgccact cagcttcctc agcggcttta tccagcgatt tcctattatg tcggcatagt |
| 13801 | tctcaagatc gacagcctgt cacggttaag cgagaaatga ataagaaggc tgataattcg |
| 13861 | gatctctgcg agggagatga tatttgatca caggcagcaa cgctctgtca tcgttacaat |
| 13921 | caacatgcta ccctccgcga gatcatccgt gtttcaaacc cggcagctta gttgccgttc |
| 13981 | ttccgaatag catcggtaac atgagcaaag tctgccgcct tacaacggct ctcccgctga |
| 14041 | cgccgtcccg gactgatggg ctgcctgtat cgagtggtga ttttgtgccg agctgccggt |
| 14101 | cggggagctg ttggctggct gg |

SEQ ID NO: 91.

| | |
|---|---|
| LOCUS | helper_vector_for_figu 21085 bp ds-DNA circular 09-MAR.-2022 |
| DEFINITION | . |
| ACCESSION | pVec1 |
| VERSION | pVec1 .1 |
| FEATURES | Location/Qualifiers |
| Agro tDNA cut site | 1 . . . 25 |
| | /label = “RB″ |
| misc_feature | 254 . . . 677 |
| | /label = “U6-26promoter″ |
| misc_feature | 678 . . . 697 |
| | /label = “gRNA to ACT8 promoter″ |
| misc_feature | 698 . . . 773 |
| | /label = “gRNA scaffold″ |
| misc_feature | 774 . . . 965 |
| | /label = “U6-26 terminator″ |
| promoter | 981 . . . 2667 |
| | /label = “Rps5a″ |
| misc_feature | 2704 . . . 4101 |
| | /label = “ORF1″ |
| terminator | 4165 . . . 4890 |
| | /label = “OCS terminator″ |
| promoter | 5073 . . . 5992 |
| | /label = “GmUbi3 Promoter″ |
| misc_feature | 6014 . . . 7459 |
| | /label = “Pong TPase LA″ |
| CDS | 6014 . . . 11677 |
| | /label = “Translation 6014-11677″ |
| misc_feature | 7463 . . . 7477 |
| | /label = “G4S linker″ |
| feature | 7481 . . . 7501 |
| | /label = “SV40 NLS″ |
| misc_feature | 7505 . . . 11674 |
| | /label = “Cas9″ |
| misc_feature | 11627 . . . 11674 |
| | /label = “NLS″ |
| terminator | 11702 . . . 12429 |
| | /label = “OCS Terminator″ |
| promoter | 12680 . . . 13421 |
| | /label = “CaMVd35S promoter″ |
| gene | 13512 . . . 14507 |
| | /label = “hygroB (variant) ″ |
| misc_feature | complement (15125 . . . 15147) |
| | /label = “LB″ |
| gene | 15263 . . . 16057 |
| | /label = “KanR1″ |
| origin | 16128 . . . 16740 |
| | /label = “pBR322_origin″ |

ORIGIN

| | |
|---|---|
| 1 | gtttacccgc caatatatcc tgtcaaacac tgatagttta aactgaaggc gggaaacgac |
| 61 | aatctgatcc aagctcaagc tgctctagca ttcgccattc aggctgcgca actgttggga |
| 121 | agggcgatcg gtgcgggcct cttcgctatt acgccagctg gcgaaagggg gatgtgctgc |
| 181 | aaggcgatta agttgggtaa cgccagggtt ttcccagtca cgacgttgta aaacgacggc |
| 241 | cagtgccaag cttcgacttg ccttccgcac aatacatcat ttcttcttag ctttttttct |
| 301 | tcttcttcgt tcatacagtt tttttttgtt tatcagctta cattttcttg aaccgtagct |
| 361 | ttcgttttct tctttttaac tttccattcg gagtttttgt atcttgtttc atagtttgtc |
| 421 | ccaggattag aatgattagg catcgaacct tcaagaattt gattgaataa aacatcttca |
| 481 | ttcttaagat atgaagataa tcttcaaaag gcccctggga atctgaaaga agagaagcag |
| 541 | gcccatttat atgggaaaga acaatagtat ttcttatata ggcccattta agttgaaaac |
| 601 | aatcttcaaa agtcccacat cgcttagata agaaaacgaa gctgagttta tatacagcta |
| 661 | gagtcgaagt agtgattGTT ACAGGAGTAG TTCATCGgtt ttagagctag aaatagcaag |
| 721 | ttaaaataag gctagtccgt tatcaacttg aaaaagtggc accgagtcgg tgcttttttt |
| 781 | tgcaaaattt tccagatcga tttcttcttc ctctgttctt cggcgttcaa tttctggggt |
| 841 | tttctcttcg ttttctgtaa ctgaaaccta aaatttgacc taaaaaaaat ctcaaataat |
| 901 | atgattcagt ggttttgtac ttttcagtta gttgagtttt gcagttccga tgagataaac |
| 961 | caataccatg ttagagagcg ctagttcgtg agtagatata ttactcaact tttgattcgc |
| 1021 | tatttgcagt gcacctgtgg cgttcatcac atcttttgtg acactgtttg cactggtcat |
| 1081 | tgctattaca aaggaccttc ctgatgttga aggagatcga aagtaagtaa ctgcacgcat |
| 1141 | aaccattttc tttccgctct ttggctcaat ccatttgaca gtcaaagaca atgtttaacc |
| 1201 | agctccgttt gatatattgt ctttatgtgt ttgttcaagc atgtttagtt aatcatgcct |
| 1261 | ttgattgatc ttgaataggt tccaaatatc aaccctggca acaaaacttg gagtgagaaa |
| 1321 | cattgcattc ctcggttctg gacttctgct agtaaattat gtttcagcca tatcactagc |
| 1381 | tttctacatg cctcaggtga attcatctat ttccgtctta actatttcgg ttaatcaaag |
| 1441 | cacgaacacc attactgcat gtagaagctt gataaactat cgccaccaat ttatttttgt |
| 1501 | tgcgatattg ttactttcct cagtatgcag ctttgaaaag accaaccctc ttatccttta |
| 1561 | acaatgaaca ggtttttaga ggtagcttga tgattcctgc acatgtgatc ttggcttcag |
| 1621 | gcttaatttt ccaggtaaag cattatgaga tactcttata tctcttacat acttttgaga |
| 1681 | taatgcacaa gaacttcata actatatgct ttagtttctg catttgacac tgccaaattc |
| 1741 | attaatctct aatatctttg ttgttgatct ttggtagaca tgggtactag aaaaagcaaa |
| 1801 | ctacaccaag gtaaaatact tttgtacaaa cataaactcg ttatcacgga acatcaatgg |
| 1861 | agtgtatatc taacggagtg tagaaacatt tgattattgc aggaagctat ctcaggatat |
| 1921 | tatcggttta tatggaatct cttctacgca gagtatctgt tattcccctt cctctagctt |
| 1981 | tcaatttcat ggtgaggata tgcagttttc tttgtatatc attcttcttc ttctttgtag |
| 2041 | cttggagtca aaatcggttc cttcatgtac atacatcaag gatatgtcct tctgaatttt |
| 2101 | tatatcttgc aataaaaatg cttgtaccaa ttgaaacacc agctttttga gttctatgat |
| 2161 | cactgacttg gttctaacca aaaaaaaaaa aatgtttaat ttacatatct aaaagtaggt |
| 2221 | ttagggaaac ctaaacagta aaatatttgt atattattcg aatttcactc atcataaaaa |
| 2281 | cttaaattgc accataaaat tttgttttac tattaatgat gtaatttgtg taacttaaga |
| 2341 | taaaaataat attccgtaag ttaaccggct aaaaccacgt ataaaccagg gaacctgtta |
| 2401 | aaccggttct ttactggata aagaaatgaa agcccatgta gacagctcca ttagagccca |
| 2461 | aaccctaaat ttctcatcta tataaaagga gtgacattag ggtttttgtt cgtcctctta |
| 2521 | aagcttctcg ttttctctgc cgtctctctc attcgcgcga cgcaaacgat cttcaggtga |
| 2581 | tcttctttct ccaaatcctc tctcataact ctgatttcgt acttgtgtat ttgagctcac |
| 2641 | gctctgtttc tctcaccaca gccggattcg agatcacaag tttgtacaaa aaagcaggct |
| 2701 | tccatggatc cgtcgccggc cgtggatccg tcgccggccg tggatccgtc gccggctgct |
| 2761 | gaaacccggc ggcgtgcaac cgggaaagga ggcaaacagc gcgggggcaa gcaactagga |
| 2821 | ttgaagaggc cgccgccgat ttctgtcccg gccaccccgc ctcctgctgc gacgtcttca |
| 2881 | tcccctgctg cgccgacggc catcccacca cgaccaccgc aatcttcgcc gattttcgtc |
| 2941 | cccgattcgc cgaatccgtc accggctgcg ccgacctcct ctcttgcttc ggggacatcg |
| 3001 | acggcaaggc caccgcaacc acaaggagga ggatggggac caacatcgac catttcccca |
| 3061 | aactttgcat ctttctttgg aaaccaacaa gacccaaatt catgtttggt caggggttat |
| 3121 | cctccaggag ggtttgtcaa ttttattcaa caaaattgtc cgccgcagcc acaacagcaa |
| 3181 | ggtgaaaatt ttcatttcgt tggtcacaat atggggttca acccaatatc tccacagcca |
| 3241 | ccaagtgcct acggaacacc aacaccccaa gctacgaacc aaggcacttc aacaaacatt |
| 3301 | atgattgatg aagaggacaa caatgatgac agtagggcag caaagaaaag atggactcat |
| 3361 | gaagaggaag agagactggc cagtgcttgg ttgaatgctt ctaaagactc aattcatggg |
| 3421 | aatgataaga aaggtgatac attttggaag gaagtcactg atgaatttaa caagaaaggg |
| 3481 | aatggaaaac gtaggaggga aattaaccaa ctgaaggttc actggtcaag gttgaagtca |
| 3541 | gcgatctctg agttcaatga ctattggagt acggttactc aaatgcatac aagcggatac |
| 3601 | tcagacgaca tgcttgagaa agaggcacag aggctgtatg caaacaggtt tggaaaacct |
| 3661 | tttgcgttgg tccattggtg gaagatactc aaaagagagc ccaaatggtg tgctcagttt |
| 3721 | gaaaagagga aaaggaagag cgaaatggat gctgttccag aacagcagaa acgtcctatt |
| 3781 | ggtagagaag cagcaaagtc tgagcgcaaa agaaagcgca agaaagaaaa tgttatggaa |
| 3841 | ggcattgtcc tcctagggga caatgtccag aaaattatca aagtgacgca agatcggaag |
| 3901 | ctggagcgtg agaaggtcac tgaagcacag attcacattt caaacgtaaa tttgaaggca |
| 3961 | gcagaacagc aaaaagaagc aaagatgttt gaggtataca attccctgct cactcaagat |
| 4021 | acaagtaaca tgtctgaaga acagaaggct cgccgagaca aggcattaca aaagctggag |
| 4081 | gaaaagttat ttgctgacta gtgacccagc tttcttgtac aaagtggtgc ctaggtgagt |
| 4141 | ctagagagtt gattaagacc cgggactggt ccctagagtc ctgctttaat gagatatgcg |
| 4201 | agacgcctat gatcgcatga tatttgcttt caattctgtt gtgcacgttg taaaaaacct |
| 4261 | gagcatgtgt agctcagatc cttaccgccg gtttcggttc attctaatga atatatcacc |
| 4321 | cgttactatc gtatttttat gaataatatt ctccgttcaa tttactgatt gtaccctact |
| 4381 | acttatatgt acaatattaa aatgaaaaca atatattgtg ctgaataggt ttatagcgac |
| 4441 | atctatgata gagcgccaca ataacaaaca attgcgtttt attattacaa atccaatttt |
| 4501 | aaaaaaagcg gcagaaccgg tcaaacctaa aagactgatt acataaatct tattcaaatt |
| 4561 | tcaaaagtgc cccaggggct agtatctacg acacaccgag cggcgaacta ataacgctca |
| 4621 | ctgaagggaa ctccggttcc ccgccggcgc gcatgggtga gattccttga agttgagtat |
| 4681 | tggccgtccg ctctaccgaa agttacgggc accattcaac ccggtccagc acggcggccg |
| 4741 | ggtaaccgac ttgctgcccc gagaattatg cagcattttt ttggtgtatg tgggccccaa |
| 4801 | atgaagtgca ggtcaaacct tgacagtgac gacaaatcgt tgggcgggtc cagggcgaat |
| 4861 | tttgcgacaa catgtcgagg ctcagcagga cctgcaggca tgcaagcttg gcactggccg |
| 4921 | tcgttttaca acgtcgtgac tgggaaaacc ctggcgttac ccaacttaat cgccttgcag |
| 4981 | cacatccccc tttcgccagc tggcgtaata gcgaagaggc ccgcaccgat cgcccttccc |
| 5041 | aacagttgcg cagcctgaat ggcgaatgct agagcagctt gagcttggat cagattgtcg |
| 5101 | tttcccgcct tcagtttctt gaaggtgcat gtgactccgt caagattacg aaaccgccaa |
| 5161 | ctaccacgca aattgcaatt ctcaatttcc tagaaggact ctccgaaaat gcatccaata |
| 5221 | ccaaatatta cccgtgtcat aggcaccaag tgacaccata catgaacacg cgtcacaata |
| 5281 | tgactggaga agggttccac accttatgct ataaaacgcc ccacacccct cctccttcct |
| 5341 | tcgcagttca attccaatat attccattct ctctgtgtat ttccctacct ctcccttcaa |
| 5401 | ggttagtcga tttcttctgt ttttcttctt cgttctttcc atgaattgtg tatgttcttt |
| 5461 | gatcaatacg atgttgattt gattgtgttt tgtttggttt catcgatctt caattttcat |
| 5521 | aatcagattc agcttttatt atctttacaa caacgtcctt aatttgatga ttctttaatc |
| 5581 | gtagatttgc tctaattaga gctttttcat gtcagatccc tttacaacaa gccttaattg |
| 5641 | ttgattcatt aatcgtagat tagggctttt ttcattgatt acttcagatc cgttaaacgt |
| 5701 | aaccatagat cagggctttt tcatgaatta cttcagatcc gttaaacaac agccttattt |
| 5761 | tttatacttc tgtggttttt caagaaattg ttcagatccg ttgacaaaaa gccttattcg |
| 5821 | ttgattctat atcgtttttc gagagatatt gctcagatct gttagcaact gccttgtttg |
| 5881 | ttgattctat tgccgtggat tagggttttt tttcacgaga ttgcttcaga tccgtactta |
| 5941 | agattacgta atggattttg attctgattt atctgtgatt gttgactcga caggtacctt |
| 6001 | caaacggcgc gccatgcaga gtttagccat ctctctactc ctctcagaaa ctcattccct |
| 6061 | cttttctcat acgaagacct cctccctttt atctttactg tttctctctt cttcaaagat |
| 6121 | gtctgagcaa aatactgatg gaagtcaagt tccagtgaac ttgttggatg agttcctggc |
| 6181 | tgaggatgag atcatagatg atcttctcac tgaagccacg gtggtagtac agtccactat |
| 6241 | agaaggtctt caaaacgagg cttctgacca tcgacatcat ccgaggaagc acatcaagag |
| 6301 | gccacgagag gaagcacatc agcaactggt gaatgattac ttttcagaaa atcctcttta |
| 6361 | cccttccaaa atttttcgtc gaagatttcg tatgtctagg ccactttttc ttcgcatcgt |
| 6421 | tgaggcatta ggccagtggt cagtgtattt cacacaaagg gtggatgctg ttaatcggaa |
| 6481 | aggactcagt ccactgcaaa agtgtactgc agctattcgc cagttggcta ctggtagtgg |
| 6541 | cgcagatgaa ctagatgaat atctgaagat aggagagact acagcaatgg aggcaatgaa |
| 6601 | gaattttgtc aaaggtcttc aagatgtgtt tggtgagagg tatcttaggc gccccactat |
| 6661 | ggaagatacc gaacggcttc tccaacttgg tgagaaacgt ggttttcctg gaatgttcgg |
| 6721 | cagcattgac tgcatgcact ggcattggga aagatgccca gtagcatgga agggtcagtt |
| 6781 | cactcgtgga gatcagaaag tgccaaccct gattcttgag gctgtggcat cgcatgatct |
| 6841 | ttggatttgg catgcatttt ttggagcagc gggttccaac aatgatatca atgtattgaa |
| 6901 | ccaatctact gtatttatca aggagctcaa aggacaagct cctagagtcc agtacatggt |
| 6961 | aaatgggaat caatacaata ctgggtattt tcttgctgat ggaatctacc ctgaatgggc |
| 7021 | agtgtttgtt aagtcaatac gactcccaaa cactgaaaag gagaaattgt atgcagatat |
| 7081 | gcaagaaggg gcaagaaaag atatcgagag agcctttggt gtattgcagc gaagattttg |
| 7141 | catcttaaaa cgaccagctc gtctatatga tcgaggtgta ctgcgagatg ttgttctagc |
| 7201 | ttgcatcata cttcacaata tgatagttga agatgagaag gaaaccagaa ttattgaaga |
| 7261 | agatgcagat gcaaatgtgc ctcctagttc atcaaccgtt caggaacctg agttctctcc |
| 7321 | tgaacagaac acaccatttg atagagtttt agaaaaagat atttctatcc gagatcgagc |
| 7381 | ggctcataac cgacttaaga aagatttggt ggaacacatt tggaataagt ttggtggtgc |
| 7441 | tgcacataga actggaaatt atggcggggg aggtagcgct ccgaagaaga agaggaaggt |
| 7501 | tggcatccac ggggtgccag ctgctgacaa gaagtactcg atcggcctcg atattgggac |
| 7561 | taactctgtt ggctgggccg tgatcaccga cgagtacaag gtgccctcaa agaagttcaa |
| 7621 | ggtcctgggc aacaccgatc ggcattccat caagaagaat ctcattggcg ctctcctgtt |
| 7681 | cgacagcggc gagacggctg aggctacgcg gctcaagcgc accgcccgca ggcggtacac |
| 7741 | gcgcaggaag aatcgcatct gctacctgca ggagattttc tccaacgaga tggcgaaggt |
| 7801 | tgacgattct ttcttccaca ggctggagga gtcattcctc gtggaggagg ataagaagca |
| 7861 | cgagcggcat ccaatcttcg gcaacattgt cgacgaggtt gcctaccacg agaagtaccc |
| 7921 | tacgatctac catctgcgga agaagctcgt ggactccaca gataaggcgg acctccgcct |
| 7981 | gatctacctc gctctggccc acatgattaa gttcaggggc catttcctga tcgaggggga |
| 8041 | tctcaacccg gacaatagcg atgttgacaa gctgttcatc cagctcgtgc agacgtacaa |
| 8101 | ccagctcttc gaggagaacc ccattaatgc gtcaggcgtc gacgcgaagg ctatcctgtc |
| 8161 | cgctaggctc tcgaagtctc ggcgcctcga gaacctgatc gcccagctgc cgggcgagaa |
| 8221 | gaagaacggc ctgttcggga atctcattgc gctcagcctg gggctcacgc ccaacttcaa |
| 8281 | gtcgaatttc gatctcgctg aggacgccaa gctgcagctc tccaaggaca catacgacga |
| 8341 | tgacctggat aacctcctgg cccagatcgg cgatcagtac gcggacctgt tcctcgctgc |
| 8401 | caagaatctg tcggacgcca tcctcctgtc tgatattctc agggtgaaca ccgagattac |
| 8461 | gaaggctccg ctctcagcct ccatgatcaa gcgctacgac gagcaccatc aggatctgac |
| 8521 | cctcctgaag gcgctggtca ggcagcagct ccccgagaag tacaaggaga tcttcttcga |
| 8581 | tcagtcgaag aacggctacg ctgggtacat tgacggcggg gcctctcagg aggagttcta |
| 8641 | caagttcatc aagccgattc tggagaagat ggacggcacg gaggagctgc tggtgaagct |
| 8701 | caatcgcgag gacctcctga ggaagcagcg gacattcgat aacggcagca tcccacacca |
| 8761 | gattcatctc ggggagctgc acgctatcct gaggaggcag gaggacttct accctttcct |
| 8821 | caaggataac cgcgagaaga tcgagaagat tctgactttc aggatcccgt actacgtcgg |
| 8881 | cccactcgct aggggcaact cccgcttcgc ttggatgacc cgcaagtcag aggagacgat |
| 8941 | cacgccgtgg aacttcgagg aggtggtcga caagggcgct agcgctcagt cgttcatcga |
| 9001 | gaggatgacg aatttcgaca agaacctgcc aaatgagaag gtgctcccta agcactcgct |
| 9061 | cctgtacgag tacttcacag tctacaacga gctgactaag gtgaagtatg tgaccgaggg |
| 9121 | catgaggaag ccggctttcc tgtctgggga gcagaagaag gccatcgtgg acctcctgtt |
| 9181 | caagaccaac cggaaggtca cggttaagca gctcaaggag gactacttca agaagattga |
| 9241 | gtgcttcgat tcggtcgaga tctctggcgt tgaggaccgc ttcaacgcct ccctggggac |
| 9301 | ctaccacgat ctcctgaaga tcattaagga taaggacttc ctggacaacg aggagaatga |
| 9361 | ggatatcctc gaggacattg tgctgacact cactctgttc gaggaccggg agatgatcga |
| 9421 | ggagcgcctg aagacttacg cccatctctt cgatgacaag gtcatgaagc agctcaagag |
| 9481 | gaggaggtac accggctggg ggaggctgag caggaagctc atcaacggca ttcgggacaa |
| 9541 | gcagtccggg aagacgatcc tcgacttcct gaagagcgat ggcttcgcga accgcaattt |
| 9601 | catgcagctg attcacgatg acagcctcac attcaaggag gatatccaga aggctcaggt |
| 9661 | gagcggccag ggggactcgc tgcacgagca tatcgcgaac ctcgctggct cgccagctat |
| 9721 | caagaagggg attctgcaga ccgtgaaggt tgtggacgag ctggtgaagg tcatgggcag |
| 9781 | gcacaagcct gagaacatcg tcattgagat ggcccgggag aatcagacca cgcagaaggg |
| 9841 | ccagaagaac tcacgcgaga ggatgaagag gatcgaggag ggcattaagg agctggggtc |
| 9901 | ccagatcctc aaggagcacc cggtggagaa cacgcagctg cagaatgaga agctctacct |
| 9961 | gtactacctc cagaatggcc gcgatatgta tgtggaccag gagctggata ttaacaggct |
| 10021 | cagcgattac gacgtcgatc atatcgttcc acagtcattc ctgaaggatg actccattga |
| 10081 | caacaaggtc ctcaccaggt cggacaagaa ccggggcaag tctgataatg ttccttcaga |
| 10141 | ggaggtcgtt aagaagatga agaactactg gcgccagctc ctgaatgcca agctgatcac |
| 10201 | gcagcggaag ttcgataacc tcacaaaggc tgagaggggc gggctctctg agctggacaa |
| 10261 | ggcgggcttc atcaagaggc agctggtcga gacacggcag atcactaagc acgttgcgca |
| 10321 | gattctcgac tcacggatga acactaagta cgatgagaat gacaagctga tccgcgaggt |
| 10381 | gaaggtcatc accctgaagt caaagctcgt ctccgacttc aggaaggatt tccagttcta |
| 10441 | caaggttcgg gagatcaaca attaccacca tgcccatgac gcgtacctga acgcggtggt |
| 10501 | cggcacagct ctgatcaaga agtacccaaa gctcgagagc gagttcgtgt acggggacta |
| 10561 | caaggtttac gatgtgagga agatgatcgc caagtcggag caggagattg gcaaggctac |
| 10621 | cgccaagtac ttcttctact ctaacattat gaatttcttc aagacagaga tcactctggc |
| 10681 | caatggcgag atccggaagc gccccctcat cgagacgaac ggcgagacgg gggagatcgt |
| 10741 | gtgggacaag ggcagggatt tcgcgaccgt caggaaggtt ctctccatgc cacaagtgaa |
| 10801 | tatcgtcaag aagacagagg tccagactgg cgggttctct aaggagtcaa ttctgcctaa |
| 10861 | gcggaacagc gacaagctca tcgcccgcaa gaaggactgg gatccgaaga agtacggcgg |
| 10921 | gttcgacagc cccactgtgg cctactcggt cctggttgtg gcgaaggttg agaagggcaa |
| 10981 | gtccaagaag ctcaagagcg tgaaggagct gctggggatc acgattatgg agcgctccag |
| 11041 | cttcgagaag aacccgatcg atttcctgga ggcgaagggc tacaaggagg tgaagaagga |
| 11101 | cctgatcatt aagctcccca agtactcact cttcgagctg gagaacggca ggaagcggat |
| 11161 | gctggcttcc gctggcgagc tgcagaaggg gaacgagctg gctctgccgt ccaagtatgt |
| 11221 | gaacttcctc tacctggcct cccactacga gaagctcaag ggcagccccg aggacaacga |
| 11281 | gcagaagcag ctgttcgtcg agcagcacaa gcattacctc gacgagatca ttgagcagat |
| 11341 | ttccgagttc tccaagcgcg tgatcctggc cgacgcgaat ctggataagg tcctctccgc |
| 11401 | gtacaacaag caccgcgaca agccaatcag ggagcaggct gagaatatca ttcatctctt |
| 11461 | caccctgacg aacctcggcg cccctgctgc tttcaagtac ttcgacacaa ctatcgatcg |
| 11521 | caagaggtac acaagcacta aggaggtcct ggacgcgacc ctcatccacc agtcgattac |
| 11581 | cggcctctac gagacgcgca tcgacctgtc tcagctcggg ggcgacaagc ggccagcggc |
| 11641 | gacgaagaag gcggggcagg cgaagaagaa gaagtgataa ttgacattct aatctagagt |
| 11701 | cctgctttaa tgagatatgc gagacgccta tgatcgcatg atatttgctt tcaattctgt |
| 11761 | tgtgcacgtt gtaaaaaacc tgagcatgtg tagctcagat ccttaccgcc ggtttcggtt |
| 11821 | cattctaatg aatatatcac ccgttactat cgtattttta tgaataatat tctccgttca |
| 11881 | atttactgat tgtaccctac tacttatatg tacaatatta aaatgaaaac aatatattgt |
| 11941 | gctgaatagg tttatagcga catctatgat agagcgccac aataacaaac aattgcgttt |
| 12001 | tattattaca aatccaattt taaaaaaagc ggcagaaccg gtcaaaccta aaagactgat |
| 12061 | tacataaatc ttattcaaat ttcaaaagtg ccccaggggc tagtatctac gacacaccga |
| 12121 | gcggcgaact aataacgttc actgaaggga actccggttc cccgccggcg cgcatgggtg |
| 12181 | agattccttg aagttgagta ttggccgtcc gctctaccga aagttacggg caccattcaa |
| 12241 | cccggtccag cacggcggcc gggtaaccga cttgctgccc cgagaattat gcagcatttt |
| 12301 | tttggtgtat gtgggcccca aatgaagtgc aggtcaaacc ttgacagtga cgacaaatcg |
| 12361 | ttgggcgggt ccagggcgaa ttttgcgaca acatgtcgag gctcagcagg acctgcaggc |
| 12421 | atgcaagatc gcgaattcgt aatcatgtca tagctgtttc ctgtgtgaaa ttgttatccg |
| 12481 | ctcacaattc cacacaacat acgagccgga agcataaagt gtaaagcctg gggtgcctaa |
| 12541 | tgagtgagct aactcacatt aattgcgttg cgctcactgc ccgctttcca gtcgggaaac |
| 12601 | ctgtcgtgcc agctgcatta atgaatcggc caacgcgcgg ggagaggcgg tttgcgtatt |
| 12661 | ggctagagca gcttgccaac atggtggagc acgacactct cgtctactcc aagaatatca |
| 12721 | aagatacagt ctcagaagac caaagggcta ttgagacttt tcaacaaagg gtaatatcgg |
| 12781 | gaaacctcct cggattccat tgcccagcta tctgtcactt catcaaaagg acagtagaaa |
| 12841 | aggaaggtgg cacctacaaa tgccatcatt gcgataaagg aaaggctatc gttcaagatg |
| 12901 | cctctgccga cagtggtccc aaagatggac ccccacccac gaggagcatc gtggaaaaag |
| 12961 | aagacgttcc aaccacgtct tcaaagcaag tggattgatg tgataacatg gtggagcacg |
| 13021 | acactctcgt ctactccaag aatatcaaag atacagtctc agaagaccaa agggctattg |
| 13081 | agacttttca acaaagggta atatcgggaa acctcctcgg attccattgc ccagctatct |
| 13141 | gtcacttcat caaaaggaca gtagaaaagg aaggtggcac ctacaaatgc catcattgcg |
| 13201 | ataaaggaaa ggctatcgtt caagatgcct ctgccgacag tggtcccaaa gatggacccc |
| 13261 | cacccacgag gagcatcgtg gaaaaagaag acgttccaac cacgtcttca aagcaagtgg |
| 13321 | attgatgtga tatctccact gacgtaaggg atgacgcaca atcccactat ccttcgcaag |
| 13381 | accttcctct atataaggaa gttcatttca tttggagagg acacgctgaa atcaccagtc |
| 13441 | tctctctaca aatctatctc tctcgagctt tcgcagatcc cggggggcaa tgagatatga |
| 13501 | aaaagcctga actcaccgcg acgtctgtcg agaagtttct gatcgaaaag ttcgacagcg |
| 13561 | tctccgacct gatgcagctc tcggagggcg aagaatctcg tgctttcagc ttcgatgtag |
| 13621 | gagggcgtgg atatgtcctg cgggtaaata gctgcgccga tggtttctac aaagatcgtt |
| 13681 | atgtttatcg gcactttgca tcggccgcgc tcccgattcc ggaagtgctt gacattgggg |
| 13741 | agtttagcga gagcctgacc tattgcatct cccgccgtgc acagggtgtc acgttgcaag |
| 13801 | acctgcctga aaccgaactg cccgctgttc tacaaccggt cgcggaggct atggatgcga |
| 13861 | tcgctgcggc cgatcttagc cagacgagcg ggttcggccc attcggaccg caaggaatcg |
| 13921 | gtcaatacac tacatggcgt gatttcatat gcgcgattgc tgatccccat gtgtatcact |
| 13981 | ggcaaactgt gatggacgac accgtcagtg cgtccgtcgc gcaggctctc gatgagctga |
| 14041 | tgctttgggc cgaggactgc cccgaagtcc ggcacctcgt gcacgcggat ttcggctcca |
| 14101 | acaatgtcct gacggacaat ggccgcataa cagcggtcat tgactggagc gaggcgatgt |
| 14161 | tcggggattc ccaatacgag gtcgccaaca tcttcttctg gaggccgtgg ttggcttgta |
| 14221 | tggagcagca gacgcgctac ttcgagcgga ggcatccgga gcttgcagga tcgccacgac |
| 14281 | tccgggcgta tatgctccgc attggtcttg accaactcta tcagagcttg gttgacggca |
| 14341 | atttcgatga tgcagcttgg gcgcagggtc gatgcgacgc aatcgtccga tccggagccg |
| 14401 | ggactgtcgg gcgtacacaa atcgcccgca gaagcgcggc cgtctggacc gatggctgtg |
| 14461 | tagaagtact cgccgatagt ggaaaccgac gccccagcac tcgtccgagg gcaaagaaat |
| 14521 | agagtagatg ccgaccggat ctgtcgatcg acaagctcga gtttctccat aataatgtgt |
| 14581 | gagtagttcc cagataaggg aattagggtt cctatagggt ttcgctcatg tgttgagcat |
| 14641 | ataagaaacc cttagtatgt atttgtattt gtaaaatact tctatcaata aaatttctaa |
| 14701 | ttcctaaaac caaaatccag tactaaaatc cagatccccc gaattaattc ggcgttaatt |
| 14761 | cagtacatta aaaacgtccg caatgtgtta ttaagttgtc taagcgtcaa tttgtttaca |
| 14821 | ccacaatata tcctgccacc agccagccaa cagctccccg accggcagct cggcacaaaa |
| 14881 | tcaccactcg atacaggcag cccatcagtc cgggacggcg tcagcgggag agccgttgta |
| 14941 | aggcggcaga ctttgctcat gttaccgatg ctattcggaa gaacggcaac taagctgccg |
| 15001 | ggtttgaaac acggatgatc tcgcggaggg tagcatgttg attgtaacga tgacagagcg |
| 15061 | ttgctgccty tgatcaccgc ggtttcaaaa tcggctccgt cgatactatg ttatacgcca |
| 15121 | actttgaaaa caactttgaa aaagctgttt tctggtattt aaggttttag aatgcaagga |
| 15181 | acagtgaatt ggagttcgtc ttgttataat tagcttcttg gggtatcttt aaatactgta |
| 15241 | gaaaagagga aggaaataat aaatggctaa aatgagaata tcaccggaat tgaaaaaact |
| 15301 | gatcgaaaaa taccgctgcg taaaagatac ggaaggaatg tctcctgcta aggtatataa |
| 15361 | gctggtggga gaaaatgaaa acctatattt aaaaatgacg gacagccggt ataaagggac |
| 15421 | cacctatgat gtggaacggg aaaaggacat gatgctatgg ctggaaggaa agctgcctgt |
| 15481 | tccaaaggtc ctgcactttg aacggcatga tggctggagc aatctgctca tgagtgaggc |
| 15541 | cgatggcgtc ctttgctcgg aagagtatga agatgaacaa agccctgaaa agattatcga |
| 15601 | gctgtatgcg gagtgcatca ggctctttca ctccatcgac atatcggatt gtccctatac |
| 15661 | gaatagctta gacagccgct tagccgaatt ggattactta ctgaataacg atctggccga |
| 15721 | tgtggattgc gaaaactggg aagaagacac tccatttaaa gatccgcgcg agctgtatga |
| 15781 | ttttttaaag acggaaaagc ccgaagagga acttgtcttt tcccacggcg acctgggaga |
| 15841 | cagcaacatc tttgtgaaag atggcaaagt aagtggcttt attgatcttg ggagaagcgg |
| 15901 | cagggcggac aagtggtatg acattgcctt ctgcgtccgg tcgatcaggg aggatatcgg |
| 15961 | ggaagaacag tatgtcgagc tattttttga cttactgggg atcaagcctg attgggagaa |
| 16021 | aataaaatat tatattttac tggatgaatt gttttagtac ctagaatgca tgaccaaaat |
| 16081 | cccttaacgt gagttttcgt tccactgagc gtcagacccc gtagaaaaga tcaaaggatc |
| 16141 | ttcttgagat cctttttttc tgcgcgtaat ctgctgcttg caaacaaaaa aaccaccgct |
| 16201 | accagcggtg gtttgtttgc cggatcaaga gctaccaact ctttttccga aggtaactgg |
| 16261 | cttcagcaga gcgcagatac caaatactgt ccttctagtg tagccgtagt taggccacca |
| 16321 | cttcaagaac tctgtagcac cgcctacata cctcgctctg ctaatcctgt taccagtggc |
| 16381 | tgctgccagt ggcggtgtct taccgggttg gactcaagac gatagttacc ggataaggcg |
| 16441 | cagcggtcgg gctgaacggg gggttcgtgc acacagccca gcttggagcg aacgacctac |
| 16501 | accgaactga gatacctaca gcgtgagcta tgagaaagcg ccacgcttcc cgaagggaga |
| 16561 | aaggcggaca ggtatccggt aagcggcagg gtcggaacag gagagcgcac gagggagctt |
| 16621 | ccagggggaa acgcctggta tctttatagt cctgtcgggt ttcgccacct ctgacttgag |
| 16681 | cgtcgatttt tgtgatgctc gtcagggggg cggagcctat ggaaaaacgc cagcaacgcg |
| 16741 | gcctttttac ggttcctggc cttttgctgg ccttttgctc acatgttctt tcctgcgtta |
| 16801 | tcccctgatt ctgtggataa ccgtattacc gcctttgagt gagctgatac cgctcgccgc |
| 16861 | agccgaacga ccgagcgcag cgagtcagtg agcgaggaag cggaagagcg cctgatgcgg |
| 16921 | tattttctcc ttacgcatct gtgcggtatt tcacaccgca tatggtgcac tctcagtaca |
| 16981 | atctgctctg atgccgcata gttaagccag tatacactcc gctatcgcta cgtgactggg |
| 17041 | tcatggctgc gccccgacac ccgccaacac ccgctgacgc gccctgacgg gcttgtctgc |
| 17101 | tcccggcatc cgcttacaga caagctgtga ccgtctccgg gagctgcatg tgtcagaggt |
| 17161 | tttcaccgtc atcaccgaaa cgcgcgaggc agggtgcctt gatgtgggcg ccggcggtcg |
| 17221 | agtggcgacg gcgcggcttg tccgcgccct ggtagattgc ctggccgtag gccagccatt |
| 17281 | tttgagcggc cagcggccgc gataggccga cgcgaagcgg cggggcgtag ggagcgcagc |
| 17341 | gaccgaaggg taggcgcttt ttgcagctct tcggctgtgc gctggccaga cagttatgca |
| 17401 | caggccaggc gggttttaag agttttaata agttttaaag agttttaggc ggaaaaatcg |
| 17461 | ccttttttct cttttatatc agtcacttac atgtgtgacc ggttcccaat gtacggcttt |
| 17521 | gggttcccaa tgtacgggtt ccggttccca atgtacggct ttgggttccc aatgtacgtg |
| 17581 | ctatccacag gaaacagacc ttttcgacct ttttcccctg ctagggcaat ttgccctagc |
| 17641 | atctgctccg tacattagga accggcggat gcttcgccct cgatcaggtt gcggtagcgc |
| 17701 | atgactagga tcgggccagc ctgccccgcc tcctccttca aatcgtactc cggcaggtca |
| 17761 | tttgacccga tcagcttgcg cacggtgaaa cagaacttct tgaactctcc ggcgctgcca |
| 17821 | ctgcgttcgt agatcgtctt gaacaaccat ctggcttctg ccttgcctgc ggcgcggcgt |
| 17881 | gccaggcggt agagaaaacg gccgatgccg ggatcgatca aaaagtaatc ggggtgaacc |
| 17941 | gtcagcacgt ccgggttctt gccttctgtg atctcgcggt acatccaatc agctagctcg |
| 18001 | atctcgatgt actccggccg cccggtttcg ctctttacga tcttgtagcg gctaatcaag |
| 18061 | gcttcaccct cggataccgt caccaggcgg ccgttcttgg ccttcttcgt acgctgcatg |
| 18121 | gcaacgtgcg tggtgtttaa ccgaatgcag gtttctacca ggtcgtcttt ctgctttccg |
| 18181 | ccatcggctc gccggcagaa cttgagtacg tccgcaacgt gtggacggaa cacgcggccg |
| 18241 | ggcttgtctc ccttcccttc ccggtatcgg ttcatggatt cggttagatg ggaaaccgcc |
| 18301 | atcagtacca ggtcgtaatc ccacacactg gccatgccgg ccggccctgc ggaaacctct |
| 18361 | acgtgcccgt ctggaagctc gtagcggatc acctcgccag ctcgtcggtc acgcttcgac |
| 18421 | agacggaaaa cggccacgtc catgatgctg cgactatcgc gggtgcccac gtcatagagc |
| 18481 | atcggaacga aaaaatctgg ttgctcgtcg cccttgggcg gcttcctaat cgacggcgca |
| 18541 | ccggctgccg gcggttgccg ggattctttg cggattcgat cagcggccgc ttgccacgat |
| 18601 | tcaccggggc gtgcttctgc ctcgatgcgt tgccgctggg cggcctgcgc ggccttcaac |
| 18661 | ttctccacca ggtcatcacc cagcgccgcg ccgatttgta ccgggccgga tggtttgcga |
| 18721 | ccgctcacgc cgattcctcg ggcttggggg ttccagtgcc attgcagggc cggcagacaa |
| 18781 | cccagccgct tacgcctggc caaccgcccg ttcctccaca catggggcat tccacggcgt |
| 18841 | cggtgcctgg ttgttcttga ttttccatgc cgcctccttt agccgctaaa attcatctac |
| 18901 | tcatttattc atttgctcat ttactctggt agctgcgcga tgtattcaga tagcagctcg |
| 18961 | gtaatggtct tgccttggcg taccgcgtac atcttcagct tggtgtgatc ctccgccggc |
| 19021 | aactgaaagt tgacccgctt catggctggc gtgtctgcca ggctggccaa cgttgcagcc |
| 19081 | ttgctgctgc gtgcgctcgg acggccggca cttagcgtgt ttgtgctttt gctcattttc |
| 19141 | tctttacctc attaactcaa atgagttttg atttaatttc agcggccagc gcctggacct |
| 19201 | cgcgggcagc gtcgccctcg ggttctgatt caagaacggt tgtgccggcg gcggcagtgc |
| 19261 | ctgggtagct cacgcgctgc gtgatacggg actcaagaat gggcagctcg tacccggcca |
| 19321 | gcgcctcggc aacctcaccg ccgatgcgcg tgcctttgat cgcccgcgac acgacaaagg |
| 19381 | ccgcttgtag ccttccatcc gtgacctcaa tgcgctgctt aaccagctcc accaggtcgg |
| 19441 | cggtggccca tatgtcgtaa gggcttggct gcaccggaat cagcacgaag tcggctgcct |
| 19501 | tgatcgcgga cacagccaag tccgccgcct ggggcgctcc gtcgatcact acgaagtcgc |
| 19561 | gccggccgat ggccttcacg tcgcggtcaa tcgtcgggcg gtcgatgccg acaacggtta |
| 19621 | gcggttgatc ttcccgcacg gccgcccaat cgcgggcact gccctgggga tcggaatcga |
| 19681 | ctaacagaac atcggccccg gcgagttgca gggcgcgggc tagatgggtt gcgatggtcg |
| 19741 | tcttgcctga cccgcctttc tggttaagta cagcgataac cttcatgcgt tccccttgcg |
| 19801 | tatttgttta tttactcatc gcatcatata cgcagcgacc gcatgacgca agctgtttta |
| 19861 | ctcaaataca catcaccttt ttagacggcg gcgctcggtt tcttcagcgg ccaagctggc |
| 19921 | cggccaggcc gccagcttgg catcagacaa accggccagg atttcatgca gccgcacggt |
| 19981 | tgagacgtgc gcgggcggct cgaacacgta cccggccgcg atcatctccg cctcgatctc |
| 20041 | ttcggtaatg aaaaacggtt cgtcctggcc gtcctggtgc ggtttcatgc ttgttcctct |
| 20101 | tggcgttcat tctcggcggc cgccagggcg tcggcctcgg tcaatgcgtc ctcacggaag |
| 20161 | gcaccgcgcc gcctggcctc ggtgggcgtc acttcctcgc tgcgctcaag tgcgcggtac |
| 20221 | agggtcgagc gatgcacgcc aagcagtgca gccgcctctt tcacggtgcg gccttcctgg |
| 20281 | tcgatcagct cgcgggcgtg cgcgatctgt gccggggtga gggtagggcg ggggccaaac |
| 20341 | ttcacgcctc gggccttggc ggcctcgcgc ccgctccggg tgcggtcgat gattagggaa |
| 20401 | cgctcgaact cggcaatgcc ggcgaacacg gtcaacacca tgcggccggc cggcgtggtg |
| 20461 | gtgtcggccc acggctctgc caggctacgc aggcccgcgc cggcctcctg gatgcgctcg |
| 20521 | gcaatgtcca gtaggtcgcg ggtgctgcgg gccaggcggt ctagcctggt cactgtcaca |
| 20581 | acgtcgccag ggcgtaggtg gtcaagcatc ctggccagct ccgggcggtc gcgcctggtg |
| 20641 | ccggtgatct tctcggaaaa cagcttggtg cagccggccg cgtgcagttc ggcccgttgg |
| 20701 | ttggtcaagt cctggtcgtc ggtgctgacg cgggcatagc ccagcaggcc agcggcggcg |
| 20761 | ctcttgttca tggcgtaatg tctccggttc tagtcgcaag tattctactt tatgcgacta |
| 20821 | aaacacgcga caagaaaacg ccaggaaaag ggcagggcgg cagcctgtcg cgtaacttag |
| 20881 | gacttgtgcg acatgtcgtt ttcagaagac ggctgcactg aacgtcagaa gccgactgca |
| 20941 | ctatagcagc ggaggggttg gatcaaagta ctttgatccc gaggggaacc ctgtggttgg |
| 21001 | catgcacata caaatggacg aacggataaa ccttttcacg cccttttaaa tatccgttat |
| 21061 | tctaataaac gctcttttct cttag |

SEQ ID NO: 92. mPing, gRNA, Pong ORF1, Pong ORF2 fused to Cas9

| | |
|---|---|
| LOCUS | The_one_component_tran 21560 bp ds-DNA circular 09-MAR.-2022 |
| DEFINITION | . |
| ACCESSION | pVec1 |
| VERSION | pVec1.1 |
| FEATURES | Location/Qualifiers |
| Agro tDNA cut site | 1 . . . 25 |
| | /label = “RB″ |
| misc_feature | 69 . . . 83 |
| | /label = “TIR″ |
| Transposon | 69 . . . 498 |
| | /label = “mPing″ |
| misc_feature | complement (484 . . . 498) |
| | /label = “TIR″ |
| misc_feature | 729 . . . 1152 |
| | /label = “U6-26promoter″ |
| misc_feature | 1153 . . . 1172 |
| | /label = “gRNA to ACT8 promoter″ |
| misc_feature | 1173 . . . 1248 |
| | /label = “gRNA scaffold″ |
| misc_feature | 1249 . . . 1440 |
| | /label = “U6-26 terminator″ |
| promoter | 1456 . . . 3142 |
| | /label = “Rps5a″ |
| misc_feature | 3179 . . . 4576 |
| | /label = “ORF1″ |
| terminator | 4640 . . . 5365 |
| | /label = “OCS terminator″ |
| promoter | 5548 . . . 6467 |
| | /label = “GmUbi3 Promoter″ |
| misc_feature | 6489 . . . 7934 |
| | /label = “Pong TPase LA″ |
| CDS | 6489 . . . 12149 |
| | /label = “Translation 6489-12149″ |
| misc_feature | 7938 . . . 7952 |
| | /label = “G4S linker″ |
| feature | 7956 . . . 7976 |
| | /label = “SV40 NLS″ |
| misc_feature | 7980 . . . 12149 |
| | /label = “Cas9″ |
| misc_feature | 12102 . . . 12149 |
| | /label = “NLS″ |
| terminator | 12177 . . . 12904 |
| | /label = “OCS Terminator″ |
| promoter | 13155 . . . 13896 |
| | /label = “CaMVd35S promoter″ |
| gene | 13987 . . . 14982 |
| | /label = “hygroB (variant) ″ |
| misc_feature | complement (15600 . . . 15622) |
| | /label = “LB″ |
| gene | 15738 . . . 16532 |
| | /label = “KanR1″ |
| origin | 16603 . . . 17215 |
| | /label = “pBR322 origin″ |

ORIGIN

| | |
|---|---|
| 1 | gtttacccgc caatatatcc tgtcaaacac tgatagtttt gttatatctc cttggatcct |
| 61 | ctagattagg ccagtcacaa tggctagtgt cattgcacgg ctacccaaaa tattatacca |
| 121 | tcttctctca aatgaaatct tttatgaaac aatccccaca gtggaggggt ttcactttga |
| 181 | cgtttccaag actaagcaaa gcatttaatt gatacaagtt gctgggatca tttgtaccca |
| 241 | aaatccggcg cggcgcggga gaatgcggag gtcgcacggc ggaggcggac gcaagagatc |
| 301 | cggtgaatga aacgaatcgg cctcaacggg ggtttcactc tgttaccgag gacttggaaa |
| 361 | cgacgctgac gagtttcacc aggatgaaac tctttccttc tctctcatcc ccatttcatg |
| 421 | caaataatca ttttttattc agtcttaccc ctattaaatg tgcatgacac accagtgaaa |
| 481 | cccccattgt gactggcctt atctagagtc ccccaaactg aaggcgggaa acgacaatct |
| 541 | gatccaagct caagctgctc tagcattcgc cattcaggct gcgcaactgt tgggaagggc |
| 601 | gatcggtgcg ggcctcttcg ctattacgcc agctggcgaa agggggatgt gctgcaaggc |
| 661 | gattaagttg ggtaacgcca gggttttccc agtcacgacg ttgtaaaacg acggccagtg |
| 721 | ccaagcttcg acttgccttc cgcacaatac atcatttctt cttagctttt tttcttcttc |
| 781 | ttcgttcata cagttttttt ttgtttatca gcttacattt tcttgaaccg tagctttcgt |
| 841 | tttcttcttt ttaactttcc attcggagtt tttgtatctt gtttcatagt ttgtcccagg |
| 901 | attagaatga ttaggcatcg aaccttcaag aatttgattg aataaaacat cttcattctt |
| 961 | aagatatgaa gataatcttc aaaaggcccc tgggaatctg aaagaagaga agcaggccca |
| 1021 | tttatatggg aaagaacaat agtatttctt atataggccc atttaagttg aaaacaatct |
| 1081 | tcaaaagtcc cacatcgctt agataagaaa acgaagctga gtttatatac agctagagtc |
| 1141 | gaagtagtga ttgttacagg agtagttcat cggttttaga gctagaaata gcaagttaaa |
| 1201 | ataaggctag tccgttatca acttgaaaaa gtggcaccga gtcggtgctt ttttttgcaa |
| 1261 | aattttccag atcgatttct tcttcctctg ttcttcggcg ttcaatttct ggggttttct |
| 1321 | cttcgttttc tgtaactgaa acctaaaatt tgacctaaaa aaaatctcaa ataatatgat |
| 1381 | tcagtggttt tgtacttttc agttagttga gttttgcagt tccgatgaga taaaccaata |
| 1441 | ccatgttaga gagcgctagt tcgtgagtag atatattact caacttttga ttcgctattt |
| 1501 | gcagtgcacc tgtggcgttc atcacatctt ttgtgacact gtttgcactg gtcattgcta |
| 1561 | ttacaaagga ccttcctgat gttgaaggag atcgaaagta agtaactgca cgcataacca |
| 1621 | ttttctttcc gctctttggc tcaatccatt tgacagtcaa agacaatgtt taaccagctc |
| 1681 | cgtttgatat attgtcttta tgtgtttgtt caagcatgtt tagttaatca tgcctttgat |
| 1741 | tgatcttgaa taggttccaa atatcaaccc tggcaacaaa acttggagtg agaaacattg |
| 1801 | cattcctcgg ttctggactt ctgctagtaa attatgtttc agccatatca ctagctttct |
| 1861 | acatgcctca ggtgaattca tctatttccg tcttaactat ttcggttaat caaagcacga |
| 1921 | acaccattac tgcatgtaga agcttgataa actatcgcca ccaatttatt tttgttgcga |
| 1981 | tattgttact ttcctcagta tgcagctttg aaaagaccaa ccctcttatc ctttaacaat |
| 2041 | gaacaggttt ttagaggtag cttgatgatt cctgcacaty tgatcttggc ttcaggctta |
| 2101 | attttccagg taaagcatta tgagatactc ttatatctct tacatacttt tgagataatg |
| 2161 | cacaagaact tcataactat atgctttagt ttctgcattt gacactgcca aattcattaa |
| 2221 | tctctaatat ctttgttgtt gatctttggt agacatgggt actagaaaaa gcaaactaca |
| 2281 | ccaaggtaaa atacttttgt acaaacataa actcgttatc acggaacatc aatggagtgt |
| 2341 | atatctaacg gagtgtagaa acatttgatt attgcaggaa gctatctcag gatattatcg |
| 2401 | gtttatatgg aatctcttct acgcagagta tctgttattc cccttcctct agctttcaat |
| 2461 | ttcatggtga ggatatgcag ttttctttgt atatcattct tcttcttctt tgtagcttgg |
| 2521 | agtcaaaatc ggttccttca tgtacataca tcaaggatat gtccttctga atttttatat |
| 2581 | cttgcaataa aaatgcttgt accaattgaa acaccagctt tttgagttct atgatcactg |
| 2641 | acttggttct aaccaaaaaa aaaaaaatgt ttaatttaca tatctaaaag taggtttagg |
| 2701 | gaaacctaaa cagtaaaata tttgtatatt attcgaattt cactcatcat aaaaacttaa |
| 2761 | attgcaccat aaaattttgt tttactatta atgatgtaat ttgtgtaact taagataaaa |
| 2821 | ataatattcc gtaagttaac cggctaaaac cacgtataaa ccagggaacc tgttaaaccg |
| 2881 | gttctttact ggataaagaa atgaaagccc atgtagacag ctccattaga gcccaaaccc |
| 2941 | taaatttctc atctatataa aaggagtgac attagggttt ttgttcgtcc tcttaaagct |
| 3001 | tctcgttttc tctgccgtct ctctcattcg cgcgacgcaa acgatcttca ggtgatcttc |
| 3061 | tttctccaaa tcctctctca taactctgat ttcgtactty tgtatttgag ctcacgctct |
| 3121 | gtttctctca ccacagccgg attcgagatc acaagtttgt acaaaaaagc aggcttccat |
| 3181 | ggatccgtcg ccggccgtgg atccgtcgcc ggccgtggat ccgtcgccgg ctgctgaaac |
| 3241 | ccggcggcgt gcaaccggga aaggaggcaa acagcgcggg ggcaagcaac taggattgaa |
| 3301 | gaggccgccg ccgatttctg tcccggccac cccgcctcct gctgcgacgt cttcatcccc |
| 3361 | tgctgcgccg acggccatcc caccacgacc accgcaatct tcgccgattt tcgtccccga |
| 3421 | ttcgccgaat ccgtcaccgg ctgcgccgac ctcctctctt gcttcgggga catcgacggc |
| 3481 | aaggccaccg caaccacaag gaggaggatg gggaccaaca tcgaccattt ccccaaactt |
| 3541 | tgcatctttc tttggaaacc aacaagaccc aaattcatgt ttggtcaggg gttatcctcc |
| 3601 | aggagggttt gtcaatttta ttcaacaaaa ttgtccgccg cagccacaac agcaaggtga |
| 3661 | aaattttcat ttcgttggtc acaatatggg gttcaaccca atatctccac agccaccaag |
| 3721 | tgcctacgga acaccaacac cccaagctac gaaccaaggc acttcaacaa acattatgat |
| 3781 | tgatgaagag gacaacaatg atgacagtag ggcagcaaag aaaagatgga ctcatgaaga |
| 3841 | ggaagagaga ctggccagtg cttggttgaa tgcttctaaa gactcaattc atgggaatga |
| 3901 | taagaaaggt gatacatttt ggaaggaagt cactgatgaa tttaacaaga aagggaatgg |
| 3961 | aaaacgtagg agggaaatta accaactgaa ggttcactgg tcaaggttga agtcagcgat |
| 4021 | ctctgagttc aatgactatt ggagtacggt tactcaaatg catacaagcg gatactcaga |
| 4081 | cgacatgctt gagaaagagg cacagaggct gtatgcaaac aggtttggaa aaccttttgc |
| 4141 | gttggtccat tggtggaaga tactcaaaag agagcccaaa tggtgtgctc agtttgaaaa |
| 4201 | gaggaaaagg aagagcgaaa tggatgctgt tccagaacag cagaaacgtc ctattggtag |
| 4261 | agaagcagca aagtctgagc gcaaaagaaa gcgcaagaaa gaaaatgtta tggaaggcat |
| 4321 | tgtcctccta ggggacaatg tccagaaaat tatcaaagtg acgcaagatc ggaagctgga |
| 4381 | gcgtgagaag gtcactgaag cacagattca catttcaaac gtaaatttga aggcagcaga |
| 4441 | acagcaaaaa gaagcaaaga tgtttgaggt atacaattcc ctgctcactc aagatacaag |
| 4501 | taacatgtct gaagaacaga aggctcgccg agacaaggca ttacaaaagc tggaggaaaa |
| 4561 | gttatttgct gactagtgac ccagctttct tgtacaaagt ggtgcctagg tgagtctaga |
| 4621 | gagttgatta agacccggga ctggtcccta gagtcctgct ttaatgagat atgcgagacg |
| 4681 | cctatgatcg catgatattt gctttcaatt ctgttgtgca cgttgtaaaa aacctgagca |
| 4741 | tgtgtagctc agatccttac cgccggtttc ggttcattct aatgaatata tcacccgtta |
| 4801 | ctatcgtatt tttatgaata atattctccg ttcaatttac tgattgtacc ctactactta |
| 4861 | tatgtacaat attaaaatga aaacaatata ttgtgctgaa taggtttata gcgacatcta |
| 4921 | tgatagagcg ccacaataac aaacaattgc gttttattat tacaaatcca attttaaaaa |
| 4981 | aagcggcaga accggtcaaa cctaaaagac tgattacata aatcttattc aaatttcaaa |
| 5041 | agtgccccag gggctagtat ctacgacaca ccgagcggcg aactaataac gctcactgaa |
| 5101 | gggaactccg gttccccgcc ggcgcgcatg ggtgagattc cttgaagttg agtattggcc |
| 5161 | gtccgctcta ccgaaagtta cgggcaccat tcaacccggt ccagcacggc ggccgggtaa |
| 5221 | ccgacttgct gccccgagaa ttatgcagca tttttttggt gtatgtgggc cccaaatgaa |
| 5281 | gtgcaggtca aaccttgaca gtgacgacaa atcgttgggc gggtccaggg cgaattttgc |
| 5341 | gacaacatgt cgaggctcag caggacctgc aggcatgcaa gcttggcact ggccgtcgtt |
| 5401 | ttacaacgtc gtgactggga aaaccctggc gttacccaac ttaatcgcct tgcagcacat |
| 5461 | ccccctttcg ccagctggcg taatagcgaa gaggcccgca ccgatcgccc ttcccaacag |
| 5521 | ttgcgcagcc tgaatggcga atgctagagc agcttgagct tggatcagat tgtcgtttcc |
| 5581 | cgccttcagt ttcttgaagg tgcatgtgac tccgtcaaga ttacgaaacc gccaactacc |
| 5641 | acgcaaattg caattctcaa tttcctagaa ggactctccg aaaatgcatc caataccaaa |
| 5701 | tattacccgt gtcataggca ccaagtgaca ccatacatga acacgcgtca caatatgact |
| 5761 | ggagaagggt tccacacctt atgctataaa acgccccaca cccctcctcc ttccttcgca |
| 5821 | gttcaattcc aatatattcc attctctctg tgtatttccc tacctctccc ttcaaggtta |
| 5881 | gtcgatttct tctgtttttc ttcttcgttc tttccatgaa ttgtgtatgt tctttgatca |
| 5941 | atacgatgtt gatttgattg tgttttgttt ggtttcatcg atcttcaatt ttcataatca |
| 6001 | gattcagctt ttattatctt tacaacaacg tccttaattt gatgattctt taatcgtaga |
| 6061 | tttgctctaa ttagagcttt ttcatgtcag atccctttac aacaagcctt aattgttgat |
| 6121 | tcattaatcg tagattaggg cttttttcat tgattacttc agatccgtta aacgtaacca |
| 6181 | tagatcaggg ctttttcatg aattacttca gatccgttaa acaacagcct tattttttat |
| 6241 | acttctgtgg tttttcaaga aattgttcag atccgttgac aaaaagcctt attcgttgat |
| 6301 | tctatatcgt ttttcgagag atattgctca gatctgttag caactgcctt gtttgttgat |
| 6361 | tctattgccg tggattaggg ttttttttca cgagattgct tcagatccgt acttaagatt |
| 6421 | acgtaatgga ttttgattct gatttatctg tgattgttga ctcgacaggt accttcaaac |
| 6481 | ggcgcgccat gcagagttta gccatctctc tactcctctc agaaactcat tccctctttt |
| 6541 | ctcatacgaa gacctcctcc cttttatctt tactgtttct ctcttcttca aagatgtctg |
| 6601 | agcaaaatac tgatggaagt caagttccag tgaacttgtt ggatgagttc ctggctgagg |
| 6661 | atgagatcat agatgatctt ctcactgaag ccacggtggt agtacagtcc actatagaag |
| 6721 | gtcttcaaaa cgaggcttct gaccatcgac atcatccgag gaagcacatc aagaggccac |
| 6781 | gagaggaagc acatcagcaa ctggtgaatg attacttttc agaaaatcct ctttaccctt |
| 6841 | ccaaaatttt tcgtcgaaga tttcgtatgt ctaggccact ttttcttcgc atcgttgagg |
| 6901 | cattaggcca gtggtcagtg tatttcacac aaagggtgga tgctgttaat cggaaaggac |
| 6961 | tcagtccact gcaaaagtgt actgcagcta ttcgccagtt ggctactggt agtggcgcag |
| 7021 | atgaactaga tgaatatctg aagataggag agactacagc aatggaggca atgaagaatt |
| 7081 | ttgtcaaagg tcttcaagat gtgtttggtg agaggtatct taggcgcccc actatggaag |
| 7141 | ataccgaacg gcttctccaa cttggtgaga aacgtggttt tcctggaatg ttcggcagca |
| 7201 | ttgactgcat gcactggcat tgggaaagat gcccagtagc atggaagggt cagttcactc |
| 7261 | gtggagatca gaaagtgcca accctgattc ttgaggctgt ggcatcgcat gatctttgga |
| 7321 | tttggcatgc attttttgga gcagcgggtt ccaacaatga tatcaatgta ttgaaccaat |
| 7381 | ctactgtatt tatcaaggag ctcaaaggac aagctcctag agtccagtac atggtaaatg |
| 7441 | ggaatcaata caatactggg tattttcttg ctgatggaat ctaccctgaa tgggcagtgt |
| 7501 | ttgttaagtc aatacgactc ccaaacactg aaaaggagaa attgtatgca gatatgcaag |
| 7561 | aaggggcaag aaaagatatc gagagagcct ttggtgtatt gcagcgaaga ttttgcatct |
| 7621 | taaaacgacc agctcgtcta tatgatcgag gtgtactgcg agatgttgtt ctagcttgca |
| 7681 | tcatacttca caatatgata gttgaagatg agaaggaaac cagaattatt gaagaagatg |
| 7741 | cagatgcaaa tgtgcctcct agttcatcaa ccgttcagga acctgagttc tctcctgaac |
| 7801 | agaacacacc atttgataga gttttagaaa aagatatttc tatccgagat cgagcggctc |
| 7861 | ataaccgact taagaaagat ttggtggaac acatttggaa taagtttggt ggtgctgcac |
| 7921 | atagaactgg aaattatggc gggggaggta gcgctccgaa gaagaagagg aaggttggca |
| 7981 | tccacggggt gccagctgct gacaagaagt actcgatcgg cctcgatatt gggactaact |
| 8041 | ctgttggctg ggccgtgatc accgacgagt acaaggtgcc ctcaaagaag ttcaaggtcc |
| 8101 | tgggcaacac cgatcggcat tccatcaaga agaatctcat tggcgctctc ctgttcgaca |
| 8161 | gcggcgagac ggctgaggct acgcggctca agcgcaccgc ccgcaggcgg tacacgcgca |
| 8221 | ggaagaatcg catctgctac ctgcaggaga ttttctccaa cgagatggcg aaggttgacg |
| 8281 | attctttctt ccacaggctg gaggagtcat tcctcgtgga ggaggataag aagcacgagc |
| 8341 | ggcatccaat cttcggcaac attgtcgacg aggttgccta ccacgagaag taccctacga |
| 8401 | tctaccatct gcggaagaag ctcgtggact ccacagataa ggcggacctc cgcctgatct |
| 8461 | acctcgctct ggcccacatg attaagttca ggggccattt cctgatcgag ggggatctca |
| 8521 | acccggacaa tagcgatgtt gacaagctgt tcatccagct cgtgcagacg tacaaccagc |
| 8581 | tcttcgagga gaaccccatt aatgcgtcag gcgtcgacgc gaaggctatc ctgtccgcta |
| 8641 | ggctctcgaa gtctcggcgc ctcgagaacc tgatcgccca gctgccgggc gagaagaaga |
| 8701 | acggcctgtt cgggaatctc attgcgctca gcctggggct cacgcccaac ttcaagtcga |
| 8761 | atttcgatct cgctgaggac gccaagctgc agctctccaa ggacacatac gacgatgacc |
| 8821 | tggataacct cctggcccag atcggcgatc agtacgcgga cctgttcctc gctgccaaga |
| 8881 | atctgtcgga cgccatcctc ctgtctgata ttctcagggt gaacaccgag attacgaagg |
| 8941 | ctccgctctc agcctccatg atcaagcgct acgacgagca ccatcaggat ctgaccctcc |
| 9001 | tgaaggcgct ggtcaggcag cagctccccg agaagtacaa ggagatcttc ttcgatcagt |
| 9061 | cgaagaacgg ctacgctggg tacattgacg gcggggcctc tcaggaggag ttctacaagt |
| 9121 | tcatcaagcc gattctggag aagatggacg gcacggagga gctgctggtg aagctcaatc |
| 9181 | gcgaggacct cctgaggaag cagcggacat tcgataacgg cagcatccca caccagattc |
| 9241 | atctcgggga gctgcacgct atcctgagga ggcaggagga cttctaccct ttcctcaagg |
| 9301 | ataaccgcga gaagatcgag aagattctga ctttcaggat cccgtactac gtcggcccac |
| 9361 | tcgctagggg caactcccgc ttcgcttgga tgacccgcaa gtcagaggag acgatcacgc |
| 9421 | cgtggaactt cgaggaggtg gtcgacaagg gcgctagcgc tcagtcgttc atcgagagga |
| 9481 | tgacgaattt cgacaagaac ctgccaaatg agaaggtgct ccctaagcac tcgctcctgt |
| 9541 | acgagtactt cacagtctac aacgagctga ctaaggtgaa gtatgtgacc gagggcatga |
| 9601 | ggaagccggc tttcctgtct ggggagcaga agaaggccat cgtggacctc ctgttcaaga |
| 9661 | ccaaccggaa ggtcacggtt aagcagctca aggaggacta cttcaagaag attgagtgct |
| 9721 | tcgattcggt cgagatctct ggcgttgagg accgcttcaa cgcctccctg gggacctacc |
| 9781 | acgatctcct gaagatcatt aaggataagg acttcctgga caacgaggag aatgaggata |
| 9841 | tcctcgagga cattgtgctg acactcactc tgttcgagga ccgggagatg atcgaggagc |
| 9901 | gcctgaagac ttacgcccat ctcttcgatg acaaggtcat gaagcagctc aagaggagga |
| 9961 | ggtacaccgg ctgggggagg ctgagcagga agctcatcaa cggcattcgg gacaagcagt |
| 10021 | ccgggaagac gatcctcgac ttcctgaaga gcgatggctt cgcgaaccgc aatttcatgc |
| 10081 | agctgattca cgatgacagc ctcacattca aggaggatat ccagaaggct caggtgagcg |
| 10141 | gccaggggga ctcgctgcac gagcatatcg cgaacctcgc tggctcgcca gctatcaaga |
| 10201 | aggggattct gcagaccgtg aaggttgtgg acgagctggt gaaggtcatg ggcaggcaca |
| 10261 | agcctgagaa catcgtcatt gagatggccc gggagaatca gaccacgcag aagggccaga |
| 10321 | agaactcacg cgagaggatg aagaggatcg aggagggcat taaggagctg gggtcccaga |
| 10381 | tcctcaagga gcacccggtg gagaacacgc agctgcagaa tgagaagctc tacctgtact |
| 10441 | acctccagaa tggccgcgat atgtatgtgg accaggagct ggatattaac aggctcagcg |
| 10501 | attacgacgt cgatcatatc gttccacagt cattcctgaa ggatgactcc attgacaaca |
| 10561 | aggtcctcac caggtcggac aagaaccggg gcaagtctga taatgttcct tcagaggagg |
| 10621 | tcgttaagaa gatgaagaac tactggcgcc agctcctgaa tgccaagctg atcacgcagc |
| 10681 | ggaagttcga taacctcaca aaggctgaga ggggggggct ctctgagctg gacaaggcgg |
| 10741 | gcttcatcaa gaggcagctg gtcgagacac ggcagatcac taagcacgtt gcgcagattc |
| 10801 | tcgactcacg gatgaacact aagtacgatg agaatgacaa gctgatccgc gaggtgaagg |
| 10861 | tcatcaccct gaagtcaaag ctcgtctccg acttcaggaa ggatttccag ttctacaagg |
| 10921 | ttcgggagat caacaattac caccatgccc atgacgcgta cctgaacgcg gtggtcggca |
| 10981 | cagctctgat caagaagtac ccaaagctcg agagcgagtt cgtgtacggg gactacaagg |
| 11041 | tttacgatgt gaggaagatg atcgccaagt cggagcagga gattggcaag gctaccgcca |
| 11101 | agtacttctt ctactctaac attatgaatt tcttcaagac agagatcact ctggccaatg |
| 11161 | gcgagatccg gaagcgcccc ctcatcgaga cgaacggcga gacgggggag atcgtgtggg |
| 11221 | acaagggcag ggatttcgcg accgtcagga aggttctctc catgccacaa gtgaatatcg |
| 11281 | tcaagaagac agaggtccag actggcgggt tctctaagga gtcaattctg cctaagcgga |
| 11341 | acagcgacaa gctcatcgcc cgcaagaagg actgggatcc gaagaagtac ggcgggttcg |
| 11401 | acagccccac tgtggcctac tcggtcctgg ttgtggcgaa ggttgagaag ggcaagtcca |
| 11461 | agaagctcaa gagcgtgaag gagctgctgg ggatcacgat tatggagcgc tccagcttcg |
| 11521 | agaagaaccc gatcgatttc ctggaggcga agggctacaa ggaggtgaag aaggacctga |
| 11581 | tcattaagct ccccaagtac tcactcttcg agctggagaa cggcaggaag cggatgctgg |
| 11641 | cttccgctgg cgagctgcag aaggggaacg agctggctct gccgtccaag tatgtgaact |
| 11701 | tcctctacct ggcctcccac tacgagaagc tcaagggcag ccccgaggac aacgagcaga |
| 11761 | agcagctgtt cgtcgagcag cacaagcatt acctcgacga gatcattgag cagatttccg |
| 11821 | agttctccaa gcgcgtgatc ctggccgacg cgaatctgga taaggtcctc tccgcgtaca |
| 11881 | acaagcaccg cgacaagcca atcagggagc aggctgagaa tatcattcat ctcttcaccc |
| 11941 | tgacgaacct cggcgcccct gctgctttca agtacttcga cacaactatc gatcgcaaga |
| 12001 | ggtacacaag cactaaggag gtcctggacg cgaccctcat ccaccagtcg attaccggcc |
| 12061 | tctacgagac gcgcatcgac ctgtctcagc tcgggggcga caagcggcca gcggcgacga |
| 12121 | agaaggcggg gcaggcgaag aagaagaagt gataattgac attctaatct agagtcctgc |
| 12181 | tttaatgaga tatgcgagac gcctatgatc gcatgatatt tgctttcaat tctgttgtgc |
| 12241 | acgttgtaaa aaacctgagc atgtgtagct cagatcctta ccgccggttt cggttcattc |
| 12301 | taatgaatat atcacccgtt actatcgtat ttttatgaat aatattctcc gttcaattta |
| 12361 | ctgattgtac cctactactt atatgtacaa tattaaaatg aaaacaatat attgtgctga |
| 12421 | ataggtttat agcgacatct atgatagagc gccacaataa caaacaattg cgttttatta |
| 12481 | ttacaaatcc aattttaaaa aaagcggcag aaccggtcaa acctaaaaga ctgattacat |
| 12541 | aaatcttatt caaatttcaa aagtgcccca ggggctagta tctacgacac accgagcggc |
| 12601 | gaactaataa cgttcactga agggaactcc ggttccccgc cggcgcgcat gggtgagatt |
| 12661 | ccttgaagtt gagtattggc cgtccgctct accgaaagtt acgggcacca ttcaacccgg |
| 12721 | tccagcacgg cggccgggta accgacttgc tgccccgaga attatgcagc atttttttgg |
| 12781 | tgtatgtggg ccccaaatga agtgcaggtc aaaccttgac agtgacgaca aatcgttggg |
| 12841 | cgggtccagg gcgaattttg cgacaacatg tcgaggctca gcaggacctg caggcatgca |
| 12901 | agatcgcgaa ttcgtaatca tgtcatagct gtttcctgtg tgaaattgtt atccgctcac |
| 12961 | aattccacac aacatacgag ccggaagcat aaagtgtaaa gcctggggtg cctaatgagt |
| 13021 | gagctaactc acattaattg cgttgcgctc actgcccgct ttccagtcgg gaaacctgtc |
| 13081 | gtgccagctg cattaatgaa tcggccaacg cgcggggaga ggcggtttgc gtattggcta |
| 13141 | gagcagcttg ccaacatggt ggagcacgac actctcgtct actccaagaa tatcaaagat |
| 13201 | acagtctcag aagaccaaag ggctattgag acttttcaac aaagggtaat atcgggaaac |
| 13261 | ctcctcggat tccattgccc agctatctgt cacttcatca aaaggacagt agaaaaggaa |
| 13321 | ggtggcacct acaaatgcca tcattgcgat aaaggaaagg ctatcgttca agatgcctct |
| 13381 | gccgacagtg gtcccaaaga tggaccccca cccacgagga gcatcgtgga aaaagaagac |
| 13441 | gttccaacca cgtcttcaaa gcaagtggat tgatgtgata acatggtgga gcacgacact |
| 13501 | ctcgtctact ccaagaatat caaagataca gtctcagaag accaaagggc tattgagact |
| 13561 | tttcaacaaa gggtaatatc gggaaacctc ctcggattcc attgcccagc tatctgtcac |
| 13621 | ttcatcaaaa ggacagtaga aaaggaaggt ggcacctaca aatgccatca ttgcgataaa |
| 13681 | ggaaaggcta tcgttcaaga tgcctctgcc gacagtggtc ccaaagatgg acccccaccc |
| 13741 | acgaggagca tcgtggaaaa agaagacgtt ccaaccacgt cttcaaagca agtggattga |
| 13801 | tgtgatatct ccactgacgt aagggatgac gcacaatccc actatccttc gcaagacctt |
| 13861 | cctctatata aggaagttca tttcatttgg agaggacacg ctgaaatcac cagtctctct |
| 13921 | ctacaaatct atctctctcg agctttcgca gatcccgggg ggcaatgaga tatgaaaaag |
| 13981 | cctgaactca ccgcgacgtc tgtcgagaag tttctgatcg aaaagttcga cagcgtctcc |
| 14041 | gacctgatgc agctctcgga gggcgaagaa tctcgtgctt tcagcttcga tgtaggaggg |
| 14101 | cgtggatatg tcctgcgggt aaatagctgc gccgatggtt tctacaaaga tcgttatgtt |
| 14161 | tatcggcact ttgcatcggc cgcgctcccg attccggaag tgcttgacat tggggagttt |
| 14221 | agcgagagcc tgacctattg catctcccgc cgtgcacagg gtgtcacgtt gcaagacctg |
| 14281 | cctgaaaccg aactgcccgc tgttctacaa ccggtcgcgg aggctatgga tgcgatcgct |
| 14341 | gcggccgatc ttagccagac gagcgggttc ggcccattcg gaccgcaagg aatcggtcaa |
| 14401 | tacactacat ggcgtgattt catatgcgcg attgctgatc cccatgtgta tcactggcaa |
| 14461 | actgtgatgg acgacaccgt cagtgcgtcc gtcgcgcagg ctctcgatga gctgatgctt |
| 14521 | tgggccgagg actgccccga agtccggcac ctcgtgcacg cggatttcgg ctccaacaat |
| 14581 | gtcctgacgg acaatggccg cataacagcg gtcattgact ggagcgaggc gatgttcggg |
| 14641 | gattcccaat acgaggtcgc caacatcttc ttctggaggc cgtggttggc ttgtatggag |
| 14701 | cagcagacgc gctacttcga gcggaggcat ccggagcttg caggatcgcc acgactccgg |
| 14761 | gcgtatatgc tccgcattgg tcttgaccaa ctctatcaga gcttggttga cggcaatttc |
| 14821 | gatgatgcag cttgggcgca gggtcgatgc gacgcaatcg tccgatccgg agccgggact |
| 14881 | gtcgggcgta cacaaatcgc ccgcagaagc gcggccgtct ggaccgatgg ctgtgtagaa |
| 14941 | gtactcgccg atagtggaaa ccgacgcccc agcactcgtc cgagggcaaa gaaatagagt |
| 15001 | agatgccgac cggatctgtc gatcgacaag ctcgagtttc tccataataa tgtgtgagta |
| 15061 | gttcccagat aagggaatta gggttcctat agggtttcgc tcatgtgttg agcatataag |
| 15121 | aaacccttag tatgtatttg tatttgtaaa atacttctat caataaaatt tctaattcct |
| 15181 | aaaaccaaaa tccagtacta aaatccagat cccccgaatt aattcggcgt taattcagta |
| 15241 | cattaaaaac gtccgcaatg tgttattaag ttgtctaagc gtcaatttgt ttacaccaca |
| 15301 | atatatcctg ccaccagcca gccaacagct ccccgaccgg cagctcggca caaaatcacc |
| 15361 | actcgataca ggcagcccat cagtccggga cggcgtcagc gggagagccg ttgtaaggcg |
| 15421 | gcagactttg ctcatgttac cgatgctatt cggaagaacg gcaactaagc tgccgggttt |
| 15481 | gaaacacgga tgatctcgcg gagggtagca tgttgattgt aacgatgaca gagcgttgct |
| 15541 | gcctgtgatc accgcggttt caaaatcggc tccgtcgata ctatgttata cgccaacttt |
| 15601 | gaaaacaact ttgaaaaagc tgttttctgg tatttaaggt tttagaatgc aaggaacagt |
| 15661 | gaattggagt tcgtcttgtt ataattagct tcttggggta tctttaaata ctgtagaaaa |
| 15721 | gaggaaggaa ataataaatg gctaaaatga gaatatcacc ggaattgaaa aaactgatcg |
| 15781 | aaaaataccg ctgcgtaaaa gatacggaag gaatgtctcc tgctaaggta tataagctgg |
| 15841 | tgggagaaaa tgaaaaccta tatttaaaaa tgacggacag ccggtataaa gggaccacct |
| 15901 | atgatgtgga acgggaaaag gacatgatgc tatggctgga aggaaagctg cctgttccaa |
| 15961 | aggtcctgca ctttgaacgg catgatggct ggagcaatct gctcatgagt gaggccgatg |
| 16021 | gcgtcctttg ctcggaagag tatgaagatg aacaaagccc tgaaaagatt atcgagctgt |
| 16081 | atgcggagtg catcaggctc tttcactcca tcgacatatc ggattgtccc tatacgaata |
| 16141 | gcttagacag ccgcttagcc gaattggatt acttactgaa taacgatctg gccgatgtgg |
| 16201 | attgcgaaaa ctgggaagaa gacactccat ttaaagatcc gcgcgagctg tatgattttt |
| 16261 | taaagacgga aaagcccgaa gaggaacttg tcttttccca cggcgacctg ggagacagca |
| 16321 | acatctttgt gaaagatggc aaagtaagtg gctttattga tcttgggaga agcggcaggg |
| 16381 | cggacaagtg gtatgacatt gccttctgcg tccggtcgat cagggaggat atcggggaag |
| 16441 | aacagtatgt cgagctattt tttgacttac tggggatcaa gcctgattgg gagaaaataa |
| 16501 | aatattatat tttactggat gaattgtttt agtacctaga atgcatgacc aaaatccctt |
| 16561 | aacgtgagtt ttcgttccac tgagcgtcag accccgtaga aaagatcaaa ggatcttctt |
| 16621 | gagatccttt ttttctgcgc gtaatctgct gcttgcaaac aaaaaaacca ccgctaccag |
| 16681 | cggtggtttg tttgccggat caagagctac caactctttt tccgaaggta actggcttca |
| 16741 | gcagagcgca gataccaaat actgtccttc tagtgtagcc gtagttaggc caccacttca |
| 16801 | agaactctgt agcaccgcct acatacctcg ctctgctaat cctgttacca gtggctgctg |
| 16861 | ccagtggcgg tgtcttaccg ggttggactc aagacgatag ttaccggata aggcgcagcg |
| 16921 | gtcgggctga acggggggtt cgtgcacaca gcccagcttg gagcgaacga cctacaccga |
| 16981 | actgagatac ctacagcgtg agctatgaga aagcgccacg cttcccgaag ggagaaaggc |
| 17041 | ggacaggtat ccggtaagcg gcagggtcgg aacaggagag cgcacgaggg agcttccagg |
| 17101 | gggaaacgcc tggtatcttt atagtcctgt cgggtttcgc cacctctgac ttgagcgtcg |
| 17161 | atttttgtga tgctcgtcag gggggcggag cctatggaaa aacgccagca acgcggcctt |
| 17221 | tttacggttc ctggcctttt gctggccttt tgctcacatg ttctttcctg cgttatcccc |
| 17281 | tgattctgtg gataaccgta ttaccgcctt tgagtgagct gataccgctc gccgcagccg |
| 17341 | aacgaccgag cgcagcgagt cagtgagcga ggaagcggaa gagcgcctga tgcggtattt |
| 17401 | tctccttacg catctgtgcg gtatttcaca ccgcatatgg tgcactctca gtacaatctg |
| 17461 | ctctgatgcc gcatagttaa gccagtatac actccgctat cgctacgtga ctgggtcatg |
| 17521 | gctgcgcccc gacacccgcc aacacccgct gacgcgccct gacgggcttg tctgctcccg |
| 17581 | gcatccgctt acagacaagc tgtgaccgtc tccgggagct gcatgtgtca gaggttttca |
| 17641 | ccgtcatcac cgaaacgcgc gaggcagggt gccttgatgt gggcgccggc ggtcgagtgg |
| 17701 | cgacggcgcg gcttgtccgc gccctggtag attgcctggc cgtaggccag ccatttttga |
| 17761 | gcggccagcg gccgcgatag gccgacgcga agcggcgggg cgtagggagc gcagcgaccg |
| 17821 | aagggtaggc gctttttgca gctcttcggc tgtgcgctgg ccagacagtt atgcacaggc |
| 17881 | caggcgggtt ttaagagttt taataagttt taaagagttt taggcggaaa aatcgccttt |
| 17941 | tttctctttt atatcagtca cttacatgtg tgaccggttc ccaatgtacg gctttgggtt |
| 18001 | cccaatgtac gggttccggt tcccaatgta cggctttggg ttcccaatgt acgtgctatc |
| 18061 | cacaggaaac agaccttttc gacctttttc ccctgctagg gcaatttgcc ctagcatctg |
| 18121 | ctccgtacat taggaaccgg cggatgcttc gccctcgatc aggttgcggt agcgcatgac |
| 18181 | taggatcggg ccagcctgcc ccgcctcctc cttcaaatcg tactccggca ggtcatttga |
| 18241 | cccgatcagc ttgcgcacgg tgaaacagaa cttcttgaac tctccggcgc tgccactgcg |
| 18301 | ttcgtagatc gtcttgaaca accatctggc ttctgccttg cctgcggcgc ggcgtgccag |
| 18361 | gcggtagaga aaacggccga tgccgggatc gatcaaaaag taatcggggt gaaccgtcag |
| 18421 | cacgtccggg ttcttgcctt ctgtgatctc gcggtacatc caatcagcta gctcgatctc |
| 18481 | gatgtactcc ggccgcccgg tttcgctctt tacgatcttg tagcggctaa tcaaggcttc |
| 18541 | accctcggat accgtcacca ggcggccgtt cttggccttc ttcgtacgct gcatggcaac |
| 18601 | gtgcgtggtg tttaaccgaa tgcaggtttc taccaggtcg tctttctgct ttccgccatc |
| 18661 | ggctcgccgg cagaacttga gtacgtccgc aacgtgtgga cggaacacgc ggccgggctt |
| 18721 | gtctcccttc ccttcccggt atcggttcat ggattcggtt agatgggaaa ccgccatcag |
| 18781 | taccaggtcg taatcccaca cactggccat gccggccggc cctgcggaaa cctctacgtg |
| 18841 | cccgtctgga agctcgtagc ggatcacctc gccagctcgt cggtcacgct tcgacagacg |
| 18901 | gaaaacggcc acgtccatga tgctgcgact atcgcgggtg cccacgtcat agagcatcgg |
| 18961 | aacgaaaaaa tctggttgct cgtcgccctt gggcggcttc ctaatcgacg gcgcaccggc |
| 19021 | tgccggcggt tgccgggatt ctttgcggat tcgatcagcg gccgcttgcc acgattcacc |
| 19081 | ggggcgtgct tctgcctcga tgcgttgccg ctgggcggcc tgcgcggcct tcaacttctc |
| 19141 | caccaggtca tcacccagcg ccgcgccgat ttgtaccggg ccggatggtt tgcgaccgct |
| 19201 | cacgccgatt cctcgggctt gggggttcca gtgccattgc agggccggca gacaacccag |
| 19261 | ccgcttacgc ctggccaacc gcccgttcct ccacacatgg ggcattccac ggcgtcggtg |
| 19321 | cctggttgtt cttgattttc catgccgcct cctttagccg ctaaaattca tctactcatt |
| 19381 | tattcatttg ctcatttact ctggtagctg cgcgatgtat tcagatagca gctcggtaat |
| 19441 | ggtcttgcct tggcgtaccg cgtacatctt cagcttggtg tgatcctccg ccggcaactg |
| 19501 | aaagttgacc cgcttcatgg ctggcgtgtc tgccaggctg gccaacgttg cagccttgct |
| 19561 | gctgcgtgcg ctcggacggc cggcacttag cgtgtttgtg cttttgctca ttttctcttt |
| 19621 | acctcattaa ctcaaatgag ttttgattta atttcagcgg ccagcgcctg gacctcgcgg |
| 19681 | gcagcgtcgc cctcgggttc tgattcaaga acggttgtgc cggcggcggc agtgcctggg |
| 19741 | tagctcacgc gctgcgtgat acgggactca agaatgggca gctcgtaccc ggccagcgcc |
| 19801 | tcggcaacct caccgccgat gcgcgtgcct ttgatcgccc gcgacacgac aaaggccgct |
| 19861 | tgtagccttc catccgtgac ctcaatgcgc tgcttaacca gctccaccag gtcggcggtg |
| 19921 | gcccatatgt cgtaagggct tggctgcacc ggaatcagca cgaagtcggc tgccttgatc |
| 19981 | gcggacacag ccaagtccgc cgcctggggc gctccgtcga tcactacgaa gtcgcgccgg |
| 20041 | ccgatggcct tcacgtcgcg gtcaatcgtc gggcggtcga tgccgacaac ggttagcggt |
| 20101 | tgatcttccc gcacggccgc ccaatcgcgg gcactgccct ggggatcgga atcgactaac |
| 20161 | agaacatcgg ccccggcgag ttgcagggcg cgggctagat gggttgcgat ggtcgtcttg |
| 20221 | cctgacccgc ctttctggtt aagtacagcg ataaccttca tgcgttcccc ttgcgtattt |
| 20281 | gtttatttac tcatcgcatc atatacgcag cgaccgcatg acgcaagctg ttttactcaa |
| 20341 | atacacatca cctttttaga cggcggcgct cggtttcttc agcggccaag ctggccggcc |
| 20401 | aggccgccag cttggcatca gacaaaccgg ccaggatttc atgcagccgc acggttgaga |
| 20461 | cgtgcgcggg cggctcgaac acgtacccgg ccgcgatcat ctccgcctcg atctcttcgg |
| 20521 | taatgaaaaa cggttcgtcc tggccgtcct ggtgcggttt catgcttgtt cctcttggcg |
| 20581 | ttcattctcg gcggccgcca gggcgtcggc ctcggtcaat gcgtcctcac ggaaggcacc |
| 20641 | gcgccgcctg gcctcggtgg gcgtcacttc ctcgctgcgc tcaagtgcgc ggtacagggt |
| 20701 | cgagcgatgc acgccaagca gtgcagccgc ctctttcacg gtgcggcctt cctggtcgat |
| 20761 | cagctcgcgg gcgtgcgcga tctgtgccgg ggtgagggta gggcgggggc caaacttcac |
| 20821 | gcctcgggcc ttggcggcct cgcgcccgct ccgggtgcgg tcgatgatta gggaacgctc |
| 20881 | gaactcggca atgccggcga acacggtcaa caccatgcgg ccggccggcg tggtggtgtc |
| 20941 | ggcccacggc tctgccaggc tacgcaggcc cgcgccggcc tcctggatgc gctcggcaat |
| 21001 | gtccagtagg tcgcgggtgc tgcgggccag gcggtctagc ctggtcactg tcacaacgtc |
| 21061 | gccagggcgt aggtggtcaa gcatcctggc cagctccggg cggtcgcgcc tggtgccggt |
| 21121 | gatcttctcg gaaaacagct tggtgcagcc ggccgcgtgc agttcggccc gttggttggt |
| 21181 | caagtcctgg tcgtcggtgc tgacgcgggc atagcccagc aggccagcgg cggcgctctt |
| 21241 | gttcatggcg taatgtctcc ggttctagtc gcaagtattc tactttatgc gactaaaaca |
| 21301 | cgcgacaaga aaacgccagg aaaagggcag ggcggcagcc tgtcgcgtaa cttaggactt |
| 21361 | gtgcgacatg tcgttttcag aagacggctg cactgaacgt cagaagccga ctgcactata |
| 21421 | gcagcggagg ggttggatca aagtactttg atcccgaggg gaaccctgtg gttggcatgc |
| 21481 | acatacaaat ggacgaacgg ataaaccttt tcacgccctt ttaaatatcc gttattctaa |
| 21541 | taaacgctct tttctcttag |

SEQ ID NO: 93.

| | |
|---|---|
| LOCUS | The_one_component_tran 21585 bp ds-DNA |
| | circular 09-MAR.-2022 |
| DEFINITION | . |
| ACCESSION | pVec1 |
| VERSION | pVec1.1 |
| FEATURES | Location/Qualifiers |
| Agro tDNA cut site | 1 . . . 25 |
| | /label = “RB″ |
| misc_feature | 69 . . . 83 |
| | /label = “TIR″ |
| Transposon | 69 . . . 512 |
| | /label = “mPing″ |
| misc_feature | 171 . . . 183 |
| | /label = “HSE″ |
| misc_feature | 216 . . . 228 |
| | /label = “HSE″ |
| misc_feature | complement (260 . . . 272) |
| | /label = “HSE″ |
| misc_feature | complement (308 . . . 320) |
| | /label = “HSE″ |
| misc_feature | complement (355 . . . 367) |
| | /label = “HSE″ |
| misc_feature | 402 . . . 414 |
| | /label = “HSE″ |
| misc_feature | complement (498 . . . 512) |
| | /label = “TIR″ |
| misc_feature | 754 . . . 1177 |
| | /label = “U6-26promoter″ |
| misc_feature | 1178 . . . 1197 |
| | /label = “gRNA to ACT8 promoter″ |
| misc_feature | 1198 . . . 1273 |
| | /label = “gRNA scaffold″ |
| misc_feature | 1274 . . . 1465 |
| | /label = “U6-26 terminator″ |
| promoter | 1481 . . . 3167 |
| | /label = “Rps5a″ |
| misc_feature | 3204 . . . 4601 |
| | /label = “ORF1″ |
| terminator | 4665 . . . 5390 |
| | /label = “OCS terminator″ |
| promoter | 5573 . . . 6492 |
| | /label = “GmUbi3 Promoter″ |
| misc_feature | 6514 . . . 7959 |
| | /label = “Pong TPase LA″ |
| misc_feature | 7963 . . . 7977 |
| | /label = “G4S linker″ |
| feature | 7981 . . . 8001 |
| | /label = “SV40 NLS″ |
| misc_feature | 8005 . . . 12174 |
| | /label = “Cas9″ |
| misc_feature | 12127 . . . 12174 |
| | /label = “NLS″ |
| terminator | 12202 . . . 12929 |
| | /label = “OCS Terminator″ |
| promoter | 13180 . . . 13921 |
| | /label = “CaMVd35S promoter″ |
| gene | 14012 . . . 15007 |
| | /label = “hygroB (variant) ″ |
| misc_feature | complement (15625 . . . 15647) |
| | /label = “LB″ |
| gene | 15763 . . . 16557 |
| | /label = “KanR1″ |
| origin | 16628 . . . 17240 |
| | /label = “pBR322_origin″ |

ORIGIN

| | |
|---|---|
| 1 | gtttacccgc caatatatcc tgtcaaacac tgatagtttc acgtgatctc cttggatcct |
| 61 | ctagattagg ccagtcacaa tggctagtgt cattgcacgg ctacccaaaa tattatacca |
| 121 | tcttctctca aatgaaatct tttatgaaac aatccccaca gtggaggggt ttcttgaacg |
| 181 | ttccaagact aagcaaagca tttaattgat acaagttcgc gaagattcat ttgtacccaa |
| 241 | aatccggcgc ggcgcgggag aatgttctgg aaggtcgcac ggcggaggcg gacgcaagag |
| 301 | atccggtgaa tgttcaagaa tcggcctcaa cgggggtttc actctgttac cgaggaactt |
| 361 | tctggaaacg acgctgacga gtttcaccag gatgaaactc tttccagaaa gttctctctc |
| 421 | atccccattt catgcaaata atcatttttt attcagtctt acccctatta aatgtgcatg |
| 481 | acacaccagt gaaaccccca ttgtgactgg ccttatctag agtcccccat actaggccta |
| 541 | aactgaaggc gggaaacgac aatctgatcc aagctcaagc tgctctagca ttcgccattc |
| 601 | aggctgcgca actgttggga agggcgatcg gtgcgggcct cttcgctatt acgccagctg |
| 661 | gcgaaagggg gatgtgctgc aaggcgatta agttgggtaa cgccagggtt ttcccagtca |
| 721 | cgacgttgta aaacgacggc cagtgccaag cttcgacttg ccttccgcac aatacatcat |
| 781 | ttcttcttag ctttttttct tcttcttcgt tcatacagtt tttttttgtt tatcagctta |
| 841 | cattttcttg aaccgtagct ttcgttttct tctttttaac tttccattcg gagtttttgt |
| 901 | atcttgtttc atagtttgtc ccaggattag aatgattagg catcgaacct tcaagaattt |
| 961 | gattgaataa aacatcttca ttcttaagat atgaagataa tcttcaaaag gcccctggga |
| 1021 | atctgaaaga agagaagcag gcccatttat atgggaaaga acaatagtat ttcttatata |
| 1081 | ggcccattta agttgaaaac aatcttcaaa agtcccacat cgcttagata agaaaacgaa |
| 1141 | gctgagttta tatacagcta gagtcgaagt agtgattgtt acaggagtag ttcatcggtt |
| 1201 | ttagagctag aaatagcaag ttaaaataag gctagtccgt tatcaacttg aaaaagtggc |
| 1261 | accgagtcgg tgcttttttt tgcaaaattt tccagatcga tttcttcttc ctctgttctt |
| 1321 | cggcgttcaa tttctggggt tttctcttcg ttttctgtaa ctgaaaccta aaatttgacc |
| 1381 | taaaaaaaat ctcaaataat atgattcagt ggttttgtac ttttcagtta gttgagtttt |
| 1441 | gcagttccga tgagataaac caataccatg ttagagagcg ctagttcgtg agtagatata |
| 1501 | ttactcaact tttgattcgc tatttgcagt gcacctgtgg cgttcatcac atcttttgtg |
| 1561 | acactgtttg cactggtcat tgctattaca aaggaccttc ctgatgttga aggagatcga |
| 1621 | aagtaagtaa ctgcacgcat aaccattttc tttccgctct ttggctcaat ccatttgaca |
| 1681 | gtcaaagaca atgtttaacc agctccgttt gatatattgt ctttatgtgt ttgttcaagc |
| 1741 | atgtttagtt aatcatgcct ttgattgatc ttgaataggt tccaaatatc aaccctggca |
| 1801 | acaaaacttg gagtgagaaa cattgcattc ctcggttctg gacttctgct agtaaattat |
| 1861 | gtttcagcca tatcactagc tttctacatg cctcaggtga attcatctat ttccgtctta |
| 1921 | actatttcgg ttaatcaaag cacgaacacc attactgcat gtagaagctt gataaactat |
| 1981 | cgccaccaat ttatttttgt tgcgatattg ttactttcct cagtatgcag ctttgaaaag |
| 2041 | accaaccctc ttatccttta acaatgaaca ggtttttaga ggtagcttga tgattcctgc |
| 2101 | acatgtgatc ttggcttcag gcttaatttt ccaggtaaag cattatgaga tactcttata |
| 2161 | tctcttacat acttttgaga taatgcacaa gaacttcata actatatgct ttagtttctg |
| 2221 | catttgacac tgccaaattc attaatctct aatatctttg ttgttgatct ttggtagaca |
| 2281 | tgggtactag aaaaagcaaa ctacaccaag gtaaaatact tttgtacaaa cataaactcg |
| 2341 | ttatcacgga acatcaatgg agtgtatatc taacggagtg tagaaacatt tgattattgc |
| 2401 | aggaagctat ctcaggatat tatcggttta tatggaatct cttctacgca gagtatctgt |
| 2461 | tattcccctt cctctagctt tcaatttcat ggtgaggata tgcagttttc tttgtatatc |
| 2521 | attcttcttc ttctttgtag cttggagtca aaatcggttc cttcatgtac atacatcaag |
| 2581 | gatatgtcct tctgaatttt tatatcttgc aataaaaatg cttgtaccaa ttgaaacacc |
| 2641 | agctttttga gttctatgat cactgacttg gttctaacca aaaaaaaaaa aatgtttaat |
| 2701 | ttacatatct aaaagtaggt ttagggaaac ctaaacagta aaatatttgt atattattcg |
| 2761 | aatttcactc atcataaaaa cttaaattgc accataaaat tttgttttac tattaatgat |
| 2821 | gtaatttgtg taacttaaga taaaaataat attccgtaag ttaaccggct aaaaccacgt |
| 2881 | ataaaccagg gaacctgtta aaccggttct ttactggata aagaaatgaa agcccatgta |
| 2941 | gacagctcca ttagagccca aaccctaaat ttctcatcta tataaaagga gtgacattag |
| 3001 | ggtttttgtt cgtcctctta aagcttctcg ttttctctgc cgtctctctc attcgcgcga |
| 3061 | cgcaaacgat cttcaggtga tcttctttct ccaaatcctc tctcataact ctgatttcgt |
| 3121 | acttgtgtat ttgagctcac gctctgtttc tctcaccaca gccggattcg agatcacaag |
| 3181 | tttgtacaaa aaagcaggct tccatggatc cgtcgccggc cgtggatccg tcgccggccg |
| 3241 | tggatccgtc gccggctgct gaaacccggc ggcgtgcaac cgggaaagga ggcaaacagc |
| 3301 | gcgggggcaa gcaactagga ttgaagaggc cgccgccgat ttctgtcccg gccaccccgc |
| 3361 | ctcctgctgc gacgtcttca tcccctgctg cgccgacggc catcccacca cgaccaccgc |
| 3421 | aatcttcgcc gattttcgtc cccgattcgc cgaatccgtc accggctgcg ccgacctcct |
| 3481 | ctcttgcttc ggggacatcg acggcaaggc caccgcaacc acaaggagga ggatggggac |
| 3541 | caacatcgac catttcccca aactttgcat ctttctttgg aaaccaacaa gacccaaatt |
| 3601 | catgtttggt caggggttat cctccaggag ggtttgtcaa ttttattcaa caaaattgtc |
| 3661 | cgccgcagcc acaacagcaa ggtgaaaatt ttcatttcgt tggtcacaat atggggttca |
| 3721 | acccaatatc tccacagcca ccaagtgcct acggaacacc aacaccccaa gctacgaacc |
| 3781 | aaggcacttc aacaaacatt atgattgatg aagaggacaa caatgatgac agtagggcag |
| 3841 | caaagaaaag atggactcat gaagaggaag agagactggc cagtgcttgg ttgaatgctt |
| 3901 | ctaaagactc aattcatggg aatgataaga aaggtgatac attttggaag gaagtcactg |
| 3961 | atgaatttaa caagaaaggg aatggaaaac gtaggaggga aattaaccaa ctgaaggttc |
| 4021 | actggtcaag gttgaagtca gcgatctctg agttcaatga ctattggagt acggttactc |
| 4081 | aaatgcatac aagcggatac tcagacgaca tgcttgagaa agaggcacag aggctgtatg |
| 4141 | caaacaggtt tggaaaacct tttgcgttgg tccattggtg gaagatactc aaaagagagc |
| 4201 | ccaaatggtg tgctcagttt gaaaagagga aaaggaagag cgaaatggat gctgttccag |
| 4261 | aacagcagaa acgtcctatt ggtagagaag cagcaaagtc tgagcgcaaa agaaagcgca |
| 4321 | agaaagaaaa tgttatggaa ggcattgtcc tcctagggga caatgtccag aaaattatca |
| 4381 | aagtgacgca agatcggaag ctggagcgtg agaaggtcac tgaagcacag attcacattt |
| 4441 | caaacgtaaa tttgaaggca gcagaacagc aaaaagaagc aaagatgttt gaggtataca |
| 4501 | attccctgct cactcaagat acaagtaaca tgtctgaaga acagaaggct cgccgagaca |
| 4561 | aggcattaca aaagctggag gaaaagttat ttgctgacta gtgacccagc tttcttgtac |
| 4621 | aaagtggtgc ctaggtgagt ctagagagtt gattaagacc cgggactggt ccctagagtc |
| 4681 | ctgctttaat gagatatgcg agacgcctat gatcgcatga tatttgcttt caattctgtt |
| 4741 | gtgcacgttg taaaaaacct gagcatgtgt agctcagatc cttaccgccg gtttcggttc |
| 4801 | attctaatga atatatcacc cgttactatc gtatttttat gaataatatt ctccgttcaa |
| 4861 | tttactgatt gtaccctact acttatatgt acaatattaa aatgaaaaca atatattgtg |
| 4921 | ctgaataggt ttatagcgac atctatgata gagcgccaca ataacaaaca attgcgtttt |
| 4981 | attattacaa atccaatttt aaaaaaagcg gcagaaccgg tcaaacctaa aagactgatt |
| 5041 | acataaatct tattcaaatt tcaaaagtgc cccaggggct agtatctacg acacaccgag |
| 5101 | cggcgaacta ataacgctca ctgaagggaa ctccggttcc ccgccggcgc gcatgggtga |
| 5161 | gattccttga agttgagtat tggccgtccg ctctaccgaa agttacgggc accattcaac |
| 5221 | ccggtccagc acggcggccg ggtaaccgac ttgctgcccc gagaattatg cagcattttt |
| 5281 | ttggtgtatg tgggccccaa atgaagtgca ggtcaaacct tgacagtgac gacaaatcgt |
| 5341 | tgggcgggtc cagggcgaat tttgcgacaa catgtcgagg ctcagcagga cctgcaggca |
| 5401 | tgcaagcttg gcactggccg tcgttttaca acgtcgtgac tgggaaaacc ctggcgttac |
| 5461 | ccaacttaat cgccttgcag cacatccccc tttcgccagc tggcgtaata gcgaagaggc |
| 5521 | ccgcaccgat cgcccttccc aacagttgcg cagcctgaat ggcgaatgct agagcagctt |
| 5581 | gagcttggat cagattgtcg tttcccgcct tcagtttctt gaaggtgcat gtgactccgt |
| 5641 | caagattacg aaaccgccaa ctaccacgca aattgcaatt ctcaatttcc tagaaggact |
| 5701 | ctccgaaaat gcatccaata ccaaatatta cccgtgtcat aggcaccaag tgacaccata |
| 5761 | catgaacacg cgtcacaata tgactggaga agggttccac accttatgct ataaaacgcc |
| 5821 | ccacacccct cctccttcct tcgcagttca attccaatat attccattct ctctgtgtat |
| 5881 | ttccctacct ctcccttcaa ggttagtcga tttcttctgt ttttcttctt cgttctttcc |
| 5941 | atgaattgtg tatgttcttt gatcaatacg atgttgattt gattgtgttt tgtttggttt |
| 6001 | catcgatctt caattttcat aatcagattc agcttttatt atctttacaa caacgtcctt |
| 6061 | aatttgatga ttctttaatc gtagatttgc tctaattaga gctttttcat gtcagatccc |
| 6121 | tttacaacaa gccttaattg ttgattcatt aatcgtagat tagggctttt ttcattgatt |
| 6181 | acttcagatc cgttaaacgt aaccatagat cagggctttt tcatgaatta cttcagatcc |
| 6241 | gttaaacaac agccttattt tttatacttc tgtggttttt caagaaattg ttcagatccg |
| 6301 | ttgacaaaaa gccttattcg ttgattctat atcgtttttc gagagatatt gctcagatct |
| 6361 | gttagcaact gccttgtttg ttgattctat tgccgtggat tagggttttt tttcacgaga |
| 6421 | ttgcttcaga tccgtactta agattacgta atggattttg attctgattt atctgtgatt |
| 6481 | gttgactcga caggtacctt caaacggcgc gccatgcaga gtttagccat ctctctactc |
| 6541 | ctctcagaaa ctcattccct cttttctcat acgaagacct cctccctttt atctttactg |
| 6601 | tttctctctt cttcaaagat gtctgagcaa aatactgatg gaagtcaagt tccagtgaac |
| 6661 | ttgttggatg agttcctggc tgaggatgag atcatagatg atcttctcac tgaagccacg |
| 6721 | gtggtagtac agtccactat agaaggtctt caaaacgagg cttctgacca tcgacatcat |
| 6781 | ccgaggaagc acatcaagag gccacgagag gaagcacatc agcaactggt gaatgattac |
| 6841 | ttttcagaaa atcctcttta cccttccaaa atttttcgtc gaagatttcg tatgtctagg |
| 6901 | ccactttttc ttcgcatcgt tgaggcatta ggccagtggt cagtgtattt cacacaaagg |
| 6961 | gtggatgctg ttaatcggaa aggactcagt ccactgcaaa agtgtactgc agctattcgc |
| 7021 | cagttggcta ctggtagtgg cgcagatgaa ctagatgaat atctgaagat aggagagact |
| 7081 | acagcaatgg aggcaatgaa gaattttgtc aaaggtcttc aagatgtgtt tggtgagagg |
| 7141 | tatcttaggc gccccactat ggaagatacc gaacggcttc tccaacttgg tgagaaacgt |
| 7201 | ggttttcctg gaatgttcgg cagcattgac tgcatgcact ggcattggga aagatgccca |
| 7261 | gtagcatgga agggtcagtt cactcgtgga gatcagaaag tgccaaccct gattcttgag |
| 7321 | gctgtggcat cgcatgatct ttggatttgg catgcatttt ttggagcagc gggttccaac |
| 7381 | aatgatatca atgtattgaa ccaatctact gtatttatca aggagctcaa aggacaagct |
| 7441 | cctagagtcc agtacatggt aaatgggaat caatacaata ctgggtattt tcttgctgat |
| 7501 | ggaatctacc ctgaatgggc agtgtttgtt aagtcaatac gactcccaaa cactgaaaag |
| 7561 | gagaaattgt atgcagatat gcaagaaggg gcaagaaaag atatcgagag agcctttggt |
| 7621 | gtattgcagc gaagattttg catcttaaaa cgaccagctc gtctatatga tcgaggtgta |
| 7681 | ctgcgagatg ttgttctagc ttgcatcata cttcacaata tgatagttga agatgagaag |
| 7741 | gaaaccagaa ttattgaaga agatgcagat gcaaatgtgc ctcctagttc atcaaccgtt |
| 7801 | caggaacctg agttctctcc tgaacagaac acaccatttg atagagtttt agaaaaagat |
| 7861 | atttctatcc gagatcgagc ggctcataac cgacttaaga aagatttggt ggaacacatt |
| 7921 | tggaataagt ttggtggtgc tgcacataga actggaaatt atggcggggg aggtagcgct |
| 7981 | ccgaagaaga agaggaaggt tggcatccac ggggtgccag ctgctgacaa gaagtactcg |
| 8041 | atcggcctcg atattgggac taactctgtt ggctgggccg tgatcaccga cgagtacaag |
| 8101 | gtgccctcaa agaagttcaa ggtcctgggc aacaccgatc ggcattccat caagaagaat |
| 8161 | ctcattggcg ctctcctgtt cgacagcggc gagacggctg aggctacgcg gctcaagcgc |
| 8221 | accgcccgca ggcggtacac gcgcaggaag aatcgcatct gctacctgca ggagattttc |
| 8281 | tccaacgaga tggcgaaggt tgacgattct ttcttccaca ggctggagga gtcattcctc |
| 8341 | gtggaggagg ataagaagca cgagcggcat ccaatcttcg gcaacattgt cgacgaggtt |
| 8401 | gcctaccacg agaagtaccc tacgatctac catctgcgga agaagctcgt ggactccaca |
| 8461 | gataaggcgg acctccgcct gatctacctc gctctggccc acatgattaa gttcaggggc |
| 8521 | catttcctga tcgaggggga tctcaacccg gacaatagcg atgttgacaa gctgttcatc |
| 8581 | cagctcgtgc agacgtacaa ccagctcttc gaggagaacc ccattaatgc gtcaggcgtc |
| 8641 | gacgcgaagg ctatcctgtc cgctaggctc tcgaagtctc ggcgcctcga gaacctgatc |
| 8701 | gcccagctgc cgggcgagaa gaagaacggc ctgttcggga atctcattgc gctcagcctg |
| 8761 | gggctcacgc ccaacttcaa gtcgaatttc gatctcgctg aggacgccaa gctgcagctc |
| 8821 | tccaaggaca catacgacga tgacctggat aacctcctgg cccagatcgg cgatcagtac |
| 8881 | gcggacctgt tcctcgctgc caagaatctg tcggacgcca tcctcctgtc tgatattctc |
| 8941 | agggtgaaca ccgagattac gaaggctccg ctctcagcct ccatgatcaa gcgctacgac |
| 9001 | gagcaccatc aggatctgac cctcctgaag gcgctggtca ggcagcagct ccccgagaag |
| 9061 | tacaaggaga tcttcttcga tcagtcgaag aacggctacg ctgggtacat tgacggcggg |
| 9121 | gcctctcagg aggagttcta caagttcatc aagccgattc tggagaagat ggacggcacg |
| 9181 | gaggagctgc tggtgaagct caatcgcgag gacctcctga ggaagcagcg gacattcgat |
| 9241 | aacggcagca tcccacacca gattcatctc ggggagctgc acgctatcct gaggaggcag |
| 9301 | gaggacttct accctttcct caaggataac cgcgagaaga tcgagaagat tctgactttc |
| 9361 | aggatcccgt actacgtcgg cccactcgct aggggcaact cccgcttcgc ttggatgacc |
| 9421 | cgcaagtcag aggagacgat cacgccgtgg aacttcgagg aggtggtcga caagggcgct |
| 9481 | agcgctcagt cgttcatcga gaggatgacg aatttcgaca agaacctgcc aaatgagaag |
| 9541 | gtgctcccta agcactcgct cctgtacgag tacttcacag tctacaacga gctgactaag |
| 9601 | gtgaagtatg tgaccgaggg catgaggaag ccggctttcc tgtctgggga gcagaagaag |
| 9661 | gccatcgtgg acctcctgtt caagaccaac cggaaggtca cggttaagca gctcaaggag |
| 9721 | gactacttca agaagattga gtgcttcgat tcggtcgaga tctctggcgt tgaggaccgc |
| 9781 | ttcaacgcct ccctggggac ctaccacgat ctcctgaaga tcattaagga taaggacttc |
| 9841 | ctggacaacg aggagaatga ggatatcctc gaggacattg tgctgacact cactctgttc |
| 9901 | gaggaccggg agatgatcga ggagcgcctg aagacttacg cccatctctt cgatgacaag |
| 9961 | gtcatgaagc agctcaagag gaggaggtac accggctggg ggaggctgag caggaagctc |
| 10021 | atcaacggca ttcgggacaa gcagtccggg aagacgatcc tcgacttcct gaagagcgat |
| 10081 | ggcttcgcga accgcaattt catgcagctg attcacgatg acagcctcac attcaaggag |
| 10141 | gatatccaga aggctcaggt gagcggccag ggggactcgc tgcacgagca tatcgcgaac |
| 10201 | ctcgctggct cgccagctat caagaagggg attctgcaga ccgtgaaggt tgtggacgag |
| 10261 | ctggtgaagg tcatgggcag gcacaagcct gagaacatcg tcattgagat ggcccgggag |
| 10321 | aatcagacca cgcagaaggg ccagaagaac tcacgcgaga ggatgaagag gatcgaggag |
| 10381 | ggcattaagg agctggggtc ccagatcctc aaggagcacc cggtggagaa cacgcagctg |
| 10441 | cagaatgaga agctctacct gtactacctc cagaatggcc gcgatatgta tgtggaccag |
| 10501 | gagctggata ttaacaggct cagcgattac gacgtcgatc atatcgttcc acagtcattc |
| 10561 | ctgaaggatg actccattga caacaaggtc ctcaccaggt cggacaagaa ccggggcaag |
| 10621 | tctgataatg ttccttcaga ggaggtcgtt aagaagatga agaactactg gcgccagctc |
| 10681 | ctgaatgcca agctgatcac gcagcggaag ttcgataacc tcacaaaggc tgagaggggc |
| 10741 | gggctctctg agctggacaa ggcgggcttc atcaagaggc agctggtcga gacacggcag |
| 10801 | atcactaagc acgttgcgca gattctcgac tcacggatga acactaagta cgatgagaat |
| 10861 | gacaagctga tccgcgaggt gaaggtcatc accctgaagt caaagctcgt ctccgacttc |
| 10921 | aggaaggatt tccagttcta caaggttcgg gagatcaaca attaccacca tgcccatgac |
| 10981 | gcgtacctga acgcggtggt cggcacagct ctgatcaaga agtacccaaa gctcgagagc |
| 11041 | gagttcgtgt acggggacta caaggtttac gatgtgagga agatgatcgc caagtcggag |
| 11101 | caggagattg gcaaggctac cgccaagtac ttcttctact ctaacattat gaatttcttc |
| 11161 | aagacagaga tcactctggc caatggcgag atccggaagc gccccctcat cgagacgaac |
| 11221 | ggcgagacgg gggagatcgt gtgggacaag ggcagggatt tcgcgaccgt caggaaggtt |
| 11281 | ctctccatgc cacaagtgaa tatcgtcaag aagacagagg tccagactgg cgggttctct |
| 11341 | aaggagtcaa ttctgcctaa gcggaacagc gacaagctca tcgcccgcaa gaaggactgg |
| 11401 | gatccgaaga agtacggcgg gttcgacagc cccactgtgg cctactcggt cctggttgtg |
| 11461 | gcgaaggttg agaagggcaa gtccaagaag ctcaagagcg tgaaggagct gctggggatc |
| 11521 | acgattatgg agcgctccag cttcgagaag aacccgatcg atttcctgga ggcgaagggc |
| 11581 | tacaaggagg tgaagaagga cctgatcatt aagctcccca agtactcact cttcgagctg |
| 11641 | gagaacggca ggaagcggat gctggcttcc gctggcgagc tgcagaaggg gaacgagctg |
| 11701 | gctctgccgt ccaagtatgt gaacttcctc tacctggcct cccactacga gaagctcaag |
| 11761 | ggcagccccg aggacaacga gcagaagcag ctgttcgtcg agcagcacaa gcattacctc |
| 11821 | gacgagatca ttgagcagat ttccgagttc tccaagcgcg tgatcctggc cgacgcgaat |
| 11881 | ctggataagg tcctctccgc gtacaacaag caccgcgaca agccaatcag ggagcaggct |
| 11941 | gagaatatca ttcatctctt caccctgacg aacctcggcg cccctgctgc tttcaagtac |
| 12001 | ttcgacacaa ctatcgatcg caagaggtac acaagcacta aggaggtcct ggacgcgacc |
| 12061 | ctcatccacc agtcgattac cggcctctac gagacgcgca tcgacctgtc tcagctcggg |
| 12121 | ggcgacaagc ggccagcggc gacgaagaag gcggggcagg cgaagaagaa gaagtgataa |
| 12181 | ttgacattct aatctagagt cctgctttaa tgagatatgc gagacgccta tgatcgcatg |
| 12241 | atatttgctt tcaattctgt tgtgcacgtt gtaaaaaacc tgagcatgtg tagctcagat |
| 12301 | ccttaccgcc ggtttcggtt cattctaatg aatatatcac ccgttactat cgtattttta |
| 12361 | tgaataatat tctccgttca atttactgat tgtaccctac tacttatatg tacaatatta |
| 12421 | aaatgaaaac aatatattgt gctgaatagg tttatagcga catctatgat agagcgccac |
| 12481 | aataacaaac aattgcgttt tattattaca aatccaattt taaaaaaagc ggcagaaccg |
| 12541 | gtcaaaccta aaagactgat tacataaatc ttattcaaat ttcaaaagtg ccccaggggc |
| 12601 | tagtatctac gacacaccga gcggcgaact aataacgttc actgaaggga actccggttc |
| 12661 | cccgccggcg cgcatgggtg agattccttg aagttgagta ttggccgtcc gctctaccga |
| 12721 | aagttacggg caccattcaa cccggtccag cacggcggcc gggtaaccga cttgctgccc |
| 12781 | cgagaattat gcagcatttt tttggtgtat gtgggcccca aatgaagtgc aggtcaaacc |
| 12841 | ttgacagtga cgacaaatcg ttgggcgggt ccagggcgaa ttttgcgaca acatgtcgag |
| 12901 | gctcagcagg acctgcaggc atgcaagatc gcgaattcgt aatcatgtca tagctgtttc |
| 12961 | ctgtgtgaaa ttgttatccg ctcacaattc cacacaacat acgagccgga agcataaagt |
| 13021 | gtaaagcctg gggtgcctaa tgagtgagct aactcacatt aattgcgttg cgctcactgc |
| 13081 | ccgctttcca gtcgggaaac ctgtcgtgcc agctgcatta atgaatcggc caacgcgcgg |
| 13141 | ggagaggcgg tttgcgtatt ggctagagca gcttgccaac atggtggagc acgacactct |
| 13201 | cgtctactcc aagaatatca aagatacagt ctcagaagac caaagggcta ttgagacttt |
| 13261 | tcaacaaagg gtaatatcgg gaaacctcct cggattccat tgcccagcta tctgtcactt |
| 13321 | catcaaaagg acagtagaaa aggaaggtgg cacctacaaa tgccatcatt gcgataaagg |
| 13381 | aaaggctatc gttcaagatg cctctgccga cagtggtccc aaagatggac ccccacccac |
| 13441 | gaggagcatc gtggaaaaag aagacgttcc aaccacgtct tcaaagcaag tggattgatg |
| 13501 | tgataacatg gtggagcacg acactctcgt ctactccaag aatatcaaag atacagtctc |
| 13561 | agaagaccaa agggctattg agacttttca acaaagggta atatcgggaa acctcctcgg |
| 13621 | attccattgc ccagctatct gtcacttcat caaaaggaca gtagaaaagg aaggtggcac |
| 13681 | ctacaaatgc catcattgcg ataaaggaaa ggctatcgtt caagatgcct ctgccgacag |
| 13741 | tggtcccaaa gatggacccc cacccacgag gagcatcgtg gaaaaagaag acgttccaac |
| 13801 | cacgtcttca aagcaagtgg attgatgtga tatctccact gacgtaaggg atgacgcaca |
| 13861 | atcccactat ccttcgcaag accttcctct atataaggaa gttcatttca tttggagagg |
| 13921 | acacgctgaa atcaccagtc tctctctaca aatctatctc tctcgagctt tcgcagatcc |
| 13981 | cggggggcaa tgagatatga aaaagcctga actcaccgcg acgtctgtcg agaagtttct |
| 14041 | gatcgaaaag ttcgacagcg tctccgacct gatgcagctc tcggagggcg aagaatctcg |
| 14101 | tgctttcagc ttcgatgtag gagggcgtgg atatgtcctg cgggtaaata gctgcgccga |
| 14161 | tggtttctac aaagatcgtt atgtttatcg gcactttgca tcggccgcgc tcccgattcc |
| 14221 | ggaagtgctt gacattgggg agtttagcga gagcctgacc tattgcatct cccgccgtgc |
| 14281 | acagggtgtc acgttgcaag acctgcctga aaccgaactg cccgctgttc tacaaccggt |
| 14341 | cgcggaggct atggatgcga tcgctgcggc cgatcttagc cagacgagcg ggttcggccc |
| 14401 | attcggaccg caaggaatcg gtcaatacac tacatggcgt gatttcatat gcgcgattgc |
| 14461 | tgatccccat gtgtatcact ggcaaactgt gatggacgac accgtcagtg cgtccgtcgc |
| 14521 | gcaggctctc gatgagctga tgctttgggc cgaggactgc cccgaagtcc ggcacctcgt |
| 14581 | gcacgcggat ttcggctcca acaatgtcct gacggacaat ggccgcataa cagcggtcat |
| 14641 | tgactggagc gaggcgatgt tcggggattc ccaatacgag gtcgccaaca tcttcttctg |
| 14701 | gaggccgtgg ttggcttgta tggagcagca gacgcgctac ttcgagcgga ggcatccgga |
| 14761 | gcttgcagga tcgccacgac tccgggcgta tatgctccgc attggtcttg accaactcta |
| 14821 | tcagagcttg gttgacggca atttcgatga tgcagcttgg gcgcagggtc gatgcgacgc |
| 14881 | aatcgtccga tccggagccg ggactgtcgg gcgtacacaa atcgcccgca gaagcgcggc |
| 14941 | cgtctggacc gatggctgtg tagaagtact cgccgatagt ggaaaccgac gccccagcac |
| 15001 | tcgtccgagg gcaaagaaat agagtagatg ccgaccggat ctgtcgatcg acaagctcga |
| 15061 | gtttctccat aataatgtgt gagtagttcc cagataaggg aattagggtt cctatagggt |
| 15121 | ttcgctcatg tgttgagcat ataagaaacc cttagtatgt atttgtattt gtaaaatact |
| 15181 | tctatcaata aaatttctaa ttcctaaaac caaaatccag tactaaaatc cagatccccc |
| 15241 | gaattaattc ggcgttaatt cagtacatta aaaacgtccg caatgtgtta ttaagttgtc |
| 15301 | taagcgtcaa tttgtttaca ccacaatata tcctgccacc agccagccaa cagctccccg |
| 15361 | accggcagct cggcacaaaa tcaccactcg atacaggcag cccatcagtc cgggacggcg |
| 15421 | tcagcgggag agccgttgta aggcggcaga ctttgctcat gttaccgatg ctattcggaa |
| 15481 | gaacggcaac taagctgccg ggtttgaaac acggatgatc tcgcggaggg tagcatgttg |
| 15541 | attgtaacga tgacagagcg ttgctgcctg tgatcaccgc ggtttcaaaa tcggctccgt |
| 15601 | cgatactatg ttatacgcca actttgaaaa caactttgaa aaagctgttt tctggtattt |
| 15661 | aaggttttag aatgcaagga acagtgaatt ggagttcgtc ttgttataat tagcttcttg |
| 15721 | gggtatcttt aaatactgta gaaaagagga aggaaataat aaatggctaa aatgagaata |
| 15781 | tcaccggaat tgaaaaaact gatcgaaaaa taccgctgcg taaaagatac ggaaggaatg |
| 15841 | tctcctgcta aggtatataa gctggtggga gaaaatgaaa acctatattt aaaaatgacg |
| 15901 | gacagccggt ataaagggac cacctatgat gtggaacggg aaaaggacat gatgctatgg |
| 15961 | ctggaaggaa agctgcctgt tccaaaggtc ctgcactttg aacggcatga tggctggagc |
| 16021 | aatctgctca tgagtgaggc cgatggcgtc ctttgctcgg aagagtatga agatgaacaa |
| 16081 | agccctgaaa agattatcga gctgtatgcg gagtgcatca ggctctttca ctccatcgac |
| 16141 | atatcggatt gtccctatac gaatagctta gacagccgct tagccgaatt ggattactta |
| 16201 | ctgaataacg atctggccga tgtggattgc gaaaactggg aagaagacac tccatttaaa |
| 16261 | gatccgcgcg agctgtatga ttttttaaag acggaaaagc ccgaagagga acttgtcttt |
| 16321 | tcccacggcg acctgggaga cagcaacatc tttgtgaaag atggcaaagt aagtggcttt |
| 16381 | attgatcttg ggagaagcgg cagggcggac aagtggtatg acattgcctt ctgcgtccgg |
| 16441 | tcgatcaggg aggatatcgg ggaagaacag tatgtcgagc tattttttga cttactgggg |
| 16501 | atcaagcctg attgggagaa aataaaatat tatattttac tggatgaatt gttttagtac |
| 16561 | ctagaatgca tgaccaaaat cccttaacgt gagttttcgt tccactgagc gtcagacccc |
| 16621 | gtagaaaaga tcaaaggatc ttcttgagat cctttttttc tgcgcgtaat ctgctgcttg |
| 16681 | caaacaaaaa aaccaccgct accagcggtg gtttgtttgc cggatcaaga gctaccaact |
| 16741 | ctttttccga aggtaactgg cttcagcaga gcgcagatac caaatactgt ccttctagtg |
| 16801 | tagccgtagt taggccacca cttcaagaac tctgtagcac cgcctacata cctcgctctg |
| 16861 | ctaatcctgt taccagtggc tgctgccagt ggcggtgtct taccgggttg gactcaagac |
| 16921 | gatagttacc ggataaggcg cagcggtcgg gctgaacggg gggttcgtgc acacagccca |
| 16981 | gcttggagcg aacgacctac accgaactga gatacctaca gcgtgagcta tgagaaagcg |
| 17041 | ccacgcttcc cgaagggaga aaggcggaca ggtatccggt aagcggcagg gtcggaacag |
| 17101 | gagagcgcac gagggagctt ccagggggaa acgcctggta tctttatagt cctgtcgggt |
| 17161 | ttcgccacct ctgacttgag cgtcgatttt tgtgatgctc gtcagggggg cggagcctat |
| 17221 | ggaaaaacgc cagcaacgcg gcctttttac ggttcctggc cttttgctgg ccttttgctc |
| 17281 | acatgttctt tcctgcgtta tcccctgatt ctgtggataa ccgtattacc gcctttgagt |
| 17341 | gagctgatac cgctcgccgc agccgaacga ccgagcgcag cgagtcagtg agcgaggaag |
| 17401 | cggaagagcg cctgatgcgg tattttctcc ttacgcatct gtgcggtatt tcacaccgca |
| 17461 | tatggtgcac tctcagtaca atctgctctg atgccgcata gttaagccag tatacactcc |
| 17521 | gctatcgcta cgtgactggg tcatggctgc gccccgacac ccgccaacac ccgctgacgc |
| 17581 | gccctgacgg gcttgtctgc tcccggcatc cgcttacaga caagctgtga ccgtctccgg |
| 17641 | gagctgcatg tgtcagaggt tttcaccgtc atcaccgaaa cgcgcgaggc agggtgcctt |
| 17701 | gatgtgggcg ccggcggtcg agtggcgacg gcgcggcttg tccgcgccct ggtagattgc |
| 17761 | ctggccgtag gccagccatt tttgagcggc cagcggccgc gataggccga cgcgaagcgg |
| 17821 | cggggcgtag ggagcgcagc gaccgaaggg taggcgcttt ttgcagctct tcggctgtgc |
| 17881 | gctggccaga cagttatgca caggccaggc gggttttaag agttttaata agttttaaag |
| 17941 | agttttaggc ggaaaaatcg ccttttttct cttttatatc agtcacttac atgtgtgacc |
| 18001 | ggttcccaat gtacggcttt gggttcccaa tgtacgggtt ccggttccca atgtacggct |
| 18061 | ttgggttccc aatgtacgtg ctatccacag gaaacagacc ttttcgacct ttttcccctg |
| 18121 | ctagggcaat ttgccctagc atctgctccg tacattagga accggcggat gcttcgccct |
| 18181 | cgatcaggtt gcggtagcgc atgactagga tcgggccagc ctgccccgcc tcctccttca |
| 18241 | aatcgtactc cggcaggtca tttgacccga tcagcttgcg cacggtgaaa cagaacttct |
| 18301 | tgaactctcc ggcgctgcca ctgcgttcgt agatcgtctt gaacaaccat ctggcttctg |
| 18361 | ccttgcctgc ggcgcggcgt gccaggcggt agagaaaacg gccgatgccg ggatcgatca |
| 18421 | aaaagtaatc ggggtgaacc gtcagcacgt ccgggttctt gccttctgtg atctcgcggt |
| 18481 | acatccaatc agctagctcg atctcgatgt actccggccg cccggtttcg ctctttacga |
| 18541 | tcttgtagcg gctaatcaag gcttcaccct cggataccgt caccaggcgg ccgttcttgg |
| 18601 | ccttcttcgt acgctgcatg gcaacgtgcg tggtgtttaa ccgaatgcag gtttctacca |
| 18661 | ggtcgtcttt ctgctttccg ccatcggctc gccggcagaa cttgagtacg tccgcaacgt |
| 18721 | gtggacggaa cacgcggccg ggcttgtctc ccttcccttc ccggtatcgg ttcatggatt |
| 18781 | cggttagatg ggaaaccgcc atcagtacca ggtcgtaatc ccacacactg gccatgccgg |
| 18841 | ccggccctgc ggaaacctct acgtgcccgt ctggaagctc gtagcggatc acctcgccag |
| 18901 | ctcgtcggtc acgcttcgac agacggaaaa cggccacgtc catgatgctg cgactatcgc |
| 18961 | gggtgcccac gtcatagagc atcggaacga aaaaatctgg ttgctcgtcg cccttgggcg |
| 19021 | gcttcctaat cgacggcgca ccggctgccg gcggttgccg ggattctttg cggattcgat |
| 19081 | cagcggccgc ttgccacgat tcaccggggc gtgcttctgc ctcgatgcgt tgccgctggg |
| 19141 | cggcctgcgc ggccttcaac ttctccacca ggtcatcacc cagcgccgcg ccgatttgta |
| 19201 | ccgggccgga tggtttgcga ccgctcacgc cgattcctcg ggcttggggg ttccagtgcc |
| 19261 | attgcagggc cggcagacaa cccagccgct tacgcctggc caaccgcccg ttcctccaca |
| 19321 | catggggcat tccacggcgt cggtgcctgg ttgttcttga ttttccatgc cgcctccttt |
| 19381 | agccgctaaa attcatctac tcatttattc atttgctcat ttactctggt agctgcgcga |
| 19441 | tgtattcaga tagcagctcg gtaatggtct tgccttggcg taccgcgtac atcttcagct |
| 19501 | tggtgtgatc ctccgccggc aactgaaagt tgacccgctt catggctggc gtgtctgcca |
| 19561 | ggctggccaa cgttgcagcc ttgctgctgc gtgcgctcgg acggccggca cttagcgtgt |
| 19621 | ttgtgctttt gctcattttc tctttacctc attaactcaa atgagttttg atttaatttc |
| 19681 | agcggccagc gcctggacct cgcgggcagc gtcgccctcg ggttctgatt caagaacggt |
| 19741 | tgtgccggcg gcggcagtgc ctgggtagct cacgcgctgc gtgatacggg actcaagaat |
| 19801 | gggcagctcg tacccggcca gcgcctcggc aacctcaccg ccgatgcgcg tgcctttgat |
| 19861 | cgcccgcgac acgacaaagg ccgcttgtag ccttccatcc gtgacctcaa tgcgctgctt |
| 19921 | aaccagctcc accaggtcgg cggtggccca tatgtcgtaa gggcttggct gcaccggaat |
| 19981 | cagcacgaag tcggctgcct tgatcgcgga cacagccaag tccgccgcct ggggcgctcc |
| 20041 | gtcgatcact acgaagtcgc gccggccgat ggccttcacg tcgcggtcaa tcgtcgggcg |
| 20101 | gtcgatgccg acaacggtta gcggttgatc ttcccgcacg gccgcccaat cgcgggcact |
| 20161 | gccctgggga tcggaatcga ctaacagaac atcggccccg gcgagttgca gggcgcgggc |
| 20221 | tagatgggtt gcgatggtcg tcttgcctga cccgcctttc tggttaagta cagcgataac |
| 20281 | cttcatgcgt tccccttgcg tatttgttta tttactcatc gcatcatata cgcagcgacc |
| 20341 | gcatgacgca agctgtttta ctcaaataca catcaccttt ttagacggcg gcgctcggtt |
| 20401 | tcttcagcgg ccaagctggc cggccaggcc gccagcttgg catcagacaa accggccagg |
| 20461 | atttcatgca gccgcacggt tgagacgtgc gcgggcggct cgaacacgta cccggccgcg |
| 20521 | atcatctccg cctcgatctc ttcggtaatg aaaaacggtt cgtcctggcc gtcctggtgc |
| 20581 | ggtttcatgc ttgttcctct tggcgttcat tctcggcggc cgccagggcg tcggcctcgg |
| 20641 | tcaatgcgtc ctcacggaag gcaccgcgcc gcctggcctc ggtgggcgtc acttcctcgc |
| 20701 | tgcgctcaag tgcgcggtac agggtcgagc gatgcacgcc aagcagtgca gccgcctctt |
| 20761 | tcacggtgcg gccttcctgg tcgatcagct cgcgggcgtg cgcgatctgt gccggggtga |
| 20821 | gggtagggcg ggggccaaac ttcacgcctc gggccttggc ggcctcgcgc ccgctccggg |
| 20881 | tgcggtcgat gattagggaa cgctcgaact cggcaatgcc ggcgaacacg gtcaacacca |
| 20941 | tgcggccggc cggcgtggtg gtgtcggccc acggctctgc caggctacgc aggcccgcgc |
| 21001 | cggcctcctg gatgcgctcg gcaatgtcca gtaggtcgcg ggtgctgcgg gccaggcggt |
| 21061 | ctagcctggt cactgtcaca acgtcgccag ggcgtaggtg gtcaagcatc ctggccagct |
| 21121 | ccgggcggtc gcgcctggtg ccggtgatct tctcggaaaa cagcttggtg cagccggccg |
| 21181 | cgtgcagttc ggcccgttgg ttggtcaagt cctggtcgtc ggtgctgacg cgggcatagc |
| 21241 | ccagcaggcc agcggcggcg ctcttgttca tggcgtaatg tctccggttc tagtcgcaag |
| 21301 | tattctactt tatgcgacta aaacacgcga caagaaaacg ccaggaaaag ggcagggcgg |
| 21361 | cagcctgtcg cgtaacttag gacttgtgcg acatgtcgtt ttcagaagac ggctgcactg |
| 21421 | aacgtcagaa gccgactgca ctatagcagc ggaggggttg gatcaaagta ctttgatccc |
| 21481 | gaggggaacc ctgtggttgg catgcacata caaatggacg aacggataaa ccttttcacg |
| 21541 | cccttttaaa tatccgttat tctaataaac gctcttttct cttag |

SEQ ID NO: 94. One component, Unfused_Cas9

| | |
|---|---|
| LOCUS | Unfused_Cas9_and_ORF1/ 23380 bp ds-DNA circular |
| | 09-MAR.-2022 |
| DEFINITION | . |
| ACCESSION | pVec1 |
| VERSION | pVec1 .1 |
| FEATURES | Location/Qualifiers |
| CDS | complement (825 . . . 1373) |
| | /label = “BlpR″ |
| promoter | complement (1565 . . . 1744) |
| | /label = “NOS promoter″ |
| misc_feature | 2201 . . . 2215 |
| | /label = “TIR″ |
| Transposon | 2201 . . . 2630 |
| | /label = “mPing″ |
| misc_feature | complement (2616 . . . 2630) |
| | /label = “TIR″ |
| misc_feature | 2861 . . . 3284 |
| | /label = “U6-26promoter″ |
| misc_feature | 3285 . . . 3304 |
| | /label = “gRNA to DD20″ |
| misc_feature | 3305 . . . 3380 |
| | /label = “gRNA scaffold″ |
| misc_feature | 3381 . . . 3572 |
| | /label = “U6-26 terminator″ |
| promoter | 3593 . . . 5279 |
| | / label = “Rps 5a″ |
| gene | 5295 . . . 6733 |
| | /label = “ORF1SC1″ |
| terminator | 6777 . . . 7502 |
| | /label = “OCS terminator″ |
| promoter | 7685 . . . 8604 |
| gene | /label = “GmUbi3 Promoter″ |
| | 8626 . . . 10074 |
| | /label = “Pong TPase LA″ |
| terminator | 10100 . . . 10827 |
| | /label = “OCS Terminator″ |
| promoter | 10857 . . . 11581 |
| | /label = “AtUBQ10 promoter″ |
| feature | 11597 . . . 11617 |
| | /label = “FLAG″ |
| feature | 11618 . . . 11638 |
| | /label = “FLAG″ |
| feature | 11639 . . . 11662 |
| | /label = “FLAG″ |
| feature | 11669 . . . 11689 |
| | /label = “SV40 NLS″ |
| misc_feature | 11693 . . . 15865 |
| | /label = “Cas9″ |
| misc_feature | 15815 . . . 15862 |
| | /label = “NLS″ |
| misc_feature | 15871 . . . 16495 |
| | /label = “Rbs Term″ |
| misc_feature | 16818 . . . 16842 |
| | /label = “RB T-DNA repeat″ |
| CDS | 18173 . . . 18802 |
| | /label = “pVS1 StaA″ |
| CDS | 19231 . . . 20304 |
| | /label = “pVS1 RepA″ |
| rep_origin | 20370 . . . 20564 |
| | /label = “pVS1 oriV″ |
| misc_feature | 20908 . . . 21048 |
| | /label = “bom″ |
| rep_origin | complement (21234 . . . 21822) |
| | /label = “ori″ |
| CDS | complement (22068 . . . 22859) |
| | /label = “SmR″ |
| misc_feature | join (23380 . . . 23380, 1 . . . 24) |
| | /label = “LB T-DNA repeat″ |

ORIGIN

| | |
|---|---|
| 1 | ggcaggatat attgtggtgt aaacaaattg acgcttagac aacttaataa cacattgcgg |
| 61 | acgtttttaa tgtactgaat taacgccgaa ttgctctagc attcgccatt caggctgcgc |
| 121 | aactgttggg aagggcgatc ggtgcgggcc tcttcgctat tacgccagct ggcgaaaggg |
| 181 | ggatgtgctg caaggcgatt aagttgggta acgccagggt tttcccagtc acgacgttgt |
| 241 | aaaacgacgg ccagtgccaa gctaattcgc ttcaagacgt gctcaaatca ctatttccac |
| 301 | acccctatat ttctattgca ctccctttta actgtttttt attacaaaaa tgccctggaa |
| 361 | aatgcactcc ctttttgtgt ttgttttttt gtgaaacgat gttgtcaggt aatttatttg |
| 421 | tcagtctact atggtggccc attatattaa tagcaactgt cggtccaata gacgacgtcg |
| 481 | attttctgca tttgtttaac cacgtggatt ttatgacatt ttatattagt taatttgtaa |
| 541 | aacctaccca attaaagacc tcatatgttc taaagactaa tacttaatga taacaatttt |
| 601 | cttttagtga agaaagggat aattagtaaa tatggaacaa gggcagaaga tttattaaag |
| 661 | ccgcgtaaga gacaacaagt aggtacgtgg agtgtcttag gtgacttacc cacataacat |
| 721 | aaagtgacat taacaaacat agctaatgct cctatttgaa tagtgcatat cagcatacct |
| 781 | tattacatat agataggagc aaactctagc tagattgttg agcagatctc ggtgacgggc |
| 841 | aggaccggac ggggcggtac cggcaggctg aagtccagct gccagaaacc cacgtcatgc |
| 901 | cagttcccgt gcttgaagcc ggccgcccgc agcatgccgc ggggggcata tccgagcgcc |
| 961 | tcgtgcatgc gcacgctcgg gtcgttgggc agcccgatga cagcgaccac gctcttgaag |
| 1021 | ccctgtgcct ccagggactt cagcaggtgg gtgtagagcg tggagcccag tcccgtccgc |
| 1081 | tggtggcggg gggagacgta cacggtcgac tcggccgtcc agtcgtaggc gttgcgtgcc |
| 1141 | ttccaggggc ccgcgtaggc gatgccggcg acctcgccgt ccacctcggc gacgagccag |
| 1201 | ggatagcgct cccgcagacg gacgaggtcg tccgtccact cctgcggttc ctgcggctcg |
| 1261 | gtacggaagt tgaccgtgct tgtctcgatg tagtggttga cgatggtgca gaccgccggc |
| 1321 | atgtccgcct cggtggcacg gcggatgtcg gccgggcgtc gttctgggct catggtagat |
| 1381 | cccccgttcg taaatggtga aaattttcag aaaattgctt ttgctttaaa agaaatgatt |
| 1441 | taaattgctg caatagaagt agaatgcttg attgcttgag attcgtttgt tttgtatatg |
| 1501 | ttgtgttgag aattaattct cgagcctaga gtcgagatct ggattgagag tgaatatgag |
| 1561 | actctaattg gataccgagg ggaatttatg gaacgtcagt ggagcatttt tgacaagaaa |
| 1621 | tatttgctag ctgatagtga ccttaggcga cttttgaacg cgcaataatg gtttctgacg |
| 1681 | tatgtgctta gctcattaaa ctccagaaac ccgcggctga gtggctcctt caacgttgcg |
| 1741 | gttctgtcag ttccaaacgt aaaacggctt gtcccgcgtc atcggcgggg gtcataacgt |
| 1801 | gactccctta attctccgct catgatcttg atcccctgcg ccatcagatc cttggcggca |
| 1861 | agaaagccat ccagtttact ttgcagggct tcccaacctt accagagggc gccccagctg |
| 1921 | gcaattccgg ttcgcttgct gtccataaaa ccgcccagtc tagctatcgc catgtaagcc |
| 1981 | cactgcaagc tacctgcttt ctctttgcgc ttgcgttttc ccttgtccag atagcccagt |
| 2041 | agctgacatt catccggggt cagcaccgtt tctgcggact ggctttctac gtgttccgct |
| 2101 | tcctttagca gcccttgcgc cctgagtgct tgcggcagcg tgaagcttgc atgcctgcag |
| 2161 | gtcgactcta gtgttatatc tccttggatc ctctagatta ggccagtcac aatggctagt |
| 2221 | gtcattgcac ggctacccaa aatattatac catcttctct caaatgaaat cttttatgaa |
| 2281 | acaatcccca cagtggaggg gtttcacttt gacgtttcca agactaagca aagcatttaa |
| 2341 | ttgatacaag ttgctgggat catttgtacc caaaatccgg cgcggcgcgg gagaatgcgg |
| 2401 | aggtcgcacg gcggaggcgg acgcaagaga tccggtgaat gaaacgaatc ggcctcaacg |
| 2461 | ggggtttcac tctgttaccg aggacttgga aacgacgctg acgagtttca ccaggatgaa |
| 2521 | actctttcct tctctctcat ccccatttca tgcaaataat cattttttat tcagtcttac |
| 2581 | ccctattaaa tgtgcatgac acaccagtga aacccccatt gtgactggcc ttatctagag |
| 2641 | tcccccaaac tgaaggcggg aaacgacaat ctgatccaag ctcaagctgc tctagcattc |
| 2701 | gccattcagg ctgcgcaact gttgggaagg gcgatcggtg cgggcctctt cgctattacg |
| 2761 | ccagctggcg aaagggggat gtgctgcaag gcgattaagt tgggtaacgc cagggttttc |
| 2821 | ccagtcacga cgttgtaaaa cgacggccag tgccaagctt cgacttgcct tccgcacaat |
| 2881 | acatcatttc ttcttagctt tttttcttct tcttcgttca tacagttttt ttttgtttat |
| 2941 | cagcttacat tttcttgaac cgtagctttc gttttcttct ttttaacttt ccattcggag |
| 3001 | tttttgtatc ttgtttcata gtttgtccca ggattagaat gattaggcat cgaaccttca |
| 3061 | agaatttgat tgaataaaac atcttcattc ttaagatatg aagataatct tcaaaaggcc |
| 3121 | cctgggaatc tgaaagaaga gaagcaggcc catttatatg ggaaagaaca atagtatttc |
| 3181 | ttatataggc ccatttaagt tgaaaacaat cttcaaaagt cccacatcgc ttagataaga |
| 3241 | aaacgaagct gagtttatat acagctagag tcgaagtagt gattggaact gacacacgac |
| 3301 | atgagtttta gagctagaaa tagcaagtta aaataaggct agtccgttat caacttgaaa |
| 3361 | aagtggcacc gagtcggtgc ttttttttgc aaaattttcc agatcgattt cttcttcctc |
| 3421 | tgttcttcgg cgttcaattt ctggggtttt ctcttcgttt tctgtaactg aaacctaaaa |
| 3481 | tttgacctaa aaaaaatctc aaataatatg attcagtggt tttgtacttt tcagttagtt |
| 3541 | gagttttgca gttccgatga gataaaccaa taccatggtt atactaggag cgctagttcg |
| 3601 | tgagtagata tattactcaa cttttgattc gctatttgca gtgcacctgt ggcgttcatc |
| 3661 | acatcttttg tgacactgtt tgcactggtc attgctatta caaaggacct tcctgatgtt |
| 3721 | gaaggagatc gaaagtaagt aactgcacgc ataaccattt tctttccgct ctttggctca |
| 3781 | atccatttga cagtcaaaga caatgtttaa ccagctccgt ttgatatatt gtctttatgt |
| 3841 | gtttgttcaa gcatgtttag ttaatcatgc ctttgattga tcttgaatag gttccaaata |
| 3901 | tcaaccctgg caacaaaact tggagtgaga aacattgcat tcctcggttc tggacttctg |
| 3961 | ctagtaaatt atgtttcagc catatcacta gctttctaca tgcctcaggt gaattcatct |
| 4021 | atttccgtct taactatttc ggttaatcaa agcacgaaca ccattactgc atgtagaagc |
| 4081 | ttgataaact atcgccacca atttattttt gttgcgatat tgttactttc ctcagtatgc |
| 4141 | agctttgaaa agaccaaccc tcttatcctt taacaatgaa caggttttta gaggtagctt |
| 4201 | gatgattcct gcacatgtga tcttggcttc aggcttaatt ttccaggtaa agcattatga |
| 4261 | gatactctta tatctcttac atacttttga gataatgcac aagaacttca taactatatg |
| 4321 | ctttagtttc tgcatttgac actgccaaat tcattaatct ctaatatctt tgttgttgat |
| 4381 | ctttggtaga catgggtact agaaaaagca aactacacca aggtaaaata cttttgtaca |
| 4441 | aacataaact cgttatcacg gaacatcaat ggagtgtata tctaacggag tgtagaaaca |
| 4501 | tttgattatt gcaggaagct atctcaggat attatcggtt tatatggaat ctcttctacg |
| 4561 | cagagtatct gttattcccc ttcctctagc tttcaatttc atggtgagga tatgcagttt |
| 4621 | tctttgtata tcattcttct tcttctttgt agcttggagt caaaatcggt tccttcatgt |
| 4681 | acatacatca aggatatgtc cttctgaatt tttatatctt gcaataaaaa tgcttgtacc |
| 4741 | aattgaaaca ccagcttttt gagttctatg atcactgact tggttctaac caaaaaaaaa |
| 4801 | aaaatgttta atttacatat ctaaaagtag gtttagggaa acctaaacag taaaatattt |
| 4861 | gtatattatt cgaatttcac tcatcataaa aacttaaatt gcaccataaa attttgtttt |
| 4921 | actattaatg atgtaatttg tgtaacttaa gataaaaata atattccgta agttaaccgg |
| 4981 | ctaaaaccac gtataaacca gggaacctgt taaaccggtt ctttactgga taaagaaatg |
| 5041 | aaagcccatg tagacagctc cattagagcc caaaccctaa atttctcatc tatataaaag |
| 5101 | gagtgacatt agggtttttg ttcgtcctct taaagcttct cgttttctct gccgtctctc |
| 5161 | tcattcgcgc gacgcaaacg atcttcaggt gatcttcttt ctccaaatcc tctctcataa |
| 5221 | ctctgatttc gtacttgtgt atttgagctc acgctctgtt tctctcacca cagccggatt |
| 5281 | cgagatcaca agtttgtaca aaaaagcagg cttccatgga tccgtcgccg gccgtggatc |
| 5341 | cgtcgccggc cgtggatccg tcgccggctg ctgaaacccg gcggcgtgca accgggaaag |
| 5401 | gaggcaaaca gcgcgggggc aagcaactag gattgaagag gccgccgccg atttctgtcc |
| 5461 | cggccacccc gcctcctgct gcgacgtctt catcccctgc tgcgccgacg gccatcccac |
| 5521 | cacgaccacc gcaatcttcg ccgattttcg tccccgattc gccgaatccg tcaccggctg |
| 5581 | cgccgacctc ctctcttgct tcggggacat cgacggcaag gccaccgcaa ccacaaggag |
| 5641 | gaggatgggg accaacatcg accatttccc caaactttgc atctttcttt ggaaaccaac |
| 5701 | aagacccaaa ttcatgtttg gtcaggggtt atcctccagg agggtttgtc aattttattc |
| 5761 | aacaaaattg tccgccgcag ccacaacagc aaggtgaaaa ttttcatttc gttggtcaca |
| 5821 | atatggggtt caacccaata tctccacagc caccaagtgc ctacggaaca ccaacacccc |
| 5881 | aagctacgaa ccaaggcact tcaacaaaca ttatgattga tgaagaggac aacaatgatg |
| 5941 | acagtagggc agcaaagaaa agatggactc atgaagagga agagagactg gccagtgctt |
| 6001 | ggttgaatgc ttctaaagac tcaattcatg ggaatgataa gaaaggtgat acattttgga |
| 6061 | aggaagtcac tgatgaattt aacaagaaag ggaatggaaa acgtaggagg gaaattaacc |
| 6121 | aactgaaggt tcactggtca aggttgaagt cagcgatctc tgagttcaat gactattgga |
| 6181 | gtacggttac tcaaatgcat acaagcggat actcagacga catgcttgag aaagaggcac |
| 6241 | agaggctgta tgcaaacagg tttggaaaac cttttgcgtt ggtccattgg tggaagatac |
| 6301 | tcaaaagaga gcccaaatgg tgtgctcagt ttgaaaagag gaaaaggaag agcgaaatgg |
| 6361 | atgctgttcc agaacagcag aaacgtccta ttggtagaga agcagcaaag tctgagcgca |
| 6421 | aaagaaagcg caagaaagaa aatgttatgg aaggcattgt cctcctaggg gacaatgtcc |
| 6481 | agaaaattat caaagtgacg caagatcgga agctggagcg tgagaaggtc actgaagcac |
| 6541 | agattcacat ttcaaacgta aatttgaagg cagcagaaca gcaaaaagaa gcaaagatgt |
| 6601 | ttgaggtata caattccctg ctcactcaag atacaagtaa catgtctgaa gaacagaagg |
| 6661 | ctcgccgaga caaggcatta caaaagctgg aggaaaagtt atttgctgac tagtgaccca |
| 6721 | gctttcttgt acaaagtggt gcctaggtga gtctagagag ttgattaaga cccgggactg |
| 6781 | gtccctagag tcctgcttta atgagatatg cgagacgcct atgatcgcat gatatttgct |
| 6841 | ttcaattctg ttgtgcacgt tgtaaaaaac ctgagcatgt gtagctcaga tccttaccgc |
| 6901 | cggtttcggt tcattctaat gaatatatca cccgttacta tcgtattttt atgaataata |
| 6961 | ttctccgttc aatttactga ttgtacccta ctacttatat gtacaatatt aaaatgaaaa |
| 7021 | caatatattg tgctgaatag gtttatagcg acatctatga tagagcgcca caataacaaa |
| 7081 | caattgcgtt ttattattac aaatccaatt ttaaaaaaag cggcagaacc ggtcaaacct |
| 7141 | aaaagactga ttacataaat cttattcaaa tttcaaaagt gccccagggg ctagtatcta |
| 7201 | cgacacaccg agcggcgaac taataacgct cactgaaggg aactccggtt ccccgccggc |
| 7261 | gcgcatgggt gagattcctt gaagttgagt attggccgtc cgctctaccg aaagttacgg |
| 7321 | gcaccattca acccggtcca gcacggcggc cgggtaaccg acttgctgcc ccgagaatta |
| 7381 | tgcagcattt ttttggtgta tgtgggcccc aaatgaagtg caggtcaaac cttgacagtg |
| 7441 | acgacaaatc gttgggcggg tccagggcga attttgcgac aacatgtcga ggctcagcag |
| 7501 | gacctgcagg catgcaagct tggcactggc cgtcgtttta caacgtcgtg actgggaaaa |
| 7561 | ccctggcgtt acccaactta atcgccttgc agcacatccc cctttcgcca gctggcgtaa |
| 7621 | tagcgaagag gcccgcaccg atcgcccttc ccaacagttg cgcagcctga atggcgaatg |
| 7681 | ctagagcagc ttgagcttgg atcagattgt cgtttcccgc cttcagtttc ttgaaggtgc |
| 7741 | atgtgactcc gtcaagatta cgaaaccgcc aactaccacg caaattgcaa ttctcaattt |
| 7801 | cctagaagga ctctccgaaa atgcatccaa taccaaatat tacccgtgtc ataggcacca |
| 7861 | agtgacacca tacatgaaca cgcgtcacaa tatgactgga gaagggttcc acaccttatg |
| 7921 | ctataaaacg ccccacaccc ctcctccttc cttcgcagtt caattccaat atattccatt |
| 7981 | ctctctgtgt atttccctac ctctcccttc aaggttagtc gatttcttct gtttttcttc |
| 8041 | ttcgttcttt ccatgaattg tgtatgttct ttgatcaata cgatgttgat ttgattgtgt |
| 8101 | tttgtttggt ttcatcgatc ttcaattttc ataatcagat tcagctttta ttatctttac |
| 8161 | aacaacgtcc ttaatttgat gattctttaa tcgtagattt gctctaatta gagctttttc |
| 8221 | atgtcagatc cctttacaac aagccttaat tgttgattca ttaatcgtag attagggctt |
| 8281 | ttttcattga ttacttcaga tccgttaaac gtaaccatag atcagggctt tttcatgaat |
| 8341 | tacttcagat ccgttaaaca acagccttat tttttatact tctgtggttt ttcaagaaat |
| 8401 | tgttcagatc cgttgacaaa aagccttatt cgttgattct atatcgtttt tcgagagata |
| 8461 | ttgctcagat ctgttagcaa ctgccttgtt tgttgattct attgccgtgg attagggttt |
| 8521 | tttttcacga gattgcttca gatccgtact taagattacg taatggattt tgattctgat |
| 8581 | ttatctgtga ttgttgactc gacaggtacc ttcaaacggc gcgccatgca gagtttagcc |
| 8641 | atctctctac tcctctcaga aactcattcc ctcttttctc atacgaagac ctcctccctt |
| 8701 | ttatctttac tgtttctctc ttcttcaaag atgtctgagc aaaatactga tggaagtcaa |
| 8761 | gttccagtga acttgttgga tgagttcctg gctgaggatg agatcataga tgatcttctc |
| 8821 | actgaagcca cggtggtagt acagtccact atagaaggtc ttcaaaacga ggcttctgac |
| 8881 | catcgacatc atccgaggaa gcacatcaag aggccacgag aggaagcaca tcagcaactg |
| 8941 | gtgaatgatt acttttcaga aaatcctctt tacccttcca aaatttttcg tcgaagattt |
| 9001 | cgtatgtcta ggccactttt tcttcgcatc gttgaggcat taggccagtg gtcagtgtat |
| 9061 | ttcacacaaa gggtggatgc tgttaatcgg aaaggactca gtccactgca aaagtgtact |
| 9121 | gcagctattc gccagttggc tactggtagt ggcgcagatg aactagatga atatctgaag |
| 9181 | ataggagaga ctacagcaat ggaggcaatg aagaattttg tcaaaggtct tcaagatgtg |
| 9241 | tttggtgaga ggtatcttag gcgccccact atggaagata ccgaacggct tctccaactt |
| 9301 | ggtgagaaac gtggttttcc tggaatgttc ggcagcattg actgcatgca ctggcattgg |
| 9361 | gaaagatgcc cagtagcatg gaagggtcag ttcactcgtg gagatcagaa agtgccaacc |
| 9421 | ctgattcttg aggctgtggc atcgcatgat ctttggattt ggcatgcatt ttttggagca |
| 9481 | gcgggttcca acaatgatat caatgtattg aaccaatcta ctgtatttat caaggagctc |
| 9541 | aaaggacaag ctcctagagt ccagtacatg gtaaatggga atcaatacaa tactgggtat |
| 9601 | tttcttgctg atggaatcta ccctgaatgg gcagtgtttg ttaagtcaat acgactccca |
| 9661 | aacactgaaa aggagaaatt gtatgcagat atgcaagaag gggcaagaaa agatatcgag |
| 9721 | agagcctttg gtgtattgca gcgaagattt tgcatcttaa aacgaccagc tcgtctatat |
| 9781 | gatcgaggtg tactgcgaga tgttgttcta gcttgcatca tacttcacaa tatgatagtt |
| 9841 | gaagatgaga aggaaaccag aattattgaa gaagatgcag atgcaaatgt gcctcctagt |
| 9901 | tcatcaaccg ttcaggaacc tgagttctct cctgaacaga acacaccatt tgatagagtt |
| 9961 | ttagaaaaag atatttctat ccgagatcga gcggctcata accgacttaa gaaagatttg |
| 10021 | gtggaacaca tttggaataa gtttggtggt gctgcacata gaactggaaa ttaattaatt |
| 10081 | gacattctaa tctagagtcc tgctttaatg agatatgcga gacgcctatg atcgcatgat |
| 10141 | atttgctttc aattctgttg tgcacgttgt aaaaaacctg agcatgtgta gctcagatcc |
| 10201 | ttaccgccgg tttcggttca ttctaatgaa tatatcaccc gttactatcg tatttttatg |
| 10261 | aataatattc tccgttcaat ttactgattg taccctacta cttatatgta caatattaaa |
| 10321 | atgaaaacaa tatattgtgc tgaataggtt tatagcgaca tctatgatag agcgccacaa |
| 10381 | taacaaacaa ttgcgtttta ttattacaaa tccaatttta aaaaaagcgg cagaaccggt |
| 10441 | caaacctaaa agactgatta cataaatctt attcaaattt caaaagtgcc ccaggggcta |
| 10501 | gtatctacga cacaccgagc ggcgaactaa taacgttcac tgaagggaac tccggttccc |
| 10561 | cgccggcgcg catgggtgag attccttgaa gttgagtatt ggccgtccgc tctaccgaaa |
| 10621 | gttacgggca ccattcaacc cggtccagca cggcggccgg gtaaccgact tgctgccccg |
| 10681 | agaattatgc agcatttttt tggtgtatgt gggccccaaa tgaagtgcag gtcaaacctt |
| 10741 | gacagtgacg acaaatcgtt gggcgggtcc agggcgaatt ttgcgacaac atgtcgaggc |
| 10801 | tcagcaggac ctgcaggcat gcaagatcgc gaattcgtaa tcatgtcata gctagtgatc |
| 10861 | aggatattct tgtttaagat gttgaactct atggaggttt gtatgaactg atgatctagg |
| 10921 | accggataag ttcccttctt catagcgaac ttattcaaag aatgttttgt gtatcattct |
| 10981 | tgttacattg ttattaatga aaaaatatta ttggtcattg gactgaacac gagtgttaaa |
| 11041 | tatggaccag gccccaaata agatccattg atatatgaat taaataacaa gaataaatcg |
| 11101 | agtcaccaaa ccacttgcct tttttaacga gacttgttca ccaacttgat acaaaagtca |
| 11161 | ttatcctatg caaatcaata atcatacaaa aatatccaat aacactaaaa aattaaaaga |
| 11221 | aatggataat ttcacaatat gttatacgat aaagaagtta cttttccaag aaattcactg |
| 11281 | attttataag cccacttgca ttagataaat ggcaaaaaaa aacaaaaagg aaaagaaata |
| 11341 | aagcacgaag aattctagaa aatacgaaat acgcttcaat gcagtgggac ccacggttca |
| 11401 | attattgcca attttcagct ccaccgtata tttaaaaaat aaaacgataa tgctaaaaaa |
| 11461 | atataaatcg taacgatcgt taaatctcaa cggctggatc ttatgacgac cgttagaaat |
| 11521 | tgtggttgtc gacgagtcag taataaacgg cgtcaaagtg gttgcagccg gcacacacga |
| 11581 | ggcgcgcctc tagatggatt acaaggacca cgacggggat tacaaggacc acgacattga |
| 11641 | ttacaaggat gatgatgaca agatggctcc gaagaagaag aggaaggttg gcatccacgg |
| 11701 | ggtgccagct gctgacaaga agtactcgat cggcctcgat attgggacta actctgttgg |
| 11761 | ctgggccgtg atcaccgacg agtacaaggt gccctcaaag aagttcaagg tcctgggcaa |
| 11821 | caccgatcgg cattccatca agaagaatct cattggcgct ctcctgttcg acagcggcga |
| 11881 | gacggctgag gctacgcggc tcaagcgcac cgcccgcagg cggtacacgc gcaggaagaa |
| 11941 | tcgcatctgc tacctgcagg agattttctc caacgagatg gcgaaggttg acgattcttt |
| 12001 | cttccacagg ctggaggagt cattcctcgt ggaggaggat aagaagcacg agcggcatcc |
| 12061 | aatcttcggc aacattgtcg acgaggttgc ctaccacgag aagtacccta cgatctacca |
| 12121 | tctgcggaag aagctcgtgg actccacaga taaggcggac ctccgcctga tctacctcgc |
| 12181 | tctggcccac atgattaagt tcaggggcca tttcctgatc gagggggatc tcaacccgga |
| 12241 | caatagcgat gttgacaagc tgttcatcca gctcgtgcag acgtacaacc agctcttcga |
| 12301 | ggagaacccc attaatgcgt caggcgtcga cgcgaaggct atcctgtccg ctaggctctc |
| 12361 | gaagtctcgg cgcctcgaga acctgatcgc ccagctgccg ggcgagaaga agaacggcct |
| 12421 | gttcgggaat ctcattgcgc tcagcctggg gctcacgccc aacttcaagt cgaatttcga |
| 12481 | tctcgctgag gacgccaagc tgcagctctc caaggacaca tacgacgatg acctggataa |
| 12541 | cctcctggcc cagatcggcg atcagtacgc ggacctgttc ctcgctgcca agaatctgtc |
| 12601 | ggacgccatc ctcctgtctg atattctcag ggtgaacacc gagattacga aggctccgct |
| 12661 | ctcagcctcc atgatcaagc gctacgacga gcaccatcag gatctgaccc tcctgaaggc |
| 12721 | gctggtcagg cagcagctcc ccgagaagta caaggagatc ttcttcgatc agtcgaagaa |
| 12781 | cggctacgct gggtacattg acggcggggc ctctcaggag gagttctaca agttcatcaa |
| 12841 | gccgattctg gagaagatgg acggcacgga ggagctgctg gtgaagctca atcgcgagga |
| 12901 | cctcctgagg aagcagcgga cattcgataa cggcagcatc ccacaccaga ttcatctcgg |
| 12961 | ggagctgcac gctatcctga ggaggcagga ggacttctac cctttcctca aggataaccg |
| 13021 | cgagaagatc gagaagattc tgactttcag gatcccgtac tacgtcggcc cactcgctag |
| 13081 | gggcaactcc cgcttcgctt ggatgacccg caagtcagag gagacgatca cgccgtggaa |
| 13141 | cttcgaggag gtggtcgaca agggcgctag cgctcagtcg ttcatcgaga ggatgacgaa |
| 13201 | tttcgacaag aacctgccaa atgagaaggt gctccctaag cactcgctcc tgtacgagta |
| 13261 | cttcacagtc tacaacgagc tgactaaggt gaagtatgtg accgagggca tgaggaagcc |
| 13321 | ggctttcctg tctggggagc agaagaaggc catcgtggac ctcctgttca agaccaaccg |
| 13381 | gaaggtcacg gttaagcagc tcaaggagga ctacttcaag aagattgagt gcttcgattc |
| 13441 | ggtcgagatc tctggcgttg aggaccgctt caachcctcc ctggggacct accacgatct |
| 13501 | cctgaagatc attaaggata aggacttcct ggacaacgag gagaatgagg atatcctcga |
| 13561 | ggacattgtg ctgacactca ctctgttcga ggaccgggag atgatcgagg agcgcctgaa |
| 13621 | gacttacgcc catctcttcg atgacaaggt catgaagcag ctcaagagga ggaggtacac |
| 13681 | cggctggggg aggctgagca ggaagctcat caacggcatt cgggacaagc agtccgggaa |
| 13741 | gacgatcctc gacttcctga agagcgatgg cttcgcgaac cgcaatttca tgcagctgat |
| 13801 | tcacgatgac agcctcacat tcaaggagga tatccagaag gctcaggtga gcggccaggg |
| 13861 | ggactcgctg cacgagcata tcgcgaacct cgctggctcg ccagctatca agaaggggat |
| 13921 | tctgcagacc gtgaaggttg tggacgagct ggtgaaggtc atgggcaggc acaagcctga |
| 13981 | gaacatcgtc attgagatgg cccgggagaa tcagaccacg cagaagggcc agaagaactc |
| 14041 | acgcgagagg atgaagagga tcgaggaggg cattaaggag ctggggtccc agatcctcaa |
| 14101 | ggagcacccg gtggagaaca cgcagctgca gaatgagaag ctctacctgt actacctcca |
| 14161 | gaatggccgc gatatgtatg tggaccagga gctggatatt aacaggctca gcgattacga |
| 14221 | cgtcgatcat atcgttccac agtcattcct gaaggatgac tccattgaca acaaggtcct |
| 14281 | caccaggtcg gacaagaacc ggggcaagtc tgataatgtt ccttcagagg aggtcgttaa |
| 14341 | gaagatgaag aactactggc gccagctcct gaatgccaag ctgatcacgc agcggaagtt |
| 14401 | cgataacctc acaaaggctg agaggggcgg gctctctgag ctggacaagg cgggcttcat |
| 14461 | caagaggcag ctggtcgaga cacggcagat cactaagcac gttgcgcaga ttctcgactc |
| 14521 | acggatgaac actaagtacg atgagaatga caagctgatc cgcgaggtga aggtcatcac |
| 14581 | cctgaagtca aagctcgtct ccgacttcag gaaggatttc cagttctaca aggttcggga |
| 14641 | gatcaacaat taccaccatg cccatgacgc gtacctgaac gcggtggtcg gcacagctct |
| 14701 | gatcaagaag tacccaaagc tcgagagcga gttcgtgtac ggggactaca aggtttacga |
| 14761 | tgtgaggaag atgatcgcca agtcggagca ggagattggc aaggctaccg ccaagtactt |
| 14821 | cttctactct aacattatga atttcttcaa gacagagatc actctggcca atggcgagat |
| 14881 | ccggaagcgc cccctcatcg agacgaacgg cgagacgggg gagatcgtgt gggacaaggg |
| 14941 | cagggatttc gcgaccgtca ggaaggttct ctccatgcca caagtgaata tcgtcaagaa |
| 15001 | gacagaggtc cagactggcg ggttctctaa ggagtcaatt ctgcctaagc ggaacagcga |
| 15061 | caagctcatc gcccgcaaga aggactggga tccgaagaag tacggcgggt tcgacagccc |
| 15121 | cactgtggcc tactcggtcc tggttgtggc gaaggttgag aagggcaagt ccaagaagct |
| 15181 | caagagcgtg aaggagctgc tggggatcac gattatggag cgctccagct tcgagaagaa |
| 15241 | cccgatcgat ttcctggagg cgaagggcta caaggaggtg aagaaggacc tgatcattaa |
| 15301 | gctccccaag tactcactct tcgagctgga gaacggcagg aagcggatgc tggcttccgc |
| 15361 | tggcgagctg cagaagggga acgagctggc tctgccgtcc aagtatgtga acttcctcta |
| 15421 | cctggcctcc cactacgaga agctcaaggg cagccccgag gacaacgagc agaagcagct |
| 15481 | gttcgtcgag cagcacaagc attacctcga cgagatcatt gagcagattt ccgagttctc |
| 15541 | caagcgcgtg atcctggccg acgcgaatct ggataaggtc ctctccgcgt acaacaagca |
| 15601 | ccgcgacaag ccaatcaggg agcaggctga gaatatcatt catctcttca ccctgacgaa |
| 15661 | cctcggcgcc cctgctgctt tcaagtactt cgacacaact atcgatcgca agaggtacac |
| 15721 | aagcactaag gaggtcctgg acgcgaccct catccaccag tcgattaccg gcctctacga |
| 15781 | gacgcgcatc gacctgtctc agctcggggg cgacaagcgg ccagcggcga cgaagaaggc |
| 15841 | ggggcaggcg aagaagaaga agtgagctca gagctttcgt tcgtatcatc ggtttcgaca |
| 15901 | acgttcgtca agttcaatgc atcagtttca ttgcgcacac accagaatcc tactgagttt |
| 15961 | gagtattatg gcattgggaa aactgttttt cttgtaccat ttgttgtgct tgtaatttac |
| 16021 | tgtgtttttt attcggtttt cgctatcgaa ctgtgaaatg gaaatggatg gagaagagtt |
| 16081 | aatgaatgat atggtccttt tgttcattct caaattaata ttatttgttt tttctcttat |
| 16141 | ttgttgtgtg ttgaatttga aattataaga gatatgcaaa cattttgttt tgagtaaaaa |
| 16201 | tgtgtcaaat cgtggcctct aatgaccgaa gttaatatga ggagtaaaac acttgtagtt |
| 16261 | gtaccattat gcttattcac taggcaacaa atatattttc agacctagaa aagctgcaaa |
| 16321 | tgttactgaa tacaagtatg tcctcttgtg ttttagacat ttatgaactt tcctttatgt |
| 16381 | aattttccag aatccttgtc agattctaat cattgcttta taattatagt tatactcatg |
| 16441 | gatttgtagt tgagtatgaa aatatttttt aatgcatttt atgacttgcc aattgattga |
| 16501 | caacgctaga ggatccccgg gtaccgagct cgaattcgta atcatgtcat agctgtttcc |
| 16561 | tgtgtgaaat tgttatccgc tcacaattcc acacaacata cgagccggaa gcataaagtg |
| 16621 | taaagcctgg ggtgcctaat gagtgagcta actcacatta attgcgttgc gctcactgcc |
| 16681 | cgctttccag tcgggaaacc tgtcgtgcca gctgcattaa tgaatcggcc aacgcgcggg |
| 16741 | gagaggcggt ttgcgtattg gagcttgagc ttggatcaga ttgtcgtttc ccgccttcag |
| 16801 | tttaaactat cagtgtttga caggatatat tggcgggtaa acctaagaga aaagagcgtt |
| 16861 | tattagaata atcggatatt taaaagggcg tgaaaaggtt tatccgttcg tccatttgta |
| 16921 | tgtgcatgcc aaccacaggg ttcccctcgg gatcaaagta ctttaaagta ctttaaagta |
| 16981 | ctttaaagta ctttgatcca acccctccgc tgctatagtg cagtcggctt ctgacgttca |
| 17041 | gtgcagccgt cttctgaaaa cgacatgtcg cacaagtcct aagttacgcg acaggctgcc |
| 17101 | gccctgccct tttcctggcg ttttcttgtc gcgtgtttta gtcgcataaa gtagaatact |
| 17161 | tgcgactaga accggagaca ttacgccatg aacaagagcg ccgccgctgg cctgctgggc |
| 17221 | tatgcccgcg tcagcaccga cgaccaggac ttgaccaacc aacgggccga actgcacgcg |
| 17281 | gccggctgca ccaagctgtt ttccgagaag atcaccggca ccaggcgcga ccgcccggag |
| 17341 | ctggccagga tgcttgacca cctacgccct ggcgacgttg tgacagtgac caggctagac |
| 17401 | cgcctggccc gcagcacccg cgacctactg gacattgccg agcgcatcca ggaggccggc |
| 17461 | gcgggcctgc gtagcctggc agagccgtgg gccgacacca ccacgccggc cggccgcatg |
| 17521 | gtgttgaccg tgttcgccgg cattgccgag ttcgagcgtt ccctaatcat cgaccgcacc |
| 17581 | cggagcgggc gcgaggccgc caaggcccga ggcgtgaagt ttggcccccg ccctaccctc |
| 17641 | accccggcac agatcgcgca cgcccgcgag ctgatcgacc aggaaggccg caccgtgaaa |
| 17701 | gaggcggctg cactgcttgg cgtgcatcgc tcgaccctgt accgcgcact tgagcgcagc |
| 17761 | gaggaagtga cgcccaccga ggccaggcgg cgcggtgcct tccgtgagga cgcattgacc |
| 17821 | gaggccgacg ccctggcggc cgccgagaat gaacgccaag aggaacaagc atgaaaccgc |
| 17881 | accaggacgg ccaggacgaa ccgtttttca ttaccgaaga gatcgaggcg gagatgatcg |
| 17941 | cggccgggta cgtgttcgag ccgcccgcgc acgtctcaac cgtgcggctg catgaaatcc |
| 18001 | tggccggttt gtctgatgcc aagctggcgg cctggccggc cagcttggcc gctgaagaaa |
| 18061 | ccgagcgccg ccgtctaaaa aggtgatgtg tatttgagta aaacagcttg cgtcatgcgg |
| 18121 | tcgctgcgta tatgatgcga tgagtaaata aacaaatacg caaggggaac gcatgaaggt |
| 18181 | tatcgctgta cttaaccaga aaggcgggtc aggcaagacg accatcgcaa cccatctagc |
| 18241 | ccgcgccctg caactcgccg gggccgatgt tctgttagtc gattccgatc cccagggcag |
| 18301 | tgcccgcgat tgggcggccg tgcgggaaga tcaaccgcta accgttgtcg gcatcgaccg |
| 18361 | cccgacgatt gaccgcgacg tgaaggccat cggccggcgc gacttcgtag tgatcgacgg |
| 18421 | agcgccccag gcggcggact tggctgtgtc cgcgatcaag gcagccgact tcgtgctgat |
| 18481 | tccggtgcag ccaagccctt acgacatatg ggccaccgcc gacctggtgg agctggttaa |
| 18541 | gcagcgcatt gaggtcacgg atggaaggct acaagcggcc tttgtcgtgt cgcgggcgat |
| 18601 | caaaggcacg cgcatcggcg gtgaggttgc cgaggcgctg gccgggtacg agctgcccat |
| 18661 | tcttgagtcc cgtatcacgc agcgcgtgag ctacccaggc actgccgccg ccggcacaac |
| 18721 | cgttcttgaa tcagaacccg agggcgacgc tgcccgcgag gtccaggcgc tggccgctga |
| 18781 | aattaaatca aaactcattt gagttaatga ggtaaagaga aaatgagcaa aagcacaaac |
| 18841 | acgctaagtg ccggccgtcc gagcgcacgc agcagcaagg ctgcaacgtt ggccagcctg |
| 18901 | gcagacacgc cagccatgaa gcgggtcaac tttcagttgc cggcggagga tcacaccaag |
| 18961 | ctgaagatgt acgcggtacg ccaaggcaag accattaccg agctgctatc tgaatacatc |
| 19021 | gcgcagctac cagagtaaat gagcaaatga ataaatgagt agatgaattt tagcggctaa |
| 19081 | aggaggcggc atggaaaatc aagaacaacc aggcaccgac gccgtggaat gccccatgtg |
| 19141 | tggaggaacg ggcggttggc caggcgtaag cggctgggtt gtctgccggc cctgcaatgg |
| 19201 | cactggaacc cccaagcccg aggaatcggc gtgagcggtc gcaaaccatc cggcccggta |
| 19261 | caaatcggcg cggcgctggg tgatgacctg gtggagaagt tgaaggccgc gcaggccgcc |
| 19321 | cagcggcaac gcatcgaggc agaagcacgc cccggtgaat cgtggcaagc ggccgctgat |
| 19381 | cgaatccgca aagaatcccg gcaaccgccg gcagccggtg cgccgtcgat taggaagccg |
| 19441 | cccaagggcg acgagcaacc agattttttc gttccgatgc tctatgacgt gggcacccgc |
| 19501 | gatagtcgca gcatcatgga cgtggccgtt ttccgtctgt cgaagcgtga ccgacgagct |
| 19561 | ggcgaggtga tccgctacga gcttccagac gggcacgtag aggtttccgc agggccggcc |
| 19621 | ggcatggcca gtgtgtggga ttacgacctg gtactgatgg cggtttccca tctaaccgaa |
| 19681 | tccatgaacc gataccggga agggaaggga gacaagcccg gccgcgtgtt ccgtccacac |
| 19741 | gttgcggacg tactcaagtt ctgccggcga gccgatggcg gaaagcagaa agacgacctg |
| 19801 | gtagaaacct gcattcggtt aaacaccacg cacgttgcca tgcagcgtac gaagaaggcc |
| 19861 | aagaacggcc gcctggtgac ggtatccgag ggtgaagcct tgattagccg ctacaagatc |
| 19921 | gtaaagagcg aaaccgggcg gccggagtac atcgagatcg agctagctga ttggatgtac |
| 19981 | cgcgagatca cagaaggcaa gaacccggac gtgctgacgg ttcaccccga ttactttttg |
| 20041 | atcgatcccg gcatcggccg ttttctctac cgcctggcac gccgcgccgc aggcaaggca |
| 20101 | gaagccagat ggttgttcaa gacgatctac gaacgcagtg gcagcgccgg agagttcaag |
| 20161 | aagttctgtt tcaccgtgcg caagctgatc gggtcaaatg acctgccgga gtacgatttg |
| 20221 | aaggaggagg cggggcaggc tggcccgatc ctagtcatgc gctaccgcaa cctgatcgag |
| 20281 | ggcgaagcat ccgccggttc ctaatgtacg gagcagatgc tagggcaaat tgccctagca |
| 20341 | ggggaaaaag gtcgaaaagg tctctttcct gtggatagca cgtacattgg gaacccaaag |
| 20401 | ccgtacattg ggaaccggaa cccgtacatt gggaacccaa agccgtacat tgggaaccgg |
| 20461 | tcacacatgt aagtgactga tataaaagag aaaaaaggcg atttttccgc ctaaaactct |
| 20521 | ttaaaactta ttaaaactct taaaacccgc ctggcctgtg cataactgtc tggccagcgc |
| 20581 | acagccgaag agctgcaaaa agcgcctacc cttcggtcgc tgcgctccct acgccccgcc |
| 20641 | gcttcgcgtc ggcctatcgc ggccgctggc cgctcaaaaa tggctggcct acggccaggc |
| 20701 | aatctaccag ggcgcggaca agccgcgccg tcgccactcg accgccggcg cccacatcaa |
| 20761 | ggcaccctgc ctcgcgcgtt tcggtgatga cggtgaaaac ctctgacaca tgcagctccc |
| 20821 | ggagacggtc acagcttgtc tgtaagcgga tgccgggagc agacaagccc gtcagggcgc |
| 20881 | gtcagcgggt gttggcgggt gtcggggcgc agccatgacc cagtcacgta gcgatagcgg |
| 20941 | agtgtatact ggcttaacta tgcggcatca gagcagattg tactgagagt gcaccatatg |
| 21001 | cggtgtgaaa taccgcacag atgcgtaagg agaaaatacc gcatcaggcg ctcttccgct |
| 21061 | tcctcgctca ctgactcgct gcgctcggtc gttcggctgc ggcgagcggt atcagctcac |
| 21121 | tcaaaggcgg taatacggtt atccacagaa tcaggggata acgcaggaaa gaacatgtga |
| 21181 | gcaaaaggcc agcaaaaggc caggaaccgt aaaaaggccg cgttgctggc gtttttccat |
| 21241 | aggctccgcc cccctgacga gcatcacaaa aatcgacgct caagtcagag gtggcgaaac |
| 21301 | ccgacaggac tataaagata ccaggcgttt ccccctggaa gctccctcgt gcgctctcct |
| 21361 | gttccgaccc tgccgcttac cggatacctg tccgcctttc tcccttcggg aagcgtggcg |
| 21421 | ctttctcata gctcacgctg taggtatctc agttcggtgt aggtcgttcg ctccaagctg |
| 21481 | ggctgtgtgc acgaaccccc cgttcagccc gaccgctgcg ccttatccgg taactatcgt |
| 21541 | cttgagtcca acccggtaag acacgactta tcgccactgg cagcagccac tggtaacagg |
| 21601 | attagcagag cgaggtatgt aggcggtgct acagagttct tgaagtggtg gcctaactac |
| 21661 | ggctacacta gaaggacagt atttggtatc tgcgctctgc tgaagccagt taccttcgga |
| 21721 | aaaagagttg gtagctcttg atccggcaaa caaaccaccg ctggtagcgg tggttttttt |
| 21781 | gtttgcaagc agcagattac gcgcagaaaa aaaggatctc aagaagatcc tttgatcttt |
| 21841 | tctacggggt ctgacgctca gtggaacgaa aactcacgtt aagggatttt ggtcatgcat |
| 21901 | gatatatctc ccaatttgtg tagggcttat tatgcacgct taaaaataat aaaagcagac |
| 21961 | ttgacctgat agtttggctg tgagcaatta tgtgcttagt gcatctaacg cttgagttaa |
| 22021 | gccgcgccgc gaagcggcgt cggcttgaac gaatttctag ctagacatta tttgccgact |
| 22081 | accttggtga tctcgccttt cacgtagtgg acaaattctt ccaactgatc tgcgcgcgag |
| 22141 | gccaagcgat cttcttcttg tccaagataa gcctgtctag cttcaagtat gacgggctga |
| 22201 | tactgggccg gcaggcgctc cattgcccag tcggcagcga catccttcgg cgcgattttg |
| 22261 | ccggttactg cgctgtacca aatgcgggac aacgtaagca ctacatttcg ctcatcgcca |
| 22321 | gcccagtcgg gcggcgagtt ccatagcgtt aaggtttcat ttagcgcctc aaatagatcc |
| 22381 | tgttcaggaa ccggatcaaa gagttcctcc gccgctggac ctaccaaggc aacgctatgt |
| 22441 | tctcttgctt ttgtcagcaa gatagccaga tcaatgtcga tcgtggctgg ctcgaagata |
| 22501 | cctgcaagaa tgtcattgcg ctgccattct ccaaattgca gttcgcgctt agctggataa |
| 22561 | cgccacggaa tgatgtcgtc gtgcacaaca atggtgactt ctacagcgcg gagaatctcg |
| 22621 | ctctctccag gggaagccga agtttccaaa aggtcgttga tcaaagctcg ccgcgttgtt |
| 22681 | tcatcaagcc ttacggtcac cgtaaccagc aaatcaatat cactgtgtgg cttcaggccg |
| 22741 | ccatccactg cggagccgta caaatgtacg gccagcaacg tcggttcgag atggcgctcg |
| 22801 | atgacgccaa ctacctctga tagttgagtc gatacttcgg cgatcaccgc ttcccccatg |
| 22861 | atgtttaact ttgttttagg gcgactgccc tgctgcgtaa catcgttgct gctccataac |
| 22921 | atcaaacatc gacccacggc gtaacgcgct tgctgcttgg atgcccgagg catagactgt |
| 22981 | accccaaaaa aacagtcata acaagccatg aaaaccgcca ctgcgccgtt accaccgctg |
| 23041 | cgttcggtca aggttctgga ccagttgcgt gagcgcatac gctacttgca ttacagctta |
| 23101 | cgaaccgaac aggcttatgt ccactgggtt cgtgcccgaa ttgatcacag gcagcaacgc |
| 23161 | tctgtcatcg ttacaatcaa catgctaccc tccgcgagat catccgtgtt tcaaacccgg |
| 23221 | cagcttagtt gccgttcttc cgaatagcat cggtaacatg agcaaagtct gccgccttac |
| 23281 | aacggctctc ccgctgacgc cgtcccggac tgatgggctg cctgtatcga gtggtgattt |
| 23341 | tgtgccgagc tgccggtcgg ggagctgttg gctggctggt |

SEQ ID NO: 95

| | |
|---|---|
| LOCUS | ORF2_Cas9_vector_for_soybean.GFP reporter, fused Cas9pORF2, |
| | targets DD20.23836 bp ds-DNA circular 09-MAR.-2022 |
| DEFINITION | . |
| ACCESSION | pVec1 |
| VERSION | pVec1.1 |
| FEATURES | Location/Qualifiers |
| misc_feature | 1 . . . 25 |
| | /label = “LB T-DNA repeat″ |
| CDS | complement (826 . . . 1374) |
| | /label = “BlpR″ |
| promoter | complement (1566 . . . 1745) |
| | / label = “NOS promoter″ |
| regulatory | complement (2173 . . . 2428) |
| | /label = “NOS Terminator″ |
| misc_feature | complement (2448 . . . 3236) |
| | /label = “eGFP5-er″ |
| Transposon | 3266 . . . 3695 |
| | /label = “mPing″ |
| promoter | complement (3712 . . . 4545) |
| | /label = “CaMV Promoter″ |
| misc_feature | 4763 . . . 5186 |
| | /label = “U6-26promoter″ |
| misc_feature | 5187 . . . 5206 |
| | /label = “gRNA to DD20″ |
| misc_feature | 5207 . . . 5282 |
| | /label = “gRNA scaffold″ |
| misc_feature | 5283 . . . 5474 |
| | /label = “U6-26 terminator″ |
| promoter | 5490 . . . 7176 |
| | / label = “Rps5a″ |
| misc_feature | 7213 . . . 8610 |
| | /label = “ORF1″ |
| terminator | 8674 . . . 9399 |
| | /label = “OCS terminator″ |
| promoter | 9582 . . . 10501 |
| | /label = “GmUbi3 Promoter″ |
| misc_feature | 10523 . . . 11968 |
| | /label = “Pong TPase LA″ |
| CDS | 10523 . . . 16186 |
| | /label = “Translation 10523-16186″ |
| misc_feature | 11972 . . . 11986 |
| | /label = “G4S linker″ |
| feature | 11990 . . . 12010 |
| | /label = “SV40 NLS″ |
| misc_feature | 12014 . . . 16183 |
| | /label = “Cas 9″ |
| misc_feature | 16136 . . . 16183 |
| | /label = “NLS″ |
| terminator | 16211 . . . 16938 |
| | /label = “OCS Terminator″ |
| misc_feature | 17275 . . . 17299 |
| | /label = “RB T-DNA repeat″ |
| CDS | 18630 . . . 19259 |
| | /label = “pVS1 StaA″ |
| CDS | 19688 . . . 20761 |
| | /label = “pVS1 RepA″ |
| rep_origin | 20827 . . . 21021 |
| | /label = “pVS1 oriV″ |
| misc_feature | 21365 . . . 21505 |
| | /label = “bom″ |
| rep origin | complement (21691 . . . 22279) |
| | /label = “ori″ |
| CDS | complement (22525 . . . 23316) |
| | / label = “SmR″ |

ORIGIN

| | |
|---|---|
| 1 | tggcaggata tattgtggtg taaacaaatt gacgcttaga caacttaata acacattgcg |
| 61 | gacgttttta atgtactgaa ttaacgccga attgctctag cattcgccat tcaggctgcg |
| 121 | caactgttgg gaagggcgat cggtgcgggc ctcttcgcta ttacgccagc tggcgaaagg |
| 181 | gggatgtgct gcaaggcgat taagttgggt aacgccaggg ttttcccagt cacgacgttg |
| 241 | taaaacgacg gccagtgcca agctaattcg cttcaagacg tgctcaaatc actatttcca |
| 301 | cacccctata tttctattgc actccctttt aactgttttt tattacaaaa atgccctgga |
| 361 | aaatgcactc cctttttgtg tttgtttttt tgtgaaacga tgttgtcagg taatttattt |
| 421 | gtcagtctac tatggtggcc cattatatta atagcaactg tcggtccaat agacgacgtc |
| 481 | gattttctgc atttgtttaa ccacgtggat tttatgacat tttatattag ttaatttgta |
| 541 | aaacctaccc aattaaagac ctcatatgtt ctaaagacta atacttaatg ataacaattt |
| 601 | tcttttagtg aagaaaggga taattagtaa atatggaaca agggcagaag atttattaaa |
| 661 | gccgcgtaag agacaacaag taggtacgtg gagtgtctta ggtgacttac ccacataaca |
| 721 | taaagtgaca ttaacaaaca tagctaatgc tcctatttga atagtgcata tcagcatacc |
| 781 | ttattacata tagataggag caaactctag ctagattgtt gagcagatct cggtgacggg |
| 841 | caggaccgga cggggcggta ccggcaggct gaagtccagc tgccagaaac ccacgtcatg |
| 901 | ccagttcccg tgcttgaagc cggccgcccg cagcatgccg cggggggcat atccgagcgc |
| 961 | ctcgtgcatg cgcacgctcg ggtcgttggg cagcccgatg acagcgacca cgctcttgaa |
| 1021 | gccctgtgcc tccagggact tcagcaggtg ggtgtagagc gtggagccca gtcccgtccg |
| 1081 | ctggtggcgg ggggagacgt acacggtcga ctcggccgtc cagtcgtagg cgttgcgtgc |
| 1141 | cttccagggg cccgcgtagg cgatgccggc gacctcgccg tccacctcgg cgacgagcca |
| 1201 | gggatagcgc tcccgcagac ggacgaggtc gtccgtccac tcctgcggtt cctgcggctc |
| 1261 | ggtacggaag ttgaccgtgc ttgtctcgat gtagtggttg acgatggtgc agaccgccgg |
| 1321 | catgtccgcc tcggtggcac ggcggatgtc ggccgggcgt cgttctgggc tcatggtaga |
| 1381 | tcccccgttc gtaaatggtg aaaattttca gaaaattgct tttgctttaa aagaaatgat |
| 1441 | ttaaattgct gcaatagaag tagaatgctt gattgcttga gattcgtttg ttttgtatat |
| 1501 | gttgtgttga gaattaattc tcgagcctag agtcgagatc tggattgaga gtgaatatga |
| 1561 | gactctaatt ggataccgag gggaatttat ggaacgtcag tggagcattt ttgacaagaa |
| 1621 | atatttgcta gctgatagtg accttaggcg acttttgaac gcgcaataat ggtttctgac |
| 1681 | gtatgtgctt agctcattaa actccagaaa cccgcggctg agtggctcct tcaacgttgc |
| 1741 | ggttctgtca gttccaaacg taaaacggct tgtcccgcgt catcggcggg ggtcataacg |
| 1801 | tgactccctt aattctccgc tcatgatctt gatcccctgc gccatcagat ccttggcggc |
| 1861 | aagaaagcca tccagtttac tttgcagggc ttcccaacct taccagaggg cgccccagct |
| 1921 | ggcaattccg gttcgcttgc tgtccataaa accgcccagt ctagctatcg ccatgtaagc |
| 1981 | ccactgcaag ctacctgctt tctctttgcg cttgcgtttt cccttgtcca gatagcccag |
| 2041 | tagctgacat tcatccgggg tcagcaccgt ttctgcggac tggctttcta cgtgttccgc |
| 2101 | ttcctttagc agcccttgcg ccctgagtgc ttgcggcagc gtgaagcttg catgcctgca |
| 2161 | ggtcgactct agcccgatct agtaacatag atgacaccgc gcgcgataat ttatcctagt |
| 2221 | ttgcgcgcta tattttgttt tctatcgcgt attaaatgta taattgcggg actctaatca |
| 2281 | taaaaaccca tctcataaat aacgtcatgc attacatgtt aattattaca tgcttaacgt |
| 2341 | aattcaacag aaattatatg ataatcatcg caagaccggc aacaggattc aatcttaaga |
| 2401 | aactttattg ccaaatgttt gaacgatcgg ggaaattcga gctcttaaag ctcatcatgt |
| 2461 | ttgtatagtt catccatgcc atgtgtaatc ccagcagctg ttacaaactc aagaaggacc |
| 2521 | atgtggtctc tcttttcgtt gggatctttc gaaagggcag attgtgtgga caggtaatgg |
| 2581 | ttgtctggta aaaggacagg gccatcgcca attggagtat tttgttgata atgatcagcg |
| 2641 | agttgcacgc cgccgtcttc gatgttgtgg cgggtcttga agttggcttt gatgccgttc |
| 2701 | ttttgcttgt cggccatgat gtatacgttg tgggagttgt agttgtattc caacttgtgg |
| 2761 | ccgaggatgt ttccgtcctc cttgaaatcg attcccttaa gctcgatcct gttgacgagg |
| 2821 | gtgtctccct caaacttgac ttcagcacgt gtcttgtagt tcccgtcgtc cttgaagaag |
| 2881 | atggtcctct cctgcacgta tccctcaggc atggcgctct tgaagaagtc gtgccgcttc |
| 2941 | atatgatctg ggtatcttga aaagcattga acaccataag agaaagtagt gacaagtgtt |
| 3001 | ggccatggaa caggtagttt tccagtagtg caaataaatt taagggtaag ttttccgtat |
| 3061 | gttgcatcac cttcaccctc tccactgaca gaaaatttgt gcccattaac atcaccatct |
| 3121 | aattcaacaa gaattgggac aactccagtg aaaagttctt ctcctttact gaattcggcc |
| 3181 | gaggataatg ataggagaag tgaaaagatg agaaagagaa aaagattagt cttcattgtt |
| 3241 | atatctcctt ggatcctcta gattaggcca gtcacaatgg ctagtgtcat tgcacggcta |
| 3301 | cccaaaatat tataccatct tctctcaaat gaaatctttt atgaaacaat ccccacagtg |
| 3361 | gaggggtttc actttgacgt ttccaagact aagcaaagca tttaattgat acaagttgct |
| 3421 | gggatcattt gtacccaaaa tccggcgcgg cgcgggagaa tgcggaggtc gcacggcgga |
| 3481 | ggcggacgca agagatccgg tgaatgaaac gaatcggcct caacgggggt ttcactctgt |
| 3541 | taccgaggac ttggaaacga cgctgacgag tttcaccagg atgaaactct ttccttctct |
| 3601 | ctcatcccca tttcatgcaa ataatcattt tttattcagt cttaccccta ttaaatgtgc |
| 3661 | atgacacacc agtgaaaccc ccattgtgac tggccttatc tagagtcccc cgtgttctct |
| 3721 | ccaaatgaaa tgaacttcct tatatagagg aagggtcttg cgaaggatag tgggattgtg |
| 3781 | cgtcatccct tacgtcagtg gagatatcac atcaatccac ttgctttgaa gacgtggttg |
| 3841 | gaacgtcttc tttttccacg atgctcctcg tgggtggggg tccatctttg ggaccactgt |
| 3901 | cggcagaggc atcttcaacg atggcctttc ctttatcgca atgatggcat ttgtaggagc |
| 3961 | caccttcctt ttccactatc ttcacaataa agtgacagat agctgggcaa tggaatccga |
| 4021 | ggaggtttcc ggatattacc ctttgttgaa aagtctcaat tgccctttgg tcttctgaga |
| 4081 | ctgtatcttt gatatttttg gagtagacaa gtgtgtcgtg ctccaccatg ttgacgaaga |
| 4141 | ttttcttctt gtcattgagt cgtaagagac tctgtatgaa ctgttcgcca gtctttacgg |
| 4201 | cgagttctgt taggtcctct atttgaatct ttgactccat ggcctttgat tcagtgggaa |
| 4261 | ctaccttttt agagactcca atctctatta cttgccttgg tttgtgaagc aagccttgaa |
| 4321 | tcgtccatac tggaatagta cttctgatct tgagaaatat atctttctct gtgttcttga |
| 4381 | tgcagttagt cctgaatctt ttgactgcat ctttaacctt cttgggaagg tatttgattt |
| 4441 | cctggagatt attgctcggg tagatcgtct tgatgagacc tgctgcgtaa gcctctctaa |
| 4501 | ccatctgtgg gttagcattc tttctgaaat tgaaaaggct aatctgggaa actgaaggcg |
| 4561 | ggaaacgaca atctgatcca agctcaagct gctctagcat tcgccattca ggctgcgcaa |
| 4621 | ctgttgggaa gggcgatcgg tgcgggcctc ttcgctatta cgccagctgg cgaaaggggg |
| 4681 | atgtgctgca aggcgattaa gttgggtaac gccagggttt tcccagtcac gacgttgtaa |
| 4741 | aacgacggcc agtgccaagc ttcgacttgc cttccgcaca atacatcatt tcttcttagc |
| 4801 | tttttttctt cttcttcgtt catacagttt ttttttgttt atcagcttac attttcttga |
| 4861 | accgtagctt tcgttttctt ctttttaact ttccattcgg agtttttgta tcttgtttca |
| 4921 | tagtttgtcc caggattaga atgattaggc atcgaacctt caagaatttg attgaataaa |
| 4981 | acatcttcat tcttaagata tgaagataat cttcaaaagg cccctgggaa tctgaaagaa |
| 5041 | gagaagcagg cccatttata tgggaaagaa caatagtatt tcttatatag gcccatttaa |
| 5101 | gttgaaaaca atcttcaaaa gtcccacatc gcttagataa gaaaacgaag ctgagtttat |
| 5161 | atacagctag agtcgaagta gtgattggaa ctgacacacg acatgagttt tagagctaga |
| 5221 | aatagcaagt taaaataagg ctagtccgtt atcaacttga aaaagtggca ccgagtcggt |
| 5281 | gctttttttt gcaaaatttt ccagatcgat ttcttcttcc tctgttcttc ggcgttcaat |
| 5341 | ttctggggtt ttctcttcgt tttctgtaac tgaaacctaa aatttgacct aaaaaaaatc |
| 5401 | tcaaataata tgattcagtg gttttgtact tttcagttag ttgagttttg cagttccgat |
| 5461 | gagataaacc aataccatgt tagagagcgc tagttcgtga gtagatatat tactcaactt |
| 5521 | ttgattcgct atttgcagtg cacctgtggc gttcatcaca tcttttgtga cactgtttgc |
| 5581 | actggtcatt gctattacaa aggaccttcc tgatgttgaa ggagatcgaa agtaagtaac |
| 5641 | tgcacgcata accattttct ttccgctctt tggctcaatc catttgacag tcaaagacaa |
| 5701 | tgtttaacca gctccgtttg atatattgtc tttatgtgtt tgttcaagca tgtttagtta |
| 5761 | atcatgcctt tgattgatct tgaataggtt ccaaatatca accctggcaa caaaacttgg |
| 5821 | agtgagaaac attgcattcc tcggttctgg acttctgcta gtaaattatg tttcagccat |
| 5881 | atcactagct ttctacatgc ctcaggtgaa ttcatctatt tccgtcttaa ctatttcggt |
| 5941 | taatcaaagc acgaacacca ttactgcatg tagaagcttg ataaactatc gccaccaatt |
| 6001 | tatttttgtt gcgatattgt tactttcctc agtatgcagc tttgaaaaga ccaaccctct |
| 6061 | tatcctttaa caatgaacag gtttttagag gtagcttgat gattcctgca catgtgatct |
| 6121 | tggcttcagg cttaattttc caggtaaagc attatgagat actcttatat ctcttacata |
| 6181 | cttttgagat aatgcacaag aacttcataa ctatatgctt tagtttctgc atttgacact |
| 6241 | gccaaattca ttaatctcta atatctttgt tgttgatctt tggtagacat gggtactaga |
| 6301 | aaaagcaaac tacaccaagg taaaatactt ttgtacaaac ataaactcgt tatcacggaa |
| 6361 | catcaatgga gtgtatatct aacggagtgt agaaacattt gattattgca ggaagctatc |
| 6421 | tcaggatatt atcggtttat atggaatctc ttctacgcag agtatctgtt attccccttc |
| 6481 | ctctagcttt caatttcatg gtgaggatat gcagttttct ttgtatatca ttcttcttct |
| 6541 | tctttgtagc ttggagtcaa aatcggttcc ttcatgtaca tacatcaagg atatgtcctt |
| 6601 | ctgaattttt atatcttgca ataaaaatgc ttgtaccaat tgaaacacca gctttttgag |
| 6661 | ttctatgatc actgacttgg ttctaaccaa aaaaaaaaaa atgtttaatt tacatatcta |
| 6721 | aaagtaggtt tagggaaacc taaacagtaa aatatttgta tattattcga atttcactca |
| 6781 | tcataaaaac ttaaattgca ccataaaatt ttgttttact attaatgatg taatttgtgt |
| 6841 | aacttaagat aaaaataata ttccgtaagt taaccggcta aaaccacgta taaaccaggg |
| 6901 | aacctgttaa accggttctt tactggataa agaaatgaaa gcccatgtag acagctccat |
| 6961 | tagagcccaa accctaaatt tctcatctat ataaaaggag tgacattagg gtttttgttc |
| 7021 | gtcctcttaa agcttctcgt tttctctgcc gtctctctca ttcgcgcgac gcaaacgatc |
| 7081 | ttcaggtgat cttctttctc caaatcctct ctcataactc tgatttcgta cttgtgtatt |
| 7141 | tgagctcacg ctctgtttct ctcaccacag ccggattcga gatcacaagt ttgtacaaaa |
| 7201 | aagcaggctt ccatggatcc gtcgccggcc gtggatccgt cgccggccgt ggatccgtcg |
| 7261 | ccggctgctg aaacccggcg gcgtgcaacc gggaaaggag gcaaacagcg cgggggcaag |
| 7321 | caactaggat tgaagaggcc gccgccgatt tctgtcccgg ccaccccgcc tcctgctgcg |
| 7381 | acgtcttcat cccctgctgc gccgacggcc atcccaccac gaccaccgca atcttcgccg |
| 7441 | attttcgtcc ccgattcgcc gaatccgtca ccggctgcgc cgacctcctc tcttgcttcg |
| 7501 | gggacatcga cggcaaggcc accgcaacca caaggaggag gatggggacc aacatcgacc |
| 7561 | atttccccaa actttgcatc tttctttgga aaccaacaag acccaaattc atgtttggtc |
| 7621 | aggggttatc ctccaggagg gtttgtcaat tttattcaac aaaattgtcc gccgcagcca |
| 7681 | caacagcaag gtgaaaattt tcatttcgtt ggtcacaata tggggttcaa cccaatatct |
| 7741 | ccacagccac caagtgccta cggaacacca acaccccaag ctacgaacca aggcacttca |
| 7801 | acaaacatta tgattgatga agaggacaac aatgatgaca gtagggcagc aaagaaaaga |
| 7861 | tggactcatg aagaggaaga gagactggcc agtgcttggt tgaatgcttc taaagactca |
| 7921 | attcatggga atgataagaa aggtgataca ttttggaagg aagtcactga tgaatttaac |
| 7981 | aagaaaggga atggaaaacg taggagggaa attaaccaac tgaaggttca ctggtcaagg |
| 8041 | ttgaagtcag cgatctctga gttcaatgac tattggagta cggttactca aatgcataca |
| 8101 | agcggatact cagacgacat gcttgagaaa gaggcacaga ggctgtatgc aaacaggttt |
| 8161 | ggaaaacctt ttgcgttggt ccattggtgg aagatactca aaagagagcc caaatggtgt |
| 8221 | gctcagtttg aaaagaggaa aaggaagagc gaaatggatg ctgttccaga acagcagaaa |
| 8281 | cgtcctattg gtagagaagc agcaaagtct gagcgcaaaa gaaagcgcaa gaaagaaaat |
| 8341 | gttatggaag gcattgtcct cctaggggac aatgtccaga aaattatcaa agtgacgcaa |
| 8401 | gatcggaagc tggagcgtga gaaggtcact gaagcacaga ttcacatttc aaacgtaaat |
| 8461 | ttgaaggcag cagaacagca aaaagaagca aagatgtttg aggtatacaa ttccctgctc |
| 8521 | actcaagata caagtaacat gtctgaagaa cagaaggctc gccgagacaa ggcattacaa |
| 8581 | aagctggagg aaaagttatt tgctgactag tgacccagct ttcttgtaca aagtggtgcc |
| 8641 | taggtgagtc tagagagttg attaagaccc gggactggtc cctagagtcc tgctttaatg |
| 8701 | agatatgcga gacgcctatg atcgcatgat atttgctttc aattctgttg tgcacgttgt |
| 8761 | aaaaaacctg agcatgtgta gctcagatcc ttaccgccgg tttcggttca ttctaatgaa |
| 8821 | tatatcaccc gttactatcg tatttttatg aataatattc tccgttcaat ttactgattg |
| 8881 | taccctacta cttatatgta caatattaaa atgaaaacaa tatattgtgc tgaataggtt |
| 8941 | tatagcgaca tctatgatag agcgccacaa taacaaacaa ttgcgtttta ttattacaaa |
| 9001 | tccaatttta aaaaaagcgg cagaaccggt caaacctaaa agactgatta cataaatctt |
| 9061 | attcaaattt caaaagtgcc ccaggggcta gtatctacga cacaccgagc ggcgaactaa |
| 9121 | taacgctcac tgaagggaac tccggttccc cgccggcgcg catgggtgag attccttgaa |
| 9181 | gttgagtatt ggccgtccgc tctaccgaaa gttacgggca ccattcaacc cggtccagca |
| 9241 | cggcggccgg gtaaccgact tgctgccccg agaattatgc agcatttttt tggtgtatgt |
| 9301 | gggccccaaa tgaagtgcag gtcaaacctt gacagtgacg acaaatcgtt gggcgggtcc |
| 9361 | agggcgaatt ttgcgacaac atgtcgaggc tcagcaggac ctgcaggcat gcaagcttgg |
| 9421 | cactggccgt cgttttacaa cgtcgtgact gggaaaaccc tggcgttacc caacttaatc |
| 9481 | gccttgcagc acatccccct ttcgccagct ggcgtaatag cgaagaggcc cgcaccgatc |
| 9541 | gcccttccca acagttgcgc agcctgaatg gcgaatgcta gagcagcttg agcttggatc |
| 9601 | agattgtcgt ttcccgcctt cagtttcttg aaggtgcatg tgactccgtc aagattacga |
| 9661 | aaccgccaac taccacgcaa attgcaattc tcaatttcct agaaggactc tccgaaaatg |
| 9721 | catccaatac caaatattac ccgtgtcata ggcaccaagt gacaccatac atgaacacgc |
| 9781 | gtcacaatat gactggagaa gggttccaca ccttatgcta taaaacgccc cacacccctc |
| 9841 | ctccttcctt cgcagttcaa ttccaatata ttccattctc tctgtgtatt tccctacctc |
| 9901 | tcccttcaag gttagtcgat ttcttctgtt tttcttcttc gttctttcca tgaattgtgt |
| 9961 | atgttctttg atcaatacga tgttgatttg attgtgtttt gtttggtttc atcgatcttc |
| 10021 | aattttcata atcagattca gcttttatta tctttacaac aacgtcctta atttgatgat |
| 10081 | tctttaatcg tagatttgct ctaattagag ctttttcatg tcagatccct ttacaacaag |
| 10141 | ccttaattgt tgattcatta atcgtagatt agggcttttt tcattgatta cttcagatcc |
| 10201 | gttaaacgta accatagatc agggcttttt catgaattac ttcagatccg ttaaacaaca |
| 10261 | gccttatttt ttatacttct gtggtttttc aagaaattgt tcagatccgt tgacaaaaag |
| 10321 | ccttattcgt tgattctata tcgtttttcg agagatattg ctcagatctg ttagcaactg |
| 10381 | ccttgtttgt tgattctatt gccgtggatt agggtttttt ttcacgagat tgcttcagat |
| 10441 | ccgtacttaa gattacgtaa tggattttga ttctgattta tctgtgattg ttgactcgac |
| 10501 | aggtaccttc aaacggcgcg ccatgcagag tttagccatc tctctactcc tctcagaaac |
| 10561 | tcattccctc ttttctcata cgaagacctc ctccctttta tctttactgt ttctctcttc |
| 10621 | ttcaaagatg tctgagcaaa atactgatgg aagtcaagtt ccagtgaact tgttggatga |
| 10681 | gttcctggct gaggatgaga tcatagatga tcttctcact gaagccacgg tggtagtaca |
| 10741 | gtccactata gaaggtcttc aaaacgaggc ttctgaccat cgacatcatc cgaggaagca |
| 10801 | catcaagagg ccacgagagg aagcacatca gcaactggtg aatgattact tttcagaaaa |
| 10861 | tcctctttac ccttccaaaa tttttcgtcg aagatttcgt atgtctaggc cactttttct |
| 10921 | tcgcatcgtt gaggcattag gccagtggtc agtgtatttc acacaaaggg tggatgctgt |
| 10981 | taatcggaaa ggactcagtc cactgcaaaa gtgtactgca gctattcgcc agttggctac |
| 11041 | tggtagtggc gcagatgaac tagatgaata tctgaagata ggagagacta cagcaatgga |
| 11101 | ggcaatgaag aattttgtca aaggtcttca agatgtgttt ggtgagaggt atcttaggcg |
| 11161 | ccccactatg gaagataccg aacggcttct ccaacttggt gagaaacgtg gttttcctgg |
| 11221 | aatgttcggc agcattgact gcatgcactg gcattgggaa agatgcccag tagcatggaa |
| 11281 | gggtcagttc actcgtggag atcagaaagt gccaaccctg attcttgagg ctgtggcatc |
| 11341 | gcatgatctt tggatttggc atgcattttt tggagcagcg ggttccaaca atgatatcaa |
| 11401 | tgtattgaac caatctactg tatttatcaa ggagctcaaa ggacaagctc ctagagtcca |
| 11461 | gtacatggta aatgggaatc aatacaatac tgggtatttt cttgctgatg gaatctaccc |
| 11521 | tgaatgggca gtgtttgtta agtcaatacg actcccaaac actgaaaagg agaaattgta |
| 11581 | tgcagatatg caagaagggg caagaaaaga tatcgagaga gcctttggtg tattgcagcg |
| 11641 | aagattttgc atcttaaaac gaccagctcg tctatatgat cgaggtgtac tgcgagatgt |
| 11701 | tgttctagct tgcatcatac ttcacaatat gatagttgaa gatgagaagg aaaccagaat |
| 11761 | tattgaagaa gatgcagatg caaatgtgcc tcctagttca tcaaccgttc aggaacctga |
| 11821 | gttctctcct gaacagaaca caccatttga tagagtttta gaaaaagata tttctatccg |
| 11881 | agatcgagcg gctcataacc gacttaagaa agatttggtg gaacacattt ggaataagtt |
| 11941 | tggtggtgct gcacatagaa ctggaaatta tggcggggga ggtagcgctc cgaagaagaa |
| 12001 | gaggaaggtt ggcatccacg gggtgccagc tgctgacaag aagtactcga tcggcctcga |
| 12061 | tattgggact aactctgttg gctgggccgt gatcaccgac gagtacaagg tgccctcaaa |
| 12121 | gaagttcaag gtcctgggca acaccgatcg gcattccatc aagaagaatc tcattggcgc |
| 12181 | tctcctgttc gacagcggcg agacggctga ggctacgcgg ctcaagcgca ccgcccgcag |
| 12241 | gcggtacacg cgcaggaaga atcgcatctg ctacctgcag gagattttct ccaacgagat |
| 12301 | ggcgaaggtt gacgattctt tcttccacag gctggaggag tcattcctcg tggaggagga |
| 12361 | taagaagcac gagcggcatc caatcttcgg caacattgtc gacgaggttg cctaccacga |
| 12421 | gaagtaccct acgatctacc atctgcggaa gaagctcgtg gactccacag ataaggcgga |
| 12481 | cctccgcctg atctacctcg ctctggccca catgattaag ttcaggggcc atttcctgat |
| 12541 | cgagggggat ctcaacccgg acaatagcga tgttgacaag ctgttcatcc agctcgtgca |
| 12601 | gacgtacaac cagctcttcg aggagaaccc cattaatgcg tcaggcgtcg acgcgaaggc |
| 12661 | tatcctgtcc gctaggctct cgaagtctcg gcgcctcgag aacctgatcg cccagctgcc |
| 12721 | gggcgagaag aagaacggcc tgttcgggaa tctcattgcg ctcagcctgg ggctcacgcc |
| 12781 | caacttcaag tcgaatttcg atctcgctga ggacgccaag ctgcagctct ccaaggacac |
| 12841 | atacgacgat gacctggata acctcctggc ccagatcggc gatcagtacg cggacctgtt |
| 12901 | cctcgctgcc aagaatctgt cggacgccat cctcctgtct gatattctca gggtgaacac |
| 12961 | cgagattacg aaggctccgc tctcagcctc catgatcaag cgctacgacg agcaccatca |
| 13021 | ggatctgacc ctcctgaagg cgctggtcag gcagcagctc cccgagaagt acaaggagat |
| 13081 | cttcttcgat cagtcgaaga acggctacgc tgggtacatt gacggcgggg cctctcagga |
| 13141 | ggagttctac aagttcatca agccgattct ggagaagatg gacggcacgg aggagctgct |
| 13201 | ggtgaagctc aatcgcgagg acctcctgag gaagcagcgg acattcgata acggcagcat |
| 13261 | cccacaccag attcatctcg gggagctgca cgctatcctg aggaggcagg aggacttcta |
| 13321 | ccctttcctc aaggataacc gcgagaagat cgagaagatt ctgactttca ggatcccgta |
| 13381 | ctacgtcggc ccactcgcta ggggcaactc ccgcttcgct tggatgaccc gcaagtcaga |
| 13441 | ggagacgatc acgccgtgga acttcgagga ggtggtcgac aagggcgcta gcgctcagtc |
| 13501 | gttcatcgag aggatgacga atttcgacaa gaacctgcca aatgagaagg tgctccctaa |
| 13561 | gcactcgctc ctgtacgagt acttcacagt ctacaacgag ctgactaagg tgaagtatgt |
| 13621 | gaccgagggc atgaggaagc cggctttcct gtctggggag cagaagaagg ccatcgtgga |
| 13681 | cctcctgttc aagaccaacc ggaaggtcac ggttaagcag ctcaaggagg actacttcaa |
| 13741 | gaagattgag tgcttcgatt cggtcgagat ctctggcgtt gaggaccgct tcaacgcctc |
| 13801 | cctggggacc taccacgatc tcctgaagat cattaaggat aaggacttcc tggacaacga |
| 13861 | ggagaatgag gatatcctcg aggacattgt gctgacactc actctgttcg aggaccggga |
| 13921 | gatgatcgag gagcgcctga agacttacgc ccatctcttc gatgacaagg tcatgaagca |
| 13981 | gctcaagagg aggaggtaca ccggctgggg gaggctgagc aggaagctca tcaacggcat |
| 14041 | tcgggacaag cagtccggga agacgatcct cgacttcctg aagagcgatg gcttcgcgaa |
| 14101 | ccgcaatttc atgcagctga ttcacgatga cagcctcaca ttcaaggagg atatccagaa |
| 14161 | ggctcaggtg agcggccagg gggactcgct gcacgagcat atcgcgaacc tcgctggctc |
| 14221 | gccagctatc aagaagggga ttctgcagac cgtgaaggtt gtggacgagc tggtgaaggt |
| 14281 | catgggcagg cacaagcctg agaacatcgt cattgagatg gcccgggaga atcagaccac |
| 14341 | gcagaagggc cagaagaact cacgcgagag gatgaagagg atcgaggagg gcattaagga |
| 14401 | gctggggtcc cagatcctca aggagcaccc ggtggagaac acgcagctgc agaatgagaa |
| 14461 | gctctacctg tactacctcc agaatggccg cgatatgtat gtggaccagg agctggatat |
| 14521 | taacaggctc agcgattacg acgtcgatca tatcgttcca cagtcattcc tgaaggatga |
| 14581 | ctccattgac aacaaggtcc tcaccaggtc ggacaagaac cggggcaagt ctgataatgt |
| 14641 | tccttcagag gaggtcgtta agaagatgaa gaactactgg cgccagctcc tgaatgccaa |
| 14701 | gctgatcacg cagcggaagt tcgataacct cacaaaggct gagaggggcg ggctctctga |
| 14761 | gctggacaag gcgggcttca tcaagaggca gctggtcgag acacggcaga tcactaagca |
| 14821 | cgttgcgcag attctcgact cacggatgaa cactaagtac gatgagaatg acaagctgat |
| 14881 | ccgcgaggtg aaggtcatca ccctgaagtc aaagctcgtc tccgacttca ggaaggattt |
| 14941 | ccagttctac aaggttcggg agatcaacaa ttaccaccat gcccatgacg cgtacctgaa |
| 15001 | cgcggtggtc ggcacagctc tgatcaagaa gtacccaaag ctcgagagcg agttcgtgta |
| 15061 | cggggactac aaggtttacg atgtgaggaa gatgatcgcc aagtcggagc aggagattgg |
| 15121 | caaggctacc gccaagtact tcttctactc taacattatg aatttcttca agacagagat |
| 15181 | cactctggcc aatggcgaga tccggaagcg ccccctcatc gagacgaacg gcgagacggg |
| 15241 | ggagatcgtg tgggacaagg gcagggattt cgcgaccgtc aggaaggttc tctccatgcc |
| 15301 | acaagtgaat atcgtcaaga agacagaggt ccagactggc gggttctcta aggagtcaat |
| 15361 | tctgcctaag cggaacagcg acaagctcat cgcccgcaag aaggactggg atccgaagaa |
| 15421 | gtacggcggg ttcgacagcc ccactgtggc ctactcggtc ctggttgtgg cgaaggttga |
| 15481 | gaagggcaag tccaagaagc tcaagagcgt gaaggagctg ctggggatca cgattatgga |
| 15541 | gcgctccagc ttcgagaaga acccgatcga tttcctggag gcgaagggct acaaggaggt |
| 15601 | gaagaaggac ctgatcatta agctccccaa gtactcactc ttcgagctgg agaacggcag |
| 15661 | gaagcggatg ctggcttccg ctggcgagct gcagaagggg aacgagctgg ctctgccgtc |
| 15721 | caagtatgtg aacttcctct acctggcctc ccactacgag aagctcaagg gcagccccga |
| 15781 | ggacaacgag cagaagcagc tgttcgtcga gcagcacaag cattacctcg acgagatcat |
| 15841 | tgagcagatt tccgagttct ccaagcgcgt gatcctggcc gacgcgaatc tggataaggt |
| 15901 | cctctccgcg tacaacaagc accgcgacaa gccaatcagg gagcaggctg agaatatcat |
| 15961 | tcatctcttc accctgacga acctcggcgc ccctgctgct ttcaagtact tcgacacaac |
| 16021 | tatcgatcgc aagaggtaca caagcactaa ggaggtcctg gacgcgaccc tcatccacca |
| 16081 | gtcgattacc ggcctctacg agacgcgcat cgacctgtct cagctcgggg gcgacaagcg |
| 16141 | gccagcggcg acgaagaagg cggggcaggc gaagaagaag aagtgataat tgacattcta |
| 16201 | atctagagtc ctgctttaat gagatatgcg agacgcctat gatcgcatga tatttgcttt |
| 16261 | caattctgtt gtgcacgttg taaaaaacct gagcatgtgt agctcagatc cttaccgccg |
| 16321 | gtttcggttc attctaatga atatatcacc cgttactatc gtatttttat gaataatatt |
| 16381 | ctccgttcaa tttactgatt gtaccctact acttatatgt acaatattaa aatgaaaaca |
| 16441 | atatattgtg ctgaataggt ttatagcgac atctatgata gagcgccaca ataacaaaca |
| 16501 | attgcgtttt attattacaa atccaatttt aaaaaaagcg gcagaaccgg tcaaacctaa |
| 16561 | aagactgatt acataaatct tattcaaatt tcaaaagtgc cccaggggct agtatctacg |
| 16621 | acacaccgag cggcgaacta ataacgttca ctgaagggaa ctccggttcc ccgccggcgc |
| 16681 | gcatgggtga gattccttga agttgagtat tggccgtccg ctctaccgaa agttacgggc |
| 16741 | accattcaac ccggtccagc acggcggccg ggtaaccgac ttgctgcccc gagaattatg |
| 16801 | cagcattttt ttggtgtatg tgggccccaa atgaagtgca ggtcaaacct tgacagtgac |
| 16861 | gacaaatcgt tgggcgggtc cagggcgaat tttgcgacaa catgtcgagg ctcagcagga |
| 16921 | cctgcaggca tgcaagatcg cgaattcgta atcatgtcat agctagagga tccccgggta |
| 16981 | ccgagctcga attcgtaatc atgtcatagc tgtttcctgt gtgaaattgt tatccgctca |
| 17041 | caattccaca caacatacga gccggaagca taaagtgtaa agcctggggt gcctaatgag |
| 17101 | tgagctaact cacattaatt gcgttgcgct cactgcccgc tttccagtcg ggaaacctgt |
| 17161 | cgtgccagct gcattaatga atcggccaac gcgcggggag aggcggtttg cgtattggag |
| 17221 | cttgagcttg gatcagattg tcgtttcccg ccttcagttt aaactatcag tgtttgacag |
| 17281 | gatatattgg cgggtaaacc taagagaaaa gagcgtttat tagaataatc ggatatttaa |
| 17341 | aagggcgtga aaaggtttat ccgttcgtcc atttgtatgt gcatgccaac cacagggttc |
| 17401 | ccctcgggat caaagtactt taaagtactt taaagtactt taaagtactt tgatccaacc |
| 17461 | cctccgctgc tatagtgcag tcggcttctg acgttcagtg cagccgtctt ctgaaaacga |
| 17521 | catgtcgcac aagtcctaag ttacgcgaca ggctgccgcc ctgccctttt cctggcgttt |
| 17581 | tcttgtcgcg tgttttagtc gcataaagta gaatacttgc gactagaacc ggagacatta |
| 17641 | cgccatgaac aagagcgccg ccgctggcct gctgggctat gcccgcgtca gcaccgacga |
| 17701 | ccaggacttg accaaccaac gggccgaact gcacgcggcc ggctgcacca agctgttttc |
| 17761 | cgagaagatc accggcacca ggcgcgaccg cccggagctg gccaggatgc ttgaccacct |
| 17821 | acgccctggc gacgttgtga cagtgaccag gctagaccgc ctggcccgca gcacccgcga |
| 17881 | cctactggac attgccgagc gcatccagga ggccggcgcg ggcctgcgta gcctggcaga |
| 17941 | gccgtgggcc gacaccacca cgccggccgg ccgcatggtg ttgaccgtgt tcgccggcat |
| 18001 | tgccgagttc gagcgttccc taatcatcga ccgcacccgg agcgggcgcg aggccgccaa |
| 18061 | ggcccgaggc gtgaagtttg gcccccgccc taccctcacc ccggcacaga tcgcgcacgc |
| 18121 | ccgcgagctg atcgaccagg aaggccgcac cgtgaaagag gcggctgcac tgcttggcgt |
| 18181 | gcatcgctcg accctgtacc gcgcacttga gcgcagcgag gaagtgacgc ccaccgaggc |
| 18241 | caggcggcgc ggtgccttcc gtgaggacgc attgaccgag gccgacgccc tggcggccgc |
| 18301 | cgagaatgaa cgccaagagg aacaagcatg aaaccgcacc aggacggcca ggacgaaccg |
| 18361 | tttttcatta ccgaagagat cgaggcggag atgatcgcgg ccgggtacgt gttcgagccg |
| 18421 | cccgcgcacg tctcaaccgt gcggctgcat gaaatcctgg ccggtttgtc tgatgccaag |
| 18481 | ctggcggcct ggccggccag cttggccgct gaagaaaccg agcgccgccg tctaaaaagg |
| 18541 | tgatgtgtat ttgagtaaaa cagcttgcgt catgcggtcg ctgcgtatat gatgcgatga |
| 18601 | gtaaataaac aaatacgcaa ggggaacgca tgaaggttat cgctgtactt aaccagaaag |
| 18661 | gcgggtcagg caagacgacc atcgcaaccc atctagcccg cgccctgcaa ctcgccgggg |
| 18721 | ccgatgttct gttagtcgat tccgatcccc agggcagtgc ccgcgattgg gcggccgtgc |
| 18781 | gggaagatca accgctaacc gttgtcggca tcgaccgccc gacgattgac cgcgacgtga |
| 18841 | aggccatcgg ccggcgcgac ttcgtagtga tcgacggagc gccccaggcg gcggacttgg |
| 18901 | ctgtgtccgc gatcaaggca gccgacttcg tgctgattcc ggtgcagcca agcccttacg |
| 18961 | acatatgggc caccgccgac ctggtggagc tggttaagca gcgcattgag gtcacggatg |
| 19021 | gaaggctaca agcggccttt gtcgtgtcgc gggcgatcaa aggcacgcgc atcggcggtg |
| 19081 | aggttgccga ggcgctggcc gggtacgagc tgcccattct tgagtcccgt atcacgcagc |
| 19141 | gcgtgagcta cccaggcact gccgccgccg gcacaaccgt tcttgaatca gaacccgagg |
| 19201 | gcgacgctgc ccgcgaggtc caggcgctgg ccgctgaaat taaatcaaaa ctcatttgag |
| 19261 | ttaatgaggt aaagagaaaa tgagcaaaag cacaaacacg ctaagtgccg gccgtccgag |
| 19321 | cgcacgcagc agcaaggctg caacgttggc cagcctggca gacacgccag ccatgaagcg |
| 19381 | ggtcaacttt cagttgccgg cggaggatca caccaagctg aagatgtacg cggtacgcca |
| 19441 | aggcaagacc attaccgagc tgctatctga atacatcgcg cagctaccag agtaaatgag |
| 19501 | caaatgaata aatgagtaga tgaattttag cggctaaagg aggcggcatg gaaaatcaag |
| 19561 | aacaaccagg caccgacgcc gtggaatgcc ccatgtgtgg aggaacgggc ggttggccag |
| 19621 | gcgtaagcgg ctgggttgtc tgccggccct gcaatggcac tggaaccccc aagcccgagg |
| 19681 | aatcggcgtg agcggtcgca aaccatccgg cccggtacaa atcggcgcgg cgctgggtga |
| 19741 | tgacctggtg gagaagttga aggccgcgca ggccgcccag cggcaacgca tcgaggcaga |
| 19801 | agcacgcccc ggtgaatcgt ggcaagcggc cgctgatcga atccgcaaag aatcccggca |
| 19861 | accgccggca gccggtgcgc cgtcgattag gaagccgccc aagggcgacg agcaaccaga |
| 19921 | ttttttcgtt ccgatgctct atgacgtggg cacccgcgat agtcgcagca tcatggacgt |
| 19981 | ggccgttttc cgtctgtcga agcgtgaccg acgagctggc gaggtgatcc gctacgagct |
| 20041 | tccagacggg cacgtagagg tttccgcagg gccggccggc atggccagtg tgtgggatta |
| 20101 | cgacctggta ctgatggcgg tttcccatct aaccgaatcc atgaaccgat accgggaagg |
| 20161 | gaagggagac aagcccggcc gcgtgttccg tccacacgtt gcggacgtac tcaagttctg |
| 20221 | ccggcgagcc gatggcggaa agcagaaaga cgacctggta gaaacctgca ttcggttaaa |
| 20281 | caccacgcac gttgccatgc agcgtacgaa gaaggccaag aacggccgcc tggtgacggt |
| 20341 | atccgagggt gaagccttga ttagccgcta caagatcgta aagagcgaaa ccgggcggcc |
| 20401 | ggagtacatc gagatcgagc tagctgattg gatgtaccgc gagatcacag aaggcaagaa |
| 20461 | cccggacgtg ctgacggttc accccgatta ctttttgatc gatcccggca tcggccgttt |
| 20521 | tctctaccgc ctggcacgcc gcgccgcagg caaggcagaa gccagatggt tgttcaagac |
| 20581 | gatctacgaa cgcagtggca gcgccggaga gttcaagaag ttctgtttca ccgtgcgcaa |
| 20641 | gctgatcggg tcaaatgacc tgccggagta cgatttgaag gaggaggcgg ggcaggctgg |
| 20701 | cccgatccta gtcatgcgct accgcaacct gatcgagggc gaagcatccg ccggttccta |
| 20761 | atgtacggag cagatgctag ggcaaattgc cctagcaggg gaaaaaggtc gaaaaggtct |
| 20821 | ctttcctgtg gatagcacgt acattgggaa cccaaagccg tacattggga accggaaccc |
| 20881 | gtacattggg aacccaaagc cgtacattgg gaaccggtca cacatgtaag tgactgatat |
| 20941 | aaaagagaaa aaaggcgatt tttccgccta aaactcttta aaacttatta aaactcttaa |
| 21001 | aacccgcctg gcctgtgcat aactgtctgg ccagcgcaca gccgaagagc tgcaaaaagc |
| 21061 | gcctaccctt cggtcgctgc gctccctacg ccccgccgct tcgcgtcggc ctatcgcggc |
| 21121 | cgctggccgc tcaaaaatgg ctggcctacg gccaggcaat ctaccagggc gcggacaagc |
| 21181 | cgcgccgtcg ccactcgacc gccggcgccc acatcaaggc accctgcctc gcgcgtttcg |
| 21241 | gtgatgacgg tgaaaacctc tgacacatgc agctcccgga gacggtcaca gcttgtctgt |
| 21301 | aagcggatgc cgggagcaga caagcccgtc agggcgcgtc agcgggtgtt ggcgggtgtc |
| 21361 | ggggcgcagc catgacccag tcacgtagcg atagcggagt gtatactggc ttaactatgc |
| 21421 | ggcatcagag cagattgtac tgagagtgca ccatatgcgg tgtgaaatac cgcacagatg |
| 21481 | cgtaaggaga aaataccgca tcaggcgctc ttccgcttcc tcgctcactg actcgctgcg |
| 21541 | ctcggtcgtt cggctgcggc gagcggtatc agctcactca aaggcggtaa tacggttatc |
| 21601 | cacagaatca ggggataacg caggaaagaa catgtgagca aaaggccagc aaaaggccag |
| 21661 | gaaccgtaaa aaggccgcgt tgctggcgtt tttccatagg ctccgccccc ctgacgagca |
| 21721 | tcacaaaaat cgacgctcaa gtcagaggtg gcgaaacccg acaggactat aaagatacca |
| 21781 | ggcgtttccc cctggaagct ccctcgtgcg ctctcctgtt ccgaccctgc cgcttaccgg |
| 21841 | atacctgtcc gcctttctcc cttcgggaag cgtggcgctt tctcatagct cacgctgtag |
| 21901 | gtatctcagt tcggtgtagg tcgttcgctc caagctgggc tgtgtgcacg aaccccccgt |
| 21961 | tcagcccgac cgctgcgcct tatccggtaa ctatcgtctt gagtccaacc cggtaagaca |
| 22021 | cgacttatcg ccactggcag cagccactgg taacaggatt agcagagcga ggtatgtagg |
| 22081 | cggtgctaca gagttcttga agtggtggcc taactacggc tacactagaa ggacagtatt |
| 22141 | tggtatctgc gctctgctga agccagttac cttcggaaaa agagttggta gctcttgatc |
| 22201 | cggcaaacaa accaccgctg gtagcggtgg tttttttgtt tgcaagcagc agattacgcg |
| 22261 | cagaaaaaaa ggatctcaag aagatccttt gatcttttct acggggtctg acgctcagtg |
| 22321 | gaacgaaaac tcacgttaag ggattttggt catgcatgat atatctccca atttgtgtag |
| 22381 | ggcttattat gcacgcttaa aaataataaa agcagacttg acctgatagt ttggctgtga |
| 22441 | gcaattatgt gcttagtgca tctaacgctt gagttaagcc gcgccgcgaa gcggcgtcgg |
| 22501 | cttgaacgaa tttctagcta gacattattt gccgactacc ttggtgatct cgcctttcac |
| 22561 | gtagtggaca aattcttcca actgatctgc gcgcgaggcc aagcgatctt cttcttgtcc |
| 22621 | aagataagcc tgtctagctt caagtatgac gggctgatac tgggccggca ggcgctccat |
| 22681 | tgcccagtcg gcagcgacat ccttcggcgc gattttgccg gttactgcgc tgtaccaaat |
| 22741 | gcgggacaac gtaagcacta catttcgctc atcgccagcc cagtcgggcg gcgagttcca |
| 22801 | tagcgttaag gtttcattta gcgcctcaaa tagatcctgt tcaggaaccg gatcaaagag |
| 22861 | ttcctccgcc gctggaccta ccaaggcaac gctatgttct cttgcttttg tcagcaagat |
| 22921 | agccagatca atgtcgatcg tggctggctc gaagatacct gcaagaatgt cattgcgctg |
| 22981 | ccattctcca aattgcagtt cgcgcttagc tggataacgc cacggaatga tgtcgtcgtg |
| 23041 | cacaacaatg gtgacttcta cagcgcggag aatctcgctc tctccagggg aagccgaagt |
| 23101 | ttccaaaagg tcgttgatca aagctcgccg cgttgtttca tcaagcctta cggtcaccgt |
| 23161 | aaccagcaaa tcaatatcac tgtgtggctt caggccgcca tccactgcgg agccgtacaa |
| 23221 | atgtacggcc agcaacgtcg gttcgagatg gcgctcgatg acgccaacta cctctgatag |
| 23281 | ttgagtcgat acttcggcga tcaccgcttc ccccatgatg tttaactttg ttttagggcg |
| 23341 | actgccctgc tgcgtaacat cgttgctgct ccataacatc aaacatcgac ccacggcgta |
| 23401 | acgcgcttgc tgcttggatg cccgaggcat agactgtacc ccaaaaaaac agtcataaca |
| 23461 | agccatgaaa accgccactg cgccgttacc accgctgcgt tcggtcaagg ttctggacca |
| 23521 | gttgcgtgag cgcatacgct acttgcatta cagcttacga accgaacagg cttatgtcca |
| 23581 | ctgggttcgt gcccgaattg atcacaggca gcaacgctct gtcatcgtta caatcaacat |
| 23641 | gctaccctcc gcgagatcat ccgtgtttca aacccggcag cttagttgcc gttcttccga |
| 23701 | atagcatcgg taacatgagc aaagtctgcc gccttacaac ggctctcccg ctgacgccgt |
| 23761 | cccggactga tgggctgcct gtatcgagtg gtgattttgt gccgagctgc cggtcgggga |
| 23821 | gctgttggct ggctgg |

What is claimed is:

1. An engineered system for generating a genetically modified cell, the system comprising:
a. a nucleic acid expression construct for expressing a tranposase, wherein the expression construct comprises a promoter operably linked to a nucleic acid sequence encoding the transposase;
b. a nucleic acid construct comprising a donor polynucleotide comprising nucleic acid transposition sequences compatible with the transposase; and
c. a nucleic acid expression construct for expressing a programmable targeting nuclease, wherein the expression construct comprises a promoter operably linked to a nucleic acid sequence encoding the programmable targeting nuclease;
wherein the targeting nuclease is engineered to introduce a cut in a target nucleic acid locus thereby guiding insertion of the donor polynucleotide at the target nucleic acid locus by the transposase to generate a genetically modified cell comprising the donor polynucleotide inserted at the target nucleic acid locus.

2. The engineered system of claim 1, wherein the transposase is linked to the targeting nuclease.

3. The engineered system of claim 1, wherein the transposase is not linked to the targeting nuclease.

4. The engineered system of any one of the preceding claims, wherein the system further comprises a nucleic acid expression construct comprising a promoter operably linked to a polynucleotide sequence encoding a reporter, wherein the donor polynucleotide is inserted in the nucleic acid expression construct, wherein the reporter is inactivated by the inserted nucleic acid construct comprising the donor polynucleotide, and wherein the reporter is activated by excision of the inserted nucleic acid construct comprising the donor polynucleotide from the expression construct comprising a promoter operably linked to a polynucleotide sequence encoding a reporter by the transposase.

5. The engineered system of claim 4, wherein the reporter is GFP, and wherein the nucleic acid expression construct comprises a nucleic acid sequence comprising at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74.

6. The engineered system of any one of the preceding claims, wherein the transposase is a split transposase.

7. The engineered system of claim 6, wherein the transposase is a Pong or Pong-like transposase comprising a Pong ORF1 protein and a Pong ORF2 protein.

8. The engineered system of claim 7, wherein the nucleic acid sequence encoding the Pong transposase comprises:
a. a Pong ORF1 protein, wherein the Pong ORF1 protein comprises an amino acid sequence comprising at least about 75% or more, at least about 85% or more, at least about 95% or more, or 100% sequence identity with the amino acid sequence of SEQ ID NO: 1, and wherein a nucleic acid sequence encoding the Pong ORF1 protein comprises at least about 75% or more, at least about 85% or more, at least about 95% or more, or 100% sequence identity with the nucleic acid sequence of SEQ ID NO: 2, and
b. a Pong ORF2 protein, wherein the Pong ORF2 protein comprises an amino acid sequence comprising at least about 75% or more, at least about 85% or more, at least about 95% or more, or 100% sequence identity with the amino acid sequence of SEQ ID NO: 3, and wherein a nucleic acid sequence encoding the Pong ORF2 protein comprises at least about 75% or more, at least about 85% or more, at least about 95% or more, or 100% sequence identity with the nucleic acid sequence of SEQ ID NO: 4.

9. The engineered system of any one of the preceding claims, wherein the transposition sequences are transposition sequences of a miniature inverted-repeat transposable element (MITE).

10. The engineered system of claim 9, wherein the MITE is an mPing MITE.

11. The engineered system of claim 10, wherein transposition sequences of the mPing MITE comprise mPing inverted repeat 1 and inverted repeat 2, wherein mPing inverted repeat 1 comprises a nucleotide sequence comprising at least about 75% or more, at least about 85% or more, at least about 95% or more, or 100% sequence identity with the nucleic acid sequence of SEQ ID NO: 7, and mPing inverted repeat 2 comprises a nucleotide sequence comprising at least about 75% or more, at least about 85% or more, at least about 95% or more, or 100% sequence identity with the nucleic acid sequence of SEQ ID NO: 8.

12. The engineered system of any one of the preceding claims, wherein the programmable targeting nuclease comprises a programmable, sequence-specific nucleic acid-binding domain and a nuclease domain.

13. The engineered system of any one of the preceding claims, wherein the programmable targeting nuclease is an RNA-guided clustered regularly interspersed short palindromic repeats (CRISPR)/CRISPR-associated (Cas) (CRISPR/Cas) nuclease system, a zinc finger nuclease (ZFN), a transcription activator-like effector nuclease (TALEN), a meganuclease, a ssDNA-guided Argonaute endonuclease, a meganuclease, a rare-cutting endonuclease, or any combination thereof.

14. The engineered system of any one of the preceding claims, wherein the programmable targeting nuclease is a CRISPR/Cas nuclease system comprising a nuclease and a guide RNA (gRNA).

15. The engineered system of claim 14, wherein the programmable targeting nuclease comprises a Cas9 nuclease comprising an amino acid sequence comprising at least about 75% or more, at least about 85% or more, at least about 95% or more, or 100% sequence identity with the amino acid sequence of SEQ ID NO: 5, and wherein the Cas9 nuclease is encoded by a nucleic acid sequence comprising at least about 75% or more, at least about 85% or more, at least about 95% or more, or 100% sequence identity with the nucleic acid sequence of SEQ ID NO: 6.

16. The engineered system of claim 14, wherein the gRNA comprises a nucleic acid sequence of SEQ ID NO: 65, SEQ ID NO: 66, SEQ ID NO: 67, SEQ ID NO: 80, or any combination thereof.

17. The engineered system of any one of the preceding claims, wherein the transposase is a Pong transposase, wherein the nucleic acid transposition sequences are mPing inverted repeat 1 and inverted repeat 2, and the programmable targeting nuclease comprises a Cas9 nuclease and a gRNA.

18. The engineered system of claim 17, wherein the gRNA comprises a nucleic acid sequence of SEQ ID NO: 65, SEQ ID NO: 66, SEQ ID NO: 67, SEQ ID NO: 80, or any combination thereof.

19. The engineered system of claim 17, wherein the nucleic acid construct comprising the donor polynucleotide comprises a nucleic acid sequence comprising at least about 75% or more, at least about 85% or more, at least about 95% or more, or 100% sequence identity with the nucleic acid sequence starting at nucleotide 69 to nucleotide 498 of SEQ ID NO: 92.

20. The engineered system of claim 17, wherein the system further comprises a nucleic acid expression construct comprising a promoter operably linked to a polynucleotide sequence encoding a GFP reporter, wherein the donor polynucleotide is inserted in the nucleic acid expression construct, and wherein the nucleic acid expression construct comprises at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74.

21. The engineered system of claim 17, wherein the nucleic acid construct comprising the donor polynucleotide comprises a nucleotide sequence comprising heat shock element (HSE) sequences flanked by mPing inverted repeat 1 and inverted repeat 2, and wherein the nucleic acid construct comprising the donor polynucleotide comprises at least about 75% or more, at least about 85% or more, at least about 95% or more, or 100% sequence identity with the nucleic acid sequence of SEQ ID NO: 81.

22. The engineered system of claim 17, wherein the Cas9 nuclease is deCas9 nickase, wherein the engineered system comprises a nucleic acid expression construct for expressing the deCas9 nickase comprises a nucleic acid sequence comprising at least about 75% or more, at least about 85% or more, at least about 95% or more, or 100% sequence identity with the nucleic acid sequence starting at nucleotide 8218 to 13856 of SEQ ID NO: 89.

23. The engineered system of claim 17, wherein the engineered system comprises a nucleic acid expression construct for expressing a Cas9 nuclease comprises a nucleic acid sequence comprising at least about 75% or more, at least about 85% or more, at least about 95% or more, or 100% sequence identity with the nucleic acid sequence starting at base 10857 to base 16495 of SEQ ID NO: 94.

24. The engineered system of claim 17, wherein the Cas9 nuclease is not fused to the Pong ORF2 protein, wherein the engineered system comprises a nucleic acid expression construct for expressing a Pong ORF2 protein comprises a nucleic acid sequence comprising at least about 75% or more, at least about 85% or more, at least about 95% or more, or 100% sequence identity with the nucleic acid sequence starting at base 5073 to base 8215 of SEQ ID NO: 89.

25. The engineered system of claim 17, wherein the Cas9 nuclease is fused to the Pong ORF2 protein, wherein the system comprises a nucleic acid nucleic acid expression construct for expressing a Pong ORF1 protein and an expression construct for expressing a Pong ORF2 protein fused to the Cas9 nuclease, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 3359 to base 7268 of SEQ ID NO: 74, and wherein an expression construct for expressing a Pong ORF2 protein fused to the Cas9 nuclease comprises a nucleic acid sequence comprising at least about 75% or more, at least about 85% or more, at least about 95% or more, or 100% sequence identity with the nucleic acid sequence starting at base 7451 to base 14807 of SEQ ID NO: 74.

26. The engineered system of claim 17, wherein the expression construct for expressing a gRNA comprises a nucleic acid sequence comprising at least about 75% or more, at least about 85% or more, at least about 95% or more, or 100% sequence identity with the nucleic acid sequence starting at base 2632 to base 3343 of SEQ ID NO: 74.

27. The engineered system of claim 17, wherein the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 89.

28. The engineered system of claim 17, wherein the expression construct for expressing the gRNA comprises a nucleic acid sequence comprising at least about 75% or more, at least about 85% or more, at least about 95% or more, or 100% sequence identity with the nucleic acid sequence starting at base 729 to base 1440 of SEQ ID NO: 92.

29. The engineered system of claim 17, wherein the system comprises a nucleic acid construct comprising:
a. a nucleic acid nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 5073 to base 8215 of SEQ ID NO: 89;
b. a nucleic acid nucleic acid expression construct for expressing a Pong ORF2 protein fused to Cas9 nuclease, wherein the expression construct for expressing the Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 7451 to base 14807 of SEQ ID NO: 74;
c. a nucleic acid expression construct comprising a promoter operably linked to a polynucleotide sequence encoding GFP, further comprising the donor polynucleotide inserted in the nucleic acid expression construct, wherein the nucleic acid expression construct comprises a nucleic acid sequence comprising at least about 75% or more, at least about 85% or more, at least about 95% or more, or 100% sequence identity with the nucleic acid sequence starting at nucleotide 2414 to nucleotide 23460 and nucleotide 1 to nucleotide 42 of SEQ ID NO: 74; and
d. an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 2632 to base 3343 of SEQ ID NO: 74.

30. The engineered system of claim 17, wherein the system comprises a nucleic acid construct comprising:
a. a nucleic acid nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 1456 to base 5362 of SEQ ID NO: 92;
b. a nucleic acid nucleic acid expression construct for expressing a Pong ORF2 protein fused to Cas9 nuclease, wherein the expression construct for expressing the Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 5548 to base 12904 of SEQ ID NO: 92;
c. a nucleic acid construct comprising the donor polynucleotide, wherein the nucleic acid construct comprises a nucleic acid sequence comprising at least about 75% or more, at least about 85% or more, at least about 95% or more, or 100% sequence identity with the nucleic acid sequence starting at nucleotide 69 to nucleotide 498 of SEQ ID NO: 92; and
d. an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 729 to base 1440 of SEQ ID NO: 92.

31. The engineered system of claim 17, wherein the system comprises a nucleic acid construct comprising:
a. a nucleic acid nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 1481 to base 5390 of SEQ ID NO: 93;
b. a nucleic acid nucleic acid expression construct for expressing a Pong ORF2 protein fused to Cas9 nuclease, wherein the expression construct for expressing the Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 1481 to base 5390 of SEQ ID NO: 93;
c. a nucleic acid construct comprising the donor polynucleotide, wherein the donor polynucleotide comprises a nucleotide sequence comprising HSE sequences flanked by mPing inverted repeat 1 and inverted repeat 2, and wherein the nucleic acid construct comprises at least about 75% or more, at least about 85% or more, at least about 95% or more, or 100% sequence identity with the nucleic acid sequence starting at base 69 to base 512 of SEQ ID NO: 93; and
d. an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 754 to base 1465 of SEQ ID NO: 93.

32. The engineered system of claim 17, wherein the system comprises a nucleic acid construct comprising:
a. a nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 981 to base 4890 of SEQ ID NO: 75;
b. a nucleic acid expression construct for expressing a Pong ORF2 protein fused to Cas9 nuclease, wherein the expression construct for expressing the Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 5073 to base 12429 of SEQ ID NO: 75; and
c. an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 75.

33. The engineered system of claim 17, wherein the system comprises a nucleic acid construct comprising:
a. a nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 981 to base 4890 of SEQ ID NO: 89;
b. a nucleic acid expression construct for expressing a Pong ORF2 protein, wherein the expression construct for expressing the Pong ORF2 protein comprises a nucleic acid sequence comprising at least about 75% or more, at least about 85% or more, at least about 95% or more, or 100% sequence identity with the nucleic acid sequence starting at base 5073 to base 8215 of SEQ ID NO: 89;
c. a nucleic acid expression construct for expressing the deCas9 nickase comprises a nucleic acid sequence comprising at least about 75% or more, at least about 85% or more, at least about 95% or more, or 100% sequence identity with the nucleic acid sequence starting at nucleotide 8218 to nucleotide 13856 of SEQ ID NO: 89; and
d. an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 89.

34. The engineered system of claim 30 or claim 31, wherein the system further comprises a donor nucleic acid construct comprising a promoter operably linked to a polynucleotide sequence encoding a GFP reporter, wherein the donor polynucleotide is inserted in the nucleic acid expression construct, wherein the nucleic acid expression construct comprises at least about 75% or more, at least about 85% or more, at least about 95% or more, or 100% sequence identity with the nucleic acid sequence starting at base 3037 clockwise to base 665 of SEQ ID NO: 90.

35. The engineered system of claim 17, wherein the system comprises:
a. a helper nucleic acid construct comprising:
i. a nucleic acid nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 981 to base 4890 of SEQ ID NO: 91;
ii. a nucleic acid nucleic acid expression construct for expressing a Pong ORF2 protein fused to Cas9 nuclease, wherein the expression construct for expressing the Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 5073 to base 12429 of SEQ ID NO: 91; and
iii. an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 254 to base 965 of SEQ ID NO: 91; and
b. a donor nucleic acid construct comprising a promoter operably linked to a polynucleotide sequence encoding a GFP reporter, wherein the donor polynucleotide is inserted in the nucleic acid expression construct, and wherein the nucleic acid expression construct comprises at least about 75% or more, at least about 85% or more, at least about 95% or more, or 100% sequence identity with the nucleic acid sequence starting at base 3037 clockwise to base 665 of SEQ ID NO: 90.

36. The engineered system of claim 17, wherein the system comprises a nucleic acid construct comprising:
a. a nucleic acid nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 3593 to base 7502 of SEQ ID NO: 94;
b. a nucleic acid nucleic acid expression construct for expressing a Pong ORF2 protein, wherein the expression construct for expressing the Pong ORF2 protein comprises a nucleic acid sequence comprising at least about 75% or more, at least about 85% or more, at least about 95% or more, or 100% sequence identity with the nucleic acid sequence starting at base 7685 to base 10827 of SEQ ID NO: 94;
c. a nucleic acid expression construct for expressing a Cas9 nuclease comprises a nucleic acid sequence comprising at least about 75% or more, at least about 85% or more, at least about 95% or more, or 100% sequence identity with the nucleic acid sequence starting at base 10857 to base 16495 of SEQ ID NO: 94;
d. a nucleic acid construct comprising the donor polynucleotide, wherein the nucleic acid construct comprising the donor polynucleotide comprises a nucleic acid sequence comprising at least about 75% or more, at least about 85% or more, at least about 95% or more, or 100% sequence identity with the nucleic acid sequence starting at base 2201 to base 2630 of SEQ ID NO: 94; and
e. an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 2861 to base 3572 of SEQ ID NO: 94.

37. The engineered system of claim 17, wherein the system comprises a nucleic acid construct comprising:
a. a nucleic acid nucleic acid expression construct for expressing a Pong ORF1 protein, wherein the expression construct for expressing a Pong ORF1 protein comprises a nucleic acid sequence comprising at least about 75% or more, at least about 85% or more, at least about 95% or more, or 100% sequence identity with the nucleic acid sequence starting at base 5490 to base 9399 of SEQ ID NO: 95;
b. a nucleic acid nucleic acid expression construct for expressing a Pong ORF2 protein fused to Cas9 nuclease, wherein the expression construct for expressing the Pong ORF2 protein fused to Cas9 nuclease comprises a nucleic acid sequence comprising at least about 75% or more, at least about 85% or more, at least about 95% or more, or 100% sequence identity with the nucleic acid sequence starting at base 9582 to base 16938 of SEQ ID NO: 95;
c. a nucleic acid expression construct comprising a promoter operably linked to a polynucleotide sequence encoding GFP, further comprising the donor polynucleotide inserted in the nucleic acid expression construct, wherein the nucleic acid expression construct comprises a nucleic acid sequence comprising at least about 75% or more, at least about 85% or more, at least about 95% or more, or 100% sequence identity with the nucleic acid sequence starting at base 4545 to base 2173 of SEQ ID NO: 95; and
d. an expression construct for expressing a gRNA, wherein the expression construct for expressing the gRNA comprises a nucleic acid sequence comprising at least about 75% or more, at least about 85% or more, at least about 95% or more, or 100% sequence identity with the nucleic acid sequence starting at base 4763 to base 5474 of SEQ ID NO: 95.

38. The engineered system of any one of the preceding claims, wherein the target nucleic acid locus is in a nuclear, organellar, or extrachromosomal nucleic acid sequence.

39. The engineered system of any one of the preceding claims, wherein the target nucleic acid locus is in a protein-coding gene, an RNA coding gene, or an intergenic region.

40. The engineered system of any one of the preceding claims, wherein the cell is a eukaryotic cell.

41. The system of any one of the preceding claims, wherein the cell is a plant cell.

42. The system of claim 41, wherein the plant is an Arabidopsis sp. or a soybean plant.

43. One or more nucleic acid constructs encoding an engineered nucleic acid modification system of one of claims 1 to 42.

44. A cell comprising the engineered system of one of claims 1 to 42 or one or more nucleic acid constructs of claim 43.

45. The cell of claim 44, wherein the cell is a eukaryotic cell.

46. The cell of claim 44, wherein the eukaryotic cell is a plant cell.

47. A method of inserting a donor polynucleotide into a target nucleic acid locus in a cell, the method comprising:
a. introducing one or more nucleic acid constructs of claim 43 into the cell;
b. maintaining the cell under conditions and for a time sufficient for the donor polynucleotide to be inserted in the target locus; and
c. optionally identifying an insertion of the donor polynucleotide in the nucleic acid locus in the cell.

48. The method of claim 47, wherein the cell is a eukaryotic cell.

49. The method of claim 47, wherein the eukaryotic cell is a plant cell.

50. The method of claim 47, wherein the cell is ex vivo.

51. A method of altering the expression of a gene of interest, the method comprising using a method of claim 47 to insert an array of six heat-shock enhancer elements flanked by mPing transposition sequences into a promoter of the gene of interest.

52. The method of claim 51, wherein the gene of interest is an Arabidopsis ACT8 gene.

53. A kit for generating a genetically modified cell, the kit comprising one or more engineered systems of claims 1-42 or one or more nucleic acid constructs of claim 43, wherein each of the engineered systems generates an engineered cell comprising an accurate insertion of the donor polynucleotide into the target nucleic acid locus.

54. The kit of claim 53, wherein the kit comprises one or more cells comprising one or more engineered systems, one or more nucleic acid constructs, or combinations thereof.

55. The kit of claim 53, wherein the one or more cells are eukaryotic.

56. The kit of claim 55, wherein the one or more eukaryotic cells comprise plant cells.
